(19) United States
(12) Patent Application Publication
(10) Pub. No.: US 2024/0082351 A1
(43) Pub. Date: Mar. 14, 2024

(54) COMPOSITIONS FOR AND METHODS OF IMPROVING FLUID FLUX IN THE BRAIN

(71) Applicants: Duke University, Durham, NC (US); The University of North Carolina at Chapel Hill, Chapel Hill, NC (US)

(72) Inventors: Aravind Asokan, Durham, NC (US); Aaron Mitchell-Dick, Durham, NC (US); Giridhar MURLIDHARAN, Chapel Hill, NC (US); Ruth Castellanos Rivera, Chapel Hill, NC (US)

(21) Appl. No.: 18/272,208

(22) PCT Filed: Jan. 13, 2022
(86) PCT No.: PCT/US22/12340

§ 371 Date: Jul. 13, 2023

Related U.S. Application Data
(60) Provisional application No. 63/136,803, filed on Jan. 13, 2021.

(51) Int. Cl. A61K 38/17; A61K 48/00; A61P 25/28; C12N 15/86

(52) U.S. Cl. CPC A61K 38/1709; A61K 48/0041; A61K 48/0058; A61K 48/0066; A61P 25/28; C12N 15/86; C12N 2750/14143; C12N 2750/14171; C12N 2830/008

(57) ABSTRACT

The present disclosure provides compositions for and methods of preventing protein aggregation, removing protein aggregates, and improving fluid flux in the brain.

### I. CROSS-REFERENCE TO RELATED APPLICATIONS

This application claim priority to U.S. Provisional Application No. 63/136,803 filed 13 Jan. 2021, which is incorporated herein in their its entirety.

### II. REFERENCE TO THE SEQUENCE LISTING

The Sequence Listing submitted 13 Jan. 2022 as a text file named “21_2037_WO_Sequence_Listing_ST25”, created on 13 Jan.y 2022 and having a size of 278 kilobytes is hereby incorporated by reference pursuant to 37 C.F.R. § 1.52(e)(5).

### III. BACKGROUND

Aggregation and accumulation of proteins in the brain can lead to a wide range of neurodegenerative protein aggregation diseases. Such aggregation diseases include Alzheimer's and Parkinson's diseases, amyotrophic lateral sclerosis, dementia with Lewy bodies, frontotemporal dementia, and Huntington's disease. Alzheimer's disease (AD) is a devasting disease characterized by progressive decline in cognitive function that ends in death within 5-12 years of disease onset.

The exact mechanisms underlying AD pathophysiology are unclear, however, the amyloid-beta (Aβ) plaque accumulation serves as an initiating event. Inhibition of Aβ monomer clearance, leading to Aβ oligomerization and plaque formation, contributes to this phenotype. Aβ plaque accumulation leads to a loss in synaptic and neuronal function and increased neuroinflammation. This triggers the accumulation of neurofibrillary tau tangles (NFT) and activation of astrocyte and microglial immune cells in the central nervous system (CNS). Together, these events lead to widespread neurodegeneration resulting in cognitive impairment and death.

Several mechanisms regulate Aβ clearance, including uptake by astrocytes, microglial phagocytosis, blood-brain barrier (BBB) transport, and glymphatic system flow. Glymphatic flux is regulated by the expression of aquaporin water channels; primarily aquaporin 4 (Aqp4), which is the most abundant aquaporin in the CNS. Therefore, Aqp4 expression plays a major role in the Aβ clearance. Proper glymphatic fluid flux promotes the clearance of Aβ oligomers from the ISF, aiding in the prevention of Aβ plaque formation.

While there are FDA-approved drugs to improve cognitive performance and global functioning in AD patients, these therapies have not been effective halting disease progression. Consequently, there remains an urgent need for a minimally invasive, definitive therapy to address the underlying cause of as well as the sequelae of symptoms associated with protein aggregation diseases in the brain including AD. Currently, the present disclosure provides compositions for and methods of preventing protein aggregation, removing protein aggregates, and improving fluid flux in the brain.

### IV. BRIEF DESCRIPTION OF THE FIGURES

FIG. 1 shows a schematic of the AAV genomic cassette driven by 4 different promoters (CBA, GFAP, Rsg5, and Syn) and used in the Aqp4 expression experiments.

FIG. 2A-FIG. 2D shows that AAV-mediated Aqp4 expression promoted clearance of synthetic Aβ deposits in the mouse brain. FIG. 2A shows that P0 WT mice were injected via ICV with AAV2g9 (7×10vg/animal) packaging an AQP4 transgene driven by the chicken beta-actin (CBA) promoter (right side) or an untreated control (left side). 30 days post-injection, Hilyte-555 labeled Aβ was injected into the mouse striatum. FIG. 2B (no injection)-FIG. 2C (AAV2g9-CBA-Aqp4) show the signal from nuclear staining DAPI (blue) and Aβ (red) when mice were sacrificed 45 min post-injection. FIG. 2D shows percent area of Aβ fluorescence normalized to total brain area (n=3) (*p<0.05).

FIG. 3A-FIG. 3B shows that glymphatic dysregulation exacerbated tau accumulation in a mouse model of Alzheimer's disease (AD) and that Aqp4 KO exacerbates tau accumulation in 3xTg AD mouse model. FIG. 3A shows the genotype confirmation of 3xTg;AQP4KO cross mice. FIG. 3B shows a Western blot of tau accumulation in whole brain of 4 month old 3xTg;AQP4KO mice and 9 month old 3xTg mice.

FIG. 4 shows that AAV-Aqp4 gene therapy promoted AO clearance in a mouse model of AD. AAV9-Aqp4 promoted tau clearance from the hippocampus in AD mice. 3xTg;AQP4KO mice were injected at PO with AAV9-GFAP-Aqp4. Four months post-injection, whole brains (WB) or hippocampus (HC) were harvested and tau levels determined by Western blots (n=2).

FIG. 5 shows that AAV-mediated Aqp4 overexpression decreased Aβ accumulation.

FIG. 6 shows that AAV-mediated Aqp4 overexpression significantly decreased phosphorylated tau accumulation.

### V. BRIEF SUMMARY

Disclosed herein is an isolated nucleic acid molecule, comprising a nucleic acid sequence encoding an aquaporin.

Disclosed herein is a vector comprising a disclosed isolated nucleic acid molecule. Disclosed herein is a vector comprising an isolated nucleic acid molecule encoding an aquaporin. Disclosed herein is vector comprising a disclosed expression cassette comprising a disclosed nucleic acid sequence operably linked to one or more disclosed expression control elements and/or message stabilizing elements.

Disclosed herein is an AAV vector comprising a disclosed isolated nucleic acid molecule encoding a disclosed aquaporin. Disclosed herein is AAV vector comprising a disclosed expression cassette comprising a disclosed nucleic acid sequence operably linked to one or more disclosed expression control elements and/or message stabilizing elements.

Disclosed herein is a recombinant AAV vector comprising a disclosed isolated nucleic acid molecule encoding a disclosed aquaporin. Disclosed herein is a recombinant AAV vector comprising a disclosed expression cassette comprising a disclosed nucleic acid sequence operably linked to one or more disclosed expression control elements and/or message stabilizing elements.

Disclosed herein is a recombinant AAV-cc47 vector comprising a disclosed isolated nucleic acid molecule encoding a disclosed aquaporin. Disclosed herein is a recombinant AAV-cc47 vector comprising a disclosed expression cassette comprising a disclosed nucleic acid sequence operably linked to one or more disclosed expression control elements and/or message stabilizing elements.

Disclosed herein is an AAV-cc47 vector comprising an isolated nucleic acid molecule encoding an aquaporin, wherein the nucleic acid sequence for aquaporin comprises the sequence set forth in any one of SEQ ID NO:20-SEQ ID NO:33 or in any one of SEQ ID NO:35-SEQ ID NO:47.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a GFAP promoter comprising the nucleic acid sequence set forth in SEQ ID NO:02.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a Syn1 promoter comprising the nucleic acid sequence set forth in SEQ ID NO:04.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a Rgs5 promoter comprising the nucleic acid sequence set forth in SEQ ID NO:03.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a CBA promoter comprising the nucleic acid sequence set forth in SEQ ID NO:01.

Disclosed herein is a recombinant AAV2g9 vector comprising a disclosed isolated nucleic acid molecule encoding a disclosed aquaporin. Disclosed herein is a recombinant AAV2g9 vector comprising a disclosed expression cassette comprising a disclosed nucleic acid sequence operably linked to one or more disclosed expression control elements and/or message stabilizing elements.

Disclosed herein is an AAV2g9 vector comprising an isolated nucleic acid molecule encoding an aquaporin, wherein the nucleic acid sequence for aquaporin comprises the sequence set forth in any one of SEQ ID NO:20-SEQ ID NO:33 or in any one of SEQ ID NO:35-SEQ ID NO:47.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a GFAP promoter comprising the nucleic acid sequence set forth in SEQ ID NO:02.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a Syn1 promoter comprising the nucleic acid sequence set forth in SEQ ID NO:04.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a Rgs5 promoter comprising the nucleic acid sequence set forth in SEQ ID NO:03.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a CBA promoter comprising the nucleic acid sequence set forth in SEQ ID NO:01.

Disclosed herein is a pharmaceutical formulation comprising a disclosed vector and/or a disclosed isolated nucleic acid molecule. Disclosed herein is a pharmaceutical formulation comprising an isolated nucleic acid molecule comprising a nucleic acid sequence encoding a polypeptide capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising an isolated nucleic acid molecule comprising a nucleic acid sequence encoding a polypeptide capable of restoring one or more aspects of the glymphatic pathway. Disclosed herein is a pharmaceutical formulation comprising an isolated nucleic acid molecule comprising a nucleic acid sequence encoding a polypeptide capable of restoring one or more aspects of the water influx in the brain of a subject. Disclosed herein is a pharmaceutical formulation comprising a vector capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a vector capable of restoring one or more aspects of the glymphatic pathway. Disclosed herein is a pharmaceutical formulation comprising a vector capable of restoring one or more aspects of the water influx in the brain of a subject. Disclosed herein is a pharmaceutical formulation comprising a vector comprising an isolated nucleic acid molecule comprising a nucleic acid sequence encoding a polypeptide capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a vector comprising an isolated nucleic acid molecule comprising a nucleic acid sequence encoding a polypeptide capable of restoring one or more aspects of the glymphatic pathway, capable of restoring one or more aspects of the water influx in the brain of a subject, or both.

Disclosed herein is a pharmaceutical formulation comprising a rAAV vector capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a rAAV vector capable of restoring one or more aspects of the glymphatic pathway. Disclosed herein is a pharmaceutical formulation comprising a rAAV vector capable of restoring one or more aspects of the water influx in the brain of a subject. Disclosed herein is a pharmaceutical formulation comprising a rAAV vector comprising an isolated nucleic acid molecule comprising a nucleic acid sequence encoding a polypeptide capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a rAAV vector comprising an isolated nucleic acid molecule comprising a nucleic acid sequence encoding a polypeptide capable of restoring one or more aspects of the glymphatic pathway, capable of restoring one or more aspects of the water influx in the brain of a subject, or both.

Disclosed herein is a plasmid comprising one or more disclosed isolated nucleic acids and one or more disclosed promoters. Disclosed herein is a plasmid comprising a disclosed expression cassette comprising a disclosed nucleic acid sequence operably linked to one or more disclosed expression control elements and/or message stabilizing elements.

Disclosed herein is a kit comprising a disclosed isolated nucleic acid molecule, a disclosed vector, a disclosed pharmaceutical formulation, or a combination thereof

Disclosed herein is a method of preventing protein aggregation in the brain of a subject, the method comprising administering to a subject in need thereof a therapeutically effective amount of a vector comprising an isolated nucleic acid sequence encoding an aquaporin.

Disclosed herein is a method of removing and/or clearing protein aggregates in the brain of a subject, the method comprising administering to a subject in need thereof a therapeutically effective amount of a vector comprising an isolated nucleic acid sequence encoding an aquaporin. Disclosed herein is a method of improving fluid flux in the brain of a subject, the method comprising administering to a subject in need thereof a therapeutically effective amount of a vector comprising an isolated nucleic acid sequence encoding an aquaporin. Disclosed herein is a method of treating a subject, the method comprising administering to a subject in need thereof a therapeutically effective amount of a vector comprising an isolated nucleic acid molecule sequence encoding an aquaporin.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a Alzheimer's disease or a neurogenerative disease characterized by protein aggregates, be suspected of having Alzheimer's disease or a neurogenerative disease characterized by protein aggregates, or be at risk of developing Alzheimer's disease or a neurogenerative disease characterized by protein aggregates. In an aspect, a subject can have Alzheimer's disease.

As used herein, the term “diagnosed” means having been subjected to an examination by a person of skill, for example, a physician, and found to have a condition that can be diagnosed or treated by one or more of the disclosed isolated nucleic acid molecules, disclosed vectors, disclosed pharmaceutical formulations, or a combination thereof, or by one or more of the disclosed methods. For example, “diagnosed with Alzheimer's disease or a neurogenerative disease characterized by protein aggregates” means having been subjected to an examination by a person of skill, for example, a physician, and found to have a condition that can be treated by one or more of the disclosed isolated nucleic acid molecules, disclosed vectors, disclosed pharmaceutical formulations, or a combination thereof, or by one or more of the disclosed methods. For example, “suspected of having Alzheimer's disease or a neurogenerative disease characterized by protein aggregates” can mean having been subjected to an examination by a person of skill, for example, a physician, and found to have a condition that can likely be treated by one or more of by one or more of the disclosed isolated nucleic acid molecules, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with Alzheimer's disease or a neurogenerative disease characterized by protein aggregates. In an aspect, a patient can refer to a subject that has been diagnosed with or is suspected of having Alzheimer's disease or a neurogenerative disease characterized by protein aggregates. In an aspect, a patient can refer to a subject that has been diagnosed with or is suspected of having Alzheimer's disease or a neurogenerative disease characterized by protein aggregates and is seeking treatment or receiving treatment for Alzheimer's disease or a neurogenerative disease characterized by protein aggregates.

As used herein, the phrase “identified to be in need of treatment for a disorder,” or the like, refers to selection of a subject based upon need for treatment of the disorder. For example, a subject can be identified as having a need for treatment of a disorder (e.g., such as Alzheimer's disease or a neurogenerative disease characterized by protein aggregates) based upon an earlier diagnosis by a person of skill and thereafter subjected to treatment for the disorder (e.g., such as Alzheimer's disease or a neurogenerative disease characterized by protein aggregates). In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having Alzheimer's disease or a neurogenerative disease characterized by protein aggregates). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a neurodegenerative disease characterized by protein aggregation or a protein-aggregating disease (such as Alzheimer's disease, Parkinson's disease, etc.) can reduce the severity of an established disease in a subject by 1%-100% as compared to a control (such as, for example, an individual not having Alzheimer's disease or a neurogenerative disease characterized by protein aggregates). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a neurodegenerative disease characterized by protein aggregation or a protein-aggregating disease (such as Alzheimer's disease, Parkinson's disease, etc.). For example, treating a neurodegenerative disease characterized by protein aggregation or a protein-aggregating disease (such as Alzheimer's disease, Parkinson's disease, etc.) can reduce one or more symptoms of the disease in a subject by 1%-100% as compared to a control (such as, for example, an individual not having Alzheimer's disease or a neurogenerative disease characterized by protein aggregates). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established Alzheimer's disease or a neurogenerative disease characterized by protein aggregates. It is understood that treatment does not necessarily refer to a cure or complete ablation or eradication of a Alzheimer's disease or a neurogenerative disease characterized by protein aggregates. However, in an aspect, treatment can refer to a cure or complete ablation or eradication of Alzheimer's disease or a neurogenerative disease characterized by protein aggregates.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing Alzheimer's disease or a neurogenerative disease characterized by protein aggregates is intended. The words “prevent” and “preventing” and “prevention” also refer to prophylactic or preventative measures for protecting or precluding a subject (e.g., an individual) not having a given Alzheimer's disease or a neurogenerative disease characterized by protein aggregates or Alzheimer's disease-related complication from progressing to that complication.

As used herein, the terms “administering” and “administration” refer to any method of providing one or more of the disclosed isolated nucleic acid molecules, disclosed vectors, disclosed pharmaceutical formulations, or a combination thereof to a subject. Such methods are well-known to those skilled in the art and include, but are not limited to, the following: oral administration, transdermal administration, administration by inhalation, nasal administration, topical administration, in utero administration, intrahepatic administration, intravaginal administration, intracerebroventricular (ICV) administration, ophthalmic administration, intraaural administration, otic administration, intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration can also include hepatic intra-arterial administration or administration through the hepatic portal vein (HPV). Administration of a disclosed isolated nucleic acid molecule, a disclosed vector, a disclosed pharmaceutical composition, a disclosed therapeutic agent, a disclosed immune modulator, or any other disclosed agent or composition can comprise administration directly into the CNS or the PNS. Administration can be continuous or intermittent. Administration can comprise a combination of one or more route. In an aspect, a disclosed nucleic acid, a disclosed vector, a disclosed pharmaceutical formulation, or any combination thereof can be concurrently and/or serially administered to a subject via multiple routes of administration. For example, in an aspect, administering a disclosed nucleic acid, a disclosed vector, a disclosed pharmaceutical formulation, or any combination thereof can comprise intravenous administration and intra-cistern magna (ICM) administration. In an aspect, administering a disclosed nucleic acid, a disclosed vector, a disclosed pharmaceutical formulation, or any combination thereof can comprise IV administration and intrathecal (ITH) administration. Various combinations of administration are known to the skilled person.

In an aspect, the skilled person can determine an efficacious dose, an efficacious schedule, and an efficacious route of administration for one or more of the disclosed isolated nucleic acid molecules, disclosed vectors, disclosed pharmaceutical formulations, or a combination thereof so as to treat or prevent a neurodegenerative disease characterized by protein aggregation or a protein-aggregating disease (such as Alzheimer's disease, Parkinson's disease, etc.). In an aspect, the skilled person can also alter, change, or modify an aspect of an administering step to improve efficacy of one or more of the disclosed isolated nucleic acid molecules, disclosed vectors, disclosed pharmaceutical formulations, or a combination thereof.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

As used herein, “concurrently” means (1) simultaneously in time, or (2) at different times during the course of a common treatment schedule.

The term “contacting” as used herein refers to bringing one or more of the disclosed isolated nucleic acid molecules, disclosed vectors, disclosed pharmaceutical formulations, or a combination thereof together with a target area or intended target area in such a manner that the one or more of the disclosed isolated nucleic acid molecules, disclosed vectors, disclosed pharmaceutical formulations, or a combination thereof exert an effect on the intended target or targeted area either directly or indirectly. A target area or intended target area can be one or more of a subject's organs (e.g., lungs, heart, liver, muscle, kidney, brain, etc.). In an aspect, a target area or intended target area can be any cell or any organ infected by a neurodegenerative disease characterized by protein aggregation or a protein-aggregating disease (such as Alzheimer's disease, Parkinson's disease, etc.). In an aspect, a target area or intended target area can be the brain. In an aspect, a target area or intended target area can be a brain cell (e.g., neurons, astrocytes, pericytes, microglia, etc.).

As used herein, “determining” can refer to measuring or ascertaining the presence and severity of Alzheimer's disease or a neurogenerative disease characterized by protein aggregates. Methods and techniques used to determine the presence and/or severity of Alzheimer's disease or a neurogenerative disease characterized by protein aggregates are typically known to the medical arts. For example, the art is familiar with the ways to identify and/or diagnose the presence, severity, or both of Alzheimer's disease or a neurogenerative disease characterized by protein aggregates.

As used herein, “effective amount” and “amount effective” can refer to an amount that is sufficient to achieve the desired result such as, for example, the treatment and/or prevention of a Alzheimer's disease or a neurogenerative disease characterized by protein aggregates or a suspected Alzheimer's disease or a neurogenerative disease characterized by protein aggregates. As used herein, the terms “effective amount” and “amount effective” can refer to an amount that is sufficient to achieve the desired an effect on an undesired condition (e.g., Alzheimer's disease or a neurogenerative disease characterized by protein aggregates). For example, a “therapeutically effective amount” refers to an amount that is sufficient to achieve the desired therapeutic result or to have an effect on undesired symptoms but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or (such as Alzheimer's disease or a neurogenerative disease characterized by protein aggregates), (ii) attenuates, ameliorates, or eliminates one or more symptoms of the particular disease, condition, or disorder (e.g., Alzheimer's disease or a neurogenerative disease characterized by protein aggregates), or (iii) delays the onset of one or more symptoms of the particular disease, condition, or disorder described herein (e.g., Alzheimer's disease or a neurogenerative disease characterized by protein aggregates). The specific therapeutically effective dose level for any particular patient will depend upon a variety of factors including the disorder being treated and the severity of the disorder; the disclosed isolated nucleic acid molecules, disclosed vectors, disclosed pharmaceutical formulations employed; the disclosed methods employed; the age, body weight, general health, sex and diet of the patient; the time of administration; the route of administration; the rate of excretion of the disclosed isolated nucleic acid molecules, disclosed vectors, or disclosed pharmaceutical formulations employed; the duration of the treatment; drugs used in combination or coincidental with the disclosed isolated nucleic acid molecules, disclosed vectors, or disclosed pharmaceutical formulations employed, and other like factors well-known in the medical arts. For example, it is well within the skill of the art to start doses of the disclosed isolated nucleic acid molecules, disclosed vectors, or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, Alzheimer's disease or a neurogenerative disease characterized by protein aggregates.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, the term “biologically active agent” or “biologic active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or a RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight.

As known to the art, miRNAs are small non-coding RNAs that are about 17 to about 25 nucleotide bases (nt) in length in their biologically active form. In an aspect, a disclosed miRNA can regulate gene expression post transcriptionally by decreasing target mRNA translation. In an aspect, a disclosed miRNA can function as a negative regulator. In an aspect, a disclosed miRNA is about 17 to about 25, about 17 to about 24, about 17 to about 23, about 17 to about 22, about 17 to about 21, about 17 to about 20, about 17 to about 19, about 18 to about 25, about 18 to about 24, about 18 to about 23, about 18 to about 22, about 18 to about 21, about 18 to about 20, about 19 to about 25, about 19 to about 24, about 19 to about 23, about 19 to about 22, about 19 to about 21, about 20 to about 25, about 20 to about 24, about 20 to about 23, about 20 to about 22, about 21 to about 25, about 21 to about 24, about 21 to about 23, about 22 to about 25, about 22 to about 24, or about 22 nucleotides in length. Generally, there are three forms of miRNAs: primary miRNAs (pri-miRNAs), premature miRNAs (pre-miRNAs), and mature miRNAs, all of which are within the scope of the present disclosure.

As used herein, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene), and a 3′ untranslated region (e.g., in eukaryotes a polyadenylation site).

As used herein, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

In an aspect, a disclosed ubiquitous promoter can be a CMV enhancer/chicken β-actin promoter (CB promoter).

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

In an aspect, a disclosed promoter can be a promoter/enhancer. As used herein, the term promoter/enhancer can refer to a segment of DNA that contains nucleotide sequences capable of providing both promoter and enhancer functions.

As discussed above, a disclosed promoter can be an endogenous promoter. Endogenous refers to a disclosed promoter or disclosed promoter/enhancer that is naturally linked with its gene. In an aspect, a disclosed endogenous promoter can generally be obtained from a non-coding region upstream of a transcription initiation site of a gene (such as, for example, a disclosed Aquaporin or some other enzyme involved in the glymphatic pathway). In an aspect, a disclosed endogenous promoter can be used for constitutive and efficient expression of a disclosed transgene (e.g., a nucleic acid sequence encoding a polypeptide capable of preventing accumulation, clearing and/or removing protein accumulation, and/or improving fluid flux).

As discussed above, a disclosed promoter can be an exogenous promoter. Exogenous (or heterologous) refers to a disclosed promoter or a disclosed promoter/enhancer that can be placed in juxtaposition to a gene by means of molecular biology techniques such that the transcription of that gene can be directed by the linked promoter or linked promoter/enhancer. In an aspect, a disclosed endogenous promoter can be an endogenous promoter/enhancer.

As used herein, an “enhancer” such as a transcription or transcriptional enhancer refers to regulatory DNA segment that is typically found in multicellular eukaryotes. An enhancer can strongly stimulate (“enhance”) the transcription of a linked transcription unit, i.e., it acts in cis. An enhancer can activate transcription over very long distances of many thousand base pairs, and from a position upstream or downstream of the site of transcription initiation. An enhancer can have a modular structure by being composed of multiple binding sites for transcriptional activator proteins. Many enhancers control gene expression in a cell type-specific fashion. Several remote enhancers can control the expression of a singular gene while a singular enhance can stimulate the transcription of one or more genes.

As used herein, the term “serotype” is a distinction used to refer to an AAV having a capsid that is serologically distinct from other AAV serotypes. Serologic distinctiveness can be determined based on the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of any aquaporin. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “CRISPR or clustered regularly interspaced short palindromic repeat” is an ideal tool for correction of genetic abnormalities as the system can be designed to target genomic DNA directly. A CRISPR system involves two main components—a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with an AAV vector. Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, any disclosed aquaporin including Aqp4) or a CpG-depleted and codon optimized ORF for any disclosed aquaporin including Aqp4.

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

In an aspect, a “self-complementary parvovirus genome” can be a single stranded polynucleotide having, in the 5′ to 3′ direction, a first parvovirus ITR sequence, a heterologous sequence (e.g., payload construct comprising, for example, any disclosed aquaporin including Aqp4), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g. as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, a disclosed immune modulator can be Tacrolimus. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., a therapeutic agent or biologic agent) to a subject having or diagnosed with a neurodegenerative disease characterized by protein aggregation or a protein-aggregating disease (such as Alzheimer's disease, Parkinson's disease, etc.).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Alzheimer's Disease

Generally, dementia refers to a decline in cognitive ability that is severe enough to interfere with daily basic activities. Alzheimer's disease (AD) is the most common type of dementia, accounting for at least two-thirds of cases of dementia in people age 65 and older. Alzheimer's disease is a neurodegenerative disease with insidious onset and progressive impairment of behavioral and cognitive functions including memory, comprehension, language, attention, reasoning, and judgment. Currently, AD is the sixth leading cause of death in the United States. Onset before 65 years of age (early onset) is unusual and seen in less than 10% of Alzheimer's disease patients.

Clinical symptoms depend on the stage of the disease. The initial and most common presenting symptom is episodic short-term memory loss with relative sparing of long-term memory and can be elicited in most patients even when not the presenting symptom. Short-term memory impairment is followed by impairment in problem-solving, judgment, executive functioning, lack of motivation and disorganization, leading to problems with multitasking and abstract thinking. In the early stages of AD, the impairment in executive functioning ranges from subtle to significant. Language disorder and impairment of visuospatial skills ensues. Neuropsychiatric symptoms like apathy, social withdrawal, disinhibition, agitation, psychosis, and wandering are also common in the mid- to late-stage AD. At the late stages of AD, difficulty performing learned motor tasks (dyspraxia), olfactory dysfunction, sleep disturbances, extrapyramidal motor signs like dystonia, akathisia, and parkinsonian symptoms typically occur. Finally, late stage AD is characterized by primitive reflexes, incontinence, and total dependence on caregivers.

Pathologically, AD is mainly characterized by amyloid-β (Aβ) and tau protein deposition. Playing a pivotal role in AD, the imbalance between Aβ production and clearance results in toxic accumulation. This protein is produced from amyloid precursor protein (APP), a transmembrane protein that undergoes post-translational processing. In physiological conditions, APP is cleaved sequentially by α- and γ-secretases, resulting in rapidly degraded peptides; however, absence of α-secretase cleavage leads to APP internalization into endocytic compartments, where it is alternatively cleaved by β-secretase 1 (BACE1). The resulting product is subsequently cleaved by γ-secretase, resulting in the more aggregating-prone isoforms Aβ40 and Aβ42.

Tau is an intracellular protein that regulates the assembly and stability of neuronal microtubules via its phosphorylation. In AD, tau is hyperphosphorylated, accumulating in the form of intracellular neurofibrillary tangles. This compromises its microtubule-binding ability and promotes neurodegeneration, and leads to accumulation of microtubule-transported APP, further contributing to neurodegeneration.

### C. Glymphatic Pathway

Tissue homeostasis relies on the clearance of excess fluid and interstitial solutes. In the peripheral tissues, the lymphatic system is responsible for returning back to the general circulation soluble material, proteins, and fluid from the interstitial space. (Liao S, et al. (2013) Lymphat Res Biol. 11:1361-1143). While the lymphatic network extends throughout all parts of the peripheral tissues, the density of lymph vessels correlates with the rate of tissue metabolism. Although the brain and spinal cord are characterized by a disproportionally high metabolic rate (Wang Z, et al. (2012) Obesity. 20:95-100), and synaptic transmission is exquisitely sensitive to changes in their environment, the central nervous system (CNS) completely lacks conventional lymphatic vessels.

Even though this appears to remain true for the brain parenchyma, functional lymphatic vessels in the brain meninges have been described (Louveau A, et al. (2015) Nature. 523:337-341; Aspelund A, et al. J Exp Med. 212:991-999). These vessels express all the molecular markers of the lymphatic endothelial cells of the conventional lymphatic vessels and play an important role in CSF drainage as they can successfully clear macromolecules and immune cells from the subarachnoid space and into the cervical lymph nodes. Since these vessels do not reach the parenchyma, complementary mechanisms are needed.

The brain has other clearance systems, one of which is interstitial solute transport across the blood-brain barrier (BBB), which is then drained into the blood stream. However, this route can be hindered by the large distance between interstitial solutes and the BBB; additionally, the tightly sealed endothelium of brain capillaries (which constitutes the BBB) precludes normal systemic interstitial and lymphatic flow into the brain. To bypass this situation, other clearance routes are favored, such as CSF-ISF bulk flow, known as the glymphatic system. The breakdown of the CSF-ISF exchange has been associated with various neurodegenerative diseases, such as cerebrovascular disease, Lewy body disease, and notably Alzheimer's disease (AD). Moreover, recent assessment of glymphatic function in old versus young mice showed a dramatic reduction by ˜80-90% in aged compared to young mice. (Kress B T, et al. (2014) Ann Neurol. 76(6):845-861). The suppression of glymphatic activity included both influx of CSF tracers and clearance of radiolabeled β-amyloid and inulin.

The CSF and interstitial fluid (ISF) continuously interchange. This exchange is facilitated by convective influx of CSF along the periarterial space (Iliff J J, et al. (2012) Sci Transl Med. 4(147):147ra111). From the subarachnoid space, CSF is driven into the Virchow-Robin spaces by a combination of arterial pulsatility, respiration, and CSF pressure gradients and the loose fibrous matrix of the perivascular space can be viewed as a low resistance highway for CSF influx. The subsequent transport of CSF into the dense and complex brain parenchyma is facilitated by AQP4 water channels expressed in a highly polarized manner in astrocytic end feet that ensheathe the brain vasculature (Iliff J J et al., 2012; Iliff J J, et al. (2013) Stroke. 44:S93-S95). CSF movement into the parenchyma drives convective interstitial fluid fluxes within the tissue toward the perivenous spaces surrounding the large deep veins. The interstitial fluid is collected in the perivenous space from where it drains out of brain toward the cervical lymphatic system. (Johnston M, et al. (2004) Cerebrospinal Fluid Res. 1(1):2; Murtha L A, et al. (201) Fluids Barriers CNS. 11:12).

### D. Compositions

### 1. Nucleic Acid Molecules

Disclosed herein is an isolated nucleic acid molecule, comprising a nucleic acid sequence encoding an aquaporin.

In an aspect, a disclosed aquaporin can prevent protein aggregation in the brain of a subject, can remove and/or clear protein aggregates in the brain of a subject, can improve fluid flux in the brain of a subject, or any combination thereof.

In an aspect, a disclosed aquaporin can restore one or more aspects of the glymphatic pathway. In an aspect, a disclosed aquaporin can restore one or more aspects of the water influx in the brain of a subject.

In an aspect, a disclosed isolated nucleic acid molecule encoding the aquaporin can be CpG-depleted and/or codon-optimized for expression in a human cell. In an aspect, “CpG-depleted” can mean “CpG-free”. In an aspect, “CpG-free” can mean “CpG-depleted”. In an aspect, “CpG-free” can mean completely free of CpGs or partially free of CpGs. In an aspect, “CpG-free” can mean completely depleted of CpGs or partially depleted of CpGs. In an aspect, “CpG-free” can mean “CpG-free” for a desired and/or ideal expression level. CpG depletion and/or optimization is known to the skilled person in the art.

In an aspect, a disclosed isolated nucleic acid molecule can comprise the nucleotide sequence for AQP1, AQP2, AQP3, AQP4, AQP5, AQP6, AQP7, AQP8, AQP9, AQP10, AQP11, AQP12, APQ13, or any combination thereof. In an aspect, a disclosed isolated nucleic acid molecule can comprise the nucleotide sequence for AQP1, AQP4, AQP5, APQ6, or APQ8. In an aspect, a disclosed isolated nucleic acid molecule can comprise the nucleotide sequence for AQP4.

In an aspect, a disclosed isolated nucleic acid molecule can comprise the nucleotide sequence for recombinant AQP1, AQP2, AQP3, AQP4, AQP5, AQP6, AQP7, AQP8, AQP9, AQP10, AQP11, AQP12, APQ13, or any combination thereof. In an aspect, a disclosed isolated nucleic acid molecule can comprise the nucleotide sequence for recombinant AQP1, AQP4, AQP5, APQ6, or APQ8. In an aspect, a disclosed isolated nucleic acid molecule can comprise the nucleotide sequence for recombinant AQP4.

In an aspect, a disclosed encoded aquaporin can comprise Aqp1, Aqp2, Aqp3, Aqp4, Aqp5, Aqp6, Apq7, Apq8, Apq9, Apq10, Apq11, Apq12, Apq13, or any combination thereof. In an aspect, a disclosed encoded aquaporin can comprise Aqp1, Aqp4, Aqp5, Apq6, or Apq8. In an aspect, a disclosed encoded aquaporin can comprise Aqp4. In an aspect, a disclosed encoded aquaporin can comprise recombinant Aqp1, Aqp2, Aqp3, Aqp4, Aqp5, Aqp6, Apq7, Apq8, Apq9, Apq10, Apq11, Apq12, Apq13, or any combination thereof. In an aspect, a disclosed encoded aquaporin can comprise recombinant Aqp1, Aqp4, Aqp5, Apq6, or Apq8. In an aspect, a disclosed encoded aquaporin can comprise recombinant Aqp4.

In an aspect, a disclosed nucleic acid sequence for Aqp1 can comprise the sequence set forth in SEQ ID NO:20 or a sequence having at least 50%, at least 60%, at least 70%, at least 80%, at least 90%, at least 95%, or at least 99% identity to the sequence set forth in SEQ ID NO:20. In an aspect, a disclosed nucleic acid sequence for Aqp2 can comprise the sequence set forth in SEQ ID NO:21 or a sequence having at least 50%, at least 60%, at least 70%, at least 80%, at least 90%, at least 95%, or at least 99% identity to the sequence set forth in SEQ ID NO:21. In an aspect, a disclosed nucleic acid sequence for Aqp3 can comprise the sequence set forth in SEQ ID NO:22 or a sequence having at least 50%, at least 60%, at least 70%, at least 80%, at least 90%, at least 95%, or at least 99% identity to the sequence set forth in SEQ ID NO:22. In an aspect, a disclosed nucleic acid sequence for Aqp4 can comprise the sequence set forth in SEQ ID NO:23 or a sequence having at least 50%, at least 60%, at least 70%, at least 80%, at least 90%, at least 95%, or at least 99% identity to the sequence set forth in SEQ ID NO:23. In an aspect, a disclosed nucleic acid sequence for Aqp5 can comprise the sequence set forth in SEQ ID NO:24 or a sequence having at least 50%, at least 60%, at least 70%, at least 80%, at least 90%, at least 95%, or at least 99% identity to the sequence set forth in SEQ ID NO:24. In an aspect, a disclosed nucleic acid sequence for Aqp6 can comprise comprises the sequence set forth in SEQ ID NO:25 or a sequence having at least 50%, at least 60%, at least 70%, at least 80%, at least 90%, at least 95%, or at least 99% identity to the sequence set forth in SEQ ID NO:25. In an aspect, a disclosed nucleic acid sequence for Aqp7 can comprise comprises the sequence set forth in SEQ ID NO:26 or a sequence having at least 50%, at least 60%, at least 70%, at least 80%, at least 90%, at least 95%, or at least 99% identity to the sequence set forth in SEQ ID NO:26. In an aspect, a disclosed nucleic acid sequence for Aqp8 can comprise the sequence set forth in SEQ ID NO:27 or a sequence having at least 50%, at least 60%, at least 70%, at least 80%, at least 90%, at least 95%, or at least 99% identity to the sequence set forth in SEQ ID NO:27. In an aspect, a disclosed nucleic acid sequence for Aqp9 can comprise comprises the sequence set forth in SEQ ID NO:28 or a sequence having at least 80% identity to the sequence set forth in SEQ ID NO:28. In an aspect, a disclosed nucleic acid sequence for Aqp10 can comprise comprises the sequence set forth in SEQ ID NO:29 or a sequence having at least 50%, at least 60%, at least 70%, at least 80%, at least 90%, at least 95%, or at least 99% identity to the sequence set forth in SEQ ID NO:29. In an aspect, a disclosed nucleic acid sequence for Aqp11 can comprise comprises the sequence set forth in SEQ ID NO:30 or a sequence having at least 50%, at least 60%, at least 70%, at least 80%, at least 90%, at least 95%, or at least 99% identity to the sequence set forth in SEQ ID NO:30. In an aspect, a disclosed nucleic acid sequence for Aqp11 can comprise comprises the sequence set forth in SEQ ID NO:31 or a sequence having at least 50%, at least 60%, at least 70%, at least 80%, at least 90%, at least 95%, or at least 99% identity to the sequence set forth in SEQ ID NO:31. In an aspect, a disclosed nucleic acid sequence for Aqp12 can comprise comprises the sequence set forth in SEQ ID NO:32 or a sequence having at least 50%, at least 60%, at least 70%, at least 80%, at least 90%, at least 95%, or at least 99% identity to the sequence set forth in SEQ ID NO:32. In an aspect, a disclosed nucleic acid sequence for Aqp12 can comprise comprises the sequence set forth in SEQ ID NO:33 or a sequence having at least 50%, at least 60%, at least 70%, at least 80%, at least 90%, at least 95%, or at least 99% identity to the sequence set forth in SEQ ID NO:33.

In an aspect, a disclosed encoded Aqp1 can comprise the sequence set forth in SEQ ID NO:07 or a sequence having at least 50%, at least 60%, at least 70%, at least 80%, at least 90%, at least 95%, or at least 99% identity to the sequence set forth in SEQ ID NO:07. In an aspect, a disclosed encoded Aqp2 can comprise the sequence set forth in SEQ ID NO:08 or a sequence having at least 50%, at least 60%, at least 70%, at least 80%, at least 90%, at least 95%, or at least 99% identity to the sequence set forth in SEQ ID NO:08. In an aspect, a disclosed encoded Aqp3 can comprise the sequence set forth in SEQ ID NO:09 or a sequence having at least 50%, at least 60%, at least 70%, at least 80%, at least 90%, at least 95%, or at least 99% identity to the sequence set forth in SEQ ID NO:09. In an aspect, a disclosed encoded Aqp4 can comprise the sequence set forth in SEQ ID NO:10 or a sequence having at least 50%, at least 60%, at least 70%, at least 80%, at least 90%, at least 95%, or at least 99% identity to the sequence set forth in SEQ ID NO:10. In an aspect, a disclosed encoded Aqp5 can comprise the sequence set forth in SEQ ID NO:11 or a sequence having at least 50%, at least 60%, at least 70%, at least 80%, at least 90%, at least 95%, or at least 99% identity to the sequence set forth in SEQ ID NO:11. In an aspect, a disclosed encoded Aqp6 can comprise the sequence set forth in SEQ ID NO:12 or a sequence having at least 50%, at least 60%, at least 70%, at least 80%, at least 90%, at least 95%, or at least 99% identity to the sequence set forth in SEQ ID NO:12. In an aspect, a disclosed encoded Aqp7 can comprise the sequence set forth in SEQ ID NO:13 or a sequence having at least 50%, at least 60%, at least 70%, at least 80%, at least 90%, at least 95%, or at least 99% identity to the sequence set forth in SEQ ID NO:13. In an aspect, a disclosed encoded Aqp8 can comprise the sequence set forth in SEQ ID NO:14 or a sequence having at least 50%, at least 60%, at least 70%, at least 80%, at least 90%, at least 95%, or at least 99% identity to the sequence set forth in SEQ ID NO:14. In an aspect, a disclosed encoded Aqp9 can comprise the sequence set forth in SEQ ID NO:15 or a sequence having at least 50%, at least 60%, at least 70%, at least 80%, at least 90%, at least 95%, or at least 99% identity to the sequence set forth in SEQ ID NO:15. In an aspect, a disclosed encoded Aqp10 can comprise the sequence set forth in SEQ ID NO:16 or a sequence having at least 50%, at least 60%, at least 70%, at least 80%, at least 90%, at least 95%, or at least 99% identity to the sequence set forth in SEQ ID NO:16. In an aspect, a disclosed encoded Aqp11 can comprise the sequence set forth in SEQ ID NO:17 or a sequence having at least 50%, at least 60%, at least 70%, at least 80%, at least 90%, at least 95%, or at least 99% identity to the sequence set forth in SEQ ID NO:17. In an aspect, a disclosed encoded Aqp12 can comprise the sequence set forth in SEQ ID NO:18 or a sequence having at least 50%, at least 60%, at least 70%, at least 80%, at least 90%, at least 95%, or at least 99% identity to the sequence set forth in SEQ ID NO:18. In an aspect, a disclosed encoded Aqp12 can comprise the sequence set forth in SEQ ID NO:19 or a sequence having at least 50%, at least 60%, at least 70%, at least 80%, at least 90%, at least 95%, or at least 99% identity to the sequence set forth in SEQ ID NO:19.

In an aspect, a disclosed encoded Aqp can comprise a recombinant Aqp.

In an aspect, a disclosed nucleic acid sequence can have a coding sequence that is less than about 4.5 kilobases.

In an aspect, a disclosed isolated nucleic acid molecule can comprise one or more expression control elements operably linked to the isolated nucleic acid sequence encoding the aquaporin. In an aspect, the disclosed expression control elements can comprise a promoter, an enhancer, a promoter/enhancer, a transcription pausing signal, a termination signal, or a combination thereof. Expression control elements are known to the art. In an aspect, a disclosed nucleic acid sequence can comprise one or more message stabilizing elements. In an aspect, the disclosed stabilizing elements can comprise a 3′ UTR noncoding region, a polyadenylation (polyA) sequence, inverted terminal repeats (ITRs), or any combination thereof. Message stabilizing elements are known to the art. For example, in an aspect, a polyA sequence can comprise the SV40 polyA sequence. In an aspect, a disclosed SV40 polyA sequence can comprise the sequence set forth in SEQ ID NO:34 or a sequence having at least 50%, at least 60%, at least 70%,at least 80%, at least 90%, at least 95%, or at least 99%identity to the sequence set forth in SEQ ID NO:34. In an aspect, the disclosed ITRs can comprise AAV2 ITRs.

In an aspect, a disclosed promoter can comprise a constitutive promoter, a ubiquitous promoter, or a tissue-specific promoter.

In an aspect, a disclosed constitutive promoter can be a chicken beta actin (CBA) promoter. In an aspect, a disclosed CBA promoter can comprise the sequence set forth in SEQ ID NO:01 or a sequence having at least 50%, at least 60%, at least 70%, at least 80%, at least 90%, at least 95%, or at least 99% identity to the sequence set forth in SEQ ID NO:01.

In an aspect, a disclosed tissue-specific promoter can comprise a brain cell specific promoter. In an aspect, a disclosed brain cell specific promoter can comprise a synapsin 1 (Syn1) promoter, a calmodulin/calcium dependent kinase II (CAMKII) promoter, a glial fibrillary acidic protein (GFAP) promoter, a Rgs5 promoter, a S100 beta promoter, a neuron-specific enolase (NSE) promoter, a Thy1 promoter, or any combination thereof.

In an aspect, a disclosed GFAP promoter can comprise the sequence set forth in SEQ ID NO:02 or a sequence having at least 50%, at least 60%, at least 70%, at least 80%, at least 90%, at least 95%, or at least 99% identity to the sequence set forth in SEQ ID NO:02. In an aspect, a disclosed Syn1 promoter can comprise the sequence set forth in SEQ ID NO:04 or a sequence having at least 50%, at least 60%, at least 70%, at least 80%, at least 90%, at least 95%, or at least 99% identity to the sequence set forth in SEQ ID NO:04. In an aspect, a disclosed Rgs5 promoter can comprise the sequence set forth in SEQ ID NO:03 or a sequence having at least 50%, at least 60%, at least 70%, at least 80%, at least 90%, at least 95%, or at least 99% identity to the sequence set forth in SEQ ID NO:03.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed aquaporin or some other enzyme involved in glymphatic transport or metabolism). In an aspect, a disclosed endogenous promoter or a disclosed endogenous promoter/enhancer can be used for constitutive and efficient expression of a disclosed gene (e.g., a nucleic acid sequence encoding an aquaporin).

In an aspect, a disclosed endogenous promoter or a disclosed endogenous promoter/enhancer can be used for constitutive and efficient expression of an aquaporin. In an aspect, a disclosed endogenous promoter or a disclosed endogenous promoter/enhancer can comprise the promoter for the gene encoding Aqp1, Aqp2, Aqp3, Aqp4, Aqp5, Aqp6, Aqp7, Aqp8, Aqp9, Aqp10, Aqp11, Aqp12, or Aqp 13. For example, in an aspect, when an encoded polypeptide comprises the Aqp1, the disclosed endogenous promoter or a disclosed endogenous promoter/enhancer can comprise the promoter or promoter/enhancer for the gene encoding Aqp1. Similarly, when an encoded polypeptide comprises Aqp4, the disclosed endogenous promoter or a disclosed endogenous promoter/enhancer can comprise the promoter or promoter/enhancer for the gene encoding Aqp4. For example, in an aspect, a disclosed endogenous promoter or a disclosed endogenous promoter/enhancer can comprise the sequence set forth in SEQ ID NO:06 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the endogenous promoter sequence for any one of Aqp1, Aqp2, Aqp3, Aqp4, Aqp5, Aqp6, Aqp7, Aqp8, Aqp9, Aqp10, Aqp11, Aqp12, or Aqp 13.

In an aspect, a disclosed nucleic acid molecule can be packaged into a non-viral or a viral vector. In an aspect, a disclosed non-viral vector can comprise a polymer-based vector, a peptide-based vector, a lipid nanoparticle, a solid lipid nanoparticle, or a cationic lipid-based vector. In an aspect, a disclosed viral vector can comprise an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can comprise bovine AAV, caprine AAV, canine AAV, equine AAV, ovine AAV, avian AAV, primate AAV, or non-primate AAV.

In an aspect, a disclosed AAV vector can comprise a recombinant AAV (rAAV) vector. In an aspect, a disclosed AAV vector can be self-complementary.

In an aspect, a disclosed AAV vector or disclosed recombinant rAAV vector can comprise AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed AAV vector can comprise AAV2g9. In an aspect, a disclosed AAV2g9 can comprise the chimera described in Shen S, et al. (2013) J Biol Chem. 288(40):28814-28823. In an aspect, a disclosed AAV vector can comprise AAV-cc47.

In an aspect, a disclosed AAV vector can comprise a promoter operably linked to the nucleic acid sequence encoding a disclosed aquaporin.

In an aspect, a disclosed isolated nucleic acid sequence encoding an aquaporin can be in an expression cassette. In an aspect, a disclosed expression cassette can comprise a disclosed nucleic acid sequence encoding a disclosed aquaporin operably linked to one or more disclosed expression control elements and/or message stabilizing elements. As known to the art, a functional expression unit that is capable of properly driving the expression of an incorporated polynucleotide (i.e., a nucleic acid sequence encoding an aquaporin) can be referred to as an “expression cassette”. The skilled person knows how to design an expression cassette to allow the expression in a eukaryotic cell, such as preferably in a mammalian or human cell.

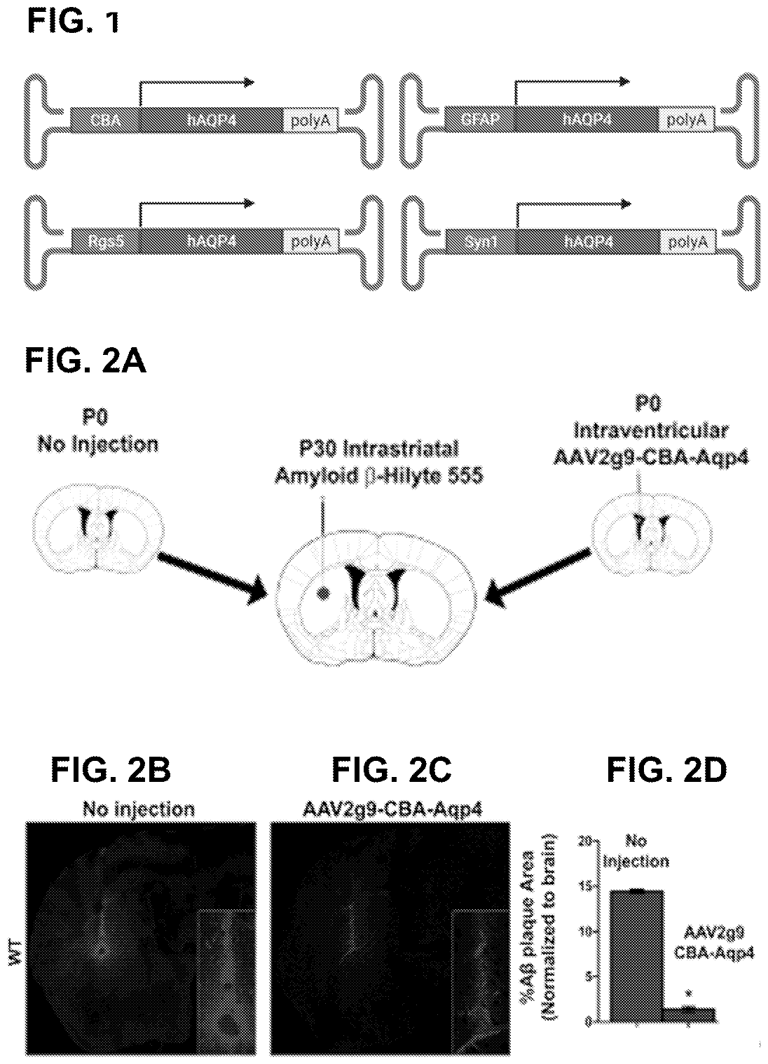

In an aspect, a disclosed expression cassette can be one shown in FIG. 1.

### a. Nucleotide Sequences

In an aspect, a disclosed AQP1 can comprise the following sequence or a fragment thereof:

(SEQ ID NO: 20)
ATGGCCAGCGAGTTCAAGAAGAAGCTCTTCTGGAGGGCAGTGGTGGCC
GAGTTCCTGGCCACGACCCTCTTTGTCTTCATCAGCATCGGTTCTGCC
CTGGGCTTCAAATACCCGGTGGGGAACAACCAGACGGCGGTCCAGGAC
AACGTGAAGGTGTCGCTGGCCTTCGGGCTGAGCATCGCCACGCTGGCG
CAGAGTGTGGGCCACATCAGCGGCGCCCACCTCAACCCGGCTGTCACA
CTGGGGCTGCTGCTCAGCTGCCAGATCAGCATCTTCCGTGCCCTCATG
TACATCATCGCCCAGTGCGTGGGGGCCATCGTCGCCACCGCCATCCTC
TCAGGCATCACCTCCTCCCTGACTGGGAACTCGCTTGGCCGCAATGAC
CTGGCTGATGGTGTGAACTCGGGCCAGGGCCTGGGCATCGAGATCATC
GGGACCCTCCAGCTGGTGCTATGCGTGCTGGCTACTACCGACCGGAGG
CGCCGTGACCTTGGTGGCTCAGCCCCCCTTGCCATCGGCCTCTCTGTA
GCCCTTGGACACCTCCTGGCTATTGACTACACTGGCTGTGGGATTAAC
CCTGCTCGGTCCTTTGGCTCCGCGGTGATCACACACAACTTCAGCAAC
CACTGGATTTTCTGGGTGGGGCCATTCATCGGGGGAGCCCTGGCTGTA
CTCATCTACGACTTCATCCTGGCCCCACGCAGCAGTGACCTCACAGAC
CGCGTGAAGGTGTGGACCAGCGGCCAGGTGGAGGAGTATGACCTGGAT
GCCGACGACATCAACTCCAGGGTGGAGATGAAGCCCAAATAG.

In an aspect, a disclosed AQP2 can comprise the following sequence or a fragment thereof:

(SEQ ID NO: 21)
ATGTGGGAGCTCCGCTCCATAGCCTTCTCCAGGGCTGTGTTCGCAGAG
TTCCTGGCCACACTCCTCTTCGTCTTCTTTGGCCTCGGCTCTGCCCTC
AACTGGCCACAGGCCCTGCCCTCTGTGCTACAGATTGCCATGGCGTTT
GGCTTGGGTATTGGCACCCTGGTACAGGCTCTGGGCCACATAAGCGGG
GCCCACATCAACCCTGCCGTGACTGTGGCCTGCCTGGTGGGCTGCCAC
GTCTCCGTTCTCCGAGCCGCCTTCTACGTGGCTGCCCAGCTGCTGGGG
GCTGTGGCCGGAGCCGCTCTGCTCCATGAGATCACGCCAGCAGACATC
CGCGGGGACCTGGCTGTCAATGCTCTCAGCAACAGCACGACGGCTGGC
CAGGCGGTGACTGTGGAGCTCTTCCTGACACTGCAGCTGGTGCTCTGC
ATCTTCGCCTCCACCGATGAGCGCCGCGGAGAGAACCCGGGCACCCCT
GCTCTCTCCATAGGCTTCTCTGTGGCCCTGGGCCACCTCCTTGGGATC
CATTACACCGGCTGCTCTATGAATCCTGCCCGCTCCCTGGCTCCAGCT
GTCGTCACTGGCAAATTTGATGACCACTGGGTCTTCTGGATCGGACCC
CTGGTGGGCGCCATCCTGGGCTCCCTCCTCTACAACTACGTGCTGTTT
CCGCCAGCCAAGAGCCTGTCGGAGCGCCTGGCAGTGCTGAAGGGCCTG
GAGCCGGACACCGATTGGGAGGAGCGCGAGGTGCGACGGCGGCAGTCG
GTGGAGCTGCACTCGCCGCAGAGCCTGCCACGGGGTACCAAGGCCTGA.

In an aspect, a disclosed AQP3 can comprise the following sequence or a fragment thereof:

(SEQ ID NO: 22)
ATGGGTCGACAGAAGGAGCTGGTGTCCCGCTGCGGGGAGATGCTCCAC
ATCCGCTACCGGCTGCTCCGACAGGCGCTGGCCGAGTGCCTGGGGACC
CTCATCCTGGTGATGTTTGGCTGTGGCTCCGTGGCCCAGGTTGTGCTC
AGCCGGGGCACCCACGGTGGTTTCCTCACCATCAACCTGGCCTTTGGC
TTTGCTGTCACTCTGGGCATCCTCATCGCTGGCCAGGTCTCTGGGGCC
CACCTGAACCCTGCCGTGACCTTTGCCATGTGCTTCCTGGCTCGTGAG
CCCTGGATCAAGCTGCCCATCTACACCCTGGCACAGACGCTGGGAGCC
TTCTTGGGTGCTGGAATAGTTTTTGGGCTGTATTATGATGCAATCTGG
CACTTCGCCGACAACCAGCTTTTTGTTTCGGGCCCCAATGGCACAGCC
GGCATCTTTGCTACCTACCCCTCTGGACACTTGGATATGATCAATGGC
TTCTTTGACCAGTTCATAGGCACAGCCTCCCTTATCGTGTGTGTGCTG
GCCATTGTTGACCCCTACAACAACCCCGTCCCCCGAGGCCTGGAGGCC
TTCACCGTGGGCCTGGTGGTCCTGGTCATTGGCACCTCCATGGGCTTC
AACTCCGGCTATGCCGTCAACCCTGCCCGGGACTTTGGCCCCCGCCTT
TTTACAGCCCTTGCGGGCTGGGGCTCTGCAGTCTTCACGACCGGCCAG
CATTGGTGGTGGGTGCCCATCGTGTCCCCACTCCTGGGCTCCATTGCG
GGTGTCTTCGTGTACCAGCTGATGATCGGCTGCCACCTGGAGCAGCCC
CCACCCTCCAACGAGGAAGAGAATGTGAAGCTGGCCCATGTGAAGCAC
AAGGAGCAGATCTGA.

In an aspect, a disclosed AQP4 can comprise the following sequence or a fragment thereof:

(SEQ ID NO: 23)
ATGGTGGCTTTCAAAGGGGTCTGGACTCAAGCTTTCTGGAAAGCAGTC
ACAGCGGAATTTCTGGCCATGCTTATTTTTGTTCTCCTCAGCCTGGGA
TCCACCATCAACTGGGGTGGAACAGAAAAGCCTTTACCGGTCGACATG
GTTCTCATCTCCCTTTGCTTTGGACTCAGCATTGCAACCATGGTGCAG
TGCTTTGGCCATATCAGCGGTGGCCACATCAACCCTGCAGTGACTGTG
GCCATGGTGTGCACCAGGAAGATCAGCATCGCCAAGTCTGTCTTCTAC
ATCGCAGCCCAGTGCCTGGGGGCCATCATTGGAGCAGGAATCCTCTAT
CTGGTCACACCTCCCAGTGTGGTGGGAGGCCTGGGAGTCACCATGGTT
CATGGAAATCTTACCGCTGGTCATGGTCTCCTGGTTGAGTTGATAATC
ACATTTCAATTGGTGTTTACTATCTTTGCCAGCTGTGATTCCAAACGG
ACTGATGTCACTGGCTCAATAGCTTTAGCAATTGGATTTTCTGTTGCA
ATTGGACATTTATTTGCAATCAATTATACTGGTGCCAGCATGAATCCC
GCCCGATCCTTTGGACCTGCAGTTATCATGGGAAATTGGGAAAACCAT
TGGATATATTGGGTTGGGCCCATCATAGGAGCTGTCCTCGCTGGTGGC
CTTTATGAGTATGTCTTCTGTCCAGATGTTGAATTCAAACGTCGTTTT
AAAGAAGCCTTCAGCAAAGCTGCCCAGCAAACAAAAGGAAGCTACATG
GAGGTGGAGGACAACAGGAGTCAGGTAGAGACGGATGACCTGATTCTA
AAACCTGGAGTGGTGCATGTGATTGACGTTGACCGGGGAGAGGAGAAG
AAGGGGAAAGACCAATCTGGAGAGGTATTGTCTTCAGTATGA.

In an aspect, a disclosed AQP5 can comprise the following sequence or a fragment thereof:

(SEQ ID NO: 24)
ATGAAGAAGGAGGTGTGCTCCGTGGCCTTCCTCAAGGCCGTGTTCGCAG
AGTTCTTGGCCACCCTCATCTTCGTCTTCTTTGGCCTGGGCTCGGCCCT
CAAGTGGCCGTCGGCGCTGCCTACCATCCTGCAGATCGCGCTGGCGTTT
GGCCTGGCCATAGGCACGCTGGCCCAGGCCCTGGGACCCGTGAGCGGCG
GCCACATCAACCCCGCCATCACCCTGGCCCTCTTGGTGGGCAACCAGAT
CTCGCTGCTCCGGGCTTTCTTCTACGTGGCGGCCCAGCTGGTGGGCGCC
ATTGCCGGGGCTGGCATCCTCTACGGTGTGGCACCGCTCAATGCCCGGG
GCAATCTGGCCGTCAACGCGCTCAACAACAACACAACGCAGGGCCAGGC
CATGGTGGTGGAGCTGATTCTGACCTTCCAGCTGGCACTCTGCATCTTC
GCCTCCACTGACTCCCGCCGCACCAGCCCTGTGGGCTCCCCAGCCCTGT
CCATTGGCCTGTCTGTCACCCTGGGCCACCTTGTCGGAATCTACTTCAC
TGGCTGCTCCATGAACCCAGCCCGCTCTTTTGGCCCTGCGGTGGTCATG
AATCGGTTCAGCCCCGCTCACTGGGTTTTCTGGGTAGGGCCCATCGTGG
GGGCGGTCCTGGCTGCCATCCTTTACTTCTACCTGCTCTTCCCCAACTC
CCTGAGCCTGAGTGAGCGTGTGGCCATCATCAAAGGCACGTATGAGCCT
GACGAGGACTGGGAGGAGCAGCGGGAAGAGCGGAAGAAGACCATGGAGC
TGACCACCCGCTGA.

In an aspect, a disclosed AQP6 can comprise the following sequence or a fragment thereof:

(SEQ ID NO: 25)
ATGGATGCAGTGGAGCCAGGGGGACGTGGCTGGGCCAGCATGTTGGCGT
GCAGGCTTTGGAAAGCCATCAGCAGGGCGCTGTTTGCAGAGTTCCTGGC
CACGGGGCTGTATGTGTTCTTTGGCGTGGGCTCAGTCATGCGCTGGCCC
ACAGCACTTCCCTCCGTGCTACAGATTGCCATCACCTTCAACCTGGTCA
CCGCCATGGCTGTGCAGGTCACCTGGAAGGCCAGCGGGGCCCACGCCAA
CCCCGCCGTGACGCTGGCCTTCCTCGTAGGCTCCCACATCTCTCTGCCC
CGTGCTGTGGCCTATGTGGCTGCCCAGCTGGTGGGGGCCACGGTGGGGG
CTGCTCTGCTTTATGGGGTCATGCCGGGAGACATCCGAGAGACCCTTGG
GATCAACGTGGTCCGGAACAGTGTCTCAACTGGCCAGGCGGTGGCAGTG
GAGCTGCTTCTGACCCTGCAGCTGGTGCTCTGTGTCTTCGCTTCCACCG
ACAGCCGTCAGACATCAGGCTCCCCGGCCACCATGATTGGGATCTCTGT
GGCACTGGGCCACCTCATTGGGATCCACTTCACTGGCTGCTCCATGAAT
CCAGCCCGCTCCTTCGGCCCTGCCATCATCATTGGGAAGTTCACAGTCC
ACTGGGTCTTCTGGGTGGGGCCCCTGATGGGAGCCCTCCTGGCCTCACT
GATCTACAACTTCGTCCTGTTCCCCGACACCAAGACCCTGGCGCAGCGG
CTGGCTATCCTCACAGGCACCGTAGAGGTGGGGACAGGGGCAGGGGCAG
GGGCGGAGCCCCTGAAGAAGGAATCCCAGCCGGGTTCGGGAGCCGTGGA
GATGGAGAGTGTGTGA.

In an aspect, a disclosed AQP7 can comprise the following sequence or a fragment thereof:

(SEQ ID NO: 26)
ATGGTTCAAGCATCCGGGCACAGGCGGTCCACCCGTGGCTCCAAAATGG
TCTCCTGGTCCGTGATAGCAAAGATCCAGGAAATACTGCAGAGGAAGAT
GGTGCGAGAGTTCCTGGCCGAGTTCATGAGCACATATGTCATGATGGTA
TTCGGCCTTGGTTCCGTGGCCCATATGGTTCTAAATAAAAAATATGGGA
GCTACCTTGGTGTCAACTTGGGTTTTGGCTTCGGAGTCACCATGGGAGT
GCACGTGGCAGGCCGCATCTCTGGAGCCCACATGAACGCAGCTGTGACC
TTTGCTAACTGTGCGCTGGGCCGCGTGCCCTGGAGGAAGTTTCCGGTCT
ATGTGCTGGGGCAGTTCCTGGGCTCCTTCCTGGCGGCTGCCACCATCTA
CAGTCTCTTCTACACGGCCATTCTCCACTTTTCGGGTGGACAGCTGATG
GTGACCGGTCCCGTCGCTACAGCTGGCATTTTTGCCACCTACCTTCCTG
ATCACATGACATTGTGGCGGGGCTTCCTGAATGAGGCGTGGCTGACCGG
GATGCTCCAGCTGTGTCTCTTCGCCATCACGGACCAGGAGAACAACCCA
GCACTGCCAGGAACAGAGGCGCTGGTGATAGGCATCCTCGTGGTCATCA
TCGGGGTGTCCCTTGGCATGAACACAGGATATGCCATCAACCCGTCCCG
GGACCTGCCCCCCCGCATCTTCACCTTCATTGCTGGTTGGGGCAAACAG
GTCTTCAGCAATGGGGAGAACTGGTGGTGGGTGCCAGTGGTGGCACCAC
TTCTGGGTGCCTATCTAGGTGGCATCATCTACCTGGTCTTCATTGGCTC
CACCATCCCACGGGAGCCCCTGAAATTGGAGGATTCTGTGGCGTATGAA
GACCACGGGATAACCGTATTGCCCAAGATGGGATCTCATGAACCCACGA
TCTCTCCCCTCACCCCCGTCTCTGTGAGCCCTGCCAACAGATCTTCAGT
CCACCCTGCCCCACCCTTACATGAATCCATGGCCCTAGAGCACTTCTAA.

In an aspect, a disclosed AQP8 can comprise the following sequence or a fragment thereof:

(SEQ ID NO: 27)
ATGTCTGGAGAGATAGCCATTGTGAGCCTGAATTTGGCAATGACAAGGC
CAGGGAGCCGAGCGTGGGTGGCAGGTGGCGAGTGTCCTGGTACGAACGG
TTTGTGCAGCCATGTCTGGTCGAACTGCTGGGCTCTGCTCTCTTCATCT
TCATCGGGTGCCTGTCGGTCATTGAGAATGGGACGGACACTGGGCTGCT
GCAGCCGGCCCTGGCCCACGGGCTGGCTTTGGGGCTCGTGATTGCCACG
CTGGGGAATATCAGTGGTGGACACTTCAACCCTGCGGTGTCCCTGGCAG
CCATGCTGATCGGAGGCCTCAACCTGGTGATGCTCCTCCCGTACTGGGT
CTCACAGCTGCTCGGGGGGATGCTCGGGGCTGCCTTGGCCAAGGCGGTG
AGTCCTGAGGAGAGGTTCTGGAATGCATCTGGGGCGGCCTTTGTGACAG
TCCAGGAGCAGGGGCAGGTGGCAGGGGCGTTGGTGGCAGAGATCATCCT
GACGACGCTGCTGGCCCTGGCTGTATGCATGGGTGCCATCAATGAGAAG
ACAAAGGGCCCTCTGGCCCCGTTCTCCATCGGCTTTGCCGTCACCGTGG
ATATCCTGGCTGGGGGCCCTGTGTCTGGAGGCTGCATGAATCCCGCCCG
TGCTTTTGGACCTGCGGTGGTGGCCAACCACTGGAACTTCCACTGGATC
TACTGGCTGGGCCCACTCCTGGCTGGCCTGCTTGTTGGACTGCTCATTA
GGTGCTTCATTGGAGATGGGAAGACCCGCCTCATCCTGAAGGCTCGGTG
A.

In an aspect, a disclosed AQP9 can comprise the following sequence or a fragment thereof:

(SEQ ID NO: 28)
ATGCAGCCTGAGGGAGCAGAAAAGGGAAAAAGCTTCAAGCAGAGACTGG
TCTTGAAGAGCAGCTTAGCGAAAGAAACCCTCTCTGAGTTCTTGGGCAC
GTTCATCTTGATTGTCCTTGGATGTGGCTGTGTTGCCCAAGCTATTCTC
AGTCGAGGACGTTTTGGAGGGGTCATCACTATCAATGTTGGATTTTCAA
TGGCAGTTGCAATGGCCATTTATGTGGCTGGCGGTGTCTCTGGTGGTCA
CATCAACCCAGCTGTGTCTTTAGCAATGTGTCTCTTTGGACGGATGAAA
TGGTTCAAATTGCCATTTTATGTGGGAGCCCAGTTCTTGGGAGCCTTTG
TGGGGGCTGCAACCGTCTTTGGCATTTACTATGATGGACTTATGTCCTT
TGCTGGTGGAAAACTGCTGATCGTGGGAGAAAATGCAACAGCACACATT
TTTGCAACATACCCAGCTCCGTATCTATCTCTGGCGAACGCATTTGCAG
ATCAAGTGGTGGCCACCATGATACTCCTCATAATCGTCTTTGCCATCTT
TGACTCCAGAAACTTGGGAGCCCCCAGAGGCCTAGAGCCCATTGCCATC
GGCCTCCTGATTATTGTCATTGCTTCCTCCCTGGGACTGAACAGTGGCT
GTGCCATGAACCCAGCTCGAGACCTGAGTCCCAGACTTTTCACTGCCTT
GGCAGGCTGGGGGTTTGAAGTCTTCAGAGCTGGAAACAACTTCTGGTGG
ATTCCTGTAGTGGGCCCTTTGGTTGGTGCTGTCATTGGAGGCCTCATCT
ATGTTCTTGTCATTGAAATCCACCATCCAGAGCCTGACTCAGTCTTTAA
GACAGAACAATCTGAGGACAAACCAGAGAAATATGAACTCAGTGTCATC
ATGTAG.

In an aspect, a disclosed AQP10 can comprise the following sequence or a fragment thereof:

(SEQ ID NO: 29)
ATGGTCTTCACTCAGGCCCCGGCTGAAATCATGGGCCACCTCCGGATAC
GCAGCCTCCTGGCCCGGCAGTGCCTGGCAGAGTTTCTGGGTGTGTTTGT
ACTCATGCTCCTCACCCAAGGAGCTGTGGCCCAGGCTGTCACCAGTGGA
GAAACCAAAGGCAACTTCTTCACCATGTTTCTGGCTGGCTCTCTGGCCG
TTACGATAGCCATCTACGTGGGTGGTAACGTCTCAGGGGCCCACCTGAA
TCCAGCCTTCTCCCTGGCCATGTGCATCGTTGGACGCCTCCCCTGGGTC
AAGCTCCCCATTTACATCTTGGTGCAGTTGCTGTCTGCTTTCTGTGCTT
CGGGAGCCACCTATGTTCTCTACCATGATGCCCTACAGAACTATACAGG
TGGGAACCTGACAGTGACTGGCCCCAAGGAGACAGCCTCCATTTTTGCC
ACCTATCCTGCCCCCTATCTGTCCCTGAACAATGGCTTCCTGGATCAGG
TTCTGGGCACTGGGATGCTGATTGTGGGGCTCTTGGCCATCCTGGACAG
ACGGAACAAGGGAGTCCCTGCGGGTCTGGAGCCTGTGGTGGTGGGGATG
CTGATCCTGGCCCTCGGGTTATCCATGGGTGCCAACTGCGGGATTCCAC
TCAACCCTGCCCGGGACCTGGGCCCACGTCTCTTCACCTACGTGGCTGG
CTGGGGTCCTGAAGTCTTCAGTGCTGGTAATGGCTGGTGGTGGGTGCCT
GTGGTGGCCCCTCTGGTGGGGGCCACCGTTGGCACAGCCACTTACCAGC
TGTTGGTGGCTCTGCACCACCCTGAGGGCCCAGAGCCAGCTCAGGATCT
GGTGTCTGCTCAACACAAAGCCTCAGAGTTGGAAACTCCTGCCTCAGCT
CAGATGCTGGAGTGTAAGCTATGA.

In an aspect, a disclosed AQP11 can comprise the following sequence or a fragment thereof:

(SEQ ID NO: 30)
AGAGCCGGAGCCCGCAACCCGCTCAGGCGGCGACGGAGCCATGTCGCCG
CTGCTGGGGCTCCGGTCCGAGCTGCAGGACACCTGCACCTCGCTGGGAC
TGATGCTGTCGGTGGTGCTGCTCATGGGGCTGGCCCGCGTAGTCGCCCG
GCAGCAGCTGCACAGGCCGGTGGCCCACGCCTTCGTCCTGGAGTTTCTA
GCCACCTTCCAGCTCTGCTGCTGCACCCACGAGCTGCAACTGCTGAGCG
AACAGCACCCCGCGCACCCCACCTGGACGCTGACGCTCGTCTACTTCTT
CTCGCTTGTGCATGGCCTGACTCTGGTGGGCACGTCCAGCAACCCGTGC
GGCGTGATGATGCAGATGATGCTGGGGGGCATGTCCCCCGAGACGGGTG
CGGTGAGGCTATTGGCTCAGCTGGTTAGTGCCCTGTGCAGCAGGTACTG
CACAAGCGCCTTGTGGAGCTTGGGTCTGACCCAGTATCACGTCAGCGAG
AGGAGCTTCGCTTGCAAGAATCCCATCCGAGTCGACTTGCTCAAAGCGG
TCATCACAGAGGCCGTCTGCTCCTTTCTCTTCCACAGCGCTCTGCTGCA
CTTCCAGGAAGTCCGAACCAAGCTTCGTATCCACCTGCTGGCTGCACTC
ATCACCTTTTTGGTCTATGCAGGAGGAAGTCTAACAGGAGCTGTATTTA
ATCCAGCTTTGGCACTTTCGCTACATTTCATGTGTTTTGATGAAGCATT
CCCTCAGTTTTTTATAGTATACTGGCTGGCTCCTTCTTTAGGTATATTG
TTGATGATTTTGATGTTCAGCTTTTTCCTTCCATGGCTGCATAACAACC
ATACAATTAATAAAAAGGAATAACTGTTCCAAAGACTCAGACTAACATA
CAGGACAGTCCAGCTGGATGTGATAAAGATTTTATCACCTCATATGGAA
AACACCGGCTGCACTGGATTCATCAGTGTTAACTTCCTTTGAGGAAGCT
GCCTTATAGTTTTCATCACTGGGACTTTAAAAAAAAATTACTGTGAAAA
TGAGGTATTCTGTACTTCTCAGTTAAGACTTGTTCTTTGAGTGATGTAT
TAAATGCTGCTAGAAAAGCCTCATTACATTAAATATAAATCAATCTTAA
ATGATAATTGTTAACTTTGATGAAAAACGAGTACAGGATGAGAAGGGAA
GTAAAGGTGATAGTAAGATCAACGAATTTGTGTATCAAGTGTCACCCAA
ATGAACAGAATTTTTAACTATAAAAGTACAAGGCGTCAACTTACAGAAC
TGGGGAGAGGATACTTTCAGCCACCACCTCACAAAATGATACCATCAAG
CAGTGTCATCTTTCAACAGGAAAGTTGCTTTTAGAAGTAGAATATATAT
TCACTCACATGCAAGCGATTTCCTACATTTTAAGTACATTAAAATCTAA
TACCTAAAATAGTTTCTCTAAATAGACAATCTCACTAGCTCCAGCCAAG
TGGTTATTCTTTAATAGAATATAATAAAACAAAACACCCATACAAAGTC
AGGATTTAAGCTTTTCACATTCTCCTTTGAACCAACAGCTAGCTACCTT
ATGAAGTAGGGAGGGCATGTATTTTTAATCTCCATTTTACAGATAAGGA
AACTAAGCCTAGATGGGTTTGCTGCCCTGGCTTAGATCACATAGAGCCA
AATACCATCTCTAGTCTTCAACTTCAATCCAAAAATTCTTCTCGGTCAC
GATTATGACTAAAGGGAAATCAAGGGTTCATAGAAACATTAAATACTTT
ATAATAACGTTAAAACTTGTTTAGAAATTAACTAGTATTTTTTGAGGTT
TAAAGAACCTTCCAGTTTTACT.

In an aspect, a disclosed AQP11 can comprise the following sequence or a fragment thereof:

(SEQ ID NO: 31)
AGAGCCGGAGCCCGCAACCCGCTCAGGCGGCGACGGAGCCATGTCGCCG
CTGCTGGGGCTCCGGTCCGAGCTGCAGGACACCTGCACCTCGCTGGGAC
TGATGCTGTCGGTGGTGCTGCTCATGGGGCTGGCCCGCGTAGTCGCCCG
GCAGCAGCTGCACAGGCCGGTGGCCCACGCCTTCGTCCTGGAGTTTCTA
GCCACCTTCCAGCTCTGCTGCTGCACCCACGAGCTGCAACTGCTGAGCG
AACAGCACCCCGCGCACCCCACCTGGACGCTGACGCTCGTCTACTTCTT
CTCGCTTGTGCATGGCCTGACTCTGGTGGGCACGTCCAGCAACCCGTGC
GGCGTGATGATGCAGATGATGCTGGGGGGCATGTCCCCCGAGACGGGTG
CGGTGAGGCTATTGGCTCAGCTGGTTAGTGCCCTGTGCAGCAGGTACTG
CACAAGCGCCTTGTGGAGCTTGGGTCTGACCCAGTATCACGTCAGCGAG
AGGAGCTTCGCTTGCAAGAATCCCATCCGAGTCGACTTGCTCAAAGCGG
TCATCACAGAGGCCGTCTGCTCCTTTCTCTTCCACAGCGCTCTGCTGCA
CTTCCAGGAAGTCCGAACCAAGCTTCGTATCCACCTGCTGGCTGCACTC
ATCACCTTTTTGGTCTATGCAGGTATATTGTTGATGATTTTGATGTTCA
GCTTTTTCCTTCCATGGCTGCATAACAACCATACAATTAATAAAAAGGA
ATAACTGTTCCAAAGACTCAGACTAACATACAGGACAGTCCAGCTGGAT
GTGATAAAGATTTTATCACCTCATATGGAAAACACCGGCTGCACTGGAT
TCATCAGTGTTAACTTCCTTTGAGGAAGCTGCCTTATAGTTTTCATCAC
TGGGACTTTAAAAAAAAATTACTGTGAAAATGAGGTATTCTGTACTTCT
CAGTTAAGACTTGTTCTTTGAGTGATGTATTAAATGCTGCTAGAAAAGC
CTCATTACATTAAATATAAATCAATCTTAAATGATAATTGTTAACTTTG
ATGAAAAACGAGTACAGGATGAGAAGGGAAGTAAAGGTGATAGTAAGAT
CAACGAATTTGTGTATCAAGTGTCACCCAAATGAACAGAATTTTTAACT
ATAAAAGTACAAGGCGTCAACTTACAGAACTGGGGAGAGGATACTTTCA
GCCACCACCTCACAAAATGATACCATCAAGCAGTGTCATCTTTCAACAG
GAAAGTTGCTTTTAGAAGTAGAATATATATTCACTCACATGCAAGCGAT
TTCCTACATTTTAAGTACATTAAAATCTAATACCTAAAATAGTTTCTCT
AAATAGACAATCTCACTAGCTCCAGCCAAGTGGTTATTCTTTAATAGAA
TATAATAAAACAAAACACCCATACAAAGTCAGGATTTAAGCTTTTCACA
TTCTCCTTTGAACCAACAGCTAGCTACCTTATGAAGTAGGGAGGGCATG
TATTTTTAATCTCCATTTTACAGATAAGGAAACTAAGCCTAGATGGGTT
TGCTGCCCTGGCTTAGATCACATAGAGCCAAATACCATCTCTAGTCTTC
AACTTCAATCCAAAAATTCTTCTCGGTCACGATTATGACTAAAGGGAAA
TCAAGGGTTCATAGAAACATTAAATACTTTATAATAACGTTAAAACTTG
TTTAGAAATTAACTAGTATTTTTTGAGGTTTAAAGAACCTTCCAGTTTT
ACT.

In an aspect, a disclosed AQP12 can comprise the following sequence or a fragment thereof:

(SEQ ID NO: 32)
GTCCCCTCAGGTGTCCTGCAGGCACAGCTCCTCGGGGGGCCCAGGCCGA
TGGCAGGTCTTAACGTGTCCCTCTCCTTCTTCTTTGCCACCTTCGCCCT
CTGTGAGGCGGCCAGGCGGGCCTCCAAGGCCCTGCTCCCAGTGGGCGCC
TATGAAGTCTTCGCCCGGGAGGCGATGAGGACGCTGGTCGAGCTCGGGC
CCTGGGCTGGGGACTTTGGGCCTGACCTGCTGCTCACCCTGCTCTTCCT
GCTCTTCCTGGCGCACGGGGTCACCTTGGACGGGGCCTCGGCCAACCCC
ACTGTGTCCCTGCAGGAGTTCCTCATGGCCGAGCAGTCTCTGCCTGGCA
CGCTGTTGAAGCTGGCGGCACAGGGGCTGGGCATGCAGGCCGCCTGCAC
CCTGATGCGCCTCTGCTGGGCCTGGGAGCTCAGTGACCTGCACCTGCTG
CAGAGCCTCATGGCCCAGAGCTGCAGCTCGGCCCTGCGCACATCCGTGC
CCCACGGGGCGCTTGTGGAGGCCGCCTGCGCCTTTTGTTTCCATCTGAC
CCTCCTGCACCTGCGGCACAGTCCTCCCGCCTACAGCGGGCCCGCTGTG
GCTCTGTTGGTCACCGTCACGGCCTACACGGCCGGGCCCTTCACGTCTG
CCTTCTTCAACCCTGCCCTGGCCGCCTCTGTGACCTTTGCCTGCTCGGG
ACACACCTTACTGGAGTACGTGCAGGTGTACTGGCTGGGCCCTCTGACA
GGGATGGTCCTGGCTGTGCTGCTGCACCAGGGCCGCCTTCCCCACCTTT
TCCAGAGGAACCTGTTCTACGGCCAGAAGAACAAGTACCGAGCACCCCG
AGGGAAGCCGGCCCCGGCCTCAGGGGACACCCAGACCCCTGCAAAGGGG
TCCAGTGTCCGGGAGCCTGGGCGCAGTGGTGTTGAGGGGCCACATTCCA
GCTGAGTGGCCTTGCTCTGTGTGAGCCCCGTGCGAGGGCCCTGCTTGTA
GCTGGACCCTGGAACCTTCTGTAGCTAAGAGGGAATCCTGGCCCCCTCC
CCAGAAGCCATTTGTCAATAAACCATTTCTAAGA.

In an aspect, a disclosed AQP12 can comprise the following sequence or a fragment thereof:

(SEQ ID NO: 33)
GTCCCCTCAGGTGTCCTGCAGGCACAGCATCTCGGGGGGCCCAGGCCGA
TGGCAGGTCTTAACGTGTCCCTCTCCTTCTTCTTTGCCACCTTCACCCT
CTGTGAGGCAGCCAGGCGGGCCTCCAAGGCCCTGCTCCCAGTGGGCGCC
TATGAAGTCTTCGCCCGGGAGGCGGTGGGCGCGGTGCAGCTCGGGGCCT
GCTTCCTGGAGATGAGGACGCTGGTCGAGCTCGGGCCCTGGGCTGGGGA
CTTTGGGCCTGACCTGCTGCTCACCCTGCTCTTCCTGCTCTTCCTGGCG
CACGGGGTCACCTTGGACGGGGCCTCGGCCAACCCCACCGTGTCCCTGC
AGGAGTTCCTCATGGCCGAGGAGTCTCTGCCTGGCACGCTGCTGAAGCT
GGCGGCACAGGGGCTGGGCATGCAGGCCGCCTGCACCCTGACGCGCCTC
TGCTGGGCCTGGGAGCTCAGTGACCTGCACCTGCTGCAGAGCCTCATGG
CCCAGAGCTGCAGCTCGGCCCTGCGCACATCCGTGCCCCACGGGGCGCT
TGTGGAGGCCGCCTGCGCCTTTTGTTTCCATCTGACCCTCCTGCACCTG
CGGCACAGTCCTCCCGCCTACAGCGGGCCCGCTGTGGCTCTGTTGGTCA
CCGTCACGGCCTACACGGCCGGGCCCTTCACGTCTGCCTTCTTCAACCC
TGCCCTGGCCGCCTCTGTGACCTTTGCCTGCTCGGGACACACCTTACTG
GAGTACGTGCAGGTGTACTGGCTGGGCCCTCTGACAGGGATGGTCCTGG
CTGTGCTGCTGCACCAGGGCCGCCTTCCCCACCTTTTCCAGAGGAACCT
GTTCTACGGCCAGAAGAACAAGTACCGAGCACCCCGAGGGAAGCCGGCC
CCGGCCTCAGGGGACACCCAGACCCCTGCAAAGGGGTCCAGTGTCCGGG
AGCCTGGGCGCAGTGGTGTTGAGGGGCCACATTCCAGCTGAGTGGCCTT
GCTCTGTGTGAGCCCCGTGCGAGGGCCCTGCTTGTAGCTGGACCCTGGA
ACCTTCTGTAGCTAAGAGGGAATCCTGGCCCCCTCCCCAGAAGCCATTT
GTCAATAAACCATTTCTAAGA.

In an aspect, a disclosed chicken beta actin (CBA) promoter can comprise the following sequence or a fragment thereof:

(SEQ ID NO: 01)
TTCAATATTGGCCATTAGCCATATTATTCATTGGTTATATAGCATAAAT
CAATATTGGATATTGGCCATTGCATACGTTGTATCTATATCATAATATG
TACATTTATATTGGCTCATGTCCAATATGACCGCCATGTTGGCATTGAT
TATTGACTAGTTATTAATAGTAATCAATTACGGGGTCATTAGTTCATAG
CCCATATATGGAGTTCCGCGTTACATAACTTACGGTAAATGGCCCGCCT
GGCTGACCGCCCAACGACCCCCGCCCATTGACGTCAATAATGACGTATG
TTCCCATAGTAACGCCAATAGGGACTTTCCATTGACGTCAATGGGTGGA
GTATTTACGGTAAACTGCCCACTTGGCAGTACATCAAGTGTATCATATG
CCAAGTCCGCCCCCTATTGACGTCAATGACGGTAAATGGCCCGCCTGGC
ATTATGCCCAGTACATGACCTTACGGGACTTTCCTACTTGGCAGTACAT
CTACGTATTAGTCATCGCTATTACCATGGTCGAGGTGAGCCCCACGTTC
TGCTTCACTCTCCCCATCTCCCCCCCCTCCCCACCCCCAATTTTGTATT
TATTTATTTTTTAATTATTTTGTGCAGCGATGGGGGCGGGGGGGGGGGG
GGGGCGCGCGCCAGGCGGGGCGGGGCGGGGCGAGGGGCGGGGCGGGGCG
AGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAG
CGCGCGGCGGGCGGGAGTCGCTGCGACGCTGCCTTCGCCCCGTGCCCCG
CTCCGCCGCCGCCTCGCGCCGCCCGCCCCGGCTCTGACTGACCGCGTTA
CTCCCACAGGTGAGCGGGCGGGACGGCCCTTCTCCTCCGGGCTGTAATT
AGCGCTTGGTTTAATGACGGCTTGTTTCTTTTCTGTGGCTGCGTGAAAG
CCTTGAGGGGCTCCGGGAGGGCCCTTTGTGCGGGGGGGAGCGGCTCGGG
GGGTGCGTGCGTGTGTGTGTGCGTGGGGAGCGCCGCGTGCGGCCCGCGC
TGCCCGGCGGCTGTGAGCGCTGCGGGCGCGGCGCGGGGCTTTGTGCGCT
CCGCAGTGTGCGCGAGGGGAGCGCGGCCGGGGGCGGTGCCCCGCGGTGC
GGGGGGGGCTGCGAGGGGAACAAAGGCTGCGTGCGGGGTGTGTGCGTGG
GGGGGTGAGCAGGGGGTATGGGCGCGGCGGTCGGGCTGTAACCCCCCCC
TGCACCCCCCTCCCCGAGTTGCTGAGCACGGCCCGGCTTCGGGTGCGGG
GCTCCGTACGGGGCGTGGCGCGGGGCTCGCCGTGCCGGGCGGGGGGTGG
CGGCAGGTGGGGGTGCCGGGCGGGGGGGGGCCGCCTCGGGCCGGGGAGG
GCTCGGGGGAGGGGCGCGGCGGCCCCCGGAGCGCCGGCGGCTGTCGAGG
CGCGGCGAGCCGCAGCCATTGCCTTTTATGGTAATCGTGCGAGAGGGCG
CAGGGACTTACTTTGTCCCAAATCTGTGCGGAGCCGAAATCTGGGAGGC
GCCGCCGCACCCCCTCTAGCGGGCGCGGGGCGAAGCGGTGCGGCGCCGG
CAGGAAGGAAATGGGCGGGGAGGGCCTTCGTGCGTCGCCGCGCCGCCGT
CCCCTTCTCCCTCTCCAGCCTCGGGGCTGTCCGCGGGGGGACGGCTGCC
TTCGGGGGGGACGGGGCAGGGCGGGGTTCGGCTTCTGGCGTGTGACCGG
CGGCTCTAGAGCCTCTGCTAACCATGTTCATGCCTTCTTCTTTTTCCTA
CAGCTCCTGGGCAACGTGCTGGTTATTGTGCTGTCTCATCATTTTGGCA
AAG.

In an aspect, a disclosed GFAP promoter can comprise the following sequence or a fragment thereof:

(SEQ ID NO: 02)
GATCTAACATATCCTGGTGTGGAGTAGGGGACGCTGCTCTGACAGAGGC
TCGGGGGCCTGAGCTGGCTCTGTGAGCTGGGGAGGAGGCAGACAGCCAG
GCCTTGTCTGCAAGCAGACCTGGCAGCATTGGGCTGGCCGCCCCCCAGG
GCCTCCTCTTCATGCCCAGTGAATGACTCACCTTGGCACAGACACAATG
TTCGGGGTGGGCACAGTGCCTGCTTCCCGCCGCACCCCAGCCCCCCTCA
AATGCCTTCCGAGAAGCCCATTGAGCAGGGGGCTTGCATTGCACCCCAG
CCTGACAGCCTGGCATCTTGGGATAAAAGCAGCACAGCCCCCTAGGGGC
TGCCCTTGCTGTGTGGCGCCACCGGCGGTGGAGAACAAGGCTCTATTCA
GCCTGTGCCCAGGAAAGGGGATCAGGGGATGCCCAGGCATGGACAGTGG
GTGGCAGGGGGGGAGAGGAGGGCTGTCTGCTTCCCAGAAGTCCAAGGAC
ACAAATGGGTGAGGGGAGAGCTCTCCCCATAGCTGGGCTGCGGCCCAAC
CCCACCCCCTCAGGCTATGCCAGGGGGTGTTGCCAGGGGCACCCGGGCA
TCGCCAGTCTAGCCCACTCCTTCATAAAGCCCTCGCATCCCAGGAGCGA
GCAGAGCCAGAGCAGGTTGGAGAGGAGACGCATCACCTCCGCTGCTCGC
GGGG.

In an aspect, a disclosed Rgs5 promoter can comprise the following sequence or a fragment thereof:

(SEQ ID NO: 03)
AGGCACTAATGATCTTATCTCAGTTTAGGCTCCAACACAGCCCTAACTA
AACAAACAAGAGACTGTGGGGACAGAAGCCACCAGAAACATGCCCCAGC
AGTGCCTGAAGTCAGGAAAGACTCCCAGCCCCCACCGAAGGCAAACTGC
TGCTGAGCTCCAGCCCACAATTTTCGCCCAGGCTGCTGATTGGTCCTCG
GAGCCACGGAGCTGTGAGAGAGCTTGTATATATTCCTCAAAGGGAAAGG
CTGCCCCAGATTATTGAAGTTTCCACAGACGGTCAGCTGTTGAGAGGTT
CGTGCTCAAGTTGAGGATCTAAGCCGCCAGCCAAAATGTGT.

In an aspect, a disclosed Syn1 promoter can comprise the following sequence or a fragment thereof:

(SEQ ID NO: 04)
AGTGCAAGTGGGTTTTAGGACCAGGATGAGGCGGGGTGGGGGTGCCTAC
CTGACGACCGACCCCGACCCACTGGACAAGCACCCAACCCCCATTCCCC
AAATTGCGCATCCCCTATCAGAGAGGGGGAGGGGAAACAGGATGCGGCG
AGGCGCGTGCGCACTGCCAGCTTCAGCACCGCGGACAGTGCCTTCGCCC
CCGCCTGGCGGCGCGCGCCACCGCCGCCTCAGCACTGAAGGCGCGCTGA
CGTCACTCGCCGGTCCCCCGCAAACTCCCCTTCCCGGCCACCTTGGTCG
CGTCCGCGCCGCCGCCGGCCCAGCCGGACCGCACCACGCGAGGCGCGAG
ATAGGGGGGCACGGGCGCGACCATCTGCGCTGCGGCGCCGGCGACTCAG
CGCTGCCTCAGTCTGCGGTGGGCAGCGGAGGAGTCGTGTCGTGCCTGAG
AGCGCAG.

In an aspect, a disclosed S100 beta promoter can comprise the following sequence or a fragment thereof:

(SEQ ID NO: 05)
ATCTTGTGGGAAACCCTTTCACGGCCACTTTGAAACTGGGTTGGGAGAA
GGACGTGATGCTGGGTCAGGCATCCATTGGGGACCATGGACATTTTTGG
AGAGTTTGGGCCAATGTTCTTGTGTAATTTGTCAGAAGGAACACCTACT
TCTTCATTCTTGTGAAGAAGAACCCATAAACTGGCCCCCGTGTTCAGAG
GCTCCAGTCGAGCGAGTTGAGGGGACAGCTGGACTTCTCCTTCCTCAGG
CTGGCTGGAGGCTTCAGCAAGGGGGCACACCGTCCACACGCCTCTGTAA
AACAGCGTTCGAACGGGACATCTCTAAGGCATCGTCCAACTCTGAGAGT
CTGTGACCTTCCACCCTGGGGTGGAGGGAAAGGCCTTGGGATACAGTCC
ACACATCACGTTTTCCCCTAAAAGTACCACCCGTTTTAGTCCCTGCAGA
CACTGGCCTCAGCAGCTTCTCTGAGGCAGCCCCGTTGAGCCCAGGGCTG
ACCACAGACAAGCACTACTGTCCACAGAACTTCACGCCCAGTGGGGCCA
GACTTGGAGGATGGCAGAGGAGAGAAGCTCCAGGGGCCTCTCCATTAGA
AACCAACTTGCAGGCCACTTAATCTCCTATGCTCAGCCTGTACTTGGAA
GCTGCTTGTTCTTGGCTGCACATTTGCTTTGTTTGAATCAATTAATCCA
AGTCTCTCTCCTCTCCCCACCCCCAACCTTGCCTTTAGGGTGACATCAA
TATTCATGTAATAAAGAAATCGCACAAAAAGCTGACTCCCCACTTCCTG
CCCTACAGGCCCTTTTTTCTTCCTCAGCCCATGTGCAATCCTGGCTCCC
AGCAAGTCCCCGGGGCTGCTTGGTCAATGCAGCCCTGTGTGCAGGCCTG
GCAGCCCTGCCACCCCCGCCTTCGGCTCCCATTGGCTGCCACGGCCTGC
AGTGGGCTGCACCAGGGTTCATCCATCCTCCCTGGGCAGAGGGAATAAG
AGGCTGCCTCTGCCCACCAGTCCTGCCGCCCAGGACCCGCAGCAGAGAC
GACGCCTGCAGCAAGGAGACCAGGAAGGGGTGAGACAAGGAAGAGGTGA
GAAAGAGCCAGGCCAAGAGGACGCTCAGGAAGAAATGGTTCTTTTCTTT
TGGGTGGAACGGAATGGAGGGGTAGAAACTAAGTGGTAGCTTAAAAAGC
CCCTTTAGGACAATTGGCAGCATTTCAGAAGTGTCAATAGGATGATGTG
TTTTAATCTCCACATTACTGCTGCTTTGTGGACACCTGACTGCATCAGC
CCTAGACAGCTAGAGGTGTGTTTTGCCATAAATCAGAGAAACGTCAGGT
TTCATGGCCCAGAAGTGATTGTTGACATTTTCCCAGCGGTACTACAAAT
ACTGCCTCTTCTGTAACTATTTGAAGAGTAAAGATTTTGCTTCCCACTG
GGGCTAAAAATGATGGAGAACCTAATGCTAGTTTAGTCCTATATCATAA
ATAAATGCTTCCCTTTTCTCCTTATATTTCTTCCACCGCCCTGGGTAAG
TAACTCTTCAGTTTTCCAGTTTCCCTCAGTTTGAAGTGCCAGGGTCCCC
ACAGCCCCAGGACGCCACTCAGAAATTACGTAACAATTACAAATAAATT
GGGTCAAAGAAATCGGGGTTTTGGTGGGTTTTAACTTTCCTCAGTCTCA
CAGTTTCTCAGGGAGGTCGAACCCCTTCTTTAGAGGGATCCTC.

In an aspect, a disclosed AQP4 promoter can comprise the following sequence or a fragment thereof:

(SEQ ID NO: 06)
CTGGTTTTTTGTACCAGCTTCAGATGAAATACAGTCACTCAATAAAGTC
CTTCTGTTTTCTCTTTCCCTCCCTCTCGTTCTCTCTCCTTATTTGCCTA
GTGAGGGGAACCCTAAACCCTTTGGAGCTTTTCCAGCAGCTCCAAAATA
ATCTCTTGATTATTTTCCCAGGGAATTCCTGCCTTAAATGTCAGCAGAG
TACAACTACCCCACCAAAGCAGTCCGCCTGGATCTGAATCAAGAAAAAT
GTGCCTGCTACACCAAACATAAAAACCTGGATCTGTGGGGCGTGGGGCA
CAGAGAGAGATTTATGGTGTTTGTGAAAGGTGTCTCAAAAAAAAAAAAA
TTAAGTAAATGTTTTTGCATTCCTGAAATGCCCTGTGTCTACAGTGATC
AGGTACAGAAAAGCTACTTCAAGTTCAAATATAACTTAGCGATTGCCAC
ATGGTGCAGAATCTTTCCACCCCTAACACTCCAAAAACCCAATCAGACA
AGTGGCCGTAATCTGACTCCCAGCACACAGGGAGCTGCGGGGCAGGCAA
TGAGAGCTGCACTCTGGCTGGGAGTGACAGACCCACAGCAAGGCGGTGG
GGGTAAGTCCTTCTTTGCCTTAAGGGAACAACGCTTAGGGCCAAAAGAT
GAAACTGT.

In an aspect, a disclosed polyadenylation signal can comprise the following sequence or a fragment thereof:

(SEQ ID NO: 34)
AGACATGATAAGATACATTGATGAGTTTGGACAAACCACAACTAGAATG
CAGTGAAAAAAATGCTTTATTTGTGAAATTTGTGATGCTATTGCTTTAT
TTGTAACCATTATAAGCTGCAATAAACAAGTTAACAACAACAATTGCAT
TCATTTTATGTTTCAGGTTCAGGGGGAGATGTGGGAGGTTTTTTAAAGC
AAGTAAAACCTCTACAAATGTGGTA.

### b. Polypeptide Sequence

In an aspect, a disclosed encoded Aqp1 can comprise the following sequence or a fragment thereof:

(SEQ ID NO: 07)
MASEFKKKLFWRAVVAEFLATTLFVFISIGSALGFKYPVGNNQTAVQDN
VKVSLAFGLSIATLAQSVGHISGAHLNPAVTLGLLLSCQISIFRALMYI
IAQCVGAIVATAILSGITSSLTGNSLGRNDLADGVNSGQGLGIEIIGTL
QLVLCVLATTDRRRRDLGGSAPLAIGLSVALGHLLAIDYTGCGINPARS
FGSAVITHNFSNHWIFWVGPFIGGALAVLIYDFILAPRSSDLTDRVKVW
TSGQVEEYDLDADDINSRVEMKPK.

In an aspect, a disclosed encoded Aqp2 can comprise the following sequence or a fragment thereof:

(SEQ ID NO: 08)
MWELRSIAFSRAVFAEFLATLLFVFFGLGSALNWPQALPSVLQIAMAFG
LGIGTLVQALGHISGAHINPAVTVACLVGCHVSVLRAAFYVAAQLLGAV
AGAALLHEITPADIRGDLAVNALSNSTTAGQAVTVELFLTLQLVLCIFA
STDERRGENPGTPALSIGFSVALGHLLGIHYTGCSMNPARSLAPAVVTG
KFDDHWVFWIGPLVGAILGSLLYNYVLFPPAKSLSERLAVLKGLEPDTD
WEEREVRRRQSVELHSPQSLPRGTKA.

In an aspect, a disclosed encoded Aqp3 can comprise the following sequence or a fragment thereof:

(SEQ ID NO: 09)
MGRQKELVSRCGEMLHIRYRLLRQALAECLGTLILVMFGCGSVAQVVLS
RGTHGGFLTINLAFGFAVTLGILIAGQVSGAHLNPAVTFAMCFLAREPW
IKLPIYTLAQTLGAFLGAGIVFGLYYDAIWHFADNQLFVSGPNGTAGIF
ATYPSGHLDMINGFFDQFIGTASLIVCVLAIVDPYNNPVPRGLEAFTVG
LVVLVIGTSMGFNSGYAVNPARDFGPRLFTALAGWGSAVFTTGQHWWWV
PIVSPLLGSIAGVFVYQLMIGCHLEQPPPSNEEENVKLAHVKHKEQI.

In an aspect, a disclosed encoded Aqp4 can comprise the following sequence or a fragment thereof:

(SEQ ID NO: 10)
MVAFKGVWTQAFWKAVTAEFLAMLIFVLLSLGSTINWGGTEKPLPVDMV
LISLCFGLSIATMVQCFGHISGGHINPAVTVAMVCTRKISIAKSVFYIA
AQCLGAIIGAGILYLVTPPSVVGGLGVTMVHGNLTAGHGLLVELIITFQ
LVFTIFASCDSKRTDVTGSIALAIGFSVAIGHLFAINYTGASMNPARSF
GPAVIMGNWENHWIYWVGPIIGAVLAGGLYEYVFCPDVEFKRRFKEAFS
KAAQQTKGSYMEVEDNRSQVETDDLILKPGVVHVIDVDRGEEKKGKDQS
GEVLSSV.

In an aspect, a disclosed encoded Aqp5 can comprise the following sequence or a fragment thereof:

(SEQ ID NO: 11)
MKKEVCSVAFLKAVFAEFLATLIFVFFGLGSALKWPSALPTILQIALAF
GLAIGTLAQALGPVSGGHINPAITLALLVGNQISLLRAFFYVAAQLVGA
IAGAGILYGVAPLNARGNLAVNALNNNTTQGQAMVVELILTFQLALCIF
ASTDSRRTSPVGSPALSIGLSVTLGHLVGIYFTGCSMNPARSFGPAVVM
NRFSPAHWVFWVGPIVGAVLAAILYFYLLFPNSLSLSERVAIIKGTYEP
DEDWEEQREERKKTMELTTR.

In an aspect, a disclosed encoded Aqp6 can comprise the following sequence or a fragment thereof:

(SEQ ID NO: 12)
MDAVEPGGRGWASMLACRLWKAISRALFAEFLATGLYVFFGVGSVMRWP
TALPSVLQIAITFNLVTAMAVQVTWKASGAHANPAVTLAFLVGSHISLP
RAVAYVAAQLVGATVGAALLYGVMPGDIRETLGINVVRNSVSTGQAVAV
ELLLTLQLVLCVFASTDSRQTSGSPATMIGISVALGHLIGIHFTGCSMN
PARSFGPAIIIGKFTVHWVFWVGPLMGALLASLIYNFVLFPDTKTLAQR
LAILTGTVEVGTGAGAGAEPLKKESQPGSGAVEMESV.

In an aspect, a disclosed encoded Aqp7 can comprise the following sequence or a fragment thereof:

(SEQ ID NO: 13)
MVQASGHRRSTRGSKMVSWSVIAKIQEILQRKMVREFLAEFMSTYVMMV
FGLGSVAHMVLNKKYGSYLGVNLGFGFGVTMGVHVAGRISGAHMNAAVT
FANCALGRVPWRKFPVYVLGQFLGSFLAAATIYSLFYTAILHFSGGQLM
VTGPVATAGIFATYLPDHMTLWRGFLNEAWLTGMLQLCLFAITDQENNP
ALPGTEALVIGILVVIIGVSLGMNTGYAINPSRDLPPRIFTFIAGWGKQ
VFSNGENWWWVPVVAPLLGAYLGGIIYLVFIGSTIPREPLKLEDSVAYE
DHGITVLPKMGSHEPTISPLTPVSVSPANRSSVHPAPPLHESMALEHF.

In an aspect, a disclosed encoded Aqp8 can comprise the following sequence or a fragment thereof:

(SEQ ID NO: 14)
MSGEIAMCEPEFGNDKAREPSVGGRWRVSWYERFVQPCLVELLGSALFI
FIGCLSVIENGTDTGLLQPALAHGLALGLVIATLGNISGGHFNPAVSLA
AMLIGGLNLVMLLPYWVSQLLGGMLGAALAKAVSPEERFWNASGAAFVT
VQEQGQVAGALVAEIILTTLLALAVCMGAINEKTKGPLAPFSIGFAVTV
DILAGGPVSGGCMNPARAFGPAVVANHWNFHWIYWLGPLLAGLLVGLLI
RCFIGDGKTRLILKAR.

In an aspect, a disclosed encoded Aqp9 can comprise the following sequence or a fragment thereof:

(SEQ ID NO: 15)
MQPEGAEKGKSFKQRLVLKSSLAKETLSEFLGTFILIVLGCGCVAQAIL
SRGRFGGVITINVGFSMAVAMAIYVAGGVSGGHINPAVSLAMCLFGRMK
WFKLPFYVGAQFLGAFVGAATVFGIYYDGLMSFAGGKLLIVGENATAHI
FATYPAPYLSLANAFADQVVATMILLIIVFAIFDSRNLGAPRGLEPIAI
GLLIIVIASSLGLNSGCAMNPARDLSPRLFTALAGWGFEVFRAGNNFWW
IPVVGPLVGAVIGGLIYVLVIEIHHPEPDSVFKTEQSEDKPEKYELSVI
M.

In an aspect, a disclosed encoded Aqp10 can comprise the following sequence or a fragment thereof:

(SEQ ID NO: 16)
MVFTQAPAEIMGHLRIRSLLARQCLAEFLGVFVLMLLTQGAVAQAVTSG
ETKGNFFTMFLAGSLAVTIAIYVGGNVSGAHLNPAFSLAMCIVGRLPWV
KLPIYILVQLLSAFCASGATYVLYHDALQNYTGGNLTVTGPKETASIFA
TYPAPYLSLNNGFLDQVLGTGMLIVGLLAILDRRNKGVPAGLEPVVVGM
LILALGLSMGANCGIPLNPARDLGPRLFTYVAGWGPEVFSAGNGWWWVP
VVAPLVGATVGTATYQLLVALHHPEGPEPAQDLVSAQHKASELETPASA
QMLECKL.

In an aspect, a disclosed encoded Aqp11 can comprise the following sequence or a fragment thereof:

(SEQ ID NO: 17)
MSPLLGLRSELQDTCTSLGLMLSVVLLMGLARVVARQQLHRPVAHAFVL
EFLATFQLCCCTHELQLLSEQHPAHPTWTLTLVYFFSLVHGLTLVGTSS
NPCGVMMQMMLGGMSPETGAVRLLAQLVSALCSRYCTSALWSLGLTQYH
VSERSFACKNPIRVDLLKAVITEAVCSFLFHSALLHFQEVRTKLRIHLL
AALITFLVYAGGSLTGAVFNPALALSLHFMCFDEAFPQFFIVYWLAPSL
GILLMILMFSFFLPWLHNNHTINKKE.

In an aspect, a disclosed encoded Aqp12 can comprise the following sequence or a fragment thereof:

(SEQ ID NO: 18)
MSPLLGLRSELQDTCTSLGLMLSVVLLMGLARVVARQQLHRPVAHAFVL
EFLATFQLCCCTHELQLLSEQHPAHPTWTLTLVYFFSLVHGLTLVGTSS
NPCGVMMQMMLGGMSPETGAVRLLAQLVSALCSRYCTSALWSLGLTQYH
VSERSFACKNPIRVDLLKAVITEAVCSFLFHSALLHFQEVRTKLRIHLL
AALITFLVYAGGSLTGAVFNPALALSLHFMCFDEAFPQFFIVYWLAPSL
GILLMILMFSFFLPWLHNNHTINKKE.

In an aspect, a disclosed encoded Aqp12 can comprise the following sequence or a fragment thereof:

(SEQ ID NO: 19)
MAGLNVSLSFFFATFTLCEAARRASKALLPVGAYEVFAREAMRTLVELG
PWAGDFGPDLLLTLLFLLFLAHGVTLDGASANPTVSLQEFLMAEESLPG
TLLKLAAQGLGMQAACTLTRLCWAWELSDLHLLQSLMAQSCSSALRTSV
PHGALVEAACAFCFHLTLLHLRHSPPAYSGPAVALLVTVTAYTAGPFTS
AFFNPALAASVTFACSGHTLLEYVQVYWLGPLTGMVLAVLLHQGRLPHL
FQRNLFYGQKNKYRAPRGKPAPASGDTQTPAKGSSVREPGRSGVEGPHS
S.

| SEQ ID | | |
|---|---|---|
| NO: | Name (Type) | Notes |
| 1 | Chicken beta actin promoter | |
| | (nucleotide) | |
| 2 | GFAP promoter (nucleotide) | |
| 3 | Rgs5 promoter (nucleotide) | |
| 4 | Syn1 promoter (nucleotide) | |
| 5 | S100 Beta promoter | |
| | (nucleotide) | |
| 6 | Aqp4 promoter (nucleotide) | |
| 7 | Aquaporin 1 (amino acid) | Uniprot P29972 |
| 8 | Aquaporin 2 (amino acid) | Uniprot P41181 |
| 9 | Aquaporin 3 (amino acid) | Uniprot Q92482 |
| 10 | Aquaporin 4 (amino acid) | Uniprot P55087 |
| 11 | Aquaporin 5 (amino acid) | Uniprot P55064 |
| 12 | Aquaporin 6 (amino acid) | Uniprot Q13520 |
| 13 | Aquaporin 7 (amino acid) | Uniprot O14520 |
| 14 | Aquaporin 8 (amino acid) | Uniprot O94778 |
| 15 | Aquaporin 9(amino acid) | Uniprot O43315 |
| 16 | Aquaporin 10 (amino acid) | Uniprot Q96PS8 |
| 17 | Aquaporin 11 (amino acid) | Uniprot Q8NBQ7 |
| 18 | Aquaporin 12A (amino acid) | Uniprot Q8IXF9 |
| 19 | Aquaporin 12B (amino acid) | Uniprot A6NM10 |
| 20 | Aquaporin 1 (mRNA) | |
| 21 | Aquaporin 2 (mRNA) | |
| 22 | Aquaporin 3 (mRNA) | |
| 23 | Aquaporin 4 (mRNA) | |
| 24 | Aquaporin 5 (mRNA) | |
| 25 | Aquaporin 6 (mRNA) | |
| 26 | Aquaporin 7 (mRNA) | |
| 27 | Aquaporin 8 (mRNA) | |
| 28 | Aquaporin 9 (mRNA) | |
| 29 | Aquaporin 10 (mRNA) | |
| 30 | Aquaporin 11 (mRNA) | Transcript 1 (NM_173039.3) |
| 31 | Aquaporin 11 (mRNA) | Transcript 2 |
| | | (NM_001363477.2) |
| 32 | Aquaporin 12A (mRNA) | NM_198998.3 |
| 33 | Aquaporin 12B (mRNA) | NM_001102467.2 |
| 34 | SV40 PolyA (nucleotide) | |
| 35 | Aquaporin 1 (nucleotide) | NG_007475.2 (63301 . . . |
| | | 77124, chrm. 7) |
| 36 | Aquaporin 2 (nucleotide) | NG_008913.1: (5001 . . . |
| | | 13141, chrm. 12) |
| 37 | Aquaporin 3 (nucleotide) | NG_007476.1 (5000 . . . |
| | | 11433, chrm. 9) |
| 38 | Aquaporin 4 (nucleotide) | NG_029560.1 (5046 . . . |
| | | 18710, chrm. 18) |
| 39 | Aquaporin 5 (nucleotide) | NG_033883.1 (5377 . . . |
| | | 9187, chrm. 12) |
| 40 | Aquaporin 6 (nucleotide) | NC_000012.12 |
| | | (49972947 . . . 49977139, |
| | | chrm. 12) |
| 41 | Aquaporin 7 (nucleotide) | NG_027764.1 (4952 . . . |
| | | 24329, chrm. 9) |
| 42 | Aquaporin 8 (nucleotide) | NC_000016.10 |
| | | (25216917 . . . 25228932, |
| | | chrm. 16) |
| 43 | Aquaporin 9 (nucleotide) | NG_011975.2 (5173 . . . |
| | | 52703, chromosome 15) |
| 44 | Aquaporin 10 (nucleotide) | NC_000001.11 |
| | | (154321059 . . . 154325325, |
| | | chrm. 1) |
| 45 | Aquaporin 11 (nucleotide) | NC_000011.10 |
| | | (77589953 . . . 77610356, |
| | | chrm. 11) |
| 46 | Aquaporin 12A (nucleotide) | NC_000002.12 |
| | | (240691866 . . . 240698483, |
| | | chrm. 2) |
| 47 | Aquaporin 12B (nucleotide) | NC_000002.12 |
| | | (c240685743 . . . 240676418, |

### 2. Vectors

Disclosed herein is a vector comprising a disclosed isolated nucleic acid molecule. Disclosed herein is a vector comprising an isolated nucleic acid molecule encoding an aquaporin. Disclosed herein is vector comprising a disclosed expression cassette comprising a disclosed nucleic acid sequence operably linked to one or more disclosed expression control elements and/or message stabilizing elements. Disclosed herein is a vector comprising an expression cassette shown in FIG. 1. Disclosed herein is a AAV vector comprising an expression cassette shown in FIG. 1.

In an aspect, a therapeutically effective amount of disclosed vector can restore one or more aspects of the glymphatic pathway. In an aspect, a therapeutically effective amount of disclosed vector can restore one or more aspects of the water influx in the brain of a subject. In an aspect, a therapeutically effective amount of a disclosed vector can prevent protein aggregation in the brain of a subject, can remove and/or clear protein aggregates in the brain of a subject, can improve fluid flux in the brain of a subject, or any combination thereof.

In an aspect, a disclosed isolated nucleic acid molecule encoding the aquaporin can be CpG-depleted and/or codon-optimized for expression in a human cell. In an aspect, “CpG-depleted” can mean “CpG-free”. In an aspect, “CpG-free” can mean “CpG-depleted”. In an aspect, “CpG-free” can mean completely free of CpGs or partially free of CpGs. In an aspect, “CpG-free” can mean completely depleted of CpGs or partially depleted of CpGs. In an aspect, “CpG-free” can mean “CpG-free” for a desired and/or ideal expression level. CpG depletion and/or optimization is known to the skilled person in the art.

In an aspect, a disclosed nucleic acid molecule can comprise the nucleotide sequence for AQP1, AQP2, AQP3, AQP4, AQP5, AQP6, AQP7, AQP8, AQP9, AQP10, AQP11, AQP12, APQ13, or any combination thereof. In an aspect, a disclosed nucleic acid molecule can comprise the nucleotide sequence for AQP1, AQP4, AQP5, AQP6, or, AQP8, or any combination thereof. In an aspect, a disclosed nucleic acid molecule can comprise the nucleotide sequence for AQP4.

In an aspect, a disclosed nucleic acid molecule can comprise the nucleotide sequence for a recombinant AQP1, AQP2, AQP3, AQP4, AQP5, AQP6, AQP7, AQP8, AQP9, AQP10, AQP11, AQP12, APQ13, or any combination thereof. In an aspect, a disclosed nucleic acid molecule can comprise the nucleotide sequence for a recombinant AQP1, AQP4, AQP5, AQP6, or, AQP8, or any combination thereof. In an aspect, a disclosed nucleic acid molecule can comprise the nucleotide sequence for a recombinant AQP4.

In an aspect, a disclosed encoded aquaporin can comprise Aqp1, Aqp2, Aqp3, Aqp4, Aqp5, Aqp6, Apq7, Apq8, Apq9, Apq10, Apq11, Apq12, Apq13, or any combination thereof. In an aspect, a disclosed encoded aquaporin can comprise Aqp1, Aqp4, Aqp5, Apq6, or Apq8. In an aspect, a disclosed encoded aquaporin can comprise Aqp4. In an aspect, a disclosed encoded aquaporin can comprise recombinant Aqp1, Aqp2, Aqp3, Aqp4, Aqp5, Aqp6, Apq7, Apq8, Apq9, Apq10, Apq11, Apq12, Apq13, or any combination thereof. In an aspect, a disclosed encoded aquaporin can comprise recombinant Aqp1, Aqp4, Aqp5, Apq6, or Apq8. In an aspect, a disclosed encoded aquaporin can comprise recombinant Aqp4.

In an aspect, a disclosed nucleic acid molecule can be packaged into a viral or non-viral vector. In an aspect, a disclosed vector can be a non-viral vector, and wherein the non-viral vector comprises a polymer-based vector, a peptide-based vector, a lipid nanoparticle, a solid lipid nanoparticle, or a cationic lipid-based vector. In an aspect, a disclosed vector the vector is can be a viral vector, and the viral vector can comprise an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can comprise bovine AAV, caprine AAV, canine AAV, equine AAV, ovine AAV, avian AAV, primate AAV, or non-primate AAV.

In an aspect, a disclosed AAV vector can comprise a recombinant AAV (rAAV) vector. In an aspect, a disclosed AAV vector can be self-complementary.

In an aspect, a disclosed AAV vector or disclosed recombinant rAAV vector can comprise AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed AAV vector can comprise a recombinant AAV vector.

In an aspect, a disclosed AAV vector can comprise AAV2g9. In an aspect, a disclosed AAV2g9 can comprise the chimera described in Shen S, et al. (2013) J Biol Chem. 288(40):28814-28823. In an aspect, a disclosed AAV vector can comprise AAV-cc47.

In an aspect, a disclosed AAV vector can comprise a promoter operably linked to the nucleic acid sequence encoding a disclosed aquaporin.

In an aspect, a therapeutically effective amount of disclosed vector can comprise a range of about 1×10 10 vg/kg to about 2 x 10′ vg/kg. In an aspect, therapeutically effective amount of disclosed vector can comprise a dose of about 1×10¹¹ vg/kg to about 8×10¹³ vg/kg or about 1×10¹² vg/kg to about 8×10¹³ vg/kg. In an aspect, a therapeutically effective amount of disclosed vector can comprise a dose of about 1×10¹³ vg/kg to about 6×10¹³ vg/kg. In an aspect, a therapeutically effective amount of disclosed vector can comprise a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a therapeutically effective amount of disclosed vector can comprise a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10 ¹² vg/kg, no more than about 1×10¹³ vg/kg, no more than about 5×10¹³ vg/kg, or no more than about 1×10¹⁴ vg/kg. In an aspect, a therapeutically effective amount of disclosed vector can comprise a dose of about 1×10¹² vg/kg. In an aspect, a therapeutically effective amount of disclosed vector can comprise a dose of about 1×10¹¹ vg/kg.

In an aspect, a disclosed vector can be administered via intravenous administration, intra-CSF administration, intracerebroventricular (ICV) administration, intraventricular administration, intra-cisterna magna (ICM) administration, intraparenchymal administration, intrathecal (lumbar, cisternal, or both) administration, or a combination thereof.

In an aspect, a disclosed vector can be administered in a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed vector can be administered via multiple routes of administration.

In an aspect, a disclosed promoter can comprise a constitutive promoter, a ubiquitous promoter, or a tissue-specific promoter.

In an aspect, a disclosed constitutive promoter can be a chicken beta actin (CBA) promoter. In an aspect, a disclosed CBA promoter can comprise the sequence set forth in SEQ ID NO:01 or a sequence having at least 50%, at least 60%, at least 70%, at least 80%, at least 90%, at least 95%, or at least 99% identity to the sequence set forth in SEQ ID NO:01.

In an aspect, a disclosed tissue-specific promoter can comprise a brain cell specific promoter. In an aspect, a disclosed brain cell specific promoter can comprise a synapsin 1 (Syn1) promoter, a calmodulin/calcium dependent kinase II (CAMKII) promoter, a glial fibrillary acidic protein (GFAP) promoter, a Rgs5 promoter, a S100 beta promoter, a neuron-specific enolase (NSE) promoter, a Thy1 promoter, or any combination thereof.

In an aspect, a disclosed GFAP promoter can comprise the sequence set forth in SEQ ID NO:02 or a sequence having at least 50%, at least 60%, at least 70%, at least 80%, at least 90%, at least 95%, or at least 99% identity to the sequence set forth in SEQ ID NO:02. In an aspect, a disclosed Syn1 promoter can comprise the sequence set forth in SEQ ID NO:04 or a sequence having at least 50%, at least 60%, at least 70%, at least 80%, at least 90%, at least 95%, or at least 99% identity to the sequence set forth in SEQ ID NO:04. In an aspect, a disclosed Rgs5 promoter can comprise the sequence set forth in SEQ ID NO:03 or a sequence having at least 50%, at least 60%, at least 70%, at least 80%, at least 90%, at least 95%, or at least 99% identity to the sequence set forth in SEQ ID NO:03.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed aquaporin or some other enzyme involved in glymphatic transport or metabolism). In an aspect, a disclosed endogenous promoter or a disclosed endogenous promoter/enhancer can be used for constitutive and efficient expression of a disclosed gene (e.g., a nucleic acid sequence encoding an aquaporin).

In an aspect, a disclosed endogenous promoter or a disclosed endogenous promoter/enhancer can be used for constitutive and efficient expression of an aquaporin. In an aspect, a disclosed endogenous promoter or a disclosed endogenous promoter/enhancer can comprise the promoter for the gene encoding Aqp1, Aqp2, Aqp3, Aqp4, Aqp5, Aqp6, Aqp7, Aqp8, Aqp9, Aqp10, Aqp 11, Aqp12, or Aqp 13. For example, in an aspect, when an encoded polypeptide comprises the Aqp1, the disclosed endogenous promoter or a disclosed endogenous promoter/enhancer can comprise the promoter or promoter/enhancer for the gene encoding Aqp1. Similarly, when an encoded polypeptide comprises Aqp4, the disclosed endogenous promoter or a disclosed endogenous promoter/enhancer can comprise the promoter or promoter/enhancer for the gene encoding Aqp4. For example, in an aspect, a disclosed endogenous promoter or a disclosed endogenous promoter/enhancer can comprise the sequence set forth in SEQ ID NO:06 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the endogenous promoter sequence for any one of Aqp1, Aqp2, Aqp3, Aqp4, Aqp5, Aqp6, Aqp7, Aqp8, Aqp9, Aqp10, Aqp11, Aqp12, or Aqp 13.

In an aspect, a disclosed viral vector can comprise a disclosed nucleic acid molecule encoding a disclosed aquaporin, a disclosed brain cell specific promoter, a pair of ITRs, and a polyadenylation sequence.

In an aspect, a disclosed viral vector can comprise a disclosed expression cassette. In an aspect, a disclosed expression cassette can comprise a disclosed nucleic acid sequence operably linked to one or more disclosed expression control elements and/or message stabilizing elements. As known to the art, a functional expression unit that is capable of properly driving the expression of an incorporated polynucleotide (i.e., a nucleic acid sequence encoding an aquaporin) can be referred to as an “expression cassette”. The skilled person knows how to design an expression cassette to allow the expression in a eukaryotic cell, such as preferably in a mammalian or human cell. For example, in an aspect, a disclosed expression cassette can comprise an expression cassette of FIG. 1.

Disclosed herein is an AAV vector comprising a disclosed isolated nucleic acid molecule encoding a disclosed aquaporin. Disclosed herein is AAV vector comprising a disclosed expression cassette comprising a disclosed nucleic acid sequence operably linked to one or more disclosed expression control elements and/or message stabilizing elements.

Disclosed herein is a recombinant AAV vector comprising a disclosed isolated nucleic acid molecule encoding a disclosed aquaporin. Disclosed herein is a recombinant AAV vector comprising a disclosed expression cassette comprising a disclosed nucleic acid sequence operably linked to one or more disclosed expression control elements and/or message stabilizing elements.

Disclosed herein is a recombinant AAV-cc47 vector comprising a disclosed isolated nucleic acid molecule encoding a disclosed aquaporin. Disclosed herein is a recombinant AAV-cc47 vector comprising a disclosed expression cassette comprising a disclosed nucleic acid sequence operably linked to one or more disclosed expression control elements and/or message stabilizing elements.

Disclosed herein is an AAV-cc47 vector comprising an isolated nucleic acid molecule encoding an aquaporin, wherein the nucleic acid sequence for aquaporin comprises the sequence set forth in any one of SEQ ID NO:20-SEQ ID NO:33 or in any one of SEQ ID NO:35-SEQ ID NO:47 or a fragment thereof.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47 or a fragment thereof, which is operably linked to a GFAP promoter comprising the nucleic acid sequence set forth in SEQ ID NO:02.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47 or a fragment thereof, which is operably linked to a Syn1 promoter comprising the nucleic acid sequence set forth in SEQ ID NO:04.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47 or a fragment thereof, which is operably linked to a Rgs5 promoter comprising the nucleic acid sequence set forth in SEQ ID NO:03.

Disclosed herein is AAV-cc47 vector comprising a disclosed expression cassette comprising the nucleic acid sequence set forth in any one of SEQ ID NO:20-SEQ ID NO:33 or in any one of SEQ ID NO:35-SEQ ID NO:47 or a fragment thereof, which is operably linked to a CBA promoter comprising the nucleic acid sequence set forth in SEQ ID NO:01.

Disclosed herein is a recombinant AAV2g9 vector comprising a disclosed isolated nucleic acid molecule encoding a disclosed aquaporin. Disclosed herein is a recombinant AAV2g9 vector comprising a disclosed expression cassette comprising a disclosed nucleic acid sequence operably linked to one or more disclosed expression control elements and/or message stabilizing elements.

Disclosed herein is an AAV2g9 vector comprising an isolated nucleic acid molecule encoding an aquaporin, wherein the nucleic acid sequence for aquaporin comprises the sequence set forth in any one of SEQ ID NO:20-SEQ ID NO:33 or in any one of SEQ ID NO:35-SEQ ID NO:47 or a fragment thereof.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47 or a fragment thereof, which is operably linked to a GFAP promoter comprising the nucleic acid sequence set forth in SEQ ID NO:02.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47 or a fragment thereof, which is operably linked to a Syn1 promoter comprising the nucleic acid sequence set forth in SEQ ID NO:04.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47 or a fragment thereof, which is operably linked to a Rgs5 promoter comprising the nucleic acid sequence set forth in SEQ ID NO:03.

Disclosed herein is AAV2g9 vector comprising a disclosed expression cassette comprising the nucleic acid sequence set forth in any one of SEQ ID NO:20-SEQ ID NO:33 or in any one of SEQ ID NO:35-SEQ ID NO:47, which is operably linked to a CBA promoter comprising the nucleic acid sequence set forth in SEQ ID NO:01.

### 3. Formulations

Disclosed herein is a pharmaceutical formulation comprising a disclosed vector and/or a disclosed isolated nucleic acid molecule. Disclosed herein is a pharmaceutical formulation comprising an isolated nucleic acid molecule comprising a nucleic acid sequence encoding a polypeptide capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising an isolated nucleic acid molecule comprising a nucleic acid sequence encoding a polypeptide capable of restoring one or more aspects of the glymphatic pathway. Disclosed herein is a pharmaceutical formulation comprising an isolated nucleic acid molecule comprising a nucleic acid sequence encoding a polypeptide capable of restoring one or more aspects of the water influx in the brain of a subject. Disclosed herein is a pharmaceutical formulation comprising a vector capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a vector capable of restoring one or more aspects of the glymphatic pathway. Disclosed herein is a pharmaceutical formulation comprising a vector capable of restoring one or more aspects of the water influx in the brain of a subject. Disclosed herein is a pharmaceutical formulation comprising a vector comprising an isolated nucleic acid molecule comprising a nucleic acid sequence encoding a polypeptide capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a vector comprising an isolated nucleic acid molecule comprising a nucleic acid sequence encoding a polypeptide capable of restoring one or more aspects of the glymphatic pathway, capable of restoring one or more aspects of the water influx in the brain of a subject, or both.

Disclosed herein is a pharmaceutical formulation comprising a rAAV vector capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a rAAV vector capable of restoring one or more aspects of the glymphatic pathway. Disclosed herein is a pharmaceutical formulation comprising a rAAV vector capable of restoring one or more aspects of the water influx in the brain of a subject. Disclosed herein is a pharmaceutical formulation comprising a rAAV vector comprising an isolated nucleic acid molecule comprising a nucleic acid sequence encoding a polypeptide capable of preventing protein aggregation in the brain of a subject, removing and/or clearing protein aggregates in the brain of a subject, improving fluid flux in the brain of a subject, or any combination thereof. Disclosed herein is a pharmaceutical formulation comprising a rAAV vector comprising an isolated nucleic acid molecule comprising a nucleic acid sequence encoding a polypeptide capable of restoring one or more aspects of the glymphatic pathway, capable of restoring one or more aspects of the water influx in the brain of a subject, or both.

In an aspect, a disclosed nucleic acid molecule can comprise the nucleotide sequence for a disclosed aquaporin. In an aspect, a disclosed aquaporin can comprise AQP1, AQP2, AQP3, AQP4, AQP5, AQP6, AQP7, AQP8, AQP9, AQP10, AQP11, AQP12, APQ13, or any combination thereof, or recombinant AQP1, AQP2, AQP3, AQP4, AQP5, AQP6, AQP7, AQP8, AQP9, AQP10, AQP11, AQP12, APQ13, or any combination thereof. In an aspect, a disclosed encoded aquaporin can comprise Aqp1, Aqp2, Aqp3, Aqp4, Aqp5, Aqp6, Apq7, Apq8, Apq9, Apq10, Apq11, Apq12, Apq13, or any combination thereof, or a recombinant Aqp1, Aqp2, Aqp3, Aqp4, Aqp5, Aqp6, Apq7, Apq8, Apq9, Apq10, Apq11, Apq12, Apq13, or any combination thereof. In an aspect, a disclosed encoded aquaporin can comprise Aqp1, Aqp4, Aqp5, Apq6, or Apq8 or a recombinant Aqp1, Aqp4, Aqp5, Apq6, or Apq8. In an aspect, a disclosed encoded aquaporin can comprise Aqp4 or a recombinant Aqp4.

In an aspect, a disclosed pharmaceutical formulation can restore one or more aspects of the glymphatic pathway, can restore one or more aspects of the water influx in the brain of a subject, or both. In an aspect, a disclosed pharmaceutical formulation can prevent protein aggregation in the brain of a subject, can remove and/or clear protein aggregates in the brain of a subject, can improve fluid flux in the brain of a subject, or any combination thereof. In an aspect, a disclosed pharmaceutical formulation can treat a neurodegenerative disease characterized by protein aggregation. In an aspect, a disclosed pharmaceutical formulation can treat Alzheimer's disease. In an aspect, a disclosed pharmaceutical formulation can clear and/or remove NFTs and/or AP plaques from a subject's brain. In an aspect, a disclosed pharmaceutical formulation can prevent and/or slow formation of NFTs and/or AP plaques in a subject's brain. In an aspect, a disclosed pharmaceutical formulation can treat Parkinson's disease. In an aspect, a disclosed pharmaceutical formulation can clear and/or remove Lewy bodies and/or α-synuclein aggregates from a subject's brain. In an aspect, a disclosed pharmaceutical formulation can prevent and/or slow formation of Lewy bodies and/or α-synuclein aggregates in a subject's brain.

In an aspect, a disclosed formulation can comprise (i) one or more active agents, (ii) biologically active agents, (iii) one or more pharmaceutically active agents, (iv) one or more immune-based therapeutic agents, (v) one or more immune modulators, (vi) one or more clinically approved agents, or (vii) a combination thereof. In an aspect, a disclosed composition can comprise one or more proteasome inhibitors.

In an aspect, a disclosed formulation can be packaged into some type of container and/or device for distribution and/or or administration.

### 4. Plasmids

Disclosed herein is a plasmid comprising one or more disclosed isolated nucleic acids and one or more disclosed promoters.

Disclosed herein is a plasmid comprising a disclosed expression cassette comprising a disclosed nucleic acid sequence operably linked to one or more disclosed expression control elements and/or message stabilizing elements. As known to the art, a functional expression unit that is capable of properly driving the expression of an incorporated polynucleotide (i.e., a nucleic acid sequence encoding an aquaporin) can be referred to as an “expression cassette”. The skilled person knows how to design an expression cassette to allow the expression in a eukaryotic cell, such as preferably in a mammalian or human cell.

Disclosed herein is a plasmid comprising a nucleic acid sequence encoding the aquaporin sequence set forth in any one of SEQ ID NO:07-SEQ ID NO:19 or a fragment thereof.

In an aspect, a plasmid comprising a nucleic acid sequence encoding a CBA promoter and Aqp4 ORF can comprise the CBA promoter sequence set forth in SEQ ID NO:01 or a sequence having at least 30%, at least 40%, at least 50%, at least 60%, at least 70%, at least 80%, or at least 90% identity to the sequence set forth in SEQ ID NO:01, and an Aqp4 sequence set forth in SEQ ID NO:23 or a sequence having at least 30%, at least 40%, at least 50%, at least 60%, at least 70%, at least 80%, or at least 90% identity to the sequence set forth in SEQ ID NO:23.

In an aspect, a plasmid comprising a nucleic acid sequence encoding a GFAP promoter and an Aqp4 ORF can comprise the GFAP promoter sequence set forth in SEQ ID NO:02 or a sequence having at least 30%, at least 40%, at least 50%, at least 60%, at least 70%, at least 80%, or at least 90% identity to the sequence set forth in SEQ ID NO:02, and an Aqp4 sequence set forth in SEQ ID NO:23 or a sequence having at least 30%, at least 40%, at least 50%, at least 60%, at least 70%, at least 80%, or at least 90% identity to the sequence set forth in SEQ ID NO:23.

In an aspect, a plasmid comprising a nucleic acid sequence encoding a Rgs5 promoter and Aqp4 ORF can comprise the Rgs5 promoter sequence set forth in SEQ ID NO:03 or a sequence having at least 30%, at least 40%, at least 50%, at least 60%, at least 70%, at least 80%, or at least 90% identity to the sequence set forth in SEQ ID NO:03, and an Aqp4 sequence set forth in SEQ ID NO:23 or a sequence having at least 30%, at least 40%, at least 50%, at least 60%, at least 70%, at least 80%, or at least 90% identity to the sequence set forth in SEQ ID NO:23.

In an aspect, a plasmid comprising a nucleic acid sequence encoding a Syn1 promoter and Aqp4 ORF can comprise the Syn1 promoter sequence set forth in SEQ ID NO:04 or a sequence having at least 30%, at least 40%, at least 50%, at least 60%, at least 70%, at least 80%, or at least 90% identity to the sequence set forth in SEQ ID NO:04, and an Aqp4 sequence set forth in SEQ ID NO:23 or a sequence having at least 30%, at least 40%, at least 50%, at least 60%, at least 70%, at least 80%, or at least 90% identity to the sequence set forth in SEQ ID NO:23.

### 5. Cells

Disclosed herein are cells comprising a disclosed isolated nucleic acid molecule, a disclosed vector, and/or a disclosed plasmid. Cells are known to the art. In an aspect, a disclosed cell can comprise a plasmid having the aquaporin sequence set forth in any one of SEQ ID NO:20-SEQ ID NO:33 or a sequence having at least 30%, at least 40%, at least 50%, at least 60%, at least 70%, at least 80%, or at least 90% identity to the sequence set forth in any one of SEQ ID NO:20-SEQ ID NO:33.

In an aspect, a disclosed cell can comprise the plasmid having the aquaporin sequence set forth in any one of SEQ ID NO:35-SEQ ID NO:47 or a sequence having at least 30%, at least 40%, at least 50%, at least 60%, at least 70%, at least 80%, or at least 90% identity to the sequence set forth in any one of SEQ ID NO:35-SEQ ID NO:47.

### 6. Animals

Disclosed herein are animals treated with one or more disclosed isolated nucleic acid molecules, disclosed vectors, disclosed pharmaceutical formulations, and/or disclosed plasmids. In an aspect, a disclosed animal such as, for example, an aquaporin knockout mouse has been treated with a disclosed isolated nucleic acid molecule, disclosed vector, disclosed pharmaceutical formulation, and/or disclosed plasmid.

### 7. Kits

Disclosed herein is a kit comprising a disclosed isolated nucleic acid molecule, a disclosed vector, a disclosed pharmaceutical formulation, or a combination thereof. In an aspect, a kit can comprise a disclosed isolated nucleic acid molecule, a disclosed vector, a disclosed pharmaceutical formulation, or a combination thereof, and one or more agents.

“Agents” and “Therapeutic Agents” are known to the art and are described supra. In an aspect, the one or more agents can treat, prevent, inhibit, and/or ameliorate one or more comorbidities in a subject. In an aspect, one or more active agents can treat, inhibit, prevent, and/or ameliorate protein aggregation in the brain, symptoms associated with protein aggregation in the brain, and/or complications associated with protein aggregation in the brain.

In an aspect, a disclosed kit can comprise at least two components constituting the kit. Together, the components constitute a functional unit for a given purpose such as, for example, treating a subject diagnosed with a protein aggregating disease in the brain or suspected of having a protein aggregating disease in the brain (e.g., Alzheimer's disease, Parkinson's disease, etc.).

Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed isolated nucleic acid molecule, a disclosed vector, a disclosed pharmaceutical formulation, or a combination thereof, and a label or package insert with instructions for use. In an aspect, suitable containers include, for example, bottles, vials, syringes, blister pack, etc. The containers can be formed from a variety of materials such as glass or plastic. The container can hold a disclosed isolated nucleic acid molecule, a disclosed vector, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed isolated nucleic acid molecule, a disclosed vector, a disclosed pharmaceutical formulation, or a combination thereof can be used for treating, inhibiting, preventing, and/or ameliorating protein aggregation in the brain, symptoms associated with protein aggregation in the brain, and/or complications associated with protein aggregation in the brain. A kit can comprise additional components necessary for administration such as, for example, other buffers, diluents, filters, needles, and syringes.

### A. Methods of Preventing Protein Aggregation

Disclosed herein is a method of preventing protein aggregation in the brain of a subject, the method comprising administering to a subject in need thereof a therapeutically effective amount of a vector comprising an isolated nucleic acid sequence encoding an aquaporin.

In an aspect, the expression of the encoded aquaporin can prevent protein aggregation in the subject's brain. In an aspect, the expression of the encoded aquaporin can clear and/or remove protein aggregates from the subject's brain. In an aspect, the expression of the encoded aquaporin can improve fluid flux in the subject's brain. In an aspect, the improved fluid flux can prevent protein aggregation in the subject's brain, can clear and/or remove protein aggregates from the subject's brain, or the combination thereof.

In an aspect, a disclosed method can restore one or more aspects of the glymphatic pathway, can restore one or more aspects of the water influx in the brain of a subject, or both. In an aspect, a disclosed method can further comprise removing and/or clearing protein aggregates in the brain of a subject. In an aspect, a disclosed method can further comprise improving fluid flux in the brain of a subject.

In an aspect of a disclosed method, a subject has been diagnosed with a neurodegenerative disease characterized by protein aggregation, is suspected of having a neurodegenerative disease characterized by protein aggregation, or is at risk of developing a neurodegenerative disease characterized by protein aggregation. In an aspect, a subject has been diagnosed with Alzheimer's disease, is suspected of having Alzheimer's disease, or is at risk of developing Alzheimer's disease. In an aspect of a disclosed method, protein aggregates can comprise NFTs and/or β-amyloid plaques. In an aspect, a subject has been diagnosed with Parkinson's disease, is suspected of having Parkinson's disease, or is at risk of developing Parkinson's disease. In an aspect of a disclosed method, protein aggregates can comprise Lewy bodies and/or α-synuclein.

In an aspect, a disclosed vector can comprise an isolated nucleic acid molecule encoding an disclosed aquaporin. In an aspect, a disclosed nucleic acid molecule encoding the aquaporin can be CpG-depleted and/or codon-optimized for expression in a human cell.

In an aspect, a disclosed method can further comprise administering to the subject a therapeutically effective amount of a therapeutic agent. Therapeutic agents are known to the art.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof

In an aspect, a disclosed method can further comprise repeating the administering of the vector one or more times. In an aspect, a disclosed method can further comprise repeating the administering of the therapeutic agent one or more times. In an aspect, a disclosed method can further comprise repeating the administering of the one or more immune modulators one or more times.

In an aspect, a disclosed method can further comprise monitoring the subject for adverse effects. In an aspect, wherein in the absence of adverse effects, the method can further comprise continuing to treat the subject. In an aspect, wherein in the presence of adverse effects, the method can further comprise modifying one or more steps of the method. In an aspect, modifying can comprise modifying the treating step, modifying the administering step, or both.

In an aspect, a disclosed method can further comprise improving the subject's cognitive function. In an aspect, improving cognitive function can comprise improving memory function, improving the ability to plan, improving the ability to solve problems, improving performance of familiar tasks, improving recognition of faces and places, improving the understanding of visual images and spatial relationships, improving the ability to speak, improving the ability to write, improving decision-making, and/or improving judgment.

In an aspect, a disclosed nucleic acid molecule can be packaged into a viral or non-viral vector. In an aspect, a disclosed non-viral vector can comprise a polymer-based vector, a peptide-based vector, a lipid nanoparticle, a solid lipid nanoparticle, or a cationic lipid-based vector. In an aspect, a disclosed viral vector can comprise an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can comprise bovine AAV, caprine AAV, canine AAV, equine AAV, ovine AAV, avian AAV, primate AAV, or non-primate AAV.

In an aspect, a disclosed AAV vector can comprise a recombinant AAV (rAAV) vector. In an aspect, a disclosed AAV vector can be self-complementary.

In an aspect, a disclosed AAV vector or disclosed recombinant rAAV vector can comprise AAV1, AAV2, AAV3 (including 3a and 3b), AAV4, AAVS, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed AAV vector can comprise AAV2g9. In an aspect, a disclosed AAV2g9 can comprise the chimera described in Shen S, et al. (2013) J Biol Chem. 288(40):28814-28823. In an aspect, a disclosed AAV vector can comprise AAV-cc47.

In an aspect, administering a therapeutically effective amount of disclosed vector can comprise administering a range of about 1×10¹⁰ vg/kg to about 2×10¹⁴ vg/kg. In an aspect, administering a therapeutically effective amount of disclosed vector can comprise administering a dose of about 1×10¹¹ vg/kg to about 8×10¹³ vg/kg or about 1×10¹² vg/kg to about 8×10¹³ vg/kg. In an aspect, administering a therapeutically effective amount of disclosed vector can comprise administering a dose of about 1×10¹³ vg/kg to about 6×10¹³ vg/kg. In an aspect, administering a therapeutically effective amount of disclosed vector can comprise administering a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, administering a therapeutically effective amount of disclosed vector can comprise administering a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³ vg/kg, or no more than about 1×10¹⁴ vg/kg. In an aspect, administering a therapeutically effective amount of disclosed vector can comprise administering a dose of about 1×10¹² vg/kg. In an aspect, a disclosed vector can be administered at a dose of about 1×10¹¹ vg/kg.

In an aspect of a disclosed method, administering a disclosed vector can be administered via intravenous administration, intra-CSF administration, intracerebroventricular (ICV) administration, intraventricular administration, intra-cisterna magna (ICM) administration, intraparenchymal administration, intrathecal (lumbar, cisternal, or both) administration, or a combination thereof.

In an aspect of a disclosed method, administering a disclosed vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed vector can be administered via multiple routes of administration.

In an aspect, a disclosed nucleic acid molecule can comprise the nucleotide sequence for AQP1, AQP2, AQP3, AQP4, AQP5, AQP6, AQP7, AQP8, AQP9, AQP10, AQP11, AQP12, APQ13, or any combination thereof. In an aspect, a disclosed nucleic acid molecule can comprise the nucleotide sequence for recombinant AQP1, AQP2, AQP3, AQP4, AQP5, AQP6, AQP7, AQP8, AQP9, AQP10, AQP11, AQP12, APQ13, or any combination thereof.

In an aspect, a disclosed AAV vector or a disclosed rAAV vector can comprise a promoter operably linked to the nucleic acid sequence.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed aquaporin) or some other enzyme involved in glymphatic transport or metabolism). In an aspect, a disclosed endogenous promoter or a disclosed endogenous promoter/enhancer can be used for constitutive and efficient expression of a disclosed gene (e.g., a nucleic acid sequence encoding an aquaporin).

In an aspect, a disclosed endogenous promoter or a disclosed endogenous promoter/enhancer can be used for constitutive and efficient expression of an aquaporin. In an aspect, a disclosed endogenous promoter or a disclosed endogenous promoter/enhancer can comprise the promoter for the gene encoding Aqp1, Aqp2, Aqp3, Aqp4, Aqp5, Aqp6, Aqp7, Aqp8, Aqp9, Aqp10, Aqp11, Aqp12, or Aqp 13. For example, in an aspect, when an encoded polypeptide comprises the Aqp1, the disclosed endogenous promoter or a disclosed endogenous promoter/enhancer can comprise the promoter or promoter/enhancer for the gene encoding Aqp1. Similarly, when an encoded polypeptide comprises Aqp4, the disclosed endogenous promoter or a disclosed endogenous promoter/enhancer can comprise the promoter or promoter/enhancer for the gene encoding Aqp4. For example, in an aspect, a disclosed endogenous promoter or a disclosed endogenous promoter/enhancer can comprise the sequence set forth in SEQ ID NO:06 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the endogenous promoter sequence for any one of Aqp1, Aqp2, Aqp3, Aqp4, Aqp5, Aqp6, Aqp7, Aqp8, Aqp9, Aqp10, Aqp11, Aqp12, or Aqp 13.

In an aspect, a disclosed viral vector can comprise a disclosed nucleic acid molecule encoding a disclosed aquaporin, a disclosed brain cell specific promoter, a pair of ITRs, and a polyadenylation sequence.

### B. Methods of Clearing and/or Removing Protein Aggregates

Disclosed herein is a method of removing and/or clearing protein aggregates in the brain of a subject, the method comprising administering to a subject in need thereof a therapeutically effective amount of a vector comprising an isolated nucleic acid sequence encoding an aquaporin.

In an aspect, the expression of the encoded aquaporin can clear and/or remove protein aggregates from the subject's brain. In an aspect, the expression of the encoded aquaporin can prevent protein aggregation in the subject's brain. In an aspect, the expression of the encoded aquaporin can improve fluid flux in the subject's brain. In an aspect, the improved fluid flux can prevent protein aggregation in the subject's brain, can clear and/or remove protein aggregates from the subject's brain, or the combination thereof.

In an aspect, a disclosed method can restore one or more aspects of the glymphatic pathway, can restore one or more aspects of the water influx in the brain of a subject, or both. In an aspect, a disclosed method can further comprise removing and/or clearing protein aggregates in the brain of a subject. In an aspect, a disclosed method can further comprise improving fluid flux in the brain of a subject.

In an aspect of a disclosed method, a subject has been diagnosed with a neurodegenerative disease characterized by protein aggregation, is suspected of having a neurodegenerative disease characterized by protein aggregation, or is at risk of developing a neurodegenerative disease characterized by protein aggregation. In an aspect, a subject has been diagnosed with Alzheimer's disease, is suspected of having Alzheimer's disease, or is at risk of developing Alzheimer's disease. In an aspect of a disclosed method, protein aggregates can comprise NFTs and/or β-amyloid plaques. In an aspect, a subject has been diagnosed with Parkinson's disease, is suspected of having Parkinson's disease, or is at risk of developing Parkinson's disease. In an aspect of a disclosed method, protein aggregates can comprise Lewy bodies and/or α-synuclein.

In an aspect, a disclosed vector can comprise an isolated nucleic acid molecule encoding an disclosed aquaporin. In an aspect, a disclosed nucleic acid molecule encoding the aquaporin can be CpG-depleted and/or codon-optimized for expression in a human cell.

In an aspect, a disclosed method can further comprise administering to the subject a therapeutically effective amount of a therapeutic agent. Therapeutic agents are known to the art.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

In an aspect, a disclosed method can further comprise repeating the administering of the vector one or more times. In an aspect, a disclosed method can further comprise repeating the administering of the therapeutic agent one or more times. In an aspect, a disclosed method can further comprise repeating the administering of the one or more immune modulators one or more times.

In an aspect, a disclosed method can further comprise monitoring the subject for adverse effects. In an aspect, wherein in the absence of adverse effects, the method can further comprise continuing to treat the subject. In an aspect, wherein in the presence of adverse effects, the method can further comprise modifying one or more steps of the method. In an aspect, modifying can comprise modifying the treating step, modifying the administering step, or both.

In an aspect, a disclosed method can further comprise improving the subject's cognitive function. In an aspect, improving cognitive function can comprise improving memory function, improving the ability to plan, improving the ability to solve problems, improving performance of familiar tasks, improving recognition of faces and places, improving the understanding of visual images and spatial relationships, improving the ability to speak, improving the ability to write, improving decision-making, and/or improving judgment.

In an aspect, a disclosed nucleic acid molecule can be packaged into a viral or non-viral vector. In an aspect, a disclosed non-viral vector can comprise a polymer-based vector, a peptide-based vector, a lipid nanoparticle, a solid lipid nanoparticle, or a cationic lipid-based vector. In an aspect, a disclosed viral vector can comprise an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can comprise bovine AAV, caprine AAV, canine AAV, equine AAV, ovine AAV, avian AAV, primate AAV, or non-primate AAV.

In an aspect, a disclosed AAV vector can comprise a recombinant AAV (rAAV) vector. In an aspect, a disclosed AAV vector can be self-complementary.

In an aspect, a disclosed AAV vector or disclosed recombinant rAAV vector can comprise AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed AAV vector can comprise AAV2g9. In an aspect, a disclosed AAV2g9 can comprise the chimera described in Shen S, et al. (2013) J Biol Chem. 288(40):28814-28823. In an aspect, a disclosed AAV vector can comprise AAV-cc47.

In an aspect, administering a therapeutically effective amount of disclosed vector can comprise administering a range of about 1×10¹⁰ vg/kg to about 2×10¹⁴ vg/kg. In an aspect, administering a therapeutically effective amount of disclosed vector can comprise administering a dose of about 1×10¹¹ vg/kg to about 8×10¹³ vg/kg or about 1×10¹² vg/kg to about 8×10¹³ vg/kg. In an aspect, administering a therapeutically effective amount of disclosed vector can comprise administering a dose of about 1×10¹³ vg/kg to about 6×10¹³ vg/kg. In an aspect, administering a therapeutically effective amount of disclosed vector can comprise administering a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, administering a therapeutically effective amount of disclosed vector can comprise administering a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³ vg/kg, or no more than about 1×10¹⁴ vg/kg. In an aspect, administering a therapeutically effective amount of disclosed vector can comprise administering a dose of about 1×10¹² vg/kg. In an aspect, a disclosed vector can be administered at a dose of about 1×10¹¹ vg/kg.

In an aspect of a disclosed method, administering a disclosed vector can be administered via intravenous administration, intra-CSF administration, intracerebroventricular (ICV) administration, intraventricular administration, intra-cisterna magna (ICM) administration, intraparenchymal administration, intrathecal (lumbar, cisternal, or both) administration, or a combination thereof.

In an aspect of a disclosed method, administering a disclosed vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed vector can be administered via multiple routes of administration.

In an aspect, a disclosed nucleic acid molecule can comprise the nucleotide sequence for AQP1, AQP2, AQP3, AQP4, AQPS, AQP6, AQP7, AQP8, AQP9, AQP10, AQP11, AQP12, APQ13, or any combination thereof. In an aspect, a disclosed nucleic acid molecule can comprise the nucleotide sequence for recombinant AQP1, AQP2, AQP3, AQP4, AQP5, AQP6, AQP7, AQP8, AQP9, AQP10, AQP11, AQP12, APQ13, or any combination thereof

In an aspect, a disclosed AAV vector or a disclosed rAAV vector can comprise a promoter operably linked to the nucleic acid sequence.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed aquaporin) or some other enzyme involved in glymphatic transport or metabolism). In an aspect, a disclosed endogenous promoter or a disclosed endogenous promoter/enhancer can be used for constitutive and efficient expression of a disclosed gene (e.g., a nucleic acid sequence encoding an aquaporin).

In an aspect, a disclosed endogenous promoter or a disclosed endogenous promoter/enhancer can be used for constitutive and efficient expression of an aquaporin. In an aspect, a disclosed endogenous promoter or a disclosed endogenous promoter/enhancer can comprise the promoter or the promoter/enhancer for the gene encoding AQP1, AQP2, AQP3, AQP4, AQP5, AQP6, AQP7, AQP8, AQP9, AQP10, AQP11, AQP12, APQ13, or any combination thereof. For example, in an aspect, a disclosed endogenous promoter or a disclosed endogenous promoter/enhancer can comprise the sequence set forth in SEQ ID NO:06 or a fragment thereof. In an aspect, a disclosed endogenous promoter or a disclosed endogenous promoter/enhancer can be used for constitutive and efficient expression of an aquaporin. In an aspect, a disclosed endogenous promoter or a disclosed endogenous promoter/enhancer can comprise the promoter for the gene encoding Aqp1, Aqp2, Aqp3, Aqp4, Aqp5, Aqp6, Aqp7, Aqp8, Aqp9, Aqp10, Aqp11, Aqp12, or Aqp 13. For example, in an aspect, when an encoded polypeptide comprises the Aqp1, the disclosed endogenous promoter or a disclosed endogenous promoter/enhancer can comprise the promoter or promoter/enhancer for the gene encoding Aqp1. Similarly, when an encoded polypeptide comprises Aqp4, the disclosed endogenous promoter or a disclosed endogenous promoter/enhancer can comprise the promoter or promoter/enhancer for the gene encoding Aqp4. For example, in an aspect, a disclosed endogenous promoter or a disclosed endogenous promoter/enhancer can comprise the sequence set forth in SEQ ID NO:06 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the endogenous promoter sequence for any one of Aqp1, Aqp2, Aqp3, Aqp4, Aqp5, Aqp6, Aqp7, Aqp8, Aqp9, Aqp10, Aqp11, Aqp12, or Aqp 13.

In an aspect, a disclosed viral vector can comprise a disclosed nucleic acid molecule encoding a disclosed aquaporin, a disclosed brain cell specific promoter, a pair of ITRs, and a polyadenylation sequence.

### C. Methods of Improving Fluid Flux in the Brain

Disclosed herein is a method of improving fluid flux in the brain of a subject, the method comprising administering to a subject in need thereof a therapeutically effective amount of a vector comprising an isolated nucleic acid sequence encoding an aquaporin.

In an aspect, the expression of the encoded aquaporin can improve fluid flux in the subject's brain. In an aspect, the improved fluid flux can prevent protein aggregation in the subject's brain, can clear and/or remove protein aggregates from the subject's brain, or the combination thereof. In an aspect, the expression of the encoded aquaporin can clear and/or remove protein aggregates from the subject's brain. In an aspect, the expression of the encoded aquaporin can prevent protein aggregation in the subject's brain.

In an aspect, a disclosed method can restore one or more aspects of the glymphatic pathway, can restore one or more aspects of the water influx in the brain of a subject, or both. In an aspect, a disclosed method can further comprise removing and/or clearing protein aggregates in the brain of a subject. In an aspect, a disclosed method can further comprise improving fluid flux in the brain of a subject.

In an aspect of a disclosed method, a subject has been diagnosed with a neurodegenerative disease characterized by protein aggregation, is suspected of having a neurodegenerative disease characterized by protein aggregation, or is at risk of developing a neurodegenerative disease characterized by protein aggregation. In an aspect, a subject has been diagnosed with Alzheimer's disease, is suspected of having Alzheimer's disease, or is at risk of developing Alzheimer's disease. In an aspect of a disclosed method, protein aggregates can comprise NFTs and/or β-amyloid plaques. In an aspect, a subject has been diagnosed with Parkinson's disease, is suspected of having Parkinson's disease, or is at risk of developing Parkinson's disease. In an aspect of a disclosed method, protein aggregates can comprise Lewy bodies and/or α-synuclein.

In an aspect, a disclosed vector can comprise an isolated nucleic acid molecule encoding a disclosed aquaporin. In an aspect, a disclosed nucleic acid molecule encoding the aquaporin can be CpG-depleted and/or codon-optimized for expression in a human cell.

In an aspect, a disclosed method can further comprise administering to the subject a therapeutically effective amount of a therapeutic agent. Therapeutic agents are known to the art.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

In an aspect, a disclosed method can further comprise repeating the administering of the vector one or more times. In an aspect, a disclosed method can further comprise repeating the administering of the therapeutic agent one or more times. In an aspect, a disclosed method can further comprise repeating the administering of the one or more immune modulators one or more times.

In an aspect, a disclosed method can further comprise monitoring the subject for adverse effects. In an aspect, wherein in the absence of adverse effects, the method can further comprise continuing to treat the subject. In an aspect, wherein in the presence of adverse effects, the method can further comprise modifying one or more steps of the method. In an aspect, modifying can comprise modifying the treating step, modifying the administering step, or both.

In an aspect, a disclosed method can further comprise improving the subject's cognitive function. In an aspect, improving cognitive function can comprise improving memory function, improving the ability to plan, improving the ability to solve problems, improving performance of familiar tasks, improving recognition of faces and places, improving the understanding of visual images and spatial relationships, improving the ability to speak, improving the ability to write, improving decision-making, and/or improving judgment.

In an aspect, a disclosed nucleic acid molecule can be packaged into a viral or non-viral vector. In an aspect, a disclosed non-viral vector can comprise a polymer-based vector, a peptide-based vector, a lipid nanoparticle, a solid lipid nanoparticle, or a cationic lipid-based vector. In an aspect, a disclosed viral vector can comprise an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can comprise bovine AAV, caprine AAV, canine AAV, equine AAV, ovine AAV, avian AAV, primate AAV, or non-primate AAV.

In an aspect, a disclosed AAV vector can comprise a recombinant AAV (rAAV) vector. In an aspect, a disclosed AAV vector can be self-complementary.

In an aspect, a disclosed AAV vector or disclosed recombinant rAAV vector can comprise AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed AAV vector can comprise AAV2g9. In an aspect, a disclosed AAV2g9 can comprise the chimera described in Shen S, et al. (2013) J Biol Chem. 288(40):28814-28823. In an aspect, a disclosed AAV vector can comprise AAV-cc47.

In an aspect, administering a therapeutically effective amount of disclosed vector can comprise administering a range of about 1×10¹⁰ vg/kg to about 2×10¹⁴ vg/kg. In an aspect, administering a therapeutically effective amount of disclosed vector can comprise administering a dose of about 1×10¹¹ vg/kg to about 8×10¹³ vg/kg or about 1×10¹² vg/kg to about 8×10¹³ vg/kg. In an aspect, administering a therapeutically effective amount of disclosed vector can comprise administering a dose of about 1×10¹³ vg/kg to about 6×10¹³ vg/kg. In an aspect, administering a therapeutically effective amount of disclosed vector can comprise administering a dose of at least about 1×10 10 vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, administering a therapeutically effective amount of disclosed vector can comprise administering a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³ vg/kg, or no more than about 1×10¹⁴ vg/kg. In an aspect, administering a therapeutically effective amount of disclosed vector can comprise administering a dose of about 1×10¹² vg/kg. In an aspect, a disclosed vector can be administered at a dose of about 1×10¹¹ vg/kg.

In an aspect of a disclosed method, administering a disclosed vector can be administered via intravenous administration, intra-CSF administration, intracerebroventricular (ICV) administration, intraventricular administration, intra-cisterna magna (ICM) administration, intraparenchymal administration, intrathecal (lumbar, cisternal, or both) administration, or a combination thereof.

In an aspect of a disclosed method, administering a disclosed vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed vector can be administered via multiple routes of administration.

In an aspect, a disclosed nucleic acid molecule can comprise the nucleotide sequence for AQP1, AQP2, AQP3, AQP4, AQPS, AQP6, AQP7, AQP8, AQP9, AQP10, AQP11, AQP12, APQ13, or any combination thereof. In an aspect, a disclosed nucleic acid molecule can comprise the nucleotide sequence for recombinant AQP1, AQP2, AQP3, AQP4, AQP5, AQP6, AQP7, AQP8, AQP9, AQP10, AQP11, AQP12, APQ13, or any combination thereof

In an aspect, a disclosed AAV vector or a disclosed rAAV vector can comprise a promoter operably linked to the nucleic acid sequence.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed aquaporin) or some other enzyme involved in glymphatic transport or metabolism). In an aspect, a disclosed endogenous promoter or a disclosed endogenous promoter/enhancer can be used for constitutive and efficient expression of a disclosed gene (e.g., a nucleic acid sequence encoding an aquaporin).

In an aspect, a disclosed endogenous promoter or a disclosed endogenous promoter/enhancer can be used for constitutive and efficient expression of an aquaporin. In an aspect, a disclosed endogenous promoter or a disclosed endogenous promoter/enhancer can comprise the promoter for the gene encoding Aqp1, Aqp2, Aqp3, Aqp4, Aqp5, Aqp6, Aqp7, Aqp8, Aqp9, Aqp10, Aqp11, Aqp12, or Aqp 13. For example, in an aspect, when an encoded polypeptide comprises the Aqp1, the disclosed endogenous promoter or a disclosed endogenous promoter/enhancer can comprise the promoter or promoter/enhancer for the gene encoding Aqp1. Similarly, when an encoded polypeptide comprises Aqp4, the disclosed endogenous promoter or a disclosed endogenous promoter/enhancer can comprise the promoter or promoter/enhancer for the gene encoding Aqp4. For example, in an aspect, a disclosed endogenous promoter or a disclosed endogenous promoter/enhancer can comprise the sequence set forth in SEQ ID NO:06 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the endogenous promoter sequence for any one of Aqp1, Aqp2, Aqp3, Aqp4, Aqp5, Aqp6, Aqp7, Aqp8, Aqp9, Aqp10, Aqp11, Aqp12, or Aqp 13.

In an aspect, a disclosed viral vector can comprise a disclosed nucleic acid molecule encoding a disclosed aquaporin, a disclosed brain cell specific promoter, a pair of ITRs, and a polyadenylation sequence.

### D. Methods of Treating a Subject

Disclosed herein is a method of treating a subject, the method comprising administering to a subject in need thereof a therapeutically effective amount of a vector comprising an isolated nucleic acid molecule sequence encoding an aquaporin.

In an aspect, the expression of the encoded aquaporin can prevent protein aggregation in the subject's brain. In an aspect, the expression of the encoded aquaporin can clear and/or remove protein aggregates from the subject's brain. In an aspect, the expression of the encoded aquaporin can improve fluid flux in the subject's brain. In an aspect, the improved fluid flux can prevent protein aggregation in the subject's brain, can clear and/or remove protein aggregates from the subject's brain, or the combination thereof.

In an aspect, a disclosed method can restore one or more aspects of the glymphatic pathway, can restore one or more aspects of the water influx in the brain of a subject, or both. In an aspect, a disclosed method can further comprise removing and/or clearing protein aggregates in the brain of a subject. In an aspect, a disclosed method can further comprise improving fluid flux in the brain of a subject.

In an aspect of a disclosed method, a subject has been diagnosed with a neurodegenerative disease characterized by protein aggregation, is suspected of having a neurodegenerative disease characterized by protein aggregation, or is at risk of developing a neurodegenerative disease characterized by protein aggregation. In an aspect, a subject has been diagnosed with Alzheimer's disease, is suspected of having Alzheimer's disease, or is at risk of developing Alzheimer's disease. In an aspect of a disclosed method, protein aggregates can comprise NFTs and/or β-amyloid plaques. In an aspect, a subject has been diagnosed with Parkinson's disease, is suspected of having Parkinson's disease, or is at risk of developing Parkinson's disease. In an aspect of a disclosed method, protein aggregates can comprise Lewy bodies and/or α-synuclein.

In an aspect, a disclosed vector can comprise an isolated nucleic acid molecule encoding a disclosed aquaporin. In an aspect, a disclosed nucleic acid molecule encoding the aquaporin can be CpG-depleted and/or codon-optimized for expression in a human cell.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

In an aspect, a disclosed method can further comprise repeating the administering of the vector one or more times. In an aspect, a disclosed method can further comprise repeating the administering of the therapeutic agent one or more times. In an aspect, a disclosed method can further comprise repeating the administering of the one or more immune modulators one or more times.

In an aspect, a disclosed method can further comprise monitoring the subject for adverse effects. In an aspect, wherein in the absence of adverse effects, the method can further comprise continuing to treat the subject. In an aspect, wherein in the presence of adverse effects, the method can further comprise modifying one or more steps of the method. In an aspect, modifying can comprise modifying the treating step, modifying the administering step, or both.

In an aspect, a disclosed method can further comprise improving the subject's cognitive function. In an aspect, improving cognitive function can comprise improving memory function, improving the ability to plan, improving the ability to solve problems, improving performance of familiar tasks, improving recognition of faces and places, improving the understanding of visual images and spatial relationships, improving the ability to speak, improving the ability to write, improving decision-making, and/or improving judgment.

In an aspect, a disclosed nucleic acid molecule can be packaged into a viral or non-viral vector. In an aspect, a disclosed non-viral vector can comprise a polymer-based vector, a peptide-based vector, a lipid nanoparticle, a solid lipid nanoparticle, or a cationic lipid-based vector. In an aspect, a disclosed viral vector can comprise an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can comprise bovine AAV, caprine AAV, canine AAV, equine AAV, ovine AAV, avian AAV, primate AAV, or non-primate AAV.

In an aspect, a disclosed AAV vector can comprise a recombinant AAV (rAAV) vector. In an aspect, a disclosed AAV vector can be self-complementary.

In an aspect, a disclosed AAV vector or disclosed recombinant rAAV vector can comprise AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed AAV vector can comprise AAV2g9. In an aspect, a disclosed AAV2g9 can comprise the chimera described in Shen S, et al. (2013) J Biol Chem. 288(40):28814-28823. In an aspect, a disclosed AAV vector can comprise AAV-cc47.

In an aspect, administering a therapeutically effective amount of disclosed vector can comprise administering a range of about 1×10¹⁰ vg/kg to about 2×10¹⁴ vg/kg. In an aspect, administering a therapeutically effective amount of disclosed vector can comprise administering a dose of about 1×10¹¹ vg/kg to about 8×10¹³ vg/kg or about 1×10¹² vg/kg to about 8×10¹³ vg/kg. In an aspect, administering a therapeutically effective amount of disclosed vector can comprise administering a dose of about 1×10¹³ vg/kg to about 6×10¹³ vg/kg. In an aspect, administering a therapeutically effective amount of disclosed vector can comprise administering a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, administering a therapeutically effective amount of disclosed vector can comprise administering a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³ vg/kg, or no more than about 1×10¹⁴ vg/kg. In an aspect, administering a therapeutically effective amount of disclosed vector can comprise administering a dose of about 1×10¹² vg/kg. In an aspect, a disclosed vector can be administered at a dose of about 1×10¹¹ vg/kg.

In an aspect of a disclosed method, administering a disclosed vector can be administered via intravenous administration, intra-CSF administration, intracerebroventricular (ICV) administration, intraventricular administration, intra-cisterna magna (ICM) administration, intraparenchymal administration, intrathecal (lumbar, cisternal, or both) administration, or a combination thereof.

In an aspect of a disclosed method, administering a disclosed vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed vector can be administered via multiple routes of administration.

In an aspect, a disclosed nucleic acid molecule can comprise the nucleotide sequence for AQP1, AQP2, AQP3, AQP4, AQPS, AQP6, AQP7, AQP8, AQP9, AQP10, AQP11, AQP12, APQ13, or any combination thereof. In an aspect, a disclosed nucleic acid molecule can comprise the nucleotide sequence for recombinant AQP1, AQP2, AQP3, AQP4, AQP5, AQP6, AQP7, AQP8, AQP9, AQP10, AQP11, AQP12, APQ13, or any combination thereof

In an aspect, a disclosed AAV vector or a disclosed rAAV vector can comprise a promoter operably linked to the nucleic acid sequence.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed aquaporin) or some other enzyme involved in glymphatic transport or metabolism). In an aspect, a disclosed endogenous promoter or a disclosed endogenous promoter/enhancer can be used for constitutive and efficient expression of a disclosed gene (e.g., a nucleic acid sequence encoding an aquaporin).

In an aspect, a disclosed endogenous promoter or a disclosed endogenous promoter/enhancer can be used for constitutive and efficient expression of an aquaporin. In an aspect, a disclosed endogenous promoter or a disclosed endogenous promoter/enhancer can comprise the promoter for the gene encoding Aqp1, Aqp2, Aqp3, Aqp4, Aqp5, Aqp6, Aqp7, Aqp8, Aqp9, Aqp10, Aqp11, Aqp12, or Aqp 13. For example, in an aspect, when an encoded polypeptide comprises the Aqp1, the disclosed endogenous promoter or a disclosed endogenous promoter/enhancer can comprise the promoter or promoter/enhancer for the gene encoding Aqp1. Similarly, when an encoded polypeptide comprises Aqp4, the disclosed endogenous promoter or a disclosed endogenous promoter/enhancer can comprise the promoter or promoter/enhancer for the gene encoding Aqp4. For example, in an aspect, a disclosed endogenous promoter or a disclosed endogenous promoter/enhancer can comprise the sequence set forth in SEQ ID NO:06 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the endogenous promoter sequence for any one of Aqp1, Aqp2, Aqp3, Aqp4, Aqp5, Aqp6, Aqp7, Aqp8, Aqp9, Aqp10, Aqp11, Aqp12, or Aqp 13.

In an aspect, a disclosed viral vector can comprise a disclosed nucleic acid molecule encoding a disclosed aquaporin, a disclosed brain cell specific promoter, a pair of ITRs, and a polyadenylation sequence.

### VII. EXAMPLES

The Examples that follow are illustrative of specific embodiments of the invention, and various uses thereof. They set forth for explanatory purposes only and are not to be taken as limiting the invention.

Although AQP4 is the most abundant water channel in the brain, it has only detected in the plasma membrane of astrocytes and ependymal membranes since its discovery over two decades. Its location can be characterized as the cell surfaces of the blood-brain barrier (BBB) and cerebrospinal fluid (CSF)-brain barrier. Therefore, AQP4 is expressed in astrocyte foot processes surrounding capillaries, astrocyte processes which are comprised of the glial limiting membrane, ependymal cells and subependymal astrocytes (Nielsen S, et al. (1997) J. Neurosci. 17:171-180; Rash J E, et al. (1998) Proc. Natl. Acad. Sci. USA. 95:11981-11986). Reactive microglial cells also expressed AQP4 mRNA.

The polarized distribution of AQP4 depends on some proteins also with polarized expression in astrocytes. α-syntrophin, a member of the dystrophin associated protein complex, plays an important role in anchoring of AQP4 to astrocyte end-foot processes. (Neely J D, et al. (2001) Proc. Natl. Acad. Sci. USA. 98:14108-14113; Enger R, et al. (2012) Glia. 60:2018-2026). AQP4 monomers consist of six helical, membrane-spanning domains and two highly conserved Asn-Pro-Ala (NPA) motifs that create a narrow aqueous pathway (Papadopoulos M C, et al. (2013) Nat. Rev. Neurosci. 14:265-277). Like other aquaporins, AQP4 monomers also assemble as tetramers. Importantly, AQP4 tetramers further cluster in the plasma membrane forming crystal-like supramo-lecular assemblies, termed orthogonal arrays of particles (OAPs). OAPs can be visualized in membranes by freeze-fracture electron microscopy that are originally confirmed to be formed by AQP4 in AQP4-transfected Chinese hamster ovary cells. AQP4 has two major isoforms: M1 and M23, which are transcribed from two different initiation sites on the same gene. M1 is a relatively long isoform with translation initiation at Met-1, while M23 is a shorter one with translation initiation at Met-23.

### Example 1

### Characterization of AAV Genomic Cassettes for Aqp4 Expression

Several AAV genomic cassettes were constructed. The first cassette contained a ubiquitous promoter (chicken beta actin; CBA) to drive the expression of the human AQP4 open reading frame (ORF), which preceded a polyadenylation signal. (FIG. 1, upper left). The second cassette contained an astrocyte-specific promoter (GFAP) to drive the expression of the human AQP4 open reading frame (ORF), which preceded a polyadenylation signal. (FIG. 1, top right). The third cassette contained a pericyte-specific promoter (Rsg5) to drive the expression of the human AQP4 open reading frame (ORF), which preceded a polyadenylation signal. (FIG. 1, bottom left). The fourth cassette contained a neuron-specific promoter (Syn1) to drive the expression of the human AQP4 open reading frame (ORF), which preceded a polyadenylation signal. (FIG. 1, bottom right). Arrows indicate the start of transcription from the genomic cassette.

### Example 2

### AAV-Mediated Aqp4 Expression Promoted Clearance of Synthetic Aβ Deposits

While tau accumulation is a hallmark of advanced Alzheimer's disease (AD) pathology, disease is initiated by the accumulation of Aβ plaques in the neuronal ISF. Proper glymphatic fluid flux promotes the clearance of Aβ oligomers from the ISF, aiding in the prevention of Aβ plaque formation. Thus, whether the overexpression of Aqp4 enhanced glymphatic function and promoted clearance of Aβ plaques was examined.

P0 mouse pups were treated via intracerebroventricular (ICV) injection with either AAV9-GFP or AAV9 packaging Aqp4 under control of the ubiquitous chicken beta actin (CBA) promoter at a dose of 7×10⁹ vector genomes (vg)/animal. At P30, a fluorescently labeled synthetic Aβ peptide (Hilyte-555 Aβ) into the mouse striatum (FIG. 2A). Then, 45 min following the injection a fluorescently labeled synthetic Aβ peptide (Hilyte-555 Aβ), brains were harvested, and signal from nuclear staining DAPI (blue) and Aβ (red) were imaged for both the no injection group (FIG. 2B) and the AAV2g9 group (FIG. 2C). The % Aβ fluorescence was normalized to total brain area (FIG. 2D). These data show that the overall Aβ plaque area was significantly decreased in mice expressing Aqp4 compared to the control mice, indicating that AAV-mediated Aqp4 overexpression could mediate clearance of Aβ plaques from the CNS.

### Example 3

### Glymphatic Dysregulation Exacerbates Tau Accumulation in a Mouse Model of Alzheimer's Disease (AD)

The exact mechanisms underlying AD pathophysiology are unclear, however, the accumulation of Aβ plaques is an initiating event. Aβ plaque accumulation leads to a loss in synaptic and neuronal function and increased neuroinflammation. This triggers the accumulation of tau and activation of astrocyte and microglial immune cells in the CNS. Together, these events lead to widespread neurodegeneration resulting in cognitive impairment and death. Impaired glymphatic flux can contribute to lack of Aβ clearance from the ISF.

Aqp4KO mice are characterized in Solenov E, et al. (2004) Am J Physiol Cell Physiol. 286(2):C426-432. Adult AQP4KO mice display ˜70% reduction in interstitial solute clearance compared to wild type (WT) littermates. This effect was more pronounced in both WT and AQP4KO aged mice compared to their younger counterparts. Further, other groups have shown that AD mice lacking Aqp4 expression (5xFAD;AQP4KO) present with increased Aβ accumulation and exacerbated cognitive defects. Taken together, these data suggest that aging and Aqp4 dysregulation lead to an impairment in the ISF bulk flow preventing solute diffusion and perivascular drainage.

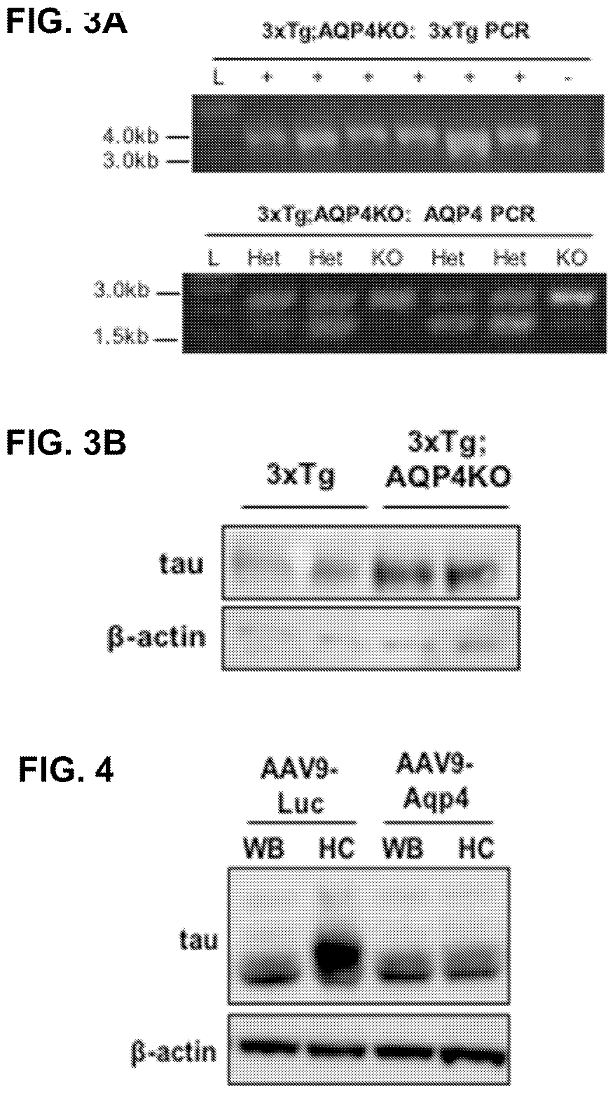

The work described herein developed additional rationale for connecting Aqp4 dysfunction to AD. To determine the role of glymphatic flux on tau accumulation in a mouse model of AD (3xTg), AD mice were crossed with AQP4KO mice to create an AD model lacking Aqp4 expression (3xTg;AQP4KO) (Jackson Lab, MMRRC Strain 034830-JAX). Briefly, these mice were generated as follows. Single-cell embryos from mice bearing the presenilin PS1M146V knock-in mutation on a mixed C57BL/6;129X1/SvJ;129S1/Sv genetic background (B6;129-Psen1^{tm1Mpm}) were co-injected with two independent mutant human transgenes; amyloid beta precursor protein (APPSwe) and microtubule-associated protein tau (tauP30IL). Both transgenes integrated at the same locus and are under the control of the mouse Thy1.2 regulatory element. Founder mice (line B1) were mated to B6;129-Psen1^{tm1Mpm} mice. Offspring from this cross were bred together, resulting in mice homozygous for all three alleles (3xTg-AD; homozygous for the Psen1 mutation and homozygous for the co-injected APPSwe and tauP301L transgenes (Tg(APPSwe,tauP301L)1Lfa)). Both male and female 3xTg-AD mice on the mixed C57BL/6;129X1/SvJ;129S1/Sv genetic background were sent to The Jackson Laboratory and bred together to establish this colony for the MMRRC. The transgene inserted on Chromosome 2 causing a 3 bp deletion. FIG. 3A shows the genotype confirmation of this mouse model. The level of tau accumulation was then assessed in whole brain lysates of the two models by Western blot. Strikingly, tau levels were much greater in 3xTg;AQP4KO animals at 4 months of age than 3xTg animals at 9 months of age. (FIG. 3B). The resulting data were consistent with results shown in the 5xFAD;AQP4KO model, there was increased AD pathology (tau accumulation) in animals with impaired glymphatic systems. These results indicate that improved glymphatic function can alleviate AD pathology, and importantly, underscore the involvement of Aqp4 in AD pathology. These data confirmed that restoring Aqp4 function can alleviate AD pathology.

### Example 4

### AAV-Aqp4 Gene Therapy Promoted Aβ Clearance in a Mouse Model of AD

Aqp4 is normally expressed on astrocytic endfeet. In response to age or disease, however, Aqp4 mislocalization results in impaired glymphatic flux. Thus, when designing a gene therapy vector for AD, the precise expression of Aqp4 in astrocytes must be considered.

To examine whether AAV9 vector packaging Aqp4 under control of the GFAP promoter could serve as a gene therapy strategy for the treatment of AD, PO 3xTg;AQP4KO mice pups were injected with AAV9-GFAP-Aqp4 or a control vector (AAV9-GFAP-luciferase) via ICV injection. At 4 months post-injection, levels of tau accumulation in whole brain lysates (WB) and hippocampal lysates (HC) were determined via Western blot (FIG. 4).

In the animals overexpressing Aqp4, the level of tau accumulation in the hippocampus was less than that of control animals overexpressing a control protein (luciferase). These data indicate that the enhancement of glymphatic function via Aqp4 overexpression promoted protein aggregate clearance in AD and improved pathological outcomes.

### Example 5

### AAV-Mediated Aqp4 Overexpression Decreased Aβ Accumulation

The 5xFAD Alzheimer's mouse model (Jackson Labs #034840) carries 5 human patient derived mutations across two genes important for production of amyloid: PSEN (Presinilin) and APP (Amyloid precursor protein). The transgenic mouse was generated by inserting cDNA for human PSEN and human APP with expression driven by a Thy1 promoter. As a result, within months, amyloid plaques begin accumulating in the mouse brain, and thus, this mouse was used to model plaque accumulation.

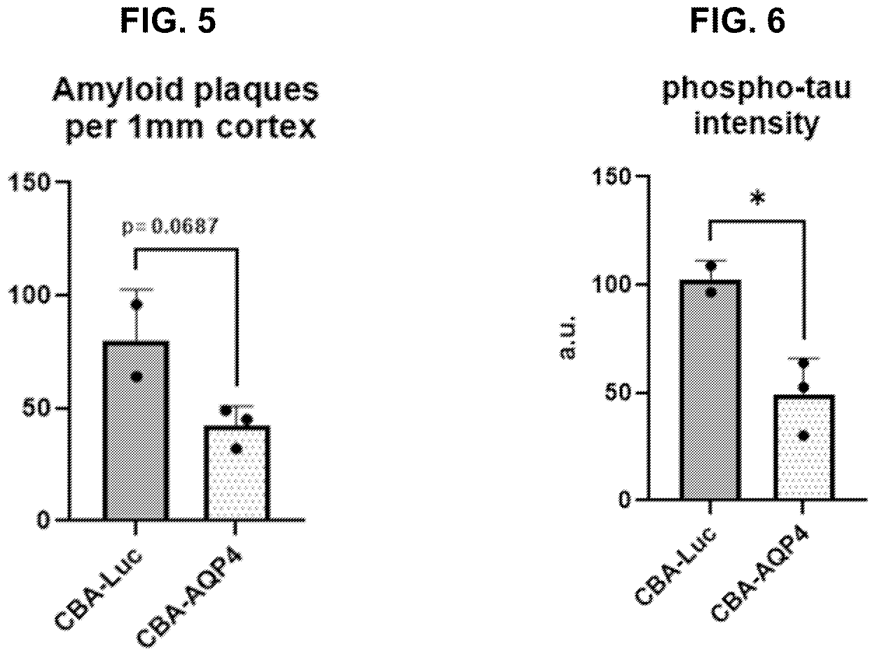

To determine whether increased CSF flux can impact plaque formation or removal, an AAV cassette was designed to overexpress Aquaporin-4 (AQP4) under a ubiquitous CBA promoter (FIG. 1). At P0, 5xFAD tg/tg pups (Jackson labs) were treated with recombinant AAV-cc47 packaging either CBA-AQP4 or CBA-Luciferase at 1×10¹⁰ vg/animal via intracerebroventricular injection. Following treatment with AAV-AQP4, the number of Aβ plaques per 1 mm of cortex. At 3 months of age, brains were harvested and the number of Aβ plaques analyzed (antibody: Millipore Sigma AB2286). Within 30 μm sagittal sections, the number of AP plaques across 1 mm of cortex rostral to the lateral ventricle were tallied and averaged across 4 or more sections per animal, then combined to measure the amount of plaque formation in either control or CBA-AQP4 treated conditions. Control CBA-Luciferase treated animals showed on average 80 plaques/mm cortex, while CBA-AQP4 treated animals showed on average 42 plaques/mm cortex. (FIG. 5, p<0.0687).

These data indicate that overexpression of AQP4 promoted plaque clearance in an Alzheimer's disease model, demonstrating that clearing aggregates by manipulating CSF flux is a promising treatment for Alzheimer's.

### Example 6

### AAV-Mediated Aqp4 Overexpression Significantly Decreased Phosphorylated Tau Accumulation

To determine whether increased CSF flux impacted plaque formation or plaque removal, an AAV cassette was designed to overexpress Aquaporin-4 (AQP4) under a ubiquitous CBA promoter. At P0, 5xFAD tg/tg pups were treated with recombinant AAV-cc47 packaging either CBA-AQP4 or CBA-Luciferase (control) at 1×10¹⁰ vg/animal via intracerebroventricular injection. At 3 months of age, brains were harvested and the intensity of phospho-tau was analyzed (Antibody: Thermo Fisher MN1020, AT8). 30 μm sagittal sections were converted to 8-bit, then thresholded at 17-18%. Intensity measurements were acquired in ImageJ. Background intensity was averaged across 5 measurements then subtracted from all measured cell intensity values. Averages per image per brain are of >40 cells per cortical section. (*p<0.05). Control CBA-Luciferase treated animals showed an average phospho-tau intensity of 103 (a.u.) per cell and CBA-AQP4 treated animals showed an average intensity of 49 (a.u.) per cell.

These data indicate that the overexpression of AQP4 suppressed tau phosphorylation and lowered tau burden in an Alzheimer's disease model. Thus, manipulating CSF flux affected tau phosphorylation status and served as a promising treatment for Alzheimer's disease.

### Example 7

### AAV-Mediated Cell-Specific Aqp4 Overexpression

To test whether increased CSF flux is driven by cell-type specific expression of AQP4, AAV cassettes are designed to express AQP4 specifically in pericytes, astrocytes, or neurons. As shown in FIG. 1, AAV constructs having the APQ4 ORF under the control of pericyte-specific promoter (Rgs5 promoter), astrocyte-specific promoter (GFAP promoter), and neuron-specific promoter (Syn1 promoter) are constructed.

P0 3xTg;AQP4KO mice pups are injected with the various constructs of FIG. 1 and a control vector (luciferase) via ICV injection. At 4 months post-injection, levels of tau accumulation in whole brain lysates (WB) and hippocampal lysates (HC) are determined via Western blot.

To determine whether increased CSF flux impacted plaque formation or plaque removal, an AAV cassette is designed to overexpress Aquaporin-4 (AQP4) under a ubiquitous CBA promoter. At P0, 5xFAD tg/tg pups were treated with recombinant AAV-cc47 packaging either CBA-AQP4, GFAP-AQP4, Rgs5-AQP4, Syn1-AQP4, or Luciferase (control) at 1×10¹⁰ vg/animal via intracerebroventricular injection. At 3 months of age, brains are harvested and the intensity of phospho-tau was analyzed (Antibody: Thermo Fisher MN1020, AT8). 30 μm sagittal sections are converted to 8-bit, then thresholded at 17-18%. Intensity measurements are acquired in ImageJ. Background intensity is averaged across 5 measurements and then subtracted from all measured cell intensity values. Averages per image per brain are of >40 cells per cortical section. (*p<0.05).

1. A method of treating a subject, the method comprising:
administering to a subject in need thereof a therapeutically effective amount of the AAV vector or recombinant AAV vector of claim 38, wherein, following the expression of the encoded aquaporin, fluid flux in the brain is improved.

2. (canceled)

3. The method of claim 1, wherein the improved fluid flux prevents protein aggregation in the subject's brain.

4. The method of claim 1, wherein the improved fluid flux clears and/or removes protein aggregates from the subject's brain.

5. The method of claim 1, wherein the subject has been diagnosed with Alzheimer's disease, is suspected of having Alzheimer's disease, or is at risk of developing Alzheimer's disease.

6. The method of claim 5, wherein the protein aggregates comprise neurofibrillary tangles and/or β-amyloid plaques.

7. The method of claim 1, wherein the encoded aquaporin comprises Aqp4 or a recombinant Aqp4.

8.-9. (canceled)

10. The method of claim 1, wherein the therapeutically effective amount of the vector comprises about 1×10¹⁰ vg/kg to about 2×10¹⁴ vg/kg.

11. The method of claim 1, wherein administering comprises intravenous administration, intra-CSF administration, intracerebroventricular (ICV) administration, intraventricular administration, intra-ci sterna magna (ICM) administration, intraparenchymal administration, intrathecal administration, or a combination thereof.

12-18. (canceled)

19. The method of claim 1, further comprising improving wherein the subject's cognitive function is improved.

20. The method of claim 19, wherein cognitive function comprises remembering, planning, solving problems, performing familiar tasks, recognizing faces and places, understanding visual images and spatial relationships, speaking, writing, decision-making, exercising judgment, or any combination thereof.

21.-37. (canceled)

38. An AAV vector or a recombinant AAV vector, comprising an isolated nucleic acid sequence encoding an aquaporin.

39. (canceled)

40. The AAV vector or recombinant AAV vector of claim 38, wherein the nucleic acid sequence encoding the aquaporin is CpG depleted and/or codon-optimized for expression in a human cell.

41. The AAV vector or recombinant AAV vector of claim 38, wherein the encoded aquaporin comprises Aqp4 or a recombinant Aqp4.

42. The AAV vector or recombinant AAV vector of claim 41, wherein the encoded Aqp4 comprises a sequence having at least 80% identity to the sequence set forth in SEQ ID NO:23.

43. The AAV vector or recombinant AAV vector of claim 38, further comprising one or more expression control elements.

44. The AAV vector or recombinant AAV vector of claim 43, wherein the one or more expression control elements comprise a promoter, an enhancer, a promoter/enhancer, a transcription pausing signal, a termination signal, or any combination thereof.

45. The AAV vector or recombinant AAV vector of claim 44, wherein the promoter comprises a constitutive promoter, a ubiquitous promoter, or a tissue-specific promoter.

46. The AAV vector or recombinant AAV vector of claim 45, wherein the ubiquitous promoter comprises a chicken beta actin promoter.

47. The AAV vector or recombinant AAV vector of claim 45, wherein the tissue-specific promoter comprises a brain-specific promoter.

48. The AAV vector or recombinant AAV vector of claim 47, wherein the brain-specific promoter comprises a synapsin 1 (Syn1) promoter, a calmodulin/calcium dependent kinase II (CAMKII) promoter, a Rgs5 promoter, a glial fibrillary acidic protein (GFAP) promoter, S100 beta promoter, neuron-specific enolase (NSE) promoter, or a Thy 1 promoter.


SEQUENCE LISTING (47 sequences)

SEQ ID NO: 1 - DNA, 1865 bp, Gallus gallus
ttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 2 - DNA, 690 bp, Homo sapiens
gatctaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcgggg

SEQ ID NO: 3 - DNA, 335 bp, Homo sapiens
aggcactaatgatcttatctcagtttaggctccaacacagccctaactaaacaaacaagagactgtggggacagaagccaccagaaacatgccccagcagtgcctgaagtcaggaaagactcccagcccccaccgaaggcaaactgctgctgagctccagcccacaattttcgcccaggctgctgattggtcctcggagccacggagctgtgagagagcttgtatatattcctcaaagggaaaggctgccccagattattgaagtttccacagacggtcagctgttgagaggttcgtgctcaagttgaggatctaagccgccagccaaaatgtgt

SEQ ID NO: 4 - DNA, 448 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 5 - DNA, 1709 bp, Homo sapiens
atcttgtgggaaaccctttcacggccactttgaaactgggttgggagaaggacgtgatgctgggtcaggcatccattggggaccatggacatttttggagagtttgggccaatgttcttgtgtaatttgtcagaaggaacacctacttcttcattcttgtgaagaagaacccataaactggcccccgtgttcagaggctccagtcgagcgagttgaggggacagctggacttctccttcctcaggctggctggaggcttcagcaagggggcacaccgtccacacgcctctgtaaaacagcgttcgaacgggacatctctaaggcatcgtccaactctgagagtctgtgaccttccaccctggggtggagggaaaggccttgggatacagtccacacatcacgttttcccctaaaagtaccacccgttttagtccctgcagacactggcctcagcagcttctctgaggcagccccgttgagcccagggctgaccacagacaagcactactgtccacagaacttcacgcccagtggggccagacttggaggatggcagaggagagaagctccaggggcctctccattagaaaccaacttgcaggccacttaatctcctatgctcagcctgtacttggaagctgcttgttcttggctgcacatttgctttgtttgaatcaattaatccaagtctctctcctctccccacccccaaccttgcctttagggtgacatcaatattcatgtaataaagaaatcgcacaaaaagctgactccccacttcctgccctacaggcccttttttcttcctcagcccatgtgcaatcctggctcccagcaagtccccggggctgcttggtcaatgcagccctgtgtgcaggcctggcagccctgccacccccgccttcggctcccattggctgccacggcctgcagtgggctgcaccagggttcatccatcctccctgggcagagggaataagaggctgcctctgcccaccagtcctgccgcccaggacccgcagcagagacgacgcctgcagcaaggagaccaggaaggggtgagacaaggaagaggtgagaaagagccaggccaagaggacgctcaggaagaaatggttcttttcttttgggtggaacggaatggaggggtagaaactaagtggtagcttaaaaagcccctttaggacaattggcagcatttcagaagtgtcaataggatgatgtgttttaatctccacattactgctgctttgtggacacctgactgcatcagccctagacagctagaggtgtgttttgccataaatcagagaaacgtcaggtttcatggcccagaagtgattgttgacattttcccagcggtactacaaatactgcctcttctgtaactatttgaagagtaaagattttgcttcccactggggctaaaaatgatggagaacctaatgctagtttagtcctatatcataaataaatgcttcccttttctccttatatttcttccaccgccctgggtaagtaactcttcagttttccagtttccctcagtttgaagtgccagggtccccacagccccaggacgccactcagaaattacgtaacaattacaaataaattgggtcaaagaaatcggggttttggtgggttttaactttcctcagtctcacagtttctcagggaggtcgaaccccttctttagagggatcctc

SEQ ID NO: 6 - DNA, 645 bp, Homo sapiens
ctggttttttgtaccagcttcagatgaaatacagtcactcaataaagtccttctgttttctctttccctccctctcgttctctctccttatttgcctagtgaggggaaccctaaaccctttggagcttttccagcagctccaaaataatctcttgattattttcccagggaattcctgccttaaatgtcagcagagtacaactaccccaccaaagcagtccgcctggatctgaatcaagaaaaatgtgcctgctacaccaaacataaaaacctggatctgtggggcgtggggcacagagagagatttatggtgtttgtgaaaggtgtctcaaaaaaaaaaaaattaagtaaatgtttttgcattcctgaaatgccctgtgtctacagtgatcaggtacagaaaagctacttcaagttcaaatataacttagcgattgccacatggtgcagaatctttccacccctaacactccaaaaacccaatcagacaagtggccgtaatctgactcccagcacacagggagctgcggggcaggcaatgagagctgcactctggctgggagtgacagacccacagcaaggcggtgggggtaagtccttctttgccttaagggaacaacgcttagggccaaaagatgaaactgt

SEQ ID NO: 7 - protein, 269 aa, Homo sapiens
MASEFKKKLFWRAVVAEFLATTLFVFISIGSALGFKYPVGNNQTAVQDNVKVSLAFGLSIATLAQSVGHISGAHLNPAVTLGLLLSCQISIFRALMYIIAQCVGAIVATAILSGITSSLTGNSLGRNDLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 8 - protein, 271 aa, Homo sapiens
MWELRSIAFSRAVFAEFLATLLFVFFGLGSALNWPQALPSVLQIAMAFGLGIGTLVQALGHISGAHINPAVTVACLVGCHVSVLRAAFYVAAQLLGAVAGAALLHEITPADIRGDLAVNALSNSTTAGQAVTVELFLTLQLVLCIFASTDERRGENPGTPALSIGFSVALGHLLGIHYTGCSMNPARSLAPAVVTGKFDDHWVFWIGPLVGAILGSLLYNYVLFPPAKSLSERLAVLKGLEPDTDWEEREVRRRQSVELHSPQSLPRGTKA

SEQ ID NO: 9 - protein, 292 aa, Homo sapiens
MGRQKELVSRCGEMLHIRYRLLRQALAECLGTLILVMFGCGSVAQVVLSRGTHGGFLTINLAFGFAVTLGILIAGQVSGAHLNPAVTFAMCFLAREPWIKLPIYTLAQTLGAFLGAGIVFGLYYDAIWHFADNQLFVSGPNGTAGIFATYPSGHLDMINGFFDQFIGTASLIVCVLAIVDPYNNPVPRGLEAFTVGLVVLVIGTSMGFNSGYAVNPARDFGPRLFTALAGWGSAVFTTGQHWWWVPIVSPLLGSIAGVFVYQLMIGCHLEQPPPSNEEENVKLAHVKHKEQI

SEQ ID NO: 10 - protein, 301 aa, Homo sapiens
MVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 11 - protein, 265 aa, Homo sapiens
MKKEVCSVAFLKAVFAEFLATLIFVFFGLGSALKWPSALPTILQIALAFGLAIGTLAQALGPVSGGHINPAITLALLVGNQISLLRAFFYVAAQLVGAIAGAGILYGVAPLNARGNLAVNALNNNTTQGQAMVVELILTFQLALCIFASTDSRRTSPVGSPALSIGLSVTLGHLVGIYFTGCSMNPARSFGPAVVMNRFSPAHWVFWVGPIVGAVLAAILYFYLLFPNSLSLSERVAIIKGTYEPDEDWEEQREERKKTMELTTR

SEQ ID NO: 12 - protein, 282 aa, Homo sapiens
MDAVEPGGRGWASMLACRLWKAISRALFAEFLATGLYVFFGVGSVMRWPTALPSVLQIAITFNLVTAMAVQVTWKASGAHANPAVTLAFLVGSHISLPRAVAYVAAQLVGATVGAALLYGVMPGDIRETLGINVVRNSVSTGQAVAVELLLTLQLVLCVFASTDSRQTSGSPATMIGISVALGHLIGIHFTGCSMNPARSFGPAIIIGKFTVHWVFWVGPLMGALLASLIYNFVLFPDTKTLAQRLAILTGTVEVGTGAGAGAEPLKKESQPGSGAVEMESV

SEQ ID NO: 13 - protein, 342 aa, Homo sapiens
MVQASGHRRSTRGSKMVSWSVIAKIQEILQRKMVREFLAEFMSTYVMMVFGLGSVAHMVLNKKYGSYLGVNLGFGFGVTMGVHVAGRISGAHMNAAVTFANCALGRVPWRKFPVYVLGQFLGSFLAAATIYSLFYTAILHFSGGQLMVTGPVATAGIFATYLPDHMTLWRGFLNEAWLTGMLQLCLFAITDQENNPALPGTEALVIGILVVIIGVSLGMNTGYAINPSRDLPPRIFTFIAGWGKQVFSNGENWWWVPVVAPLLGAYLGGIIYLVFIGSTIPREPLKLEDSVAYEDHGITVLPKMGSHEPTISPLTPVSVSPANRSSVHPAPPLHESMALEHF

SEQ ID NO: 14 - protein, 261 aa, Homo sapiens
MSGEIAMCEPEFGNDKAREPSVGGRWRVSWYERFVQPCLVELLGSALFIFIGCLSVIENGTDTGLLQPALAHGLALGLVIATLGNISGGHFNPAVSLAAMLIGGLNLVMLLPYWVSQLLGGMLGAALAKAVSPEERFWNASGAAFVTVQEQGQVAGALVAEIILTTLLALAVCMGAINEKTKGPLAPFSIGFAVTVDILAGGPVSGGCMNPARAFGPAVVANHWNFHWIYWLGPLLAGLLVGLLIRCFIGDGKTRLILKAR

SEQ ID NO: 15 - protein, 295 aa, Homo sapiens
MQPEGAEKGKSFKQRLVLKSSLAKETLSEFLGTFILIVLGCGCVAQAILSRGRFGGVITINVGFSMAVAMAIYVAGGVSGGHINPAVSLAMCLFGRMKWFKLPFYVGAQFLGAFVGAATVFGIYYDGLMSFAGGKLLIVGENATAHIFATYPAPYLSLANAFADQVVATMILLIIVFAIFDSRNLGAPRGLEPIAIGLLIIVIASSLGLNSGCAMNPARDLSPRLFTALAGWGFEVFRAGNNFWWIPVVGPLVGAVIGGLIYVLVIEIHHPEPDSVFKTEQSEDKPEKYELSVIM

SEQ ID NO: 16 - protein, 301 aa, Homo sapiens
MVFTQAPAEIMGHLRIRSLLARQCLAEFLGVFVLMLLTQGAVAQAVTSGETKGNFFTMFLAGSLAVTIAIYVGGNVSGAHLNPAFSLAMCIVGRLPWVKLPIYILVQLLSAFCASGATYVLYHDALQNYTGGNLTVTGPKETASIFATYPAPYLSLNNGFLDQVLGTGMLIVGLLAILDRRNKGVPAGLEPVVVGMLILALGLSMGANCGIPLNPARDLGPRLFTYVAGWGPEVFSAGNGWWWVPVVAPLVGATVGTATYQLLVALHHPEGPEPAQDLVSAQHKASELETPASAQMLECKL

SEQ ID NO: 17 - protein, 271 aa, Homo sapiens
MSPLLGLRSELQDTCTSLGLMLSVVLLMGLARVVARQQLHRPVAHAFVLEFLATFQLCCCTHELQLLSEQHPAHPTWTLTLVYFFSLVHGLTLVGTSSNPCGVMMQMMLGGMSPETGAVRLLAQLVSALCSRYCTSALWSLGLTQYHVSERSFACKNPIRVDLLKAVITEAVCSFLFHSALLHFQEVRTKLRIHLLAALITFLVYAGGSLTGAVFNPALALSLHFMCFDEAFPQFFIVYWLAPSLGILLMILMFSFFLPWLHNNHTINKKE

SEQ ID NO: 18 - protein, 271 aa, Homo sapiens; identical to SEQ ID NO: 17

SEQ ID NO: 19 - protein, 295 aa, Homo sapiens
MAGLNVSLSFFFATFTLCEAARRASKALLPVGAYEVFAREAMRTLVELGPWAGDFGPDLLLTLLFLLFLAHGVTLDGASANPTVSLQEFLMAEESLPGTLLKLAAQGLGMQAACTLTRLCWAWELSDLHLLQSLMAQSCSSALRTSVPHGALVEAACAFCFHLTLLHLRHSPPAYSGPAVALLVTVTAYTAGPFTSAFFNPALAASVTFACSGHTLLEYVQVYWLGPLTGMVLAVLLHQGRLPHLFQRNLFYGQKNKYRAPRGKPAPASGDTQTPAKGSSVREPGRSGVEGPHSS

SEQ ID NO: 20 - DNA, 810 bp, Homo sapiens
atggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctattgactacactggctgtgggattaaccctgctcggtcctttggctccgcggtgatcacacacaacttcagcaaccactggattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaatag

SEQ ID NO: 21 - DNA, 816 bp, Homo sapiens
atgtgggagctccgctccatagccttctccagggctgtgttcgcagagttcctggccacactcctcttcgtcttctttggcctcggctctgccctcaactggccacaggccctgccctctgtgctacagattgccatggcgtttggcttgggtattggcaccctggtacaggctctgggccacataagcggggcccacatcaaccctgccgtgactgtggcctgcctggtgggctgccacgtctccgttctccgagccgccttctacgtggctgcccagctgctgggggctgtggccggagccgctctgctccatgagatcacgccagcagacatccgcggggacctggctgtcaatgctctcagcaacagcacgacggctggccaggcggtgactgtggagctcttcctgacactgcagctggtgctctgcatcttcgcctccaccgatgagcgccgcggagagaacccgggcacccctgctctctccataggcttctctgtggccctgggccacctccttgggatccattacaccggctgctctatgaatcctgcccgctccctggctccagctgtcgtcactggcaaatttgatgaccactgggtcttctggatcggacccctggtgggcgccatcctgggctccctcctctacaactacgtgctgtttccgccagccaagagcctgtcggagcgcctggcagtgctgaagggcctggagccggacaccgattgggaggagcgcgaggtgcgacggcggcagtcggtggagctgcactcgccgcagagcctgccacggggtaccaaggcctga

SEQ ID NO: 22 - DNA, 879 bp, Homo sapiens
atgggtcgacagaaggagctggtgtcccgctgcggggagatgctccacatccgctaccggctgctccgacaggcgctggccgagtgcctggggaccctcatcctggtgatgtttggctgtggctccgtggcccaggttgtgctcagccggggcacccacggtggtttcctcaccatcaacctggcctttggctttgctgtcactctgggcatcctcatcgctggccaggtctctggggcccacctgaaccctgccgtgacctttgccatgtgcttcctggctcgtgagccctggatcaagctgcccatctacaccctggcacagacgctgggagccttcttgggtgctggaatagtttttgggctgtattatgatgcaatctggcacttcgccgacaaccagctttttgtttcgggccccaatggcacagccggcatctttgctacctacccctctggacacttggatatgatcaatggcttctttgaccagttcataggcacagcctcccttatcgtgtgtgtgctggccattgttgacccctacaacaaccccgtcccccgaggcctggaggccttcaccgtgggcctggtggtcctggtcattggcacctccatgggcttcaactccggctatgccgtcaaccctgcccgggactttggcccccgcctttttacagcccttgcgggctggggctctgcagtcttcacgaccggccagcattggtggtgggtgcccatcgtgtccccactcctgggctccattgcgggtgtcttcgtgtaccagctgatgatcggctgccacctggagcagcccccaccctccaacgaggaagagaatgtgaagctggcccatgtgaagcacaaggagcagatctga

SEQ ID NO: 23 - DNA, 906 bp, Homo sapiens
atggtggctttcaaaggggtctggactcaagctttctggaaagcagtcacagcggaatttctggccatgcttatttttgttctcctcagcctgggatccaccatcaactggggtggaacagaaaagcctttaccggtcgacatggttctcatctccctttgctttggactcagcattgcaaccatggtgcagtgctttggccatatcagcggtggccacatcaaccctgcagtgactgtggccatggtgtgcaccaggaagatcagcatcgccaagtctgtcttctacatcgcagcccagtgcctgggggccatcattggagcaggaatcctctatctggtcacacctcccagtgtggtgggaggcctgggagtcaccatggttcatggaaatcttaccgctggtcatggtctcctggttgagttgataatcacatttcaattggtgtttactatctttgccagctgtgattccaaacggactgatgtcactggctcaatagctttagcaattggattttctgttgcaattggacatttatttgcaatcaattatactggtgccagcatgaatcccgcccgatcctttggacctgcagttatcatgggaaattgggaaaaccattggatatattgggttgggcccatcataggagctgtcctcgctggtggcctttatgagtatgtcttctgtccagatgttgaattcaaacgtcgttttaaagaagccttcagcaaagctgcccagcaaacaaaaggaagctacatggaggtggaggacaacaggagtcaggtagagacggatgacctgattctaaaacctggagtggtgcatgtgattgacgttgaccggggagaggagaagaaggggaaagaccaatctggagaggtattgtcttcagtatga

SEQ ID NO: 24 - DNA, 798 bp, Homo sapiens
atgaagaaggaggtgtgctccgtggccttcctcaaggccgtgttcgcagagttcttggccaccctcatcttcgtcttctttggcctgggctcggccctcaagtggccgtcggcgctgcctaccatcctgcagatcgcgctggcgtttggcctggccataggcacgctggcccaggccctgggacccgtgagcggcggccacatcaaccccgccatcaccctggccctcttggtgggcaaccagatctcgctgctccgggctttcttctacgtggcggcccagctggtgggcgccattgccggggctggcatcctctacggtgtggcaccgctcaatgcccggggcaatctggccgtcaacgcgctcaacaacaacacaacgcagggccaggccatggtggtggagctgattctgaccttccagctggcactctgcatcttcgcctccactgactcccgccgcaccagccctgtgggctccccagccctgtccattggcctgtctgtcaccctgggccaccttgtcggaatctacttcactggctgctccatgaacccagcccgctcttttggccctgcggtggtcatgaatcggttcagccccgctcactgggttttctgggtagggcccatcgtgggggcggtcctggctgccatcctttacttctacctgctcttccccaactccctgagcctgagtgagcgtgtggccatcatcaaaggcacgtatgagcctgacgaggactgggaggagcagcgggaagagcggaagaagaccatggagctgaccacccgctga

SEQ ID NO: 25 - DNA, 849 bp, Homo sapiens
atggatgcagtggagccagggggacgtggctgggccagcatgttggcgtgcaggctttggaaagccatcagcagggcgctgtttgcagagttcctggccacggggctgtatgtgttctttggcgtgggctcagtcatgcgctggcccacagcacttccctccgtgctacagattgccatcaccttcaacctggtcaccgccatggctgtgcaggtcacctggaaggccagcggggcccacgccaaccccgccgtgacgctggccttcctcgtaggctcccacatctctctgccccgtgctgtggcctatgtggctgcccagctggtgggggccacggtgggggctgctctgctttatggggtcatgccgggagacatccgagagacccttgggatcaacgtggtccggaacagtgtctcaactggccaggcggtggcagtggagctgcttctgaccctgcagctggtgctctgtgtcttcgcttccaccgacagccgtcagacatcaggctccccggccaccatgattgggatctctgtggcactgggccacctcattgggatccacttcactggctgctccatgaatccagcccgctccttcggccctgccatcatcattgggaagttcacagtccactgggtcttctgggtggggcccctgatgggagccctcctggcctcactgatctacaacttcgtcctgttccccgacaccaagaccctggcgcagcggctggctatcctcacaggcaccgtagaggtggggacaggggcaggggcaggggcggagcccctgaagaaggaatcccagccgggttcgggagccgtggagatggagagtgtgtga

SEQ ID NO: 26 - DNA, 1029 bp, Homo sapiens
atggttcaagcatccgggcacaggcggtccacccgtggctccaaaatggtctcctggtccgtgatagcaaagatccaggaaatactgcagaggaagatggtgcgagagttcctggccgagttcatgagcacatatgtcatgatggtattcggccttggttccgtggcccatatggttctaaataaaaaatatgggagctaccttggtgtcaacttgggttttggcttcggagtcaccatgggagtgcacgtggcaggccgcatctctggagcccacatgaacgcagctgtgacctttgctaactgtgcgctgggccgcgtgccctggaggaagtttccggtctatgtgctggggcagttcctgggctccttcctggcggctgccaccatctacagtctcttctacacggccattctccacttttcgggtggacagctgatggtgaccggtcccgtcgctacagctggcatttttgccacctaccttcctgatcacatgacattgtggcggggcttcctgaatgaggcgtggctgaccgggatgctccagctgtgtctcttcgccatcacggaccaggagaacaacccagcactgccaggaacagaggcgctggtgataggcatcctcgtggtcatcatcggggtgtcccttggcatgaacacaggatatgccatcaacccgtcccgggacctgcccccccgcatcttcaccttcattgctggttggggcaaacaggtcttcagcaatggggagaactggtggtgggtgccagtggtggcaccacttctgggtgcctatctaggtggcatcatctacctggtcttcattggctccaccatcccacgggagcccctgaaattggaggattctgtggcgtatgaagaccacgggataaccgtattgcccaagatgggatctcatgaacccacgatctctcccctcacccccgtctctgtgagccctgccaacagatcttcagtccaccctgccccacccttacatgaatccatggccctagagcacttctaa

SEQ ID NO: 27 - DNA, 785 bp, Homo sapiens
atgtctggagagatagccattgtgagcctgaatttggcaatgacaaggccagggagccgagcgtgggtggcaggtggcgagtgtcctggtacgaacggtttgtgcagccatgtctggtcgaactgctgggctctgctctcttcatcttcatcgggtgcctgtcggtcattgagaatgggacggacactgggctgctgcagccggccctggcccacgggctggctttggggctcgtgattgccacgctggggaatatcagtggtggacacttcaaccctgcggtgtccctggcagccatgctgatcggaggcctcaacctggtgatgctcctcccgtactgggtctcacagctgctcggggggatgctcggggctgccttggccaaggcggtgagtcctgaggagaggttctggaatgcatctggggcggcctttgtgacagtccaggagcaggggcaggtggcaggggcgttggtggcagagatcatcctgacgacgctgctggccctggctgtatgcatgggtgccatcaatgagaagacaaagggccctctggccccgttctccatcggctttgccgtcaccgtggatatcctggctgggggccctgtgtctggaggctgcatgaatcccgcccgtgcttttggacctgcggtggtggccaaccactggaacttccactggatctactggctgggcccactcctggctggcctgcttgttggactgctcattaggtgcttcattggagatgggaagacccgcctcatcctgaaggctcggtga

SEQ ID NO: 28 - DNA, 888 bp, Homo sapiens
atgcagcctgagggagcagaaaagggaaaaagcttcaagcagagactggtcttgaagagcagcttagcgaaagaaaccctctctgagttcttgggcacgttcatcttgattgtccttggatgtggctgtgttgcccaagctattctcagtcgaggacgttttggaggggtcatcactatcaatgttggattttcaatggcagttgcaatggccatttatgtggctggcggtgtctctggtggtcacatcaacccagctgtgtctttagcaatgtgtctctttggacggatgaaatggttcaaattgccattttatgtgggagcccagttcttgggagcctttgtgggggctgcaaccgtctttggcatttactatgatggacttatgtcctttgctggtggaaaactgctgatcgtgggagaaaatgcaacagcacacatttttgcaacatacccagctccgtatctatctctggcgaacgcatttgcagatcaagtggtggccaccatgatactcctcataatcgtctttgccatctttgactccagaaacttgggagcccccagaggcctagagcccattgccatcggcctcctgattattgtcattgcttcctccctgggactgaacagtggctgtgccatgaacccagctcgagacctgagtcccagacttttcactgccttggcaggctgggggtttgaagtcttcagagctggaaacaacttctggtggattcctgtagtgggccctttggttggtgctgtcattggaggcctcatctatgttcttgtcattgaaatccaccatccagagcctgactcagtctttaagacagaacaatctgaggacaaaccagagaaatatgaactcagtgtcatcatgtag

SEQ ID NO: 29 - DNA, 906 bp, Homo sapiens
atggtcttcactcaggccccggctgaaatcatgggccacctccggatacgcagcctcctggcccggcagtgcctggcagagtttctgggtgtgtttgtactcatgctcctcacccaaggagctgtggcccaggctgtcaccagtggagaaaccaaaggcaacttcttcaccatgtttctggctggctctctggccgttacgatagccatctacgtgggtggtaacgtctcaggggcccacctgaatccagccttctccctggccatgtgcatcgttggacgcctcccctgggtcaagctccccatttacatcttggtgcagttgctgtctgctttctgtgcttcgggagccacctatgttctctaccatgatgccctacagaactatacaggtgggaacctgacagtgactggccccaaggagacagcctccatttttgccacctatcctgccccctatctgtccctgaacaatggcttcctggatcaggttctgggcactgggatgctgattgtggggctcttggccatcctggacagacggaacaagggagtccctgcgggtctggagcctgtggtggtggggatgctgatcctggccctcgggttatccatgggtgccaactgcgggattccactcaaccctgcccgggacctgggcccacgtctcttcacctacgtggctggctggggtcctgaagtcttcagtgctggtaatggctggtggtgggtgcctgtggtggcccctctggtgggggccaccgttggcacagccacttaccagctgttggtggctctgcaccaccctgagggcccagagccagctcaggatctggtgtctgctcaacacaaagcctcagagttggaaactcctgcctcagctcagatgctggagtgtaagctatga

SEQ ID NO: 30 - DNA, 1835 bp, Homo sapiens
agagccggagcccgcaacccgctcaggcggcgacggagccatgtcgccgctgctggggctccggtccgagctgcaggacacctgcacctcgctgggactgatgctgtcggtggtgctgctcatggggctggcccgcgtagtcgcccggcagcagctgcacaggccggtggcccacgccttcgtcctggagtttctagccaccttccagctctgctgctgcacccacgagctgcaactgctgagcgaacagcaccccgcgcaccccacctggacgctgacgctcgtctacttcttctcgcttgtgcatggcctgactctggtgggcacgtccagcaacccgtgcggcgtgatgatgcagatgatgctggggggcatgtcccccgagacgggtgcggtgaggctattggctcagctggttagtgccctgtgcagcaggtactgcacaagcgccttgtggagcttgggtctgacccagtatcacgtcagcgagaggagcttcgcttgcaagaatcccatccgagtcgacttgctcaaagcggtcatcacagaggccgtctgctcctttctcttccacagcgctctgctgcacttccaggaagtccgaaccaagcttcgtatccacctgctggctgcactcatcacctttttggtctatgcaggaggaagtctaacaggagctgtatttaatccagctttggcactttcgctacatttcatgtgttttgatgaagcattccctcagttttttatagtatactggctggctccttctttaggtatattgttgatgattttgatgttcagctttttccttccatggctgcataacaaccatacaattaataaaaaggaataactgttccaaagactcagactaacatacaggacagtccagctggatgtgataaagattttatcacctcatatggaaaacaccggctgcactggattcatcagtgttaacttcctttgaggaagctgccttatagttttcatcactgggactttaaaaaaaaattactgtgaaaatgaggtattctgtacttctcagttaagacttgttctttgagtgatgtattaaatgctgctagaaaagcctcattacattaaatataaatcaatcttaaatgataattgttaactttgatgaaaaacgagtacaggatgagaagggaagtaaaggtgatagtaagatcaacgaatttgtgtatcaagtgtcacccaaatgaacagaatttttaactataaaagtacaaggcgtcaacttacagaactggggagaggatactttcagccaccacctcacaaaatgataccatcaagcagtgtcatctttcaacaggaaagttgcttttagaagtagaatatatattcactcacatgcaagcgatttcctacattttaagtacattaaaatctaatacctaaaatagtttctctaaatagacaatctcactagctccagccaagtggttattctttaatagaatataataaaacaaaacacccatacaaagtcaggatttaagcttttcacattctcctttgaaccaacagctagctaccttatgaagtagggagggcatgtatttttaatctccattttacagataaggaaactaagcctagatgggtttgctgccctggcttagatcacatagagccaaataccatctctagtcttcaacttcaatccaaaaattcttctcggtcacgattatgactaaagggaaatcaagggttcatagaaacattaaatactttataataacgttaaaacttgtttagaaattaactagtattttttgaggtttaaagaaccttccagttttact

SEQ ID NO: 31 - DNA, 1718 bp, Homo sapiens
agagccggagcccgcaacccgctcaggcggcgacggagccatgtcgccgctgctggggctccggtccgagctgcaggacacctgcacctcgctgggactgatgctgtcggtggtgctgctcatggggctggcccgcgtagtcgcccggcagcagctgcacaggccggtggcccacgccttcgtcctggagtttctagccaccttccagctctgctgctgcacccacgagctgcaactgctgagcgaacagcaccccgcgcaccccacctggacgctgacgctcgtctacttcttctcgcttgtgcatggcctgactctggtgggcacgtccagcaacccgtgcggcgtgatgatgcagatgatgctggggggcatgtcccccgagacgggtgcggtgaggctattggctcagctggttagtgccctgtgcagcaggtactgcacaagcgccttgtggagcttgggtctgacccagtatcacgtcagcgagaggagcttcgcttgcaagaatcccatccgagtcgacttgctcaaagcggtcatcacagaggccgtctgctcctttctcttccacagcgctctgctgcacttccaggaagtccgaaccaagcttcgtatccacctgctggctgcactcatcacctttttggtctatgcaggtatattgttgatgattttgatgttcagctttttccttccatggctgcataacaaccatacaattaataaaaaggaataactgttccaaagactcagactaacatacaggacagtccagctggatgtgataaagattttatcacctcatatggaaaacaccggctgcactggattcatcagtgttaacttcctttgaggaagctgccttatagttttcatcactgggactttaaaaaaaaattactgtgaaaatgaggtattctgtacttctcagttaagacttgttctttgagtgatgtattaaatgctgctagaaaagcctcattacattaaatataaatcaatcttaaatgataattgttaactttgatgaaaaacgagtacaggatgagaagggaagtaaaggtgatagtaagatcaacgaatttgtgtatcaagtgtcacccaaatgaacagaatttttaactataaaagtacaaggcgtcaacttacagaactggggagaggatactttcagccaccacctcacaaaatgataccatcaagcagtgtcatctttcaacaggaaagttgcttttagaagtagaatatatattcactcacatgcaagcgatttcctacattttaagtacattaaaatctaatacctaaaatagtttctctaaatagacaatctcactagctccagccaagtggttattctttaatagaatataataaaacaaaacacccatacaaagtcaggatttaagcttttcacattctcctttgaaccaacagctagctaccttatgaagtagggagggcatgtatttttaatctccattttacagataaggaaactaagcctagatgggtttgctgccctggcttagatcacatagagccaaataccatctctagtcttcaacttcaatccaaaaattcttctcggtcacgattatgactaaagggaaatcaagggttcatagaaacattaaatactttataataacgttaaaacttgtttagaaattaactagtattttttgaggtttaaagaaccttccagttttact

SEQ ID NO: 32 - DNA, 1063 bp, Homo sapiens
gtcccctcaggtgtcctgcaggcacagctcctcggggggcccaggccgatggcaggtcttaacgtgtccctctccttcttctttgccaccttcgccctctgtgaggcggccaggcgggcctccaaggccctgctcccagtgggcgcctatgaagtcttcgcccgggaggcgatgaggacgctggtcgagctcgggccctgggctggggactttgggcctgacctgctgctcaccctgctcttcctgctcttcctggcgcacggggtcaccttggacggggcctcggccaaccccactgtgtccctgcaggagttcctcatggccgagcagtctctgcctggcacgctgttgaagctggcggcacaggggctgggcatgcaggccgcctgcaccctgatgcgcctctgctgggcctgggagctcagtgacctgcacctgctgcagagcctcatggcccagagctgcagctcggccctgcgcacatccgtgccccacggggcgcttgtggaggccgcctgcgccttttgtttccatctgaccctcctgcacctgcggcacagtcctcccgcctacagcgggcccgctgtggctctgttggtcaccgtcacggcctacacggccgggcccttcacgtctgccttcttcaaccctgccctggccgcctctgtgacctttgcctgctcgggacacaccttactggagtacgtgcaggtgtactggctgggccctctgacagggatggtcctggctgtgctgctgcaccagggccgccttccccaccttttccagaggaacctgttctacggccagaagaacaagtaccgagcaccccgagggaagccggccccggcctcaggggacacccagacccctgcaaaggggtccagtgtccgggagcctgggcgcagtggtgttgaggggccacattccagctgagtggccttgctctgtgtgagccccgtgcgagggccctgcttgtagctggaccctggaaccttctgtagctaagagggaatcctggccccctccccagaagccatttgtcaataaaccatttctaaga

SEQ ID NO: 33 - DNA, 1099 bp, Homo sapiens
gtcccctcaggtgtcctgcaggcacagcatctcggggggcccaggccgatggcaggtcttaacgtgtccctctccttcttctttgccaccttcaccctctgtgaggcagccaggcgggcctccaaggccctgctcccagtgggcgcctatgaagtcttcgcccgggaggcggtgggcgcggtgcagctcggggcctgcttcctggagatgaggacgctggtcgagctcgggccctgggctggggactttgggcctgacctgctgctcaccctgctcttcctgctcttcctggcgcacggggtcaccttggacggggcctcggccaaccccaccgtgtccctgcaggagttcctcatggccgaggagtctctgcctggcacgctgctgaagctggcggcacaggggctgggcatgcaggccgcctgcaccctgacgcgcctctgctgggcctgggagctcagtgacctgcacctgctgcagagcctcatggcccagagctgcagctcggccctgcgcacatccgtgccccacggggcgcttgtggaggccgcctgcgccttttgtttccatctgaccctcctgcacctgcggcacagtcctcccgcctacagcgggcccgctgtggctctgttggtcaccgtcacggcctacacggccgggcccttcacgtctgccttcttcaaccctgccctggccgcctctgtgacctttgcctgctcgggacacaccttactggagtacgtgcaggtgtactggctgggccctctgacagggatggtcctggctgtgctgctgcaccagggccgccttccccaccttttccagaggaacctgttctacggccagaagaacaagtaccgagcaccccgagggaagccggccccggcctcaggggacacccagacccctgcaaaggggtccagtgtccgggagcctgggcgcagtggtgttgaggggccacattccagctgagtggccttgctctgtgtgagccccgtgcgagggccctgcttgtagctggaccctggaaccttctgtagctaagagggaatcctggccccctccccagaagccatttgtcaataaaccatttctaaga

SEQ ID NO: 34 - DNA, 221 bp, Artificial Sequence
  Synthetic
agacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 35 - DNA, 13824 bp, Homo sapiens
aaggaaaaagttggcaggccgacagcacagccgtgtctgcatccatccagaggaggtctgtgtggtgtggggcgggccaggagcgaagagaggccttcctccctttgtgctccccccgccccccggccctataaataggcccagcccaggctgtggctcagctctcagagggaattgagcacccggcagcggtctcaggccaagccccctgccagcatggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacgtgagtggggtgtccctgggcttgggggggttctagaatgatgctgaaaggcactggttccatcctctgcccattgtgcagatggggacactgaggaacggagaggacaagaggttgctggaggtcacgtagagagctggggggaagagctggggctggaactcagctatgcatgcctcccaaagcctgttttctgccaggcactgtgggaagctgagatccagagaataatggtcttgccaatgtcccctgcagggcatctattatggggaataagcttggccagcagttcctcgccccttggctgtagctttttggcccttctcagtcctgcctgtgccgccagctccctcctcctgcagccctgcaccctggggaaactttgcccgctgtccccagccaccctgaaccaaatgcccagcctgtctgcagctggctctgcaagtgtgtgagcaggactccctcagggagccccccatattcacactagtttccaaataaaccacctctaggaatcaagagaaaggccctcttccactcttttgcccctgagattagacaaagataagggagtgaaaacactctctggggctgtttgaaggcagttttcctggattacagtatgggctggggggcaagcgctgggctccatggtctctaggcactgagaggtgtggcgtgtgtgtgcatgtgcgtgtgcgtgtgtgtgtgtgaaggtgtgtatgtgtgcatgtgtttgccagcatgagtctctgtgcttgtgtcaactgtgtgcatgtttgtgctgctgtgtaagggtgtgtgggggtgagtttgcaacacgtgcccatggctcctggcttgaatggggtctggactggggtcactaatgacaagcagatgctggtgactgggtggcagctgtggaaaagaatgtctcctgggctgcttagcatggcaaattggccctggtggcagctgcctgctgtcccagacacggcctttcccagctcctcagacaagtgcaaactcaggccgggcttgggccacagcaggggaggatttggcctgagagactggggaaggcttggaggatgtctctgagggcccaagggacaggtgagcaggcctgggggtgcccttacagggagggagagaagccaaggagcagagaggaggactcgtctagagagatgaggctggcggggctgtgggcagggcatgtgggtgtggggacggtgtgggccatctctgagggcgcctcagagatggcagatagtgattacagcagctgtcctctcgcttcccagccaaagttcagatcctgcggctggcagcccaggagaggctaggtgaaggtctggtgcagagggcagctggctcagcctggccatggggggcttagcacttctgtgtcacggtgacacctcacccaacctcctctgcagccaacctctgctaggcccaagattgaagattagctccgtgggctggagtttagactttagccagtttagacgtggccgcgggggatgtacgggacagagctcaggcaggccacatcaagctctgcccccccaggcctcagttgccagtgggtgcagagcttgtggcctcagggagttctgctgggtagggtcaagctcacactgaagcttggctgggtatgatgggctgggggagcgtccctgtaggggagtggatgtgatttccaggctttctggccccgtctcttccctcccatctcacagggcggagcctctgcacctgctggcgaaggcttcaccctctagtatgcgcatccgaggcagaggcgggagggtgggccccttctgtgccatctggaacagaggccacaccagcctcccgtgccccattccctccctggccttcattttttatagtcaccccaatacttgggctgggcgtgttgcaaatgggagagaaagaggagcagcctagtgtggggtccggccctgggctgagaggcagacacctgggttcaagacgtgtccactgctaacttgttgagcttctttggagctgcggaccctgaccctcgagagggaactcaggtcttgtgacctcacagccacacccagacagccagctcacttcattgctttgagcttatcacccatcccacgaaagctggctaagagcagcccagcaggggcagtgctgaacataagggcgcttagcatgatggtgggggctcaggcgctgtcctgtcccaacagtggaagggtggggttcaggacctcaactctgggcctagaaatagagaaggtacccctctaagggctcccgtcagacactggccctggaactggaggggcctcaggggtggcctggatgcctcaagaggagcagggaacagtctgggcagcaggtgacacactctctttcatgacctggggtaggctcctgcagcctctctggccctgagccccagcccagtcaaaacctgggtggggcaagtggttggcatggagatttccaaggaaaacagagaaagatgtggcaggaagtggttggggtgagtcagggggtgtcccttcgggacatgctggggacaactttctcctgactgactcagggctcaggatatgccctagagagaccgtgggcctccgtccagcctgcaaggtcccggctagttctaagagccaaataccaattttggctaagggtgagaggctctgaggctggggtggacactctcacctgtccctgtcctagctcagccactaacaccatgtggcatcacacaacagactggcctgtgtggccctagttcctgtctatgagatagggacatagcagggtgtggctgggctctgagaagtaggagatgctggtggactcaggattcctggcatttgctggcttttttcattcttctgctgtctggtctgctcagccggctccctgcaagcatgaccagggactgctgggtgctcaatgcagggctgggttagcccggctccctcccgagagaagcccacagccctaggtcaagataagccagtgcccagctgcctgtgatatgaagcaggaggcagatgcgataagaggtgtcagggcgggcccgggaacttttgaacttaaggggtgggagggccaggaaggcatcagggaggaggtttcgtaggagcctgaatgtggagacactgcagagggaaggggcagagggactgtgtgagcaagcacaggctaggaggaggtgagggccgtggtacgggagttgttgggtatggtcaggctgggggtgcatgggaggagggtggtgagggctcaggattcgggccaggggctgcatccgggggaaggggtctggattcccagagcctgtgatttcagcccgctgggctcaggcagagggcaaagctaccctcttcggggtcttctcccagccctgctaattccaggcctgactgctcaagaagtgtttctacctgcagcatcaggggcctgactgaagccctaagttctcccctttatcccgtttttctgggcttcagtttccccagtcatagagtgagaggcctcttcatgccaccagttctcccgggggtgctgctgcctcctccagagctcaggggagggatggggaaaggaaggcagagtttccatgtccctgcccagcccgtcctgcccagggaggggctgggggctgggtggtgatgccacatctgggacatataattactgtttccgccagccccagatgagggggagggcacagagatggggagctttgtggtggagacttccctcaccatggaaaatcctgacgctgcttcccctttagggacctcagaacagcccagccggtcccagtgttgcccaccctgatgcttggccctgactgggatttgccctgggaactctgaaggggccatgctttgagaggggacacggcctcctgggatacctgtctgaggggctcaccccctgctcaccactcccaggccacagcctcctgtcacaccttgccttcccttctccaccccaggcaaccttctagcagcatgaagtggccattagctcactgcaagccaattccagatggtacccccacttagtctagggcccccacccagcaggggccatggcccttaccttctggaagcaccaatgagtctggagtctcctgagtggctgggatctgagccctcacaccgggaggtgaaggcaggaaaatgctgtccgcgaggccctgcaagtggccaggatcccctcagtcagggcttccccatggggatgagaggggctccttgcccacatggagtggtgcccagggggatcatgaggggctgtcttcactcagccaggagctacagttctgtggggtgggcagactctgaggctgaaatctccaacttaaaagatatccggaaaggccggaaaatgcccccggcccttccagcagctcctttcagtgcctgcaatctcgctgtcagggccccttgaaaggcagatttctactcagtgtaaagaaggttgttgttgatattgttgttgacacgaagaaggggctgtctctaagtcaataagctccctgccattaggggtgtgcaagtagatgctttctggtgaggaggctgctttgggggtgctagtctatgcgggggtccagtggggtgggtgacaggttctgcagcataggctttgtgtcatgctgagatcagcaattctgatggcctgcgggggatcctgtgcttaggtttctggatctggggctggagagtgaccaccccgatgtgaggcaggaggcacttttggagactgcgggggctcgaatctgggccaaggcctccattttactgatggggaaactgaagcccacagaggcgtggagacttgtcaaggtggcactgcctcctagcccttgattattcctgctgctccaaagtgtacacacatggcacaaacacaaacatgcacacacatgtgctcttacgtggaggacacacacacatggtacacaaagcacattctcaagactgggtggtcccactcagactctgggacccaaggagggttgggcggggattcctcaatggagccttttcctctccctgccctggagttaggaaaagaaggagggagggagggaggaagccattgctttgtgtattgtttcttgtcaggaagatgaaatggcttctgggagcaaataaaggccttgttgagacctgctgctttctcacatggggggctcatttttttctcttctcttccctctctagctggtagtggcagctatgtgggtgtgtgacacagggagacagtgaggcacccagaagtgccatcagacataccagtgcaaacacacagcacagcctccaggacctcatgtcctgcagggatgcctgtatgatatagctgttcccagggaggccgaggcacttggtcagtacctcatcagttgatgaaaaagagtgtgcattctctaattcaggggtacagggttttcatatttttctgaagtcaagcttgttaattatggggttcaaaccctccctgatcatgttattggtagttttcggctgcttcatttattctgtacatttctaatagaagattgttaacatttttcactctggtggtagggtgatccagtcctccttgtaattttgtcaattttcccctcatttatttttaggctatgttgtttggtgcatacaagtgggggatttttgcagttcctttatagatttttatttttttttttgagacggagtctcactctgtcgcccaggctggagtgcagtggcaccatctcggctcactgcaagttctgcctcccgggtttatgccattctcccacctcagcctcccgagtagctgggaccacaggccaccacacccggctaattttgtttttgtatttttagtagagacggggtttaaccgtgttagccaggatggtctcgatctcctgaccttgtgatccacccgccttggcttctcagagtgctgggattacaggcgtgagccaccgcgcctggccagttcttttatagatttttaaaatgtaattattgtgtaataaaccacttacaatctaacaaaaaagagaaagagggagtcagccctggacacatgtggtcatcacagacacagcagacacttatctacaaagacacacagaggcacacaaaaccaggaatccccccatgtccacgtgtgaacacccatggaggagtgacactctggagcccacataggtcccctcactgcccagtgctgagtcttggaggcactggggtgggcagggcaggcctcgatgtctcctatcccttgagaacgaaagctcagagcctcagtcaggcctggctctggagcctgctcttccccaaggccagggcaccagggtgcccaggccaagtcaggggccccatgccttggaagccaccctggttggcctgtctatgggtagcagggagtgcttagagcacattgcctggagagatcatccctgggctgccctagagccaggacacactggcagccacagaaccaccttctgccagctcccaagtccaagctgggaatagccaggtcccttgactgggcctaggagtggccagcccattgacctgtgagtgtagggtggcacagactatggggcaaggatcctggctttgctgcatcctccctgggcctcagcctctgcgtcatcatctcactgagccatcgcacaggctgccctctagggctcccagaggctaatgattggcccattgtttcccagctagaaagtgaagaagaccgccctgggtccggagagcatctctggagccaggctgcccctagcaagggtgggagttctagaaaattagcattctctgccacttctatcagcacacagtaagcctcccctcagaggcgtgggcagagaaatggcagaggtcccccactctgtcttcaccgagctcccaggccaggaggagatgcacccacataggccgcaggaagattgatttcctacaagatgcacagagaattcagtgagggcatggtgggtgggagcagaagttaattccagccagggatctgggaaggcttctaggagctggtggggtctgaactgggccttgagtagtgggcttacaagggcttagcacaaaatggagagcttgtatctttctgacacttcagaagaaatgtgtggattattctttcggtaatacttgcattcacaaaacccagggctgcagacaaaagcgggaagacaattgagaattgagaattgagaattgttctgattcttacttttttttttttttttttgccttaataagacctgggccaggcaaaactacctggggatgtgcaaatatatatgtacatatatataaaacaagtgactacaatgggaactgtccttgtttaagcaagttttcaaatttaatgggttgtaaacagtaacgtacatttgaagtattttacctgctctttcctggacattgagtttcaacaggcctttgagtgccccttccaattctgaattatcaagtgtttccagatgtaaataatgcaagtgtattagatagttgaaggaaggttgtcagtagatgttgaagctatcaggggaggcttcctggtggtagtggtagtgggtattggagaggaaggctgtcagcccatgtggttctcagcagtcacgaaggcacattccagaattcccagggcaggtggctgacccctccccttcccaaggactcagagtacagggcctggaactatcaccctctgctctgcccactttgcatgaaaagcatgttgtctttgggtaggggctggggctctcgcacctggttaagaaccagaggagatgagctctttctggtcacatctgtgagggatggggctgggagggttctccaaggcgggagccacttacagcgatgactccattaatcatcggggaagggctggtttcaggacttctgccagctagcaattaggccaaccctggagagcagaagagagaggaatagaaggagccacgagcttcctgagggctcagatacagagggacaggggcctcacaggtgcaagtgtgggtgggcatcaggactagccgggctggcctcaccagcctcaaccccattcagcctgtcctaactctgggcaggcttctgccagcagctccacccgccgtcactccttctgcctgtccccagatcctgcacacacaatgagaagaaccaaaacaagagtcactaccactgccccatccctcctgcgtcgacaactctttccaaaagccctgagacggcccctttggcctttgaacctcccagctgctctggagagcaggcacggacaatgagatccattttgcagatgggaagactgaagaccagagggaataggccatgctcatggtgccagaaagtagaagagtcagaacctgaacacagggcttctgcctctttggcccaaccttgggacagaaagggaactgggcattggccagagggctggcggacctaagggatgtagccctggggtccgccaggaaaggcccaaagctggggctgcaaggcaggggaggggatgtgagtcattccctgtaacccagggctgcagacaaaggcgggaagagaattgttcttgtaggatggcctcagggtggggctgggaaaacctccaaggtgtgcccccctaccttggattttgcaaaattctctatggccttggagacactcccatatcctgaggatgggagctgggtagtttgtctcccagctgggaagtgtttgggtgcaccagccctgcctgggagaaggcagcctaagtggcctcaagtgggcatctttgcaagtgagcctttgctgatgtggtccatctcccctgtgtgagtgcacttagcccctgggacggccacaaagcatagtggaaggtgctggggcttcctggggaggggtcctccgggggcagctgcatggggagggccttcatgacctcacagcagcttccattcccttctctttccatgagtcatgtttctaaaagtggcccaggagtaagtggcagccagacggtggtggcggggcctatctggccaggcagagggagggtgaggctgaggccagcagtggggaggaagaagagagaaggagagacagggagggcagggccagaagcaatgggagacaggccttggaccaaggaaagagacggggtggagaaggagagagagagggtggagacaatagggaggcaggcacagaggagaaagaggggtggagggagccaggacctcaccgaaggcctcctcagccctggaatttagatgcagtcgggcatggggtggaatgttctggacttttgggtatgaagccgtgtcccctgctgggccttcccacctttttgcatctcttctcctaggagtgctcctgaccatcaccttcatgcctggggctcgccccttgcctctggtcttggtaccccagaataccctggcctggatgcagctggatgcaaaggccccagctcaccccaggcctctccagcttctaggcagagtggggcctgggtctaggcaggtattaggggctgggctggagtttcattaacacagggggtgggttcaaggcctgatttccactacctgccgtgtaggcttgcctgggaccctgtgatgggctctgaaggcccatctgagggtccgcccttcatgcctgggcctgggaggcctgggtatccttggggtcctgggacacagtccctgcctaccctcctcaccagtcctcaccacctctctcccctgcagctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctgtgagtcaggggccctcccagatggaggtgggggaagggagggcgggggctggtggggtgccctgccatgggcagccagtgggactcccgacagggctcttgccattgggtggaggatggcgggtcagcgctgggggctgggggcagggtcctgccctggagaggagcacagggacctcctgcccagcttggggtcagcactcctctttccctgggtctcattgtcccccaccctgattgttctctttctccctccaacctctccctcctctcactctctcttcacctatgactctctgccttcgcccctccctctgtttctttccctcacagattgactacactggctgtgggattaaccctgctcggtcctttggctccgcggtgatcacacacaacttcagcaaccactgggtaggagacccacggggggtggggtgggaagctttggtgtcccatggtaagcctgaccccaccctcacagtgtcccttcctgttctggaggctctgggagacagccagaggacaggaaatcaggaaactgaggcctgccatgtagaggcaggctgggggtcacactgccagcactttcaggcctagtctctgccctcccagctcggccctgccccatgctgcctggcctccaggtcttcccagctgcgtggttaaaagtggggctccaaatcctggctcagccactttcgggtttagcatgaccttgcgcagtgtgcttgagctttggtttcctgagctgcggagggggatatggtggtgcccacctctcagggtggccgagaagaggaaagggctcactccccatctgtaaactgagctcaacacagaggaggcgctcagtggattttagctcttatggttgtggatgtggatgtgctaggccagctttgcattagacaatggtctctggggcccttccaaagctgggagtgtgggggcagtggccttcctcaagaaggggctgctactgcccatgcctgagctgggccccacagaggcaaggagttgaccctaccacgtactctggcctgggccggttctgaggggcaccggaatcatgatgttaggatttggctctcctacctgcctccatcccaggtgggaaaaacctgtccaacggggtggtgggctgtggggtaacctagggaacgcttcccaggggcttttgagtggagccctctgaacacacctgctctgttcctagattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaatagaaggggtctggcccgggcatccacgtagggggcaggggcaggggcgggcggagggaggggaggggtgaaatccatactgtagacactctgacaagctggccaaagtcacttccccaagatctgccagacctgcatggtcaagcctcttatgggggtgtttctatctctttctttctctttctgtttcctggcctcagagcttcctggggaccaagatttaccaattcacccactcccttgaagttgtggaggaggtgaaagaaagggacccacctgctagtcgcccctcagagcatgatgggaggtgtgccagaaagtcccccctcgccccaaagttgctcaccgactcacctgcgcaagtgcctgggattctaccgtaattgctttgtgcctttgggcacggccctccttcttttcctaacatgcaccttgctcccaatggtgcttggagggggaagagatcccaggaggtgcagtggagggggcaagctttgctccttcagttctgcttgctcccaagcccctgacccgctcggacttactgcctgaccttggaatcgtccctatatcagggcctgagtgacctccttctgcaaagtggcagggaccggcagagctctacaggcctgcagcccctaagtgcaaacacagcatgggtccagaagacgtggtctagaccagggctgctctttccacttgccctgtgttctttccccaggggcatgactgtcgccacacgcctctgtgtacatgtgtgcagagcagacaggctacaaagcagagatcgacagacagccaggtagttggaactttctgttccctatggagaggcttccctacacagggcctgctattgcagaatgaagccatttagagggtgaaggagaaatacccatgttacttctctgagttttagttggtctttccatctatcactgcattatcttgctcattcttcagttctctactccctcttgtcagtgtagacacaggtcaccattatgctggtgtatgtttatcaaagagcacttgagctgtctgaagcccaaagcctgaggacagaaagaccctgatgcaggtcagcccatggaggcagatgcccttgctgggcctgggggttttccaagccctcagctggtcctgaccaggatggagcaagctcttcccttgctcatgagctcctgatcagaggcatttgagcagctgaataacctgcacaggcttgctgtatgacccctggccacagccttccctctgcattgacctggaggggagaggtcagccttgacctaatgaggtagctatagttgcagcccaaggacagttcagagatcaggatcagctttgaaggctggattctatctacataagtcctttcaattccaccagggccagagcagctccaccactgtgcacttagccatgatggcaacagaaaccaagagacacaattacgcaggtatttagaagcagagggacaaccagaaggcccttaactatcaccagtgcatcacatctgcacactctcttctccattccctagcaggaacttctagctcatttaacagataaagaaactgaggcccacggtttcagctagacaatgatttggccaggcctagtaaccaaggccctgtctctggctactccctggaccacgaggctgattcctctcatttccagcttctcagtttctgcctgggcaatggccaggggccaggagtggggagagttgtgatggaggggagaggggtcacacccaccccctgcctggttctaggctgctgcacaccaaggccctgcatctgtctgctctgcatatatgtctctttggagttggaatttcattatatgttaagaaaataaaggaaaatgacttgtaaggtcc

SEQ ID NO: 36 - DNA, 8141 bp, Homo sapiens
cttgagaaagagagcgatagagtgcgagagcgagtgcccggagcatcctggccctgagacagctgggccagccccgcagggctctgcagcatgtgggagctccgctccatagccttctccagggctgtgttcgcagagttcctggccacactcctcttcgtcttctttggcctcggctctgccctcaactggccacaggccctgccctctgtgctacagattgccatggcgtttggcttgggtattggcaccctggtacaggctctgggccacataagcggggcccacatcaaccctgccgtgactgtggcctgcctggtgggctgccacgtctccgttctccgagccgccttctacgtggctgcccagctgctgggggctgtggccggagccgctctgctccatgagatcacgccagcagacatccgcggggacctggctgtcaatgctgtgagtagccacaactttgccatccacaaggggcaggtcctggggaatcccttgtaaaggatgagatgggagggatgggctctgggtgatgtagggagagagatggagacagaggcagagagagaggctggagccaggaacacagccaccataggagggtcaggatgaaaggagcaatgataccagagcaaagcaggatgcagacagactgctggcttcttccaccctgaccgtcgtgcaggaagagcctcatccaggtttctgttggactcaacgcctacattacagtgaagacagggctatacctcagcaggggttggtgtcccgagcagacatgctttccaagctgcggggagccttggagtgatcatcacagggttctcaagagagaagaggggcccagcatttcttctgctttgagggtttgcaggtcccgtggcaggtgctccttacaggccaggcacggacacaccacagccctgcaagcagcaatgggcatactcacttctccaaaccttgaggaggtccccggagcccataagctggctgctgattacatgatacaggcgctaactgggccccgtgggccctttgggagctgagggtggtgccacagtcccttcccccatgtcccacccacttcccctctcaagtctgtgtgggccctgtgaatccaaggcccatgaatgtggaggaatgacctgtgcccccaacagatcaacactcactctgtacccccatcctaagccaggacagtgagtgcctcttttgtgcttgtttccggtagttcccggccttttccatccttgagccactggacaagagtcctgatgtctttgatgagtcccgcacaatctctgtgggcgcccccttctctgcacaagtggcccttcctcatctgtgggacagaggtatcccaattagggaacttctctgctttgttttgttttgtttttgagatggagtctcactctattgcccaggctggagtgcaatggtacaatctcaactcactgcaacctccacctcaggccattctcttgcctcagcctcccatgggattacacgcctgtaatcccagctaatttttgtatttttagtagagacaggactcgccacgttggtaaggctggtttcaaactcctaacctcaagtgatccacccgcctcagcctcccaaagtgctggaattacaggcatgagccaccacacccagcctctgttctttaaagaggaaaatatgacaaggctgggggccaggggccaaggaaaggctaggtgggcaagcagaggagtgggctcatttcctctgagaacaaaattagttcagtttgcagcaagaagaggtccagttagactgaaaaaggaacttcctgagagcatgaccaggaagtccttctctaatagcagggcactgggagtgaaaggccatgggaaacatgcagtcctcctcctccatcatttcacaactgactggggccctcagaagagaaaggtctgggagggctcctccatgccacttctcagcatttaccaccttgaggacctggggagaaccctgcctggatggaatggggcagtggtgggaccaacttgctctgaggctaaggagagccagatgaccctcaagacaatcccaagtgtcctttgaggacactgagagccagcctggccaccaggaagttaatcattgacatgcacctgccctgtgccaggccagctatcatctcatcattggtacaacgctataagggaaattctgtggttccccctatggtgagtggcaaattgaggctcaacagcaattgatcaaagtcacagaaccaatatgtcatttctagtcctcccacagtcctccagaatccattttcttatcccatttcacagaaggagaaactgaggccaaattaagtagtcccatggcaagtcagaggcctccaaagtgtctgcttccaaagctcttttcactgtcctctagagactctgcattggacaacaataggagggattgaggttagaccctagagagacccaagggttctgacctgatgtctcggacccaggggtgggggttcagggcctggagaaagagcgtgggagctgagagctgtcactgttccacccagtgcagtgcccagccccagaaggcagggtgtagggtacagggtgacaaaacccaaggagaaatccagtgctgggtgcggcccagttatgaaatcacctctttactcagccccgagttctctccttacccaaggcagggtctgctgagaccatgggcagcctacatgggtgtggaggcattgtccaacacagctccagggcccttaccccaacctccataggatgtgcagggagcaaactaacagggcaaacctgaggcctgaaaggcagtggggaatagacaagggttagggaaagaggtagattccggactcatagcggaaaggggcacactaggactgtcccccagacccaagtccctagaccctgtctctgggagccatttaacctcaggaagaaagaccacaggcctctcattgcaccaaggtctgatggtctacagacagaggaaggtcttcctgctaaccggcccaaagcttcctgaggcagttgaaacctcttcccttcttgttctttgggaaacagaaattccggatctcagcattcttttcctctcatactgcaggctcccagcctcaggccccaatctaatgggctacagagtcagttttgctacctctggcggggggaccatgggcatcctgggggatcaggggctgcctttgggccagggcccaggaagaagggatcagtcgttgcagctaaggcgtctggcaagcccaggtgttccggctcccagcccagaggccccctggtgcctcgactgcaggtggacaggaagatggagccagagaggaaagtgggctcagtgttcccctacccgcctcttctctgtccccagctcagcaacagcacgacggctggccaggcggtgactgtggagctcttcctgacactgcagctggtgctctgcatcttcgcctccaccgatgagcgccgcggagagaacccgggcacccctgctctctccataggcttctccgtggccctgggccacctccttggggtaggtcatggccatgggttccagcctccctggaggaacagacacacagaccactccagagacagacacagagaccccaagagggacacatacacagaactctcaagaggaacagacaccccagaggtttgactcctagataccccagagggacagatatcactccagcccatctgtaaataaaacgtgatgttaattgtccatcacgtgggttccctttaggctgaggtcaagcactgcagtgcgggacaaggacttcctgccctgtcctcacctcccttctctctttgatgccctcctcccactgcagatccattacaccggctgctctatgaatcctgcccgctccctggctccagctgtcgtcactggcaaatttgatgaccactgggtaatggctgaaaccccctgccctccccttctctagaaacccattttagagggagaacaagagctggaatagcatgggatgggggctcagcagcggtaccccaaaccctccacactcctcctggtcctggggagccttgggttccacccctcagatctgatgccaaagactcagtttccacgtctgtgaatgaggatgacaacagcttacctcactggcttcttgggaacagtaagtgaggttaccggtgtaacccagataatgcagtgtctggcacttagaactctataagtggtagtatttagcacttatctggtgcttagtatgtggtgagccttgttctgagtgctttgcaaacattaactcagttttcacaaccaccccaggaggtagacattcttagagtgaaaggcacagagagggtaagtaacttgttgaagtgcacacagcacttaagtggtggaatcaggacacacacaggcagtggcttcagaattcgcacccttaaccccgcactgacaaggcttccccagcagctggcgttgtcgttgtaattacataaataagcattttactagattaatgtcggggaggagaggtgcggccgcagagtgtgccgccggggcctgcgggctccgcgtgccggtgccggcgcgggtgccaagccgccctctccgctcgcccccaggtcttctggatcggacccctggtgggcgccatcctgggctccctcctctacaactacgtgctgtttccgccagccaagagcctgtcggagcgcctggcagtgctgaagggcctggagccggacaccgattgggaggagcgcgaggtgcgacggcggcagtcggtggagctgcactcgccgcagagcctgccacggggtaccaaggcctgagggccgccagcggcctctaaggccccgacggacgcttgtgaggcccgaggcagaagggcccaccccgtccctcctctcccgcaggtctgaagttggccccccagcgcagagtagctgcttcctggacgtgcgcgcccaggccagtgctgtgagcaggcggggaggaggctgccggagggagccctgagcctggcaggtcccctgccctgaggctgtgagcagctagtggtggcttctccagcctttttcagggaactgggaacttaggggactgagctggggagggaggcaggtgggtggtaagagggaaactctggagagcctgcacccaggtactgagtggggagtgtacagacccctgccttgggggttccgggaatgatgcaactggttttactagtgtgcaagtgtgttcatccccaagttctcttttgtcctcacatgcagagttgtgcatgcccctgagtgtgaacaggtttgcctacgttggtgcaagtgtgcatggctggggacttctcacttccccttgcaccccttcctccccaacctgcaataaatccgcttctcctgcagtggatgagtgtgggtgccagtcctcctcaggagaaggggaagggaaggaggccactttgagagggctgaagggagggccttgatttccagccgcaatctggctcctcctggaaaatttcccccacctgcagcgctgggccccaaggcttgccaagactcattgctggaggtagagaggtgtcaaccagagaacagctcccctagacatggcctctggaaaaaggaaatctttggtggccaaagatttcctttctgtggtgagggagaacctctccaaagaggaagcaggcatatgagtgcgcacgccttcacgtgtgtgtatgctgtgtgtgcccgggacccccgtctccaggggtgccccagacccagagattagcctcccacttcggcgcagaaaacaaaagccctttgtgggacccgggcctttgtcttgggtgaggggagatttgcacctagacacttcttgaaaggagacactcagtttcacaagtgagccctcttgcccagcacacacctatggctggctgggcaaggcgctcaggcatgcaaccatgggcaccaggcaatacccatccatcacccatctcactctccccagtcctcaggcagcagcttttccaccccaggaatctctgttccttctctgtctgcctctccacagcccgtgatcccctgcttctggctcctaccactggacagtcattgtggatagagcagcctgctgctctcaggaccagagaagggaaatgacttctccaggcagaggcagatctgagtctagaacccaggcttctaaatttctaggccagtactactgctctcaaaagagattaactgggattaagtgagatgacgcatgtaaagatgctgtgtaaactgtaattcttaataccattatcacgcattactagaatcatttcattatttctgcttcctgggggaaggatgcagaaggagtctgatgctcaacatcccttacctcctttcgttgatggccaaagcaaaggagagtcctgggagcccacaagatccaggcagaaggagatgggtttggggctggccccatcctggcccagcctcttaacctcttcttaccctgaatgtgtgccctatcttttctcccacctccttcctcctaccacccagccctatccattctctccttccctctccagcctgctgtataagtaacatgtgggtcataccttgaacccccctccgcccccatgctctctgtggcctccatgccttcaagacttctgtctgtatctatctcacactgcaccaagcacccactgtgcaccaggcactaagtctctagcctctttctcccttctgctcccatccaagaaagacagtgtataaggaacatctctgtaattgtgtcccctctcatctatgctctttcctgtcttccttgaccaaagctctctttgaggtccaaaaaaggctgccctgggccatatgtgctatggagaggactctcccaaacccccaatagctagtgacagccacttggcctcactaccagaagaagggtggagtcagaaaccaaagtgacctccagagctgtcctcctggcacctgagtgtccttgctctagaggttcaaaggcagcaaggcagtgagctatgagcccagcatggatgtgtctcaggcacctcatccccaccccacctcaccccaacaaactcctctagaagaagccaagaatttctctctatgtctgtttcactttttggattgcatctccccaaaaactaaatgtgaaatccctaccatttctagcctctattgcccagattggagtcagaggtcaaaaaaggattccaagtgatggaactccaggggtggagggaaattggcagttcctggcatctcttgtgtaccagctgctgtgctgggtgccttgcatgcacggacttatctgatcttcctcaaacttcagataggttagggaaggacaaaggcccccagccatgaagggttttgctatttccaaagcttccccttatcgtcccctgtgattatctcccaaccccgtgacaaaggtagagcaaatattgttcttcccattttacagattcagaaaccaaggctaagggactggtctaaagtctcacaggtagtatatggtggagccaacacttgaactcagacctttttacacaaaatcccatgatttttccactgagacaaatcctaggctcctgggaggccctggcagaagccagaacacatggctgggcctttccagcccaacccactgtcttgcagtggacggagagccttggcgagggcagcagggtagagaggacaaagaatgtggggctggtttctgccctttaagagcacccggtcagtcagagaacagagatcacacacaccaaaccagacagaggcagggcagcatggagacaggtagacagacctccaggtccctaagctggggacagagaaggttctgagctgtcccatactcccactttgtgccaaatgactttgcacctgcaaatggtgcttcaatgtcgttttgtatctttaagctgtgatgttgtatatatacaatgcctcccagctagactgtaagccctttggggacaagggctgcttccagttcttggatggcgttctcctatctaccttccttcaactgctctgcatataataagcactcaataaatgctcatttgccaataa

SEQ ID NO: 37 - DNA, 6434 bp, Homo sapiens
gctcgctgcgccaccgcctcccgccacccctgcccgcccgacagcgccgccgcctgccccgccatgggtcgacagaaggagctggtgtcccgctgcggggagatgctccacatccgctaccggctgctccgacaggcgctggccgagtgcctggggaccctcatcctggtggtgagtggagggagccggggaagcccttctctctccagcccttgcactccccaaactctcacttccccgaaggggctgtgttttccaaggtagcctggacccacctccccagctgtgacccccacgcttaaccgcggaggatcaagctgacttccaaagtcctcttccccacggttctaaccccctctctgacagctccgactcttgccagaatgacagctgttactccccagtgatagtgccgatcgtttaccctcccacaagtgactcagccaacaggctgggggcagcggtcacgcttgcagtctgggacagccagccacttcccctgcctcccacacccctccagattcccagaatcccagcccccagatgtaggggagggggcggaggcaaatgagccattattaggttatttgggtcctgggtgtctggcctctaatgaataattaagcctcaaaaagtccaaaggttgaagtgagtgaggacagtttgcacgagtgggggcagagggtgcaggccacatgggctgcgaacagcgaagtgggaagggaggcagattctgacggtagaagggtgggaggaagctaggcccaggcacagaggacgcgtgtctaaagttgctcacctggtgctcagcgactcccaggccctgtctgttatccagacagatgctgtcagcgtctgtgcagaagacgacaagcattctggttactccctggcctcttggttactctgtgggttctctgagtgagcatttccctgcctgagttactcctcagtcctagttactccttggtcacaccagtttcaccctggttttgttctgagtgactctgtttgcacttcttaggtctggcagacccaccagttatgttctatttcctgtcccagttactctgatttttcagccgctcatgtggttacacctgttttcctctgtgttcagttgctacattgcagtgagtgggttactccgttcctgccccagccctgggtagtacagaccagctggcccactgtggactaggaagcctaactaagggcaagtagggatccccaaatggttgaggatcaggatctatgggcagaggagggctgaaacccacttctggcttccaagaataggaagtggggaagccacagggggcattttatccacagcctcccataagattagtcgtcctatcagccagccctagcccctctctcctgtgccaagaaggcaataggaggtggcctttatcactctcctgcctcggagccctcactcctatcggagtcctgagtccagccacctgttttagagcagccaggctaggggaggatgatgtcttctattttcctagaggggcctcctcattccctcccaagtctcagttcactctctcacaaaggagccgttagtcctcccactgagccccaggatcatcaggcttatcccagaccacaaaagaagagtgttaaattttaggggaagggttgcagactcagatggatgtgaggtattggatatcagtgagaagcagaacttcaaggactgcatgaactggaggggcaggcctggggatccctttgtagtgggtgagtgcctgcctgcctacctgcgtgttgggagcctaggcctgcagtggcagaatcagggttggctttggggtgaggaaagcccctttctctgagatacctggtgtgagggcagggggcggtgctggccgagaactcagggcaggggttgggggaggaagaggaagattagccccaaggtggtggaactggctctgacagctcctccctccaaggcgtcctgggatggagccaggtctgagcctttggcaggcaaggagctgctgccagggcctcccaagcagggcagggcgcctcagaccctcacgtgcctgtctcccaccacccctcctcacacatacacctgcaatctctgatctttgctctcacctgcctgcacacctttgccccatgtcattgccgctggcttctccgttatctcttggtctttctgggatgataatcaaaatgcttaactatccagtacaacaggcactgacccgtcagaataaacaaggtctgtagcacaggtcctggaaactgcctgctctgtgggcataaaacctttagttatgggcaggtgggagaggagaccagggcaaggcattggagcagctttgagggacactcagggggctcagggcaacaatgctttacagctgctgcagaagtattttcaggcttgaacaaggggaacagacatagtacgttctttttcttttttaaatagagacaaagtctcactatgttgcccaggctgttctcaaactcctgggttcaagatcctcccatctcagcctccaaagtgctgggattacaggtgtgagccaccacagctggctccagtatgttctatatttgtagaacaccagggctcttagtctcttttagttcccccatttttggcagaaagtttagaaccgatctccccatttctacagccctgggtcccgggctggggatagtgagaggagccacacagatccctgtctgtaggacctccaaatctgagtggggaatctggacccctgagagcagagaggtcatgggtggagagaggagacgctgggacagggacaggtaaaggaaggggaggttgttgagggggacatggtgaaggctggggctctggcatcctggactcaggagaagtggattcaacctcacttctggtctcctccctatttatttatgccgcccattgccaccactaccaccaccacatgagcttttcttttcaactcttttttttttttttttgagatggagtcccgctctgtcgcccaggctgaagtgcagtggcgcaatcttggctcactgcaacatctgcctcccattttcaagtgattctcctgcctcagcctcccgagtagctgggattacaggcacctgccaccacgcctggctaatttttgtacttttagtagagatggggtttcactatgttggccaggctggtctcaaactcctgacctcaggtgatccacccacctcggcctaccaaagtgctgggattacagtgtgagccactgcaccgggcctacatgagcttttctttggggcagctgggggatgagtgagaggcttcccacctttatggtccaggcctgaagggctgtggagaaacttgtgctaaggtgaaagctgttcccctacacctacgcctgctgaaattcaaggaaaatcagtgaagatggttcagtcatcttctaggatcccagtcgcagaccctgcttccagccctttccatagggccctctcatattagagttgggacagtggggtgggaaggcatgtgtcctttttctggaaagtgcaattacagcagaaggggtttgggctgggttccaggaagcgcctctagtcctcccagtggtggcgagtgggcatgttgctggcttcaccccttccttctggagtgagagttgctggtcctcaccctccctgcctgttcttcttcctgacagatgtttggctgtggctccgtggcccaggttgtgctcagccggggcacccacggtggtttcctcaccatcaacctggcctttggctttgctgtcactctgggcatcctcatcgctggccaggtctctggtaaggccttaaccctgcccccagcccttggccctcaatagcattcccactaggtgtcctggcattcctaagggcaggtcacagctgtggcctctgctttggccccttgggaaaggagggtggagaagaaacttgacacttagaactttcgactctcaccttggaatcagagattatcagctgacctgttacatagaccaaccgccatcctgtgcaagaaacccctctctgcaccccttctcaggggaccctagcctgccgactgtggcaggctgcagctaataggtcccttgtcccctctgcccaggggcccacctgaaccctgccgtgacctttgccatgtgcttcctggctcgtgagccctggatcaagctgcccatctacaccctggcacagacgctgggagccttcttgggtgctggaatagtttttgggctgtattatggtaagcattccccaccctgtcctcctccactacccccgtccctctgttcaggacctgctggcaccaggccttttgatgacagacggctaggacctgcccaggccccgggctcatgactcactcattcacgcacagggtcaaggtagggggcacgaagggaaagaaacaagttgggcaataacagagtctcaggccctccaccccaccccacgccaccccctctgcctgctgcaatacagcagtattgctacttacccataactcatgggagggtggggagggcacacctgagagggaagtctgggctcaggcctctcccccgactcactgtgtgtctaatctgtcaccagatgcaatctggcacttcgccgacaaccagctttttgtttcgggccccaatggcacagccggcatctttgctacctacccctctggacacttggatatgatcaatggcttctttgaccaggtatgggctggggacgtgtgaggggaacgcagggaggggaccgagttgccttggtagctcatgggctggttgggggacaggactcctcgactgtagcagggtttctccaatctgtggggtaacccgcatcagaacatggtggcaagtacttacaaaacatgcggctctccagcgggttcttgtcacgcagacattctagcaccattgctttcaggagaagagcatgggcgggcgctgacaagagtttaagagctagagggaagacgggggatggaaggaggggtcagagaaagggagggagctgcagctcaccctgttctccccactccccagttcataggcacagcctcccttatcgtgtgtgtgctggccattgttgacccctacaacaaccccgtcccccgaggcctggaggccttcaccgtgggcctggtggtcctggtcattggcacctccatgggcttcaactccggctatgccgtcaaccctgcccgggactttggcccccgcctttttacagcccttgcgggctggggctctgcagtcttcacgtgagtacagcccccacccagctcaccccagcctgcctctcctctgccctgccccccatgtccctgactatgagtgtctgtccccccaggaccggccagcattggtggtgggtgcccatcgtgtccccactcctgggctccattgcgggtgtcttcgtgtaccagctgatgatcggctgccacctggagcagcccccaccctccaacgaggaagagaatgtgaagctggcccatgtgaagcacaaggagcagatctgagtgggcaggggccatctccccactccgctgccctggccttgagcatccactgactgtccaagggccactcccaagaagcccccttcacgatccaccctttcaggctaaggagctccctatctaccctcaccccacgagacagccccttcaggatttccactggaccttgcccaaatagcaccttaggccactgcccctaagctggggtggaaccggaatttgggtcaatacatccttttgtctcccaagggaagagaatgggcagcaggtatgtgtgtgtgtgcatgtgtgtgcatgtgtgtgcatgtgtgtgcaggggtgtgtgtgtgtggggggggttcccagatattcagggcaagggaccagtcggaagggattctggctattgggggagcccagagacaggggaaggcagcctgtccatctgtgcataaggagaggaaagttccagggtgtgtatgtttcaggggcttcacatggaggagctgcagatagatatgtgtttctgtgtatgtgtatgtctgcctttttttctaagtgggggcttctacaggcttttgggaagtagggtggatgtgggtagggctgggaggagggggccacagcttaggtttggagctctggatgtacatacataagtaggagcagtgggacgtgtttctgtcataatgcaggcatgaagggtggagtgaagtcaggtcataagtttcatgtttgcttttgttttgttttgtttttaatgtatgtagcagatgttacagtcttagggatccgggatgggagaccccactttagaaagggtcgtcactcctttaatcctctactcaacaatgtactcttttacttttatattaaaaaaaataaaataaatatgtgcctaaaa

SEQ ID NO: 38 - DNA, 13665 bp, Homo sapiens
actctggctggggaaggcatgagtgacagacccacagcaaggcggtgggggtaagtccttctttgccttaagggaacaacgcttagggccaaaagatgaaactgtctcttttattatagatgtttgtgccttcttaggcagaatctggaaggcagctatagggcaggtgggtggttcttttagatctgagtactgggcattttcagaagagaggcatagagaaggtagaagaaaagggatgctttttagccaaatcctccatatgtttcagatctgcaaaagcccaggaaaattaccctatggaggtgaatctagctatagtacatttcttcggaaaaattactcacccagacgctcagctgtcctggagtggatgaagctgagtgttcagctagaaagtaatgtgtccgattcagtacatcaacaaacccgctttgttctggggcaaaagaagcaaatctaattgtgttgatcatttccagtgactgtgaacatataagggaacactgacaataaaagcagggtgctatgggtgacgctagggtgagccctcataaggccagagatatgtatatgctgccagcaaacttcacggttcacagccaagtgaggctggtccatctgccttggaagaagatcagatttttgtggatttaaaaaaaaaaaaaagctaaaaacattcttaaagagcatgtttatgttttgtgttggtgttttgcactgttttaaatttctctggttgatgaatatttgaagctgaaagagcaactacctctggagaggtaaacaggtgcatgacaggccaagcagggagagaactccggaaatggcagaagtgagaggtgaagggggtgagcagcggggctggccttttctgggtcccacctcttctggtgtctcctgcggggctgaaaaggacgtttcctgcccacctagcttccttagcctactgttttgtcctcctccttctcccctaaccctgaccacaaacgacagcacctatgatcgcattttgccccctttgccatcccggctacagccctgggctgccggtgagttcagaagtgactctcctaacttcttcaatatgtgacttttctgagtttaccgtctttgggaatatttttagtgtctcctccagggtgccctccctgccccattgcaaccatcgtttttgtctctttacattcttttcctctagagagtatgatgtcgttgtcatcccacctggggtttttcatacttctgagattcgcttcactggggcaggggatgcgtgggatgtggctggccacccccctgtggagccagctcttcctttatcctgccacccgggcctgggcaagaaagggtttttctctctccacctctaaagcaaagccccatagcaaactaagggctaggctgagctggaaattcccctcaccctttttggcactgcatttcctcactacccacgcagaaagcaggttcccgggaacattcagtgacatggggaaatgagggccccgcaatttgtcaaagggaaagccacaataatactaatctcttaaagtacttggctttcttgcctaaccccttacgtggtcctctgcaattagattctctggtaaatactctccatacttcttggctctcatgactatcacaggcaaaggacaagggcaaaagtcttagatgatagggtaactggggtaattgccccccccccccaaaaggggaatcctagccatttcctgccagaatgagcagttggcatcaatatcgtcaaaccaccccttctctgctccaaagatctaatgacagcaagagggatatagacgggaggagaaggcgtcctccctctcccagactccctgcccaccaccctccccagagaagctccacaaggtgagacacactgtcctataggcccatgtaacagaaagccatcaaaaagaccgtagagaaaagccgttttccatagagccttctgctcaggggcccacagtttccctaaccacacccgcttttcctgacttcccgagctctgcaccatgaaggcaggaaaggtaccggggcattgctggcacaccccagcctccaggctccctttggcaggtttatctgcattgtacggtcagggtcaaggggtgagagagggtcgggcacgaggagaggctgtccttcctcccctaaaagcatcccccactcccatctctccccacctccagcggccaggatgcaccgggccggcgggcaggcgggcgagctcgcggggccgcttctgcgtgtgcacctggccgccgcccgggagcctgccacgggccgggaatggccgctgctccgccctctgagaggtgctccaggctccctggggacggaggtttacacgtccccgtgccgcgctcccagatctgggcggtgcgcgtgtcccttgggcaggctcagctcccggactcctcccgctggcagcctgctcgttcctggcgcaaaggagagggagggagtgtcgctgtcccagctgaggccctgcctgttcccatcgcccttgctagtcaccagaagctcctctcccctcaatgagttcactttctcaaaagacagccttcagcaccctggtttcctagaaggcttccccctaatccagagtctggggggagggggagattttcagaatgccatttcaaagcaatcaaatgcaaagctagaaaagacgaccccccccccccacgcacaccttgttttaatgctttatgttcgaaatgtaagtagcatataaaatgtactcgaaacttaaaaacttgctctaagggaattaagtacaaggtaaatatattttattcttaatattttaaggaatttgatttagacatttttcctttctagggacagtttggataattttccgtgaaactggatgatctttgaatattaaatgaaaggagactagaataacagtctcttgactattcatgaaggagctttagcagaagcattctttcttggtgtgcaaatcacgcctggcagcccctgcccacagtacccgaagatggcaggccttgttcccttcacctaaattcataaacctgggtgtagtggcttctgatgctgatttgtttctcttttcagtaagtgtggacctttgtgtaccagagagaacatcatggtggctttcaaaggggtctggactcaagctttctggaaagcagtcacagcggaatttctggccatgcttatttttgttctcctcagcctgggatccaccatcaactggggtggaacagaaaagcctttaccggtcgacatggttctcatctccctttgctttggactcagcattgcaaccatggtgcagtgctttggccatatcagcggtggccacatcaaccctgcagtgactgtggccatggtgtgcaccaggaagatcagcatcgccaagtctgtcttctacatcgcagcccagtgcctgggggccatcattggagcaggaatcctctatctggtcacacctcccagtgtggtgggaggcctgggagtcaccatggtagcatctttagctattttcaaactaggactccaagcttcttgcaaactctttgctaaataatgccttggtggcatgaggcacatccctaaaatagctaatttactcctagggaatttttgatgattcttaatagtgtgaaaactgattctggcagtatttaggtatatctgtaagaaatcattgtttgctcttgagtgccttaaaaataacaaaatctaggtggcccgaaaattgtgttggttttttcctccccttctcccattccaattactatattgactttatatttagcttaatgcattttccttacagttatttgttcactagcagaaaaaaactattttcttgtcatgttcataagatgtacaatgatctacataaagggttatgcttaaaaaaatagacatccgatcctagttttgttcttagctgattaggagtatcaattgttaactcagcaccagggctgattaaaaaaaaacacacatggcttaaattttaattgaaatatatattaattaatttaaacctaattaatggatttaatcattgccttataaaatcacaaggctctcaagaaattcttttacgagttaagtgctgcagtggcacattttgcttcctttgtagaaaaggacattcagcacaaactcctgtttggtcgttgtaagagtaatcacagtcatctgtgatgacccaaaatgaatataaatttattccttctttttggggggtgagagaaggaaattctcagttgaaggtggggtagaagacttacctttactttttacacagataaaatgtgcctttcacaatgatattaatacattgtcatcgaaaatactcttgcttcaattctgatggaatatttttctttctctaggttcatggaaatcttaccgctggtcatggtctcctggttgagttgataatcacatttcaattggtgtttactatctttgccagctgtgattccaaacggactgatgtcactggctcaatagctttagcaattggattttctgttgcaattggacatttatttgcagtaagtttccaagtccatttaaataatcaatcccacctcatttatcctcactctagctggcctgaagatgttgtgttttacagcagtagctctttagtatccagccatgactgtccattttaggttataatttttccaactgctcaatgagaatgttgatatagctgctaatatcttgaggttcctctaagtgacaaatgacagcaaattgcaatgaatgatacttgctagtagccctgtttcagcaatgtctgaagttgttttcttgtttcctatagatcaattatactggtgccagcatgaatcccgcccgatcctttggacctgcagttatcatgggaaattgggaaaaccattgggtaaccctcatattgatagttcccatcttcctacagttgaggacaattacctgtggggctcttttatttttcattattttgcctactactttctcttgcatttctccaaccttgaccctcactttattacatctttcataaagactagggaaagaatagcacagatctgctgtaaaaagataagagatgtaaaacatctctaactgaaggttctaggcctccaataatagtcttttccttttttgaagcactattttagacactgtatctaacagattgcagcagaatacactgtatatagaggttgcctatgtggaacaagttaaagtcagcatggtgtagtgaatatggtgtttaaataattgaaagtaaagtcttcccagctataatggaaaaaaattgcaaaaaaaagagaaagaatgaaatacttaaaagcgctgcccatagcaaagatatttaccctttttttgtttacaaatgcatgaactcaattaatagtatgtaagttgcaagtatttgaactgcagatggtgtcgaataaatagtgcaaaggtttatgttattatcaccataactgttaccggaaaaataaatgactcacagaaaaggctcacttttcagtctcaaacctaaactattataaataaatatttattaatgtagtggcagtgcttccagagaatagcagtgtccattttaaaatattataattaattttgtttcagttgtttctcacagcaaaggaatttaaatcctttaaaattaaataagtaatgcagcagttgtgaaggccaccaacatgctaacttacaatagccaataaaaatatgagacctgtaagccagcttcaaagagcgctagcttttattataatttatatattgcaaatgtcacttacaactttaaaaaattcatgaatataacagaatatgccccttgtgtttattcagacagtgattaccaacctagtcacagccattcatctatggtgtacactgcaaatgacaagaaaatgcaccgctgctatgtgcattcacttctaagaaaagtataccaaatctttaaaagtaatctacagcattaaataacagaataagagtcagcgttcaaaccaagacttagccaaaaatgccatccatgagattagtcattttgtcactgatacctccatttatgtctcaaaaactgttaccacattaacagaaatggaaatttttagtgagtagtatatgattttgttttcttctttttgagatggagtcttgctctgttgcccaggctggagtgcagtggcatgatctcggcttgctgcaacctccttctcctgggttgaagagattctccaacctcagcctcccgagtagctgggattacaggtgcatgccaccacactcagctaatttttgtattttttcatagagacagggtttcaccatgttggccaggctagtctcgaactcctgaccttaggtgatccacctgcctcagcctcccaaagtgctgggattacaggcgtgagccacagtgcccagccctgatcttacaatgaaaatacaactttcagttcaacatattaaattgaaactattttgtcattaacaacttttagttaaatttataagcacttgaaaatgtagatgaaaaaatatattttactaattacttgtcagttgcagtaaactagttaagatttaaatagtaggaacattcttagcttgttgattaaactaaaagttgccccatgttatattataccaatcttaattctactaaaaatagcaatggttgtatgaaatttagaactaatttaaagtatatttgtaggtagaaagattaggaataacattaaaatattaattgggttttttgtatagtgtaatgatattattttagttttctttatacttttacacattttctagatttttagcaataaagattctttactttaaagtacatatgacttcattttgaaatagcaacttgacactgagatttgtttatttttgagaacatgcagctgggtatcttattcacagatgttttggaaagggtagtaattattttgctgttctagtctcccttgtgggatttgtagcctgtggaatagtacatgtctaatgagaccttactaacaaagagcaaagacatttagaggagggctctcatttctcctttaaaagggcatagagaagataaaggctcatatgggttttttcatgttcaagagaggaacttgattcttccagccacactagcaacagtttccccgtaaatgttatgtcttcagcctcagcttaaattttgaattttatttaaatcttacaatgaggatgcaaactgagattaatttgcagagggtgttttaaacaaaaagcatccatcatgttttttttcaaatgagtgtgtagtacagtggaaaacagtatactgcagtgtttggaacagtgtttcaaaagagaggttttcattttgtttgtttgtttttttcttttttttgagacagggtcttactctgttgcccaggctggagtagagtgtcacgatcatggctcactgcagccgagacctccctgggctcaagtgatcctcctatctcagcctcccaagtagctgggactacaggtgcacaccaccaagcccagctaatttttttttgtattttgtagagacagggtttcaccatgttgctcaggctgaatcgaactcctgggctcaagcaatccacccaccttggcctcttaaagtgctgggattacaggtgtgagccactgtgcccagcctcaaatgagaggtttgaagtcagacaggtctgaaatctcagctcaaccatttgataagcacaaaatcttaagcaagcttattaccctctcagagtgtcactcttcttttcataaaatgggaagaataataatatttatctcattggttattgttagaattaaatgagattatacaagaaaagtgccaagtacggctggcccggggacagtgctcaacaagcagggtttctgttgcccaagtgcgtctgttaaataaatgtctaatattctacagcacacatgtgctctaaagcaaatgtctgcacttcattcaaactcaaaactttattctaggggcatagctaatatgtaataataatttccggctgggcacggtggctcacacctgtaatcccagcactttgggaggctgaggcaggtggatcacgaggtcaggagattgagaccatcctggctaacacagtgaaaccccatctctactaaaaatacaaaaaattagccaggcgtggtggcgggcacctgtagtcccagctactcaggaggctgaggcaggagaatggcgtgaacccgggaggcagagcttgcagtgagtggcgatcgcccactgcactccagcctaggtgacagagcaagactccgtctcaaaattaataataataataataataataataatttccatttattgaaatcctatggaattgctacttctcaactagccataaactcatattttttcaccaagccatcgtgagaaccgcatagatgaaagctatctgcacagtaatgaacacgaggttttcatttaaatcaaatcaccaaattttggaagcctggaatggagcagattactacgagctaaaaactatcaagtactttacaagtattgttgcatttaatccttttaccccgtgaggtttgtattattataatccccactttacagatgaggaaattaagacttggaaagttacgtaactgatacaagcactcataactctgctaagtggtggagctgggttggttttgatcatgcttacctgaataacggaatccccaaaagataaaaactgtgttatgatcatgattgtgcaggaaaccagtgtaaaatggcaatattgagaaaataagagaaggactctccagaaattttttttttattttagttaaagaattcttagtgcgcttaatttgtagtaccaccaaagaggatctatctttacattaacgattatcataagatggaaagaacataattaatgaaaagaggataggagatgaaacagttctttttgttttctgttcaaatgcagtttaataatttatggatctctgatgaggctttggtgtgttttcttatttcctctccaattcccctctttttgatgcatgacacaaatacactcttaatttaacccagctagattctacattcatgagctgctcaatggaatagcttttctctacttacactctatcctcttccatcttttccttagatatattgggttgggcccatcataggagctgtcctcgctggtggcctttatgagtatgtcttctgtccagatgttgaattcaaacgtcgttttaaagaagccttcagcaaagctgcccagcaaacaaaaggaagctacatggaggtggaggacaacaggagtcaggtagagacggatgacctgattctaaaacctggagtggtgcatgtgattgacgttgaccggggagaggagaagaaggggaaagaccaatctggagaggtattgtcttcagtatgactagaagatcgcactgaaagcagacaagactccttagaactgtcctcagatttccttccacccattaaggaaacagatttgttataaattagaaatgtgcaggtttgttgtttcatgtcatattactcagtctaaacaataaatatttcataatttacaaaggaggaacggaagaaacctattgtgaattccaaatctaaaaaaagaaatatttttaaaatgttcttaagcaaatatatacctattttatctagttacctttcattaacaaccaattttaaccgtgtgtcaagatttggttaagtcttgcctgacagaactcaaagacacgtctatcagcttattccttctctactggaatattggtatagtcaattcttatttgaatatttattctattaaactgagtttaacaatggcaaaatacagtatgtcacagtcatgcacattcaagagagaaaatataacaagttcttttatgagcaatcccttatgcatagactaccttggcaaaagagcattagcaagtgtcactgctcatcagttacttccttccatttatatcacaaatacccaagtttcaattctaacttcatttcatggtatttcttcctcctcaatgcccaaggtaatgtgggactaaagcccagaaatttgaaaagaatattcagaaatccttcccaaatcataagggcacctattgagattcaagacaagcagactcgtaaaatcttgtagaggcagaggcaaagttatcatcatacaaaaatcacaaacaaaaaggagatctgtattcgggtatcaaacggttgatctgttttcagtgcacaccctcaaatgcaccacaccagttttacgatctaagcttttaacccctttaccactttgcttatttttaaaaaatttattggcaaaactggggattttgtttgtaactttggttatctatatttaaacattagctgagacatatttttgataacaagaacttagctagttgagtcctggctttttgttgattattaagctgtgttcactctagaaggtgtaaaggccctgtcccaatctctgctctctcacttctcgtgacatttgttgataatctttactgcaagtgtaatgatatttgagtgaattaaaatgcatgacagacaaggtacaacatggttgcagaataaaagggtgaggacatcagatcactctgtattcaaagcttactagacgaacaatctttcatttctggatatattgtagctaacccaacacatttttaaactatttttgttactgactcacttacttgtcaaaataattggtttttctgtcctgctttgccttttgtcactagaatgttctccagaaaccccagtttctgtcatcttaactgtctagtctagccgttaacttagacaaagaagtgcaaatgtcaatttcactaaacaaaagctattagaagtaaagcagaaacctatcactgcaaggagtgacttgtaaataactgatgatcaagggctgtcttctctcaagcatggttccaactctgtgtcactttttagcgttaatggcagttgtgtgtctgtggcagtgagataatggaccactattaaacctgattctcttcggtgctaggaaagagtgatgtgtgagattcccagagagtcgtcacaccagtgttatgcctgggagtgccactggcataccgcaggtgcctaaatcccaggcccctttctcagggggcgtggccacaccacacactgcgccagtgacacatagttacttcctggctgggacggcacctatagaaactagggtacaaatctcccaaagatggtttgcaccgtcatttcaaatgtttgcacaatataccacttagaggcttaaaatcagggtggttcagttcaaaaggtaatatcaaccgtcagcaaacatgtcagtttaaaaataagttcggttaacaaggaagtgttaagcagattttttgaatacacttacaatatactggcttataatcatttttattctgccatatggtttatttcatatacactcacttttacactcacttccaattatagcattcaataagttacggttaaagaaaagtgaatggtgtttttgtaatttgttttctagtagctagcagagagcctggcacatagtatgtattcaaaacaagtgtgttaattaataaaaatatataaatctcatttgagatttaagaatataaataattttcttttaatctttattttgctcaatatttaataggaagtatgatttcaaggactactaaaggaaggcaatatacatgtgaaaaccataaaatacattttttgcattctactatttttaggggcatgaaatgaatgaaagctttagcacttttttgcccctacatatgagacaacatattttgtatttcactcaaggctctgccagtaaaaagtaatgaaattgtacctttctaatgacatctatgcagcaggggtctatcgccttgtggatggcaccaaattatccaagtgtattaggagactggggcttttttctttaatcccttcttattaatgaagtgcatagtgctgctcccaggagaccactgctgacagatacacagagaagagatcagagaggaaaaactgggaagacataaatgaattatacccagccatgaaacaatgccaactgtctcttccctaaggaagagtacaagtaccctaaaattgaaaggtggtccctacactgaaaacgcacatagtttgtcaaaagtgtacaaaagggaaagagtcttattttaagctttcaggctttcttaaaaacttggggaccagaatttcaatgtatgtttccattgttgaagataacattttcttcaaagagccttaaccttttgtactggaaggaaatattttctggacttaagtagttgcctaaatttaagattcctacactttatttctgccattgatgcttttcctaaacccttatactatctttttattatctgagccttttcctaatgcagctcataggtgctagctagagctgctgctcagtattgaagactttacaaggagattagaaatctttggaaaacatatgtgatgaaattgagctatatgatttatcagagatctgattccaaagagcacagaatactgttctcagaccatgaaaccagacaacacatgtattggtttaaactcgataatgacaggaaaattccaaactagagcagtaaattcaaatggtaagatgaatcctagaaggcctctgattgcagcatgttgacaccaacctcacgttacgaacaattcacagagaatttgcctttgtggcaactgaagatggaagtctggggggcacagacaaccttatcaaacaatataaaagccaatataaattctcataagcactatagaatttgcaaattcagaacattttatacctaaaagtaattctgtctttcctaaagtgtttttaacatgaaaattagtaggaagatgtggttactatttggaaagtgtaatgtaacaaaactctcttttgttaccacaaattttgtgagtttagtactctacagattgccccataagagcagtagcttttgaaactcataattctctgaaataaatgaaagacatttaattcaaggatcaaaaattgtggccatctttgcaaatgactacctatagcctgtgaaaatacatttcaaaaaatgttatgtgcaatgaacactaaatttaagagcagttacagtgtgactcactcatgtttaaaaaaaatcgaagagctaaaaaatacgtctaatttatgtaacccattggaatgtatttctaggttctcttcaggattaattaaataaacatgcaatttatgaaaacatataaacaattatttatcacttttatgacccaaatcacaataaaattgtcatttaggataaactggggagaatagactgaacatatggttatattcacagttatttattaacttaaatgttattccaacattagagctaatgttaaaaagatttaaactgtaacgtctaatatttggaataatatattaaagtattagcactgtggttgattttcttgaattatgttgcatcttgtactactaagcttgtgaaaataaacatttggatgttttaaaaggtaaa

SEQ ID NO: 39 - DNA, 3811 bp, Homo sapiens
aggccgccagcctcggagtgggcgcgggacagtgcgcggcgccccgcagccaggcccccgcccccgccgcatccacctcctccgccgcctgcgacccaacgggcgccccccgccgcggcagctgccgccgggcccccgcggccaccatgaagaaggaggtgtgctccgtggccttcctcaaggccgtgttcgcagagttcttggccaccctcatcttcgtcttctttggcctgggctcggccctcaagtggccgtcggcgctgcctaccatcctgcagatcgcgctggcgtttggcctggccataggcacgctggcccaggccctgggacccgtgagcggcggccacatcaaccccgccatcaccctggccctcttggtgggcaaccagatctcgctgctccgggctttcttctacgtggcggcccagctggtgggcgccattgccggggctggcatcctctacggtgtggcaccgctcaatgcccggggcaatctggccgtcaacgcggtgagtgccctggggggggggtgggagcctcgaccctggggtgggctcaggaccaggcctcttaccccacctggaaaaaaggggtgccgcagagtgggccagcccacacccttcaccaggaaggcaagagcctcccaaggccaggaaggcaactctccaggctgatatgtgctcccttcctgcccacctcctctccccctcccctcatgctggcccaccctggtctctctccaatgcctgctacccctcctctcctgccagaaacttctgcctcctcctgtctcacctttgagctccaccttcctcctgggcccctcacctctcccatcacctcagtggagcggttgagtagaaccaccagagaaaagcgggctgtcatcacggctttgggtgaccctggcctgtctgtgcttcattgttctcagcgcctccatggggatggttgtgtctgccacagcaggtgtcaaggggctcacatttagaaaccgtagataggaaccccctaatgtggggccagtgcagagcagtttctgttgagtattggttccctctttccccaacctgtgctcctcccctcaaccccgcacccctgcctccctcccacctcctgcagccccagagtcagcccccaaccccatgcaaactgggttcattaatcaaacacagaggaggatttgcttggccattgtcaagctgtgagtcagccgcactggagagacgggtttaaggctggcactggcatggggccaggcacgggcaggaggccgggatggggtgtgaggcaggcaaaccgggcagctgcccacaggatggagggtaaatgccagcttatgggggctgggggttgggcagagccattgataggcccgtgtccaggaaaagctaccctgacgctctgccctttggccacaggcctgagcttcaagcctgtgcaaactatgaaatgtacagtgtctttaacaaatggcttcgctggggcgatgtccacaggacttggcttccaggtgccaaggtgctctaagtgtccctggaggccaaaagccctactccccgagcccctggctatacagccagaaggtggccggggtcctaacccgctatccccttgcagctcaacaacaacacaacgcagggccaggccatggtggtggagctgattctgaccttccagctggcactctgcatcttcgcctccactgactcccgccgcaccagccctgtgggctccccagccctgtccattggcctgtctgtcaccctgggccaccttgtcggagtgagcagtaccgacattgggctggggtgagggtggggcaggcactcaaggcaaataatggagccctggagcggagcccacttcagatggatgagtccagcagagtttaaggcctggatttagggctcctggactctggccctactgggccccagaattgatccccccaaaacctctgggctgaggaaagatgggagtaagtaggaggtgggatgggacaggaatcaaacccaacctcagagcagagaagagaggagggcgtagttaagggtccatggttaaccaagcagctgggatccttggagggagaggttgccagcctgagtttgagacctggctgagcccctggactgcagtgtaaccttgagagagtccagattctgtgggagtggaccaggatgttgcctttctctccaccgccctgtctctatccagatctacttcactggctgctccatgaacccagcccgctcttttggccctgcggtggtcatgaatcggttcagccccgctcactgggtgagtctgtcccttcccctggctccctggagatgagggcctggagacccagcagtggcagctcagagctcaccagacctggattctgtgggtctagagcttgggggtgggccacggagtggtggctgacagagaaagggacacagtaggggatgggattcagctagaggggcagaggtggctttgtatggcctgaaccgctggggacagggagtctgagtatctatccgcttatggaagaggctctactgaacctggaggtgcagaggggctctgtgtcaattcccttgaggatttggtgtccgtccccctcggtgtctgcgtgctggtcctctctgatgttttatttatctgtccctgttgagggggttgggagagtcttcctgtgggtctgtccctgtggacagtctgtctgccccccctttggaagctcatggtcactctctccagggtctgcttctatccctgcgtggaggggacacgcgctctgttcatccgtctctctgaggacccacgtgtcccctctgaaggtttatttgcttctctttccggtgtggttggagggagcctgtccctctgggaatctgtttgccttctttgagggtctaggtgtctgtggtccatgtctctgtccctgggagtggtgtgtcagtatattcgtccccgaggtgggaggaagtctttctccctgaaggtctggaagtctgcatgtctgtctcctctcagggtccgtgggtctgtcctctgtggggtggggggcatgtggtcttcaaggtctggggctcagcgccctgactcctgccctgtctccaccaggttttctgggtagggcccatcgtgggggcggtcctggctgccatcctttacttctacctgctcttccccaactccctgagcctgagtgagcgtgtggccatcatcaaaggcacgtatgagcctgacgaggactgggaggagcagcgggaagagcggaagaagaccatggagctgaccacccgctgaccagtgtcaggcaggggccagcccctcagcccctgagccaagggggaaaagaagaaaaagtacctaacacaagcttcctttttgcacaaccggtcctcttggctgaggaggaggagctggtcaccctggctgcacagttagagaggggagaaggaacccatgatgggactcctggggtaggggccaggggctggggtctgctggggacaggtctctctgggacagacctcagagattgtgaatgcagtgccaagctcacaggctgcaagggccaggccagaaaagggcgggcctgcagcctgcaccccccaccttccccaacccttcctcaagagctgaagggatcccagcccctaggtgggcagaggcagaccctccccagagctccttaggaagaagacagactggttcattgaatgccgccttatttatttctggtgaggatgcatgcgtggggctgctggtgtttagagtgggggctacccaataaatcactgatactcac

SEQ ID NO: 40 - DNA, 4193 bp, Homo sapiens
atacacatgcataagcccatccgcctgcgccctgccagtctgaccagcacagtcctggggacaggagcgtggtggaggagctgcaggtgggggccagagaagcctccacagagagcaaaagccagggtcagccagagacaggacaccagaagagacaggagatcagagaccagaggaacagagaagaggccccagagcaaggcaaggaacggccaaggcaccaggacatggatgcagtggagccagggggacgtggctgggccagcatgttggcgtgcaggctttggaaagccatcagcagggcgctgtttgcagagttcctggccacggggctgtatgtgttctttggcgtgggctcagtcatgcgctggcccacagcacttccctccgtgctacagattgccatcaccttcaacctggtcaccgccatggctgtgcaggtcacctggaaggccagcggggcccacgccaaccccgccgtgacgctggccttcctcgtaggctcccacatctctctgccccgtgctgtggcctatgtggctgcccagctggtgggggccacggtgggggctgctctgctttatggggtcatgccgggagacatccgagagacccttgggatcaacgtggtaggtgcagggaggggcacctggagggccaggaggcgggaatgcgcacaagtggtgaaggtggaggccaggacggaggcgagggagcaaaggaggaaacaagtcagctgcaggctccacatcctcccgggctgggccccaggacccagaggactgagactttgacctcagtctaggcagaatagaaatgggatagtggcgcgaagaacaggtggagaaggaagaggccacataaaggagaaggaatagaaggaccaggcgtttaagggtagatgcggccaagggagtgcggggcctgtgggtagagaaggggaccccacggggacttcatttttctcccaggctccacccacgtcctctgctctccgactgttcccaagttcttccctccacctcgaggcctgcctcactcgccccttctccatttcgtaaccatctgtgttccaccttcttcccccaaatctcctccttccacacctgctgccagtctcctctcaagcaagcctcctgaaccactggccccaaccaacccactggtggggtgggaggagcaccactcacttctgcagggagcaagggtccagagcagatatggacccctctgcacaggcctcccgaagtcctgaagctgggcggcagtgaggtgtcacccccctccagctgaggagaccaggcccagtggcaggggtttctctgtctgtccgtgggcagagcccgcgtctggtccagagtaactccctctgtccaggtccggaacagtgtctcaactggccaggcggtggcagtggagctgcttctgaccctgcagctggtgctctgtgtcttcgcttccaccgacagccgtcagacatcaggctccccggccaccatgattgggatctctgtggcactgggccacctcattggggtaaggaacagaggggacaccgtgcacatgcacacaccgggtccgtccccggggaaactgtggagccctgaaggccaggcagtgtccctccctgagcccctcgtgccttggagccaaagtggtaccccctgcactctaggcccaggatatggcactacagaggccgtggctggggctaaggctctgagccaggactgagaaatggtcccctctcaggcctgccttaaaccttctcccaccctgaccctgcacttgctccccagatccacttcactggctgctccatgaatccagcccgctccttcggccctgccatcatcattgggaagttcacagtccactgggtgagcccctctgctggcagccctggggagggaagggagcctgagttgcctgtagacggtgggtttcacttgcagcttccctaagctcagaaggccagggtgaatgtcttggctgagacttcctttcttttcggcttcagttgcccaggatacccagtggactggcaagaaatgggccagggcaggagctgcagccttggcccagacttttaagtcctggctgtttccttattcactttctccagctggaccaattttcaaacttggataaagaaaaagacaaacagaaatagacacacacaccagcagagacagaaacagcaatagccaggactcctggctaaaacgggggaagtgagaggaaagaggcgggggcgggtgaggagactggccccaggccccagccacggctgcagagcctgggctcagccccagggagggcagggagagcaaggggcaaggtccccttaccttgtgggcttgggaaacacgactcgtggttctcaaatttagcaccttacagacagttgagggagacctgggagatcaagtcggcactggggaagcaggcagcggtcccagagccaccctgtggtcctgatagctcctgggtggggtgacgacatccttctcctcaggtcttctgggtggggcccctgatgggagccctcctggcctcactgatctacaacttcgtcctgttccccgacaccaagaccctggcgcagcggctggctatcctcacaggcaccgtagaggtggggacaggggcaggggcaggggcggagcccctgaagaaggaatcccagccgggttcgggagccgtggagatggagagtgtgtgaaacagcctacgcctggccgcgcccttgggcttcctgccttgcaggacctgcctggaggttctccctgggggtggcgggagggggaggcttgaccttttgtcctgaccaggtgggctggaggggacaagccctatccctggcattacgtttctaggtagaatctgggagtgaatttgtcccattccctctctgtagggcttccccacctctcggtgggacagagcagaggaaggcaggggatttaggatcgccctcccatcctctcacccactggaccccgtaggagtcctgaccccagatgcccaagtcttgggggagaaaggaatgggagcccagacagccactctgcttagacatgggagctatagggacctcagggagccccagcttcttacccacccaggaaacccttggtccagagaggacaggctttctggaggaatcaggccctgggacagcctctgagctcctgggggtgtttctctgaccttgggtgtggttttgggggctatatttcctccttcagactgggggcttcctgaggacagagactgctctaccaatagatctttgtttgttttgagacagaatcttgatctgttacccaggctggagtgcagtggcatgatctcggctcactgcaacctctacctcctgggttcaagggattctcctgcctcagcctcccaagtagctgggattacaggcgcctgccaccacgcccagctaattttttgtatttttagtagagatggggtttcatcatattggtcagggtggtctcaaactcctgacctgaggcgatctgcccacctcggcctcccaaagtgctgggattacaggcgtgagccaccgtgcccggcctctcccaataggtctttattcctcaatcctccaaatgctctggagaggcccccacccttgagaagaactgacacagagaagaacattttctcaggctcgtagagaggtgggatagaggaagggaggtttgagccagacaggcctttcccagggaagcctcctcccaggtgcttgccccagctcccaccccacagcccactctcaggcaggaacaatcctcagaggggaagtgcccatctagccattgactcatgacctgggtcaaagaagccgtctccaggctctctgtcagcattacaggatccccagaaagctgttgaataagccctaagggtggtaggaacccaggagggacccaggaaactaagatttgagattcagtgcctctgaaagactaccaaaatctaagttggtttaatagttaaaagctgctgtagatttattctgcccacaacttgggggtcctttctggatctcggcttctccagctgtaaaatggacacacaatcctcctccagggactgctgtgaaatcaagaaatcggaaaatactttcagaagttaaaatgctagaaaaacaaccccaataaatgatgattattgtt

SEQ ID NO: 41 - DNA, 19378 bp, Homo sapiens
ggaggagcaggagcagcgctcgaggccgccctgggagagctgagaggcaggctctggactggggacacagggatagctgagccccagctgggggtggaagctgagccagggacagtcacggaggaacaagatcaagatgcgctgtaactgagaagcccccaaggcggaggctgagaatcagagacatttcagcagagtgagtggggctccaggcagggtggggatggggcagcctcctcagcgcccagatctggaagggccattccctgggtaccatacagggaggaggtgactgagggattgtttggggaaggagccccgtctgggagtggaagtcccggctttcttgttatggtgcagtcctgtgttgctgtgtgacacaggcacatacaccctctctctgggcctcagtttccttacctgtaagttggttgttgggaggaccagcggtagagcagagatggcagggatgcactgagctgggctgtcagcagaccatgggggtgggacgaggagagagctgaagaccaccggcagtggaccacagcgggaggcgtgcaggcaggagacgggtcagctgccggcttgctggagtcattcctcccacgcagtcccctcctgaggggctggagctggggctggagggtttcggcagtcagggctggagataagagtctgtgctggagctagagggaactgggctagagaatcaggaggacagacagggtgaggggacttcgggctaccttcatgctgtcagttagagataaagataggagtacaaaggggaatttttgggtgaggtacacgggtgaaatgagttttcagggcctcatcctgtgtgttcaccttctgtgtgtgtgtgtgtgcatgcgtgcatgtgtgtgtgtgtgcaggtcctggacagtcgcagcttaagttagcagcaagaggacttgaggttaaaggtatagcacgcaaatatgaggctggagccactgagtagaggctgagggcatctccacagtccaaagccgggctgcagacacggaaggtcagcaggagcactggagggtctggcctggggctggggtcctggggccagcatgggtggggtggggctccagggcgtcgcctcattggctgagcaccgctcctccctccctgttccttggctgggttaagggagtggcactagcaggagctgccccagggcttctcccctggggaccaaggtctgatggaagtgtggggccaagttctgtgtcctccagccctagtgacctctctttggctcctcagcatctacaaatctgaaagacaaaacatggttcaagcatccgggcacaggcggtaagtaccccaccctcttctcaccctccagccccctgtccttcacccagcccacttcagtgccctccctgttccatcctcagcctctcccttggggcagctgtccccactcgacctcctcctccccacccacccactcgcctctgaggtcccagaagaagagcatcttccacctgttgcctgggctgggtcctggggtgaggggaggctcagaaatacttggatgagggtcaaggcatgcaggtggccttcaactcaactgcactcagcacctctcaccctctcaggctcagctgtctttggggtgaacaagagccagtccttgcaatggccaaggcccagccttgttagctttctggcctccccactccagccccctgctctccctcctccagccacactgagtttcttttcaggtttttttttttttccgagatggaatccagttctgtcgcccaggctggagtgcaatggcatgatcttggctcactgcaacctcctcctcccgggttcaagcgattctcttgcctcagcctcctgagtagctgtgattacaggattacaagcacacactaccacacccagctaattattattattattattttggtatttttagtagaaatggggtttcgccatgttcgccaggctgttcttgaactcctgacctcaggtgatccacccgccttggcctcccaaaaggttgggattacaggtgtgagccactgcacctggcctcacactgactttctttgctttcttcaaacatgctgagagtcctctggtgactagtctccctccacctgagaggctttctgcagctaacatacagcccactctcgtctccttcatggctttgctccaaggccaccttctcaacaaggattactctggctgccctatttgaaatcacaccccatctccagcccctgcactcccaattcccctctccttgctctgtttttttccgtaggagctggtaccttcgcttatacaaatgtacttacttattacatttcattgctgtcagctccacgcaagcagggatatttgcctgttgtacacttggacggagagaatgaatagtgccactctgttgacatcattgcctctagcctacatcgctctccagggctttagatcctgtttctagagacccactggtgtctcacaggcaactcagctcctagatggaaacagcctcctccgccccctgcaagtccactcctcctgtgctcttggctcagtcaggggtccccttctcctcagttgctcaagccagaagtcaaggtcatgtccttcatacttccctctcccccatgtctcacatccagtcaccaatctcctaaaatatctagaatgtgcacagcttccaccattctttctaccaccaccctactcccccatggccatgtcaggccaccacccgcccattcttcaaaacccccttcaggcctcacctcccatgccttccctgagctcccccagtcccaggctgcttctctctggtgttgttgccattacccatctcttccattaaacagagtgacccaagggtggagagtgggttttgactttgtatccctggtgcttgactcataagtagatgctcaacaaaaaaaaattttttttttttgagatggagtcttgctctgttgcccatgctggagtgcagtggcatgatctcggctcactgcaacttctgcttcttgggttcaagcgattctcatgtctcagcccctccccccccagctggtattacaggtgcctgccaccatgcccagctaattttttttttattattagtggaaatggggttctgccatgttggccaggctggtcttgaactcctgacctcaagtgatccacctgcctcggcctcctaaagtgttgggattacaggcatgagtcaccatgcctggccaaccaatgtttgttgaattaagaaatgttggttagcaggttgaattgttggctcgtggttagttggatagtttatggttggctggttgattagataattaaatgagttaatagctggttaacaggagaatcagttagttggtttttgataggttagtcaaagggttagtaagccaccaggcgtggtggctcatgcctgaaatccctgcaccttgggaggctgaggcgggaggattgcttgaaccctgggaagtctaggcttcagtgagctgtgactgtgccaccaccgcacttcagcctgggcgacagagtgagaccctgtctcaaaaaaaaaaaaaaaggaaaaagaaaaagaaaagaaacaggcttttagtgagctgttagctatttagttccttggttagcagattacttagggaatctggttggctgccagtttcttgctttgtaggctggctttcttgggcccctcttcttagtctgtaccctctccttgagctctctcatctatgcccatggctcagatgaccatctgtgatttgctgtcctccaaatgtgtctgtaagtgagaccacatgaccagctgcctcctcactggccatcccacccaagtgtctagcgggctcctcacactcaacatgcccagaaagggcctccttgtccattctcccacagcttctctgctgcgccccacactagtcaggacctgccctagccaggacctgagggcatcgctgagctgccccgctctcttccctctctaaccaaacaccaagcctgtcagcttgactgtgctcttttggccattggcgctgccatctctgtggctcaggctgccgccacttctccacccatctgtggagaagctgatgctcaccctgcctccactccacttcctccatcctggcctccacaggggaggcagctctcctgctgaaaccctccacttggcccatggccctctggtagagttcagactccctaatgtgggccatgaggccttctgccacctggccctggcccctctctgcccatctcatgccacctccactcctccgtggccagcccattcctgcacattggatactgccttagctgcaggcctttgcacatgccagtcctacgcccagagtgatccctgcctccctggaggctcctttaccctccagccttcttttcttgacctaaatttccctttctctgggagcctcctaccccctcctctaggccattcacttcctccacatgccccacggtgcccactctttcagctggggcagtgctcatcacactgcatagtaattgctcaattgattgcttttcttcccacgggctggatcgtaccagtgtcctttactgcagtgtctgaacattctaggcacagtagattcaagaaatcttgagttcattggctggttggtggttatctgttgcttactgtgtcttgccagctgtgtggtgatgaggaaggagccaggcttccaggattctcttggtgttcttggctttctgctctatccacttctctctcctcttctgtggttacctccagcatgggagctggggttggatgggggccagtggagggtcacctcgccctgctagggagagcagcagcattcctcagggctcctaaattcccctgacctcacagcagcaaaaagtaagcagagggaatgagagatgtccaggcatggaataaaacatcttccacttttcagctgagaccctctgttcatagctcacccatcccagtctttgctctattccttaccccagagaaacaaaaccagggaggagtagatggagggggatgcagagttgcgcggataatccactggcccccatccaactccagctcccatgcagcatcgtataggggtctcattatcccatgagcagcctcctcagccctgaagaagagaccctaccaatgcaagcagcagcccaaagacagtcctgggaaggtaccatagaggcaggggagaggtagacagagcagggagccaagaacaccaaggaaatcctggaagcctggctcctttctcatcagcacacggcaggcaacaaccagccaatgaaccaaagatttattgaatctactatgcctactatgttcaggcactgcagtgaaggaaactggtatgatcccagcccttaggaagaaaaaccgttaattaattattaatcaagctttttttttttttttttttgagacagggtgtcactctgtcacccatgctggagtgcagtggtgtgatctcagctcactgcaacctctgccttctgagctcaggcaatcctcccacctcagcctccccattagctgggaccacaggcgtacaccacaacacctgactaatttttgtatttttttttttgtagagatgaggttttgtcatactgcccaggctggtcttgaactcctgggctcaagcgatcagctcgcctcggcaagtgctgggattacaggcgtgagacactgtgcctggcctaactgagcaattactatgcagtgtgatgagcattgccccagctgaaagagtgggcaccgtggggcatgtggaagaagtgaatggcctagaggagagggtaggaggctctcccagagaaagggaaatttaggtcaagaaaagaaggctggtgggtaaaggagcctctggggaggcaggggaccattctgggcgtaggactggcatgtgcaaaggcctgcagctaaggcagtacgtgatgtgcaaggatgggctggccagaggagttgaggggctggtgtcagcttccgggcatgcagacagtgagatagcagagtgactaaggccacacttagaggtatctgtatccctcttcttcctcttttttttttttttttttttttgagatggagtctcgctcttgttgcccaggctggagtgcagtggcaagatctcggctcaccacaacctccatctcccatgttcaagcgattctcctgcctcagcctccaaagtagctgggattacaggcatgcgccatcatgcctggctaattttgtattttttagtagagatggggtttctctatcttggtcaggctggtctcgaactcccgacctcaggtgatctgcccaccttggcctaccaaagtgctgggattataggcgtgagccaccgggcccagcccctcttcttcctctttatcctccaggcccaccccgccaccttccccaaactccagtcctatacctctctctatatgcagatttgggccctgttactcacctgtttcatctacctttccatctaaccgtgcacttaactcaatataaatcaagtgccaggccctttgggaagctctgaggatctagttgggagtaagacagattccccagttttcactcattcaacaaatacttattgcacatctactatgtgcctggcgtggtgctgggcactgaagatacaccaggaagtagatccagtccttccttttctccctccctctctcttggtcattcattcattcactcactctctcacaaacctctgtttgcactgacctggttgcacagcctggtgcattttgctgaccccttcatttttgacagaacctggcgcttgtttcagcgctcagtgctgtttatagagctcggcagcagactttgtcacacctcatgctgtttgtagagtgcctcactgttgccaggccctggcctgtgaccccaaagttctggctctagcagataggcagacggacagacggacagacatagacaactgagctccactttccagcctgctctctcccaccctgcttttttccagaggcttgttggcaccgtggattgcactttcagctccccacccgcccatctgcaaagtggcctcattggtgtgccatttttactggtccagtcccaactagaaggggcatggcctggccatctaccccagttcctctccatgtccccgtggctctcaagtgtctccaattccagctgtcccagctgccctgcgcaagaggaggcgtgtgtgggcgaggctgctgggttacctattaactcagctgggagttgaagagccgatgggcagcaggcagacttgagtctcctttctgtccatgggctcgggccactgtcttaggtccacccgtggctccaaaatggtctcctggtccgtgatagcaaagatccaggaaatactgcagaggaagatggtgcgagagttcctggccgagttcatgagcacatatgtcatgatggtgagtgggtgggcagcacgaagtgggtggggctccgccagggccgtccatgaccccctccccatgctgaccccatgggtcacattgtccattccttgcctctgagctgggagcctggggaagcagcagaggaaagtaaggagggggggctttctcatcaagcctttttggacagaaagggctcataatatgtgggggtcaaatgaaaccatgcactggggtatccggggcacggctggaaatggggagaagggaaacccaggataaagagattgaagaggcccaggtgcggtggctcatgcctgtaattccagcactttgggaggcttaggcaggtggatcacctgaggtcaagagttcgaaaccagcctggccaacatggtgaaaccccgtctctactaaaaatacaaaaattagctgggcatggtggcggacgcctgtaatctcagttattcaggaggctgaggcaggagaatcacttgagcccaggaggcggaggttgcagtgagctgagatcacaccattgcactccagcctgggtgacaggagcgaaactctgtctcaaaaaaaaaaaagaaaaaaaaaaaaaaagagagagagagattgaagagaaacttgatgatcgggctctgataatgaatccagagggcagtgggtgatgttgaaggctggcaagcaggggagtgacatgatcagatttgggttttaaaggtaattttgggtgcagtgtggagcataagcagaacaggcaaggctggcaagagggaaccagttaagaggctgtttttgatctgggacagagagaaggtgatgactggtttggggttggagaagaaagcacatgtttgagagggctgtggaagacagaatcagggagactctgccagcagaagatgtgggcaaagcgccgatgagctgttctggagcacggggcccagcacagggtgagaggtgagatgcctgtggggaaatccaggagatagttaaacacatgcaaggggctggagctcaggagaggcttggtctggaaggaaaaagttgaagttcatcacaacacaggtggtagttgtcaatactgtccagagacagtgtacagaaagagaaaaggatgggttgaggacagagccctgaggaatgaagaagggcatgcaaaggagcctgagaaggaacggtcagagaggtgggaggagaaccagagcagactgcatgacagaggggggcagtggttccaccaggaagaagccgtcagcggcgtgggcagcggcagatgggccacgcgaggtgagtactggcaagaggcctttgggtttggcaacgtggaggttgctggtaaccttgacaagggctcttctttagtgtgtgattgggacagaatccagactgcaggcgggatgtgaggttgctccctctccacctgcttcagccctgccacttaccccagcgagcctctgcccttaacgtgactgtagccatgtttattgcatcttatgcagggtccagggtctagagaaagaaggggcagcctctgggaagggaggcaaaggcagccaggtgcatgctagaggaaggtggggtgagagaggctgtttgtgtgtgtggtggggcccatggagctcaagggagacaggaaatcggaacaccggggttcttagcctgaccctgccactgagtgaccagttggccttgggcatgtctctgcatggcttaaagcctgacttctcacttatatcatgtagaattaggctttcgtgggctttggagctgtgtttgaatcctagctctgttatcttctagctgtgcgactatccacaagtatcttaactgttcacaaatttagctttcttatttttgagacagggtctcactctgtctcctaggatggagtgcagtggtgtgatctcagctcactgcagcccccacctcccatactcaagtgactctcttgcctcagcctcctgagtagctgggactacaggcgcgtgccagtgtgctcagctaatttttctatttttagtagagatggggtttcaccatgttggccaggctggtttcgaactcccgaattcaggtgatctgcctgcctcagcctcccaaagtgctgggattgctggcgtgagtcacctcgcccggcccacaactgtagcttccttattggttaacaggagcactaacatcaaactggaggacttgtgtgaagaaggccaagtctcagcacccagtacacctgtggtacccatgtattggtcccttgttattaggacgggtgctctagctgctctctcctctctgcctctagccctcctttgctcctctcctacctccccacctgctttggctcctgagctgtgaggacagcagttggatcctgtccctccttaatccagggcaaagtaatgatcttatcacaagacattccagccccatgagggctgttaacccttggaacctcggaggcaggagggtgcgtcctctgagagctgttagggaaataggcaccgcccacatgcttgatacctgcccacatctgtgttcctcttccttttgttgagattttcattgagcacctaatgcatcccgggctctgtgatgctaagccccttacgtgcagcatcttcccaaatcctcgcaatagccctgtgaagtaggtactattattatcccagtttcacagatgggaaaactgaggctccttgagactaagccttttgcccaaggtcacactgtaagtcaagattaaatccagtgcagtctaatatcacagtctttttttttttttttgagatacagtcttgctttgtcacagtggcgcgatctcagctcactgcaacctccatctcccgggttcaagcgattcttgcgcctcagcctctggagtagctggaattacaggtgcatgccaccatgcccagccaatttttgtatttttaggaaagacgaggtttcaccatgttgtcaaggctggtcttgaactcctgacttcaagtgatcctcccacctcggcctcccaaagtgctgggattacaggcatgagtcaccgtgcccggcccagtatcacagtcttgaccgttaacctctatgctctgtcccttagcttaaatattgccagcttttgaagactctggcttgttaatgctcccccagccagggtaaggtcctcactttcaggtagttcaagatgcctctctcggcctcagtttccccatttatagagtgggagaaaaattcttgccgtgcagatttgctgtgaggattgaagacagtagcacttgtaaaagaactttgtgaggtgtaagcctatatcggacattgtgttgttgttatttttagttgccaggctgtgccaagaagtgagggctttttgtttttgtttttatatatatatacaggaatctcagtgagtcaccagggtgaaggttttgccaaaaaagctagtgtgaccttggccccatttattgcagccaggatgagggaagtggactgatccatgttgcagcttccaggggtgttgcccttggagctggcctcctggctgtgggggagagttggatgggctgggccacattcactgatcagggagaggaggggctggagccatccgggccctggaaaaccagccatacacgtgagacacggggcaagggttgtagatcacatgctacgggggccaagagagcggcaactcagggcggtggggactttggctggctgcagagtgccaatctgtgcaaggctgtagagctgccgccactccagctgactgttgccatggagagtggaatgcaggccagtgttgcctgagctgctcatttttcaagagagaaggaaacttctgttcttcaaaaccaagttatctaaacaaaacctgtgcgctggatgaattacgtgcatgagttgccagttggcaaccctgacacagggaatcatgtggggttcattcactcacccagtattttattttatttatttattttgagaccgagtctcaccctgttgcccaggctggagtgcagtggtgtgatctcagttcactactctgcctcccaggttcaagtgattctcctgcctcagcctcctgagtagctgggattacaggcatgtgccaccacgcctggctaattttttgtatctttagtagagacggggtttcaccatgtcggccaggcaggtctcgaactcctgacctcatgatcggcctgcctcggcctcccaaagtgctgggattacaggcgtgagccaccgcgcccagcctatgctcatccagtatttttagcacatggtattggaatgggggaaaggccatgggggcccctctgttttcagaccctccatgcctgctccagtccctctacctcttgaccctgccagcctgtcaacctgtcctggcctcactcccccctgcacccccatctgttcctgtcctctcctgctgtatcttatcctggatctgaagccagcctagctctgggctcccctgcttctgtcctggggcttctgagggacccagtgggccctgctcagctgcctctcccccaccatatctgggctatttcacattttctcagacttccccaaagctgctctgtactctgacttttttttttttttaaatcagcaaatggcttgatctgctgcttgacaggtaaaataatcaacacttcctgtgttcagctcaccctcttgtccctctcaccaccagacccattaaccacccatgtatccacatatcaccccttggctggggcggggtcctctccttcctggaggacccctccacttctgcaccaatccagctgtccagcctattcaggtactttactctgtcctttttctctgtcctttatcttcagcctgtccctctctcagcctataaatatactgaagtttctccactgaaaacaacacaaaatgaaacatccctcccttcaccctgtcagccccttcgcgggatcatgttctctctcccccgctcctcaggcgagttctcgaagaggagtctacactggtgtctttcaactctttttctttcttttctttttttttttttttttttgagacggagtcttgctctgtcacccaggctggagtgtagtggcgtgatctcggctcactgcaagctccgcgtcttgggttcaagcaattctcctgtctcagcctcctgagtagctgggattacaggcaagcgccaccacacctggctaatttttgtattattagtagagacggggttttgtcatgctggtcttgaactcctgacctaaagtgatccatccacctcggcctcccaaagtgctgggattacaggcatgagccaccgcatccggcctggtctgccttcttaccctgtaccctctcctggggccttctcctctgacggctttgacttcggccctcatgtctacaattctccaggttttctcttttatcaactctagaacagagttctccaggggaaatacaatacaagccatctgtataatttaaatttttctagtatccacattaaaaaggtaaaaagcaacaggtgaaattaattttaataattaacccatatatccaaaatcctatttcaagatgcagtcaatgtaaaattattaggatattctggccaggcatggtggctcacacctgtaatcccagcactctgggaggctgaggtgagaggactgcttaaggccaggagctcgagaccagcccgggcaacatagtgaaacctcatctctacacaaaataaattgaaaaaattagctgggacagggcgcagtggctcatgcctgtaatcccaggactttgggaggccaaggcaggcggatcatctgaggtcaggtgtttgagaccagtctggccaacatcgtgaaaccctgtctctactaaaaatacaaaaaaatagttgggcatggtggcgtgcacctattatctcaactactcgggaggctaaggcaggagaatcacttgaacccgggagttggaggttgcagtgagtagagatcgtgccattgcactccagcttgggcgacagagcaagactctgtctcaaaaaaaaaaaaaaaaaaaaaattggctgggtgccgaggcacatgcccatagtcccaactacttgggaggctgaggtgggaggatcacttgggcccaggagatggaggctgcaatgagccctgatcatggcactgcactccagcctgggtgatagagcaaaaccctgtctcaaacatcaaacaaacaaacaaaacagtggcacaagaaagcaaggcatgcatggagcagcacagttgtttggtttgaggccatctggcacaggtaactgcctggatttaatcctggctcaccaggtacaggctgtgtgaccttggacaagccattcaagttctctaagcttcagattaccctctgtcaagtgggggagaataatagtgcttaagtcataagattggtgttaagggttaaaggaggtgatcagtataactcgctggcatgtaggcagtgctcaataaatggttattattatttagtagtaatactattgctgaatacaaagagtattggggcagatatgaaacaggcagaaaggatgaaattatgcagggtcttcaatggtggcctactgagtttggactttatcctagaagtggaggagagccagtgagtgagtctggggaagacgcgatggggtttttgttaggaagatcactctggctgcaaggaggaggctgggtgaggtttggggagaatgtaagcagagctgattggatgctggggaggatggcgaggaagctctcatttgcaacatctgtttctggctcggccaacggggatggggaacatagagtgaggagcaggtgtgctgtgagatggcgaaccttcaacagagcctgagacgcccgcggaacaccaggagagttccagtggggatttgcaaatagggatccggctcccccgcggagatgacgggaattttagcgcatcaggcatagctgagactgtctacgtggtaaaaccatgcctgaaaagacctttggaaaatcaggaggttgctggagcccttggagagagctttggtgtctgtgacatgtggaagtggaagccagattgaggagggggtgcgagggaaggggtgaagcagctggccagcgtgttctctcctgcggcctgactgaagcagggaggaggcaaccaggaacccacagctggagaaggacgctgggcaggggtgggacagtttgctgggaaggatgagactccagtgtgtgggagccgatcagtgggaggatggaaggtatgggggagacggaggcctctgcagagggagaggatagcacaggagccagggctgaagggaacagtggctctggactgaggctaatgggcctgaaggtaggccccttcccctgtgaaggcgtcattatctgaggagccgtgagggatgggcaagaagcagcagcttgggaatgccgcctgaggtcagtggaaatgaagctgagggcaagacagttagggccccactgttccagcattgtgggaattgcggtggagtgtgtgtgtgtgtgtttgtgcagggaggggttcctggcagagtcaggcacggatggatttggaggtcccttgaagaaacttccttcaggccagcctgaagtgtgagagactctgagggggtgcaatgcatgccagccatcccctcctcgcagccctgcctcaccccaaaacttcaggtgggcctggggctgaggtgcttggatgtttgtcgtaagagcttctaactctgccgctccacccggctctcagtggctcaggtctgagaggcctcagcaggggcaaggagaggaggcagtgaggagggaaggctctggaggaagagggcgtggcagcgggtcttcgaggcaatgccagggaggcccagggcatgggggtgaggagctagaactgagctctgagccctcctctgaggttggggcttctgggcaggcagcccccttagaggccctcccttgtaggtattcggccttggttccgtggcccatatggttctaaataaaaaatatgggagctaccttggtgtcaacttgggttttggcttcggagtcaccatgggagtgcacgtggcaggccgcatctctggtgagtgagcccaggccctgccggaccgggcaagaccaggtgtccccaacaggctctttcctgcccgcctcagccagcttctttgccagcacagccagtgcctcagcctggccaccgggcgggaggaagtctcctctgaaccccgtgcctatgacgtgtttgccccagattcttcctgggccccccgacctaccattttcactggctgggtcatcttaggcaagccatcgcctctgtgttcctcagtttccttgagagtgaaacgaagatggtggcccctgcctcacggggtggttgtgagggctcaaggagagagctctgtcacggagcatgctgtcatacacactagccatcgttgttctcatactgtttgtcactgttgtttgttctgctctcactccctgacacacttgcctgctgcccgcaggagcccacatgaacgcagctgtgacctttgctaactgtgcgctgggccgcgtgccctggaggaagtttccggtctatgtgctggggcagttcctgggctccttcctggcggctgccaccatctacagtctcttctacagtgagtatcctgcccgggtgtccgcctctggcctcagccgcctcctatgaaatatgggcagattggacctcagtgtcctgatttgtaaaaaatagctgggagaaaaaagccttggagatcccccaccctctaacctataacctcatttctgggaccccggtggggcttagttgggagcaggttcgcatgacagtctgtgtctccgcagcggccattctccacttttcgggtggacagctgatggtgaccggtcccgtcgctacagctggcatttttgccacctaccttcctgatcacatgacattgtggcggggcttcctgaatgaggtcagtggtcgaggatgagtatccctccccctgccctccacccctcaggacggagccagcagggagtccctccggatagacaggacaagaactctggatggagactgtaccgagacgtgtctctgctggtgggcttgggtctggggcactgccgaggtcctgtggcttggggaggggcccaggtgagctgccacagcatctgctcctcaggcgtggctgaccgggatgctccagctgtgtctcttcgccatcacggaccaggagaacaacccagcactgccaggaacagaggcgctggtgataggcatcctcgtggtcatcatcggggtgtcccttggcatgaacacaggatatgccatcaacccgtcccgggacctgcccccccgcatcttcaccttcattgctggttggggcaaacaggtcttcaggtactgcccctgcccaggcccattcctttgagtttttctgtgggtcccctgtgtgttgaggggtggggggtgatgtgaggggcagcacaggagggtcctgctgagcccccaggtagcctggggagcaggagtgagtcccaacatttccccaggccagtacagatacagatcctgcacctgcactgagtgtcaacgctgtccctgaattgggctgaggctgaccagggccctgggttgggggtgtttcctggggtagcctggggatgactcctctgctcaacctgtcttggcccgaggtggatgagggtgctgtcctgggcatcagccccctcagcaggcctctgcctcttgcctgcagcaatggggagaactggtggtgggtgccagtggtggcaccacttctgggtgcctatctaggtggcatcatctacctggtcttcattggctccaccatcccacgggagcccctgaaattggaggattctgtggcgtatgaagaccacgggataaccgtattgcccaagatgggatctcatgaacccacgatctctcccctcacccccgtctctgtgagccctgccaacagatcttcagtccaccctgccccacccttacatgaatccatggccctagagcacttctaagcagagattatttgtgatcccatccattccccaataaagcaaggcttgtccgacagcagtacccccacttcctgggggcctcctgtggttgggcttccctcctgggttcttacaggagctccagggctatgtcttagcccaaggtgtagaggtgaggcacctcaagtctttcatgccctgggaactggggtgccccagggggagaatggggaagagatgacctgcgccctcagtaggaacagggtgagatgaaagaacgacagaatgagggattttcaggcacgggggaaggaagggtggttttggggaacagaccgtagctgactggttgggggccggctttggaaatactttgaggggatcctgagattggactctagactctcccctggttcttcccttccctgagttctggccggttcttggaccagacaaggcaaggcccaagaatgtagatcagaattttttagcctttttttcattagttccttccctagtattcttctagattttttttttcttaatcacatgaaattttaataccacagatgtactatatatctgtttatgttctgtatatgttctgtgctttatacataaaaaagagtaagatttttttttcacctccccttttaagaatcagttttaattcccttgagaatgcttgttatagattgaaggctgttaaggggttgggctcctctttcttcttcctggtgccagagtgctcccacatgaaggaataggaaaggaagatgcaaagagggaaatccttcgaccacatgaagacacaggaagaggcctcatagggctccaagggctccagggaagcaggtgcagaggttgggtggggtgaggggccaggatccactgaccctggggccaggcaggaatcactctgttgcctggggctcagaaggcagcatcacccatggttcctgtccttgctcatgcattttgcctttcaataattattgtgcatctactgtgtgcaggccctgcctggacactggggatgcgcagtggatgcgctgggctctgcctttgagggttgcagtttaatgggggacaggtaattatatggaagaaggtgagtgcagagtgggaggcttggaggctgtggggctcggggtaggggagctcacatccagcctctgggccaaggccaggaggcttcccagaggaggagacagagcagggtatggtggtggggggtgtcctttttgggggtgggagctgcactttacagtttgaggggatgggcagaggaggctgggcttcgttctggaggtggggacacggtgaggtgaggtttagaaagcacacctgagccgcagtgtgtaggatgctggaaatggtggagatgggcctgcgaaagtgatgacccaggagcagcagccgggcacctaacaatgggtcagcaccgtgggcgtggagacgagagcggggattgatcaataccggagaagtacaatgtacaggacttgggttccatttggatggagtgggtgagggagagggaggagtcagaaatggcttccggtttccagcttgggcctggggattggagatgtccccactgagagtagggcacaagtgaggaaatggtttggagaggaagataagttacatcatggacatgctgagtctgagttgcctatgggacttggaatggggggtggcaaaatgtgtgtgatcttgagcaagatattcaacccttctgggccttggtcttcttatttgtaaaatggcgataagaatattacttcccatttgtgttgctgtgaatattaaatgcgctaccacatgtaaaa

SEQ ID NO: 42 - DNA, 12016 bp, Homo sapiens
tttaaaaggggcagagcccatagtgtgatcagcaggtcctgtccctaggagataagagtatcttgcacagcaggtgcaggtttcccagcagctcaggcaagagtccgatgtttgtgccatctgatcctgatgtctggagaggtgagccctctgtcggcatcttcctctccaggctggcagagcaaggggggctgtgaattaattcaaggttgggggtcggggccttctatatctggacttgcctcccacccgtgtcctctgtccctttttccctacggcagatagccatgtgtgagcctgaatttggcaatgacaaggccagggagccgagcgtgggtggcaggtggcgagtgtcctggtacgaacggtttgtgcagccatgtctggtcgaactgctgggctctgctctcttcatcttcatcgggtgcctgtcggtcattgagaatgggacggacactgggctgctgcagccggccctggcccacgggctggctttggggctcgtgattgccacgctggggaatatcaggtgagaccagctctgaggattcagcctgatgctgacttggggcactgggtgtggaaagcaaaggcgttgtcaaggacccctgggcgcatacgtattcgggatttcaaggagcgctctggataactatggggttgggagtcagactggggtcaactccagaccccgcagcctgtgagcttgtgactttgagcaagtcattctctttttgagtagctggatcacaggcatgcgccgcagcacccaactaatttttaaattttgttgtagagatggggtctcactatgttgcccaggctagtcttgaactcctggtctcaagcgatcctcctgccttggcttcccaaagttttgggattacaggcatgagacaccatgcctggctggcaagttactctctaaacccaaagttccccgcctataaaatgggaatagtagcttttaaatgttttttttttcaagccaacatatctatattgctaaccatgtaccaggcactagtctaattactttatagatattagttatttaatcctcacaaaagccttgtggggtaggtcctgttattagccccattgtacagctgcaaaagtgaaaggccatgtaatctgtccaagcttagagggccagaaagtggcagaggcgggagtcaagaacaagctgtctggctccagcgctcaaatccaagtgcctcatgcggaagtcatgagaattaaatgaagtagagcgcctggcacacaccaaacatacaacaggcattggttgctgtgttattgttaccactagaagggggttttagggaaagcagaaatgcatagaagacctgagccaggttttcccacctgggtatgcagagatctgcagtggtcctggcttgtcaagaggtgatcaagaaggaggggcagggaaagggacaggcttctctggcttagctctcgtctgggtagaggctcaatggggaggtctgtggacatggctgctggttcccaggagagccagccatcacgacgaagatcttgagtggggtgcctggcagggtgatctgggacatggttggtctgtattaaatctcactcaaaaaatagatctgaatgaggaaacagaaatactatttctgagaaaaccagaatggaaaagagctttaacatctctttaggccgggtgcagtggctcacgcctgtaatcccagcactttgggaggctgaggcgggtggatcacctgaggtcaggagttggagagcagcctggccaacagggtgaaaccctgtctctactaaagatacaaaaattagctgggcgtgggggcaggcacctgtggtcccagctacttcggaggctgaggcaggagaattgcttgaactgggagggagaggttgcagtgagccgagattgtgccactgcactccagcctgggcaacagagtgagactccatctccaaaaaacaaacaaacaaacaaacaaacaaaaaacaaaccacatctctttagacgtccaagggaggctgcaagtgaaacaaaaagtacatgatcttccctttggacggatctgagattgaaggcaacctttatcacttcttagcagggtgaccacagcaagttgctgaagcctctgtgtgcagtggtataaatatgagtaataataatagctgatgttttttgaatgctaactacatagatactactatgggctgtacaaatattaattcttaatatctttgcaatagctttatcaggcagagatcattgaggcacagagaggttatgtaactttgccaatgccacacagctcttcagtggcagagttgagctttgaacccaggccagctggctggaaagaccatctctttggcactgcaaatactgcctctctgtttggcttttttgacaaatatttattcagcacctattgcgtgccagacactgactttgaggttgttcactggatccaggataagctatgtatgtaagttataagcacccagcacagcccctggcacacagtaagtgtcagagaaatatatgctgagtgaacaatgaatgaatgaatgaatgtgtcttgattgttttattgctaatgagtgtaaaccaggaaatgtgtggttttcgtcacctggccctcctgatagagccatgctctgggtgacagtttctctcccctttggggctcccctgggaagccgcctcaccctgacttcatctgtgtatctcagcagagcccaggctaagctacttgaggccaagtaggcctggctgcccctcttcccaacctgtggagtctgagcactggctggtgctttcaaaccatgcatggaggtcaggcgcagtggctcacgcctgtaatcccagcactttgggaggccgaggcaggtgaatcacctgaggtcaggagttcgagaccaacctggccaacatggtgaaaccccgtctctaccaaaaatacaaaaattagccgggcgtggtggcagccccgttatcccatctacttgggaggctgaagcagaagaatcgcttgaaaccaggaggcggaggttgcagtgagccgagattgcaccattgcactccagcctgggtgacaaagggagaccctgtctcaaaaaaaaaaacaaaaaacaaaaaacaaatcaaatcaaaccgtgcatggagcttatctgagggcctgggcccagagctgagccagacaagttggggtagctggtctcgagctatcagggtgttggacaaagtccttttgggcagagctgaggtcctctgcccagtctcaactgaatgtcctatgtcctttggaggatgggtaagctcattggtgggctctttacattaggggggcactttggttctcaagtgacatggggagtcttggggtccctttctctctcattggggagtatccattcctggaaaccagatggattcaggaggccaaactgtgacaatatttcctccatcagagtgggactggttgggacactgatgtgagtgcagccaccctggctctggggcctgggctggttgaacatcttggtgaacacgaaggctctttacccatgagccagggggtggctgatcaactgcaacctgccctttgacctcaggttctgacttgtctaattcccttccacaccccaaactcccaatcccaatccctaacctggtcatgaacctccaactccaccccgtgattctcaatgccattttcagcctgaactccaaatctgcattttacacccatcacactttcagaccacaagtagggttttcactggctgagcagttcaaccaatcaggaggtcttattcagggcctagctactcataaagggagacccgggccgggcacggtagctcgtgcctgtaaccccagcactttgggaggccgacgtgggtggatcgcttgaggtcaggagtttgagaccagcctgaccaacatggtgaaacctcgtctctactaaaaatacaaaaataagccaggcgtggtggcacacacctgtagttccagctacttaggaggctgagtcagaagaattgcttgaactcaaaaggtggaagctgcagtgagccgagatcacaccattgcactgcagcccgcgcgacagagcaagactccgtctcgtaaaatcaaacaaatcataaggggaggcctggataccgctcttgggctactaacttggagccttggtatctcaatctgacctgtggccacatctctgaagggctggagggaggcttggagggagtcagggaggagttaagggatggtctccctacacgctcttcctccagggccatcctgggggctgggctttggacttagaggctgccctttatctcttaccctctgcctggcatctcagggaaaatggaagcaatttggtgcctctttgtatttgagctgggccccggactgactcttgccctgagaggccagcctggtgggttggctgggacacactgtctcaagtgccagccatgtggtcagccctgggctcatgacttcctctgctcttgtgggttacagtggtggacacttcaaccctgcggtgtccctggcagccatgctgatcggaggcctcaacctggtgatgctcctcccgtactgggtctcacagctgctcggggggatgctcggggctgccttggccaaggtgggtaaaccccaggggctgggctagtcttcctctctgatggggctgctggaggtgcatatgtgggtgtgtgtgtgggacagtggggagtggggaaatgtccaatcttttgcttgtttctgccagtggaattggtggacgtttttcttctctctttctttcttattgcattttattgtattttttatttgtttgtttttttgagatggagtcttgctctgtcacccaggctggagtgcagtggcctgatcttggctcactgcaacctctgcctcctgggctcaagcaattctcccacctcagcctcctgagtaactgggattacaggcgcatgccaccatgcccagctaatttttgtatttttagtagagacggggtttcatcatgttggccaggctggtcttgaactactgagctcaagtgatctacctgccttggtctcccaaagtgctggggttacaggcatgagccactgtgcccggcctctcttttttattttagagatagggtctcactctgttgtccaggctggagtgtggtggtgcaatcatagctcactgcaggctcgacctcctgggctcaagtgatcctcctgcctcagcctcttgagtagctgggactacaggtgtgtgtcaccacacccaactaatttcctctgcctaggaaaaccagagacctttgttcacttgtttatctgctgaccttccctccactattgtcctatgaccctgccaaatccccctctgtgagaaacacccaagaattatcaataaaaaataaataaattaaaaaaaatttttttctgtagaggtgggctctcgctgtgttgaccacattggcctccaactcctggcctcaagtgatcttcctgccttggcctcccaaagtgctgggattacagacatgagccaccgagcctggccagccacagagttttcatagctttatgcataaatccatggatagactttctcccttcatccctctttttcttttttttccctctctttctttcttttcttttgcccttaagtcttggttcagccagggatacttcaagcccttcaagatgtgggccttgcttggtgtgtgtgtggggggtggtggatgggggagctctgtcagcttggctgtgatagggtctgaagctgtaagtgctgacccaccgcagactttgttctcaagcctcactttactgtcatcccctgctgtggtctctccatgtcccaatttgagagccacagcctgttcccctgtctatggaacacgcccgggaccttccttctcggcttctgctctgaaaagcagtggccagactcgagctcaagccctgccacttaaaacctgtcaaacatggttctgacagctgcggggcagcagggttccctaagcacattcttcctgctaaatgttttacacatattctcttatttcatctcccagcaactttatgaaaaaccatagtcagttcatagagagcgcttaccttgtgccaggcacaccaacggccttgtctgtgagctgggagctgtcagtatcatccctattttatagatgagaaaacagaggtgcagagaagcaggtaacttgctcaagttcagacgtgcctgtaagcagcacagccccgactggatccagagtccagaagcttgtgcactgcactgtctttccccttcggggctgtgatagccctgaggccgatcgcccatagaaataattgagctcaggcttcagggctctcctgcacatgccccttccacggtcctgtgggggcttagcaatgagttcacctgatcatatgtgtttgtaaagtttctaagagtaaggtatttttagccacgcatggcggctggtgcctgtaatcccagcattttgggaggcagaggcgggaggatcactggagcctaggagttccagaccagcctgggcaacatagtaagaccttccatctctacaaaaaattttaaaaaaaacattaggtgggcatggttggtatgtacctgtggtcccagctacatgagaggctgaggtaggaggaacactggagcccaggaggtccaggctgcagtgagctatgatcgtgtcactgcactccagcctgggtgacagagctagactctgtgtcaaagaaaaataaaaagatagtttatgttctttttctcaaagaggatcatctaagttacataagttttaggtctgtaacctggagccatgactgggttatccccatttttctttttctttttcttttttttttttttgagacggagtcttgctctgttgttcaagctggagtgcagtggtgtgatctcagctcactgcaacctctgcttccagagttcaagtgattctggtgcctcagcctcctgagcagctgggattacaggtgcctgccacttcaccccgctaatttttgtatttgtagtggagatggggtttcaccatgttggcaaggctggtctcaaactcctgggctctagtgacccacccatctcagcctcccaaagtgctggggttgctggcgtgagccaccacgcctagccccattactatttttcagctgaacaaagtgaggctcagaaaggttcagtagttcacgcagggtcgcacagtaaaatctggcagagctgggatgtggatgcaggtatatctaactgaaattggacataacccttcactggctcttaaggtgggtagcatgcgttttttaggccctctcccctcagtttccagctgtctctctgccccactgtgaggctcagcagcttctctgtgcaggcggtgagtcctgaggagaggttctggaatgcatctggggcggcctttgtgacagtccaggagcaggggcaggtggcaggggcgttggtggcagagatcatcctgacgacgctgctggccctggctgtatgcatgggtgccatcaatgagaagacaaagggccctctggccccgttctccatcggctttgccgtcaccgtggatatcctggctgggtgagtgtcccttggcagtggggtgaccatgcccagatctgtgctgttcagctctgggggatttcaggcctgagggtcacagattcagttgctattaggggccaggaaggtgctgcaaatggggaggggggctggcatcaggcagacaacagggagtggtggggactgcagtgtcagaggtgctgcagtcagttcagctcttgtcaattattgctgggtacatgggtctgggctgatgctttcattcaaaggaagtcagaaatttaaatctgtatgtgatgttttccaatgttggtagcgtaaatacaacatttctgtgaactgtgttcattcggtcagcgggcagccgggttttagcctcaggcttacaaagagcatgtgtagctgttgtctcatttatttttagactcccagcaggctttggaagggattaagtagacaagattggggatgatgtctcccgttttatagataaggaaactgaggcttagagaggacagaagtgacttatttaaggtcagtgggcagtggcaaattaagtgatagatttgggacaagaggctgagtgaacccttttaatttttgttgtagagataggggtcacgctctgttgcccaggctggtcttgaactcctggcttcaagccatcctcctgcgttggcctctcaaaagtactggaattacaggcatgagccacagtgcccagcccagggagcccctccctaatgtctgactctttcctttcttttggacacagaaccaagtctaacctttctcctgggtcagctctccctcaactttgggaggtggagcctggggcaggctggggaatgtgtgtgtaaagctgtggccatgctttgctgccccctagtggtctttgattcacttacaagcccttttttttttttttagctccgcttcccgggttcacgccattgtcctgcctcagcctcacgagtatctgggactacaggcgtccgccaccacgcccggctaatttttttttgtatttttggtagagacggggtttcaccgtattaggcaggatggtctcgatttcctgatctcatgatccgcccgccttggcctcccaaagtgctgggattacaggctgagccaccgcgccccgcctacttacaagctttttgacttcgaagctggacacagggtggggatgaggtctacaggcccccagctggagagaatggggtaaagacagagccccagaacgagaggagtgcacctggggaagagtctccctgtaggattctgggtcttaagcttgtggtgcaacaagggcctcatctggaccagctagattagttaatgctttttgtacattaattatattcgaggtgtgggctccatttgcattttctcattcactcctgatgacaacctatgggatagtcactttcacctcacctgaacacagagaggttcatggaggttcccacacctagaactatctgggctggggtttttctttctttatatgtatatttttaaagctttgtgggtgaacctagtaggtgtatatatttctgggttacatgagatgttttggtacaggcatgcaatgcataattatcacatcaaggtgaatggggtattcatcccctcaagcatttattttttgtgtcacaaacaatccacttatactcttcgttattttattttatttttagttttagtttttgagacagagtcttgctctgtcacccaggctggagtgcagtggcatgatcatggctcactgcaacttccaacccccaggttcaagcaattctcctggcttagcctccccagtagctgggactacaggcgtgtgccactattcccagctaactttgtatttttaatacagacagggtttcaccatgttggccaggctggtctctaactcctgacctcaagtgatctgcctgccttggcctcctaaactgctgggatttcaggcatgagcctctgtgtctggctgaggttacatgagatgttttgatacaggtatgcaatgtgtaataatcacgtcaaagtaaacggggtattcatcccctcaagcatttattctttgggtcacgaacaatcctatgatacactttttgttattttataatgtgtgggctgggttttgaatctaggtcagcttgactccaaaaggattgaaactgattgactttcctttgtagactttggagcaaatgtagctccagcttctcttcctgccatcacccagtaggcatttttgctgggcattctagaggagtgtgtggggagtgctggactgcacgcccagcggggagccatgccttctgcttcctggtgctaatagcccctctgccttctttaggatctacccaggagctcatgatgtaggagtttagagagggtttctgggtggtgtgtgactaggcctagttggggacatgagtgtgaggtgaggtggggtttggctgggatcctggtgaccttggtgcctgggtgtttgcaggggccctgtgtctggaggctgcatgaatcccgcccgtgcttttggacctgcggtggtggccaaccactggaacttccactggatctactggctgggcccactcctggctggcctgcttgttggactgctcattaggtaggagtgtgacacagggtcaccggcccattggatgggcacttggcaacatctcccagagagtccgggactagagggctaggctctcacttgggtttggaagagaaaagagggagcctctttagaggcaaagaaaatggctccatctaggagctttggttgcaagtgacagagatctatttcaaaatagcttatgtcataaagaggaattcattggctcacatcatcaaaaacttcacaggtagcagtggtttcaggtggcgctcaaacaatgtctttaggacttggtctttctccttttctttttttgacagtttccctctgttgcccaggctggagtgcagtggtgtgattctggcttattgcaacctccgcccccgggttcaagagattctcctgcctcagcctgccaagcagctgggactacaggcgccgccaccacgcccagctaatttttatatttttagtagagtcgggttttcaccgtgttgaccaggctggtctcgaactcctgacctcatgtgatccacctgcctgggcctcccaaagtgctaggattataggcatgagccaccgcgcccagccagtttttgtgatttttttggtagagatggggcttcaccatgttggccaggctggtctccaactcctgacctcaagtgatctgcctcccaaagcgctgggttacaggcgtgagccactgcacccggcttcatttaattctgggctttcagagtggcagcaatgagcatcttcaggaaaagggttagaaaccaagcaggacccgggcatgtggttcacgcctgtaatcccagcagtttgggaggccaaggtgggtggatcacttgaagtcaggagttggagaccagcctggccaacatggttaaaaccctgtctctactaaaaatacaaaaatcagccaggtgtggtggtgcttgtctgtagtcccagctactcgggaggctgaagtatgagaattgcttgaacccaggaggtggaagctgcagtgagctgagatcatgctactgtactccagcctgggtgagtgagactctgtctcaattaaaaaaaaaaaaagaaaccaagcaggaaactgtctctctctctgcaaggggatctggctattaaggctggggaggctcaggaagtcatctttctggagacttctgagcctggagacatgacgaagggctgggtctcacctccggggcagagaccttactgggtcatctttcttgcaggtgcttcattggagatgggaagacccgcctcatcctgaaggctcggtgaagcagagctcgtgggattcctgctgctccaggtgtcctcagctcacctgtcccagactgaggacaggggagttcctgcatttcctgccagggcagaggcccagaggagcgaccccctgcttccactgcttgggcctgctttctcagatagactgactgctgaggaggctctaggttcttggaattcctttgtgctcatcagagaccccagcctggggaacacgctgcccgcactgcccagagagcagtgcaaacaccacaacacgagcgtgtttcttgagaggaatgtccccgagttggacaaggaggctgtttctgcacatcagctcatttcccgcaccccatttcttgcttgattgctttgttgggggcctggccacttccttgcttctcaagctgacaattctcactttgcaataaatagtccagtgtttccttcca

SEQ ID NO: 43 - DNA, 47531 bp, Homo sapiens
ccaccagaagacgattaagccacagcctctaattggaacggcatttgtacagtcagagactcttaccagacatctccaggaatctgtgagccattgtcaaaacgtccattttcatctggctgtgaaagtgaggaccacaacaggtaggtattggtagaaacaggagtcctcagagaagccccaagatgcagcctgagggagcagaaaagggaaaaagcttcaagcagagactggtcttgaagagcagcttagcgaaagaaaccctctctgagttcttgggcacgttcatcttgattgtaagtatttcctgatttcctacattcagacccaataatgcctccctagctgccaggctggtagtggagagttttgtttggtttgttttatttaagttaattgggatcagattcatcttttccaggaagaaacaagttactagaggctgtttgactatttaggggttgtgatattaagttttcaattacctattgcatttacccacaaaagttgatgggaatggaagagaaggagtgagtgaagtgccttctttatttcaggtatttacttatactctaaaatggctttgtgatttaaaagtcagtcgttagtgttttcattgatcaattgatttgtaccctgcttgcttcctccaagcattggggtgactcagccacctggaataagcatcaatagtcaataaatatagttggcaaaaatatacatatggatggcaagaaacatctcagtatgacaagtacttgacaagtattgtgctgtcagatcacttgatttttaagaaattaaaaattgcactaggtagaaatagtgtaccaggcaatagtccagctttagttttgtgtcatttttggtctctatttaaagcaaagtaatctctaaatatctttggagatttacaagacaattacaagaaccttctagatataaacgagggacgacttctaagaacctcttgaggattgcccagagatttactcagctattaacaagagactggaaagtttacttaagtggtctgggctttcattttgctgcacgttctagcattctctgtatcctgaagttttacctggaaccaggccatttgtgaatagaaatgtattatcatgtggcaactattgatttcaactggttttttttatcctgcttaaggatttcctactccaggtataaataatttcattttccttaaagttatagagaatagcccataaactctgtgcattaaaagttctttgcttggggcaaatacataataatttagaagaataagcttggaatgcatcagctaagccaagaatgagaaacaaatgtgaactcaaaaaattgaactagtggaatgtgaagagagagtcataagttttctggtctctgagaaacacattagaccagattcgttacagtattttcagggagttacggacaatttcttgcactcctcatcttcccttggcattcattcctggctgctctgaccacactcactgtcataatgtagcaagacattatgtagtaagacattatgtaagtagttatttctaactaaaataaaagaaataaaagagagaagatgaaagtcagtgattcttatgattcccggtcactgcttactggaaaatctttcttagtgagactgagacccctctgtcatgttacttgacaatctggccccaaagaagcagttgtcaatacagagtgattacagcctcacagtgacgagtgagtcagaaaagtgaagcaaattcagttacaattcttagtgctctgcagaaaacagaggaggaaatgaactacacatctttttttttttttttttgcaaccaaaaagtttaaaaccctctgtatttcttaagaagaacctgtttcataatcccgtgctttctgtgttgaaagtgaaatttgctttcagcagttcactgttcaactggccttgtgtcttccccactatgcaaggtaggggctaaataaagaacatcttgatattgtctgttgaaaaattaaaacttgtatgttctggataaagccagtcatttcctgaaatatttaatttccatttatactatacatttattttctaaagtgaaccctaataaaatctgcaaccttgattatcatcttcagttttgttggagcttgtggggatgtataaacaaaaagttcctgcacaaacacaatttttttaaaccaagaaatctacatggttttgctgaaagtttatccaaagtaattttctgctttattagagaatgtgcattttgccttgtattttctagtaaaaagctttgtacattgataaggcaaagggaaggggaacatcgaaaagatggcagaggatataaaggactcagtctttcaaatatgtgagattgttttttttccgccaaccttgggagtctggttctattttttttccctcctttcaaaaactgctcttatggggagcatctcaaactaatctaacaccatggtataaattacgggtccccaacacatgggccacggactggtacaggtggtccctggtgctaaaaggttgggaacagttggtataaaggactgataaggcctactggtagcattaagagaaaagaaggtccatgaaaaattctttataaactacaactaaaaccaacaacaattaagtcataagccaaaaagctagctctaatttggccttttagtcattgggatacgtaggcctttcacctttactagtgtggggggaaagtttgcaatgcaaaaggctgatctcttccttgggctgaacttcagagaatctctagtcatgtctagtcatttctcacagggactatgctcactcctactactctatatattctcctctctcttcaaacttttcaggattctttgatacagagctcatcttattgaagatgctgaagtgaacaacccagcaagcagctgtatttcctcctgaggtggaaggacagctttccagggctggacttttgagtagataacatactgtgtagtcggacagatgctgaattcttttctcaccctgtcaagcttccgcattagaggtctaacttttggcatctcttagaatggaaaaaaatgcaagtacttctgctaaaagtagtacccttgatctttaccttcagtgccctgaaggaaatagaaagaaactatcctggaacacagactatggaggaaataggggagtgggcagtacaggaaggaggaaggaaggacagggatggttgattgtatgtgagaaatttcagacactgcagtgttcccttcttcctgctcctaccttccaccaggactgggtgagaaatcatccctccttgctggggtcacagagtttgtctctccattttgagaggattctcaatcagacatgctggttatagacactctagggctttaaaattattattacaaagtttgttcagatcttgaagtaggtctattctatcttagggagcttttgagacaaaagaatctatggtcaggatttagagtgaaatcagaaagtttataagccaaattaaaggaaacgtccactttgcagggcagaatccaggacaattttggtctagtacattatttggaatgcccaggtactggcaggagagctctgagataagggtgggcagatatcactggccctttttattggtggagaagctgagctcaggctgatgtaatcatgcatcccgggaatcagcccagagcccagttgccctgccctgggcacacactcgcccttggacttccactgtgctcttcccttgagcagcacagagtgttcgcttgtccaaattctcgttcctgtttgaatgtttcctgaagagagagaaaggagagcgatcaactgcattttgccagatggagagacaagtgtaacacaattaagagacttatcagcccttgaatgaaagagctgggcttggtatcttgactccagtcattccctaataccatcatttgatgagaattaactcctttcatgtgaaagcccaagcgaacttgacaaagtttacagtttaaaatacattagcagtgtcaaaagatgattttacagaacagacgtgatcgtggtggggatattttacaggagatctgctagcagagtcttgggatctcttcaccacctacaaacacattcaaacctcaggtaacaccagggttctgaggtggctcagtgatgtcttcagctctgataaacacattgaccaagcattgcactgtgtttttcctctagtatttctgttttctagcttttcctcaagaccactcttttggttcagagcaaaggacctataggaaagtgggcctcaagagaccccataacttctctgtcttgctttctggctcaacagccacctattctttctctgtttcctccatgcttggttcttctagcttgctattttacaaaaccatccatctctccaagtctagggtgcccaggactgagggtcacatggagctagagctgtcagttggccaacagatgctagatcagaagcccctactgtcagcagtgccctggattctgcctgtgggtggtttgtctgtccaagtcgtgcaccatcctcaggccctcgctcagaaactttggaataagatggtctctgttgcctgactcttgcatcattcaattgagagaacagacacccacagtcttgcagcattttatcaaaagctctttccaccacagcagaacatcagaggatgtctaccgtcagttttacctgcctcttgttattatgatttatgttcaatgctcagtccttcaaggattggtacttctgtacttgggagttctgtccctttaagaacgtctgaggttctaaatctgaacatgcagaagtgctaaacactggggattctccttagatccttgccttgaagcaagagtttaaagactcgaggctgtcttctgtcaaatatgatgacgaggacaagagagggcaaaggaggttaacactaagcatactctataccccagacactttgatccttaaactaaatctaaactaaatctaatgaggtaaagatactgctctcattttatagatgaggaaattaggacttagtatgtttaagtcacttgaccacaagcaagtgtgtggtggagctgagattagaatcaaatctctccagtttctcttaaccacgctacagttaacattctaatatatgcttttgaagttttactacagttgcctataagaatatatattatagttttatatgtttaaaaatttacatatatagcttcatagcttatacatcttctccaatttgagactttttcccagagacactttctgaaatttattaatttgggaacatgtatgactggtttatgtacccatcgccctctcactattgattacaatcaccaagactgctacattgaacatcttggtacatgtctccttgtacacatgttcacgagtttctctggagtatatacctagaagaggacctggtggtcctagggtatgtgcatcctcaactttatgtgtattataaaattgctctccaaagtgatttcaaagttgtacttccagtagcgacatgagttcccatttatcacatactcaaccatccttgatatcatccaacttttatattttgctaatttgagagagagaaaaatggaatttcactagtaattgtactcaattccagtgcagtgattatcttttcatgtatttactaaacgtttaggtgcctcttttgtaaattgcctgattacagcctctgcacctttctctttgggatactttactttttcttaattgttataggatatttatatttattttaaatccatacattaaggtattttgtgttttcttattgaggtttaatattttgcttttcatatttgtgtctttaaataattagaatttatttggtgtatgatgtgggattggggtctattatctatgtctgcaaggatagacaattgtcccaaggccagttttgactgtctatcttttctccactgattgtgatgacattcctatcatacacctgttcacacgcatatgtggttctatgcctagactattcagtgccattgatctgtttgtccaaacttgcactacctcatataccattttttttgttgttgagaacattgtttgttgttgttgttgtctttctacttttattaccaaggttatattagactcatgaaatgaatcaaaattttccctttttttctattttctggagcactttgtaaaatgggagttatgtatttcttgaagattactcagcatgtgcacgaaaaaagagagggatggacagatgacaagaaaaatttctttaaaagttacaggtctggccaggcgcggtggctcatgcctgtaatcccagcactttgggaggccgaggtgggcggatcaagaggtcaggagttccagaccagcctggtcaatatggtgaaaccccatctttactaaaaatacaaaaattagctgggcatgatggcatgcacctgtagtcccagctactcaggaggctgaggcaggagaatcgcttcaacccggaaggtggaggttgcagtgagctgagattgcgccactgcactctggcctgggcgatgaagtgagactccatctcaaaaaaaaaaaaaaaaaaaagataaaagaaaaaaactctactatacctgaggtgacatcctcttttcaatttttaaaaagaagtttgttttccatttcagtaatttctgctttgatcttccttatgtcctcctattgagttgatcagctttctttattcttgccttttctcctctgtgtgccctttctattaacgtatttacccttaggctgggcacaatggctgatgcctgtaatccctgcactttgggaggccgaggcaggtggatcacctaaggtcaggagttcaagaccagcctggccaacatggtgaaacctggtctctactaaaaacacaaaaattagccaggcatggtggtgtgcacctgtaatcccagctactcaggaggctgaggcaggagaattgcttgaacctgggaggcggagattgtgccaaagcactccagcctgggcaacaaaatgagactttgtgtccaaaaaaaaaaaaaaattacccatatatatatatattttttaacttaaagtaaatcaatttttcttctgaacaacacaaaaactttaaaaaattctaactccaatcaaccactctatcttacatgtcattgttgtctaatatttaaatttttccctcattattattgttgttactggtgttttaaaataaatatttttgtttgcttatttaaaaatgcattcctatcatttactggtttatttgctaatcattactgcatacacccactactttctcttgggttctgtctccttattcttgcagttcttcctttagaaaatttttcaggaagggtgtgtgacagatgaactttctcagtctttgtctgaatgtgtctatttcatgctcaaggttgaatgataaattagctgatacattttaggttgataattatttttgcttggcatttttcaggtgtctctgtcatttttccaatgcctctgttattgctgttcacaagtctctgggtctcatttttgtccttaataatctgaatttatctggtaactaataagagtttcactttgtttttttattttgaaatttcactattatgtgcctagaggtgaatttatttctagttcttaactcatttttcatcttcaatttaaagggttataatatttaccaattctggaaaattttcctctttattaccttgcctttcccccagtctgttttctccttttagaactcctactatattggtatgcatgttgactctgtcatttttttatgcctcaaccatttttttcatatttcttctcttattctcttaatccatgctgaattttgtatagttgctgtttcaaatctatctttcagttcaccaattccctatttggttctatctaatcttctgtttaacacgttcattgagtctttagtttcactatcttttcatttctatatgttttatttggttctttttttcttttagtttcttttcttgtgtgcttatttctttcatttccttaactcaccaaagcctaatatatttataatattttaaacctgttctgtcgtctcgagttgcgggggagtgaatcattctgcctattttatctactgactcttattcataaaggatcttttcctttagtgttttatacatctgtattataaattcttcagggagatttatttttccctgtgggaattccacgtggcctaaggtgtggatttgttcttccagagcaggtttgcatttgcttctgctgggttccccaggtataccgccagccagaaatgattgctgtggagtggcaaactgaaaagagaagcctgggggatgcaggtgcctgttgaagagcactctcccaaaagaacggcactctgtgacctttctcccaactgtgaccgtaaatgactaagagacctcgtgcttcctacaaatgaggccagctcttttgacatccttagactttgagtcactagcagctgagggagaattttcctacagacttccccctatcagcaatgaacgtggaggaccaggctcacctgggaaatcttacagtttcaaggcgcttgggagaaaagtccgttctggatcactgatctaagggctgcagactcctggagccaaacttgattctgccaaacaggattctgcttctgattgtgaattttaattttacccagaaaactcactgttttcattgactttcaaaggggaagtattttttttttctactttccagaagtgggtttcgtcagtttttctttagtgacgaatcccattgcaacatgttctcattacttgacgagaaaggaaaacatactaatgagagcagaagttggcccttatttactcatcagccctgtgaaagtgacctaacaacccacagatggttcaaatggctgaagcagttcaaccgtgaagaaattaggtgcccattatacctccagcaggcatctaatttcatgtctggggcaggattgctgagttcactgaaacttctaagcaaagcaagtgatttaagagaaactatgccagcaaagtgtgccggccctgccccttgcccttttctccatctcagtagcttggctggtcatgctaagatgtgagcctagaacagtcagtcaagaccataaacgacttgaaggcttagtccctctcagcgcacatgccttagagtctttctggaaattcaagctttcttatgagagtacctcccgaaaaccctatgcttctatcaacaggtgattggttaaaaaaatgggggtgcatctatgcaagactcctatttacccattaaatagaaggtttacatgctaatacaaaaatactctaagctattcaagtagaaaaagcaggcagaaatggtatgtgttaaataccctcacctgtataaacacatgcacacacacacacacacatatacatgcacacacacagaggcacatgcacactttttgacaacataccaggaagtattgatattagttacctgtggagaataatatttaaaatctggggttggaaggaaacacatttcttatattttatagtatacccttttgaaatttaaaaacttgtacatattcatatgtattttcttcacccccttcctccccatgagagagctcacccagtagagtatgtcttttcaaagcatttttgtagggagttgctcaaaatctgttctgaaaaacactagttccaaaagatgcaaacaagaattaagtgaaaaaaatacatgtggtccttcccaaatatgtttgggaaacactgggttaaaccaaaataagcagttttctcaactgcaggaattatcagagcctttaatgttctctgtaaatctctaagaggggaacagaatgtaaagctgtcttcagctctacttaaccacaaaccccttttttgaggagcatctaatggaactaatgatctgtgaaacatactttgagatttgcagaccctggaaacacatcagtcctcagaactcctccatatgcagagatctcccaggttgagagtgacaagaggcggccttggcactgttgggccctgtgggtagagggagaactgtcctcagcaacccccactcacacacagcccactcagagtcacagttatcctgatgcaaacacaacttaattaataaatgcttcattcatttcttattctggtccttgggatggtgatcaagttcccatccaggtcagcatttgggatgagttgagggatgtgaatgcctggtgaggtcagaaaaggagagcattgttcttttcagcccagtagagatagtggagttcaatcacctctcctttctgagctctgagttggttcttggtgcttttttctacttggagagcactgaggctggccatgttcccattcacctggctctcaaattgcagattcctctctttttatgagcctgccctaatatcgcctcttttcaagtacagctggttcttgggtgattgacctcttcctgatattctgtcaggctgcatgccaccacatattttatttttagccagaagatccaggtttcagtcttggttctttgtgactgtttttgttatgactgagaaagttttattttgcaattggaaaactaactgacaggaaagctgtgcataaatctgtactctgtgctaataaaggaaacaggctgacccacagaggcagcagcagttgtcacaggaggaacactgggatgggaatgaaagaatctggctccagattcagaaactgtcacctattagcaatataggtcttgtatcaagtcactcagactttcagtttttaactggggatgaagtattgggaggactcacagaagtaaagtgcgtgagagtgcctcaccatggcgcaaatgccatgtgaatgtttcctccttcacctattgctggtcaaaatcatgtgcagtcaaaccttggccccagcacgtaccacttatgggaatgtgagagcaattcttttaacttctctaagctttggtttcctagtttgtaaaatggagacagaaagagagccttagtcataggtcattgtgaggactgatgggctaatgcatggaaagtgctcaactcacaaaatttactcaatatagattagttgttattggtattattgttatcttcatatgttcaaaaggctatagtcacataagctgaataggagggaataaacaggtggagtaggatggatgggggacttctctaacacacacctatagcacaggctggagggccaaggtttgcagagagtgctgtgctgacagattgccatggattctccttcctctccagttacaaacttataggagcagggaagcctctggtaagccctaaatgactcccttctcaagaatggggattgtgttttttggaaacactatggccaccaccttagacaaagttttgcctataggcagtccttcctttgatggtagccacagatgctttctccaggagctaggactgcatcccagcccaggtgtgttatcaccatggtatctgcaaggccaggaaatcactggcaccaaaagcttaatggtttcctcttcccagaccttaagacaggagtcaacagctccaaaggtaagtgagaccgaacaggcaacacatatgagtgatgtgggcatggtggagttgcagggaaatggatagcgtatgccctgtctcagaggggcatttgccattcagacccactccactgtggccttaccccattcttgcatcaggagtttcaattacttgagctcaccacagcctgatggaaggtagtcgccatcgcaattaccttaggcatatacttcaccctcctacaagtttcagaatattccaaggccccttttactatctctgatggaatttatggctcaacattctttatagccacaggctttcatggacttcacatcattattcggtcaacatttctcactatctgcctcctctgccaactaaaatttcactttacatctaaccaccactttggctttgaagccgctgcctgatattgacacttcatagatatagtaagactattcttatatgtctctatctactgatgaggatcctactcttttagtataagcaataccattgacttccaatcaataagctttgataatatttgaaaaagagtaattagctttacactagccctagtgaccgacaccttactagcaattacattttgactcccataacttaatatttatatagaaaaatccagcccttatgaatgcggatttgactcattaacctctggccaccttcctttctctagaaaattcttcctagtggtcaccacatttctcctgttcaacttagaaattgctctactactgcccctgccatgagcccttcaaacaaacaacctgacactaataatcagcacagcccttatactaattatcattttaatcctaggcttgacttatgaatgaacccaaaaaggattagactgaattgaattggtagatagtttaagttaaaataaatgatttcgactcattagattatgatagactatatttaccaaatgccctttatttatatcaatattatattaacatacaccatagcattgctgggaatattagtctatcaatcccacttaacatcatccctattatgcctagaaggtatagtattatcaatactcattataattattcttataactttaaatatacatttcactctagcatccctaatcccattagcctcctattatttgtggcctgcgaagccgcagtatgccttgccttactagtttcaatctccaacacatacagcctagattatgtacaaaatctaaatttacttcaatgctaaaagttattattccaacaattatactgttaccaccaacatgactctgtaataactccataatctgaattaacataactatccacaacctattcatcagcctcattactatatcttacccaattaaatgataattcatctaacttctcactaactttctcctctgacccactaacatcaccccttctaatcttaacaacctattacctcttgtaattctagcaagccaacatcacctttccaatgagtcacccccacagaaaaaactctgtaatagttctcttcccctacttatcacacttacccatactcaaaataccttgggttcactaaacataataataataataatatttaccacccaagaactattaacttcctgatctaataatcttatatgactagcatgtattatgactctcatagcaaaaatacctctatatggacttcacctatgactccccaaagcccacgtggaagcccctattgctggctcaatagtacttgcagaggtactcctaaagctaggtggctatggtattatacggcttacccttaccctcaaccccctaacagaatatatagcctaccccttcctcatatccttatgggaaatagttatgacaagctctatttgtctatgacaaacggacttaaaatcactattgcatattcctccataagccatatagcacttgttattatggctattctcatttgaaccccttgaagctttacaggtgcagtcacccttataattgcccatggagtcacttcatctttactattctgcatagcaaattcaaactacgagcgagtccatagccgaattatattacttatctgaggccttcaaacactgcttccactaatagcctcttgatgacttttagcaagtcttactaaccttgcctttccccctactattaatttaatagaactcttcatcactatggctttattctcttgatcaaacatcaccattatgcttataagacttaatatactaattacagatctttactccctacatatactaatcacaacacaatgagggacacttgtgtattacattaacagtattaaatgctcatttacacaagaaaatacattaatacttatataccttgcacctatctttctattatccttaaaccctaagattattatggggtttgcatactgtaaatatagtataaccaaaacattagattgtggatctaataacagaagtctgaaacttcttatctactgagaaagtatgcaagaactgctaactcatgctcttattataccactattataaaaacactagttcccttaatcttaccaattattactaccttagccaacccctgcaaaaaagattgatacccatactacgtaaaaatatctattgcatgtgtctttattactagcctcatccctgtaacaatatacatattcatagatcaagaaataattatctcaaactgacagtaacaattcaaactcttcaactcccactaagcttcaaactagactacttttccacaatatttatcccagtggcactatttgttacctgatctattgtagagttctcaatatgatatataaactcagaccctaatactaatcaatttttcaaatatttactcattttcctcacgataatattaattctagttaccaccaataatctcttccaactttttattggatgagaaggtgtgggaattgtgtcttttttactaattggctgatgatatggccgagcagatgctaatacagcagccctccaaatggtcctgtataaacgattggcgatattggctttattttagctatagcatgattcctcttattttccaacacatgagagcgtcaacaaatatttattctaaatcttacccccaattcctttccactaattactgttctcctggcagcagcaggaaagtcagctcaatttggccttcatccctgacttccttccactgtagaaggcccaaccccagtttcagccctactccactctagcactatagttgtacagtttttctacttatctgcttctaccctttaatagaaaataatctatgaatccaaacctttacattatgtgtaggggcatcaccacattattaacagcaatttgtgctctaacacaaaatgacatcaaaaaaaaaatcgtagcattctccacctcaagccaactaggccttataatagtcacaattggtattaatcacacctagcatttctttatatctgcacccatgcctttttaaaagctatatatgttcaggatccatcatccataatctcagtgatgaacaagacatctgaaaaataggaggactattcaagactctactcctcacttcctcctcccttatcattggcagcctcacacttacaggtatgcctttcctcacaggcttttattctaaagacctcattattgaaactgcaaacacatcatacaccaacgcctgagccctttctattactcttatcgcaacctccctaacagctgtctatagtacccaaattattttcttcgctctgataggacaacctcgcttcacaactgattattattaacagaaataaccctttcctaattaacccaattaagcacctaataatcggcagtatcttcgctggattcctcattaccaataatattgttcccacttcatccccctaaacaactatgccacttcgcctaaaactcacagccctaggtgtgaccaccttaggcctcttactagcaatagaactaaattttataactaataaccttaagctaaagtatcaattacagacttttaaattctccaatatactaggtttttactcagccaccatccactgtacagctcccccaactcaagtctatttacaagccaaaatctggcctcacttctactagacataatttgattaggaaagtccataccaaaaaccatttcacaaactcatataatcagcctccattaccatacctgccccaaaaggcctaatgaaactctatttcctctccttttttattttacctcttctaactctaatcttaattacctaatcatcaccctgagtaacttcaagtgcaacataaatactgacaaataacgttcaaccagcaactgccaccaatacccgtaactgtataaagaagctgcaccgacagaatcctcacgcaaccacctcgccccctcaccctcaaatattatgcaactccatatgctattaaaattaattataatgaccatcccatcatactcattcaccaaattaacaccaactctgttaataatcctaataataaagcccctcaaatgtcaatacttgaccctcatgtttcaggatactcctcaaacaaccattataccacccaaataaattaaagactattaaccccacgaaagtcccaccaaaattcaacagaataccacaacccacagcaccactaataattaaccctatcccctccccataaataggagacagtttcgaagaaaaacctgcaaaccctataactaaaagaatacttaataataaatcataagccattattcctacatggactataaccatgactaatggtatgaaaaaccatcattgtatttcaactataagaacactaatgaccaaaatatgcacaacgcacctgctaataaaaattattagtcattcattcattgatcttcccacaccatccaacatttctacatgatgaaactttggctcacttatttgtgcctgcttaattctccaaatcactaccagattatttctggccatacactacacatcagatcccttaactgccttctcttcaatcaccaatatcagccgagatgtaaactacggctgaataattcgttatcttcgtgctaacggcgcttcagtatttttcacctgcctcttcctacacattggccaaagcttatactatggttcatttatatttctagaaacctgaaatattggtattatcctgctactcacgaccatagcaatagcatttataggttacgtactcccatgaggccaaatcttctgagtttttacacacctattctacaaacctgctatcagccattccgtatattggaactgaccttgtgcaatgaatctgaggtggattttcagtagacaaagccacccttacacgattttttgccttccattttatcttacctttcatcattgcagctttagagacttttcaccttttgttcttacatgaaacaggatctaataacccttcagggatctcatcagaccctgacaaaatcactttccacgcttactacacaatcaaagatattctaggcctaattctcctcctcctcttcctaataattctagtactattttcacctgatctcctgagctacccagataattacactttagccaaccccctcaatacctcgccccacattaaaccagagtaatactttttatttgcatatgcaatcttatgatccatccctaataaactaggaggtgtactggcccttatatcttccattcttattctagcagttatttctatacctcacatgtctaaacagcaaagcatagtattccgtccactagtctcacactcatatgaatcggagggcagccaatcagctacccttttattatcatcagacaaacagcatccattatgcacttctcaactatcttcaccttacaccaattaccgccctaattgaaaacaaattactcaaatgaaactgcccttgtagtataactcaatgctccggtcttgtaaaccagaaatggggaattccctctccaggacaactcagggaaaaagcacttctgcttcactgtcaacacccaagctgaaattctaattaaactactccttgtacgtttttcagcacacactttaactgctatgtcagtattaacgaactaatactaatacattagtgcttttatatacttcatgcattactgttaatcccatgaataatatattgcactataattgtttaatcatatatagtacattaatacattaaagtacatcaagaatataatctgcatacatataaccacgtattaacaatcccccaatcaactataacacattcaccgtatcaactgtacaactcaaactaagccaacacgaatatcaacccatactaaaaatccttaacattacatagtacattaaatcattcatcgaacatagcgcatttcagtcaagaaactccttgtcaacatggacataacctcgcagcccagtaaccacagactgggcaggaaaactgcaatcccctgtaaatagcatgcagtttatatagcattttcacttaacaccctcctcttaatgactttcacctggcaaccttcatttaactcaaaactcagggcctcattcccctgtacagcccatgttgcatggaactggccagggttcagatgtttatcatagacagggaacaaatctctaggttggccacttccagattccctagctcagaacattcaggtgcgtctgccatacaggatctggctaagggtaggcttaagttattgctatcaggtgcgcttaccctacatatgtcctcaggaaaattacttaatttctttgtccaaaggtttccttagatggaaacaggagcactttcctcctcctaaaggggattaaatgagaatatacatatcacatatatacgtgtgtacaaacacacatatatatgtgcatatatacacacagatctgttgatttaaaatgtgttgccacagatgctcttttaatgcttacttctgtttttataaatatttgcatcatttaatgacaaaatatctacttgaggatatttttgattaataatttaaatgtgttcctttcctatatccaagaagacctgcagcagggtacaaggtaaagcagtcacacagaactcatgttcttgggtcatgttgctgctgtccacatagtacggagcaaatgtgctctgaactggcaccactggatttgcttcccagaaaagtctttgaaagaacaataatgaatacataccatctgggtgagacttggaggtattggggcattttagaatgtccacctggcttacttttttgactttagctaaataaaatgtaataaagggggcaggtgggagttgggggaactgttcaactccctggttagtgcccccaaatgctgaccagtgccacagaaacccatgtggctgttgggtgtggctcatccgtgtggaaaaggccccttctaggcccatactcacttccagtgttaggcatgtttaatcagcgttagcggggagtgtgcattgataaccacaaaattggaagacactatgaggagaggagtgtgtgagagagaacaagagaggaaagggtttaactctacctttcactctagttttctaccggtgggtgcttgatcatctaacggaagttgtctgtgcaaggggagctagaaatcaagtgagcagaaaatcgattttgcataaattgaaaaagctggagataatcaccaaaatatctcagtcatctctgagaaaggaagggaaattggagctgaaaaggattttcatttctaggtccctgagaagctttgatgacaaggcacaagtgttgtatattttctgaactagggtgaccatacttattgttaccagaaaagcacttgtcctggtaaagaccaggaactataaggagacaaatagcttcatcctcagccctttctctttggactgacacatctggtttacaaatgaggccatttatactggatagattgggaacagatggctgctacctggggtcttcaagtcctcaccttcccagacagccctttgcttttcaacatgcagatagaaaaaccgatgcccctattccacatcctatggaaaacctagtatcctccctgaagatttccatggattcttgggggcattggttgcatctgatgacctcttgttccatccagtgtgtgacggctaaaagttcgggcctctgactaaaacagacctgtgatcaaatcctggttctctctccttacagtgatgtaaaattaggcaatttgctttgatctttctcagcctcaatttgctcatccatgagatggagataatactacttacctcccagaatgttataactgttaagttaataatgggatagtgataagggaatacaagtaaagcacttagcatgcaacctggcacaaagaatgaactaaatagagggtagctggtattcctttattcacactaccctaaacagatgagtcaagatggtaaattttttgtacagtatttattccaaagcgcatgacaatctttcccaaggagatccttttaactttaaaaattttcaagaactctatacactacaacattcttttagtattcattaattctgaaaatacaagatcacaaaaggtagatagaagacatgttattttttccatttttggacaatgtttgatgcacacaaggaatcacagacctcttgcggtaattctaaggttccctggtggtccctggccccaggttgcctgcattcacaaacacttcactacggaattcctaaacttcattaactactgcttttctgaaagggctcagttcttacatactcaccgaacaaatatttattgagtgtctgttatgcaccacatattctgctaggactatcttgttttacactttaaaggacataataaatataaaagagatttgagcagggttacgtgcaataaaacctgtgggctgattattccctctctccgttcccggcatttcagcttgtgataatcacttcaccatgtcgggttttacattgtgtccaataaacagcttgtcacaggggtcatggctcagttgagaactgcccagcagtcccacttggtatttcttagaaattatacctcccacttttcacatttcatatcctacccccaaattaagttgggttttttttttagtaagattcccgtctatttatcatttccacattgtttaaaggaggattattcagggtagttttgcttcctgcgcttctttttttattcatacataacagatgtacatattttgggggtacatgggacattttaacacattcatataatgtataaaaatcaaatcagtgtgattgggtgatccattgccttaaatatttgtcttttcattctgctaggaatattcaaattattttcttctggtgactttgaaatatacaatacattattgttaactatagtcatgttactgatctttcaagcactaggtcttatttcttctatcaaactgtatatttttacctattaatcaacctttcttctttcctccgccctttcttgacctctagtaactacccatctactctctgccttcatgagatccacttttttagctctcacataaaagtgagaacatttgatgtctgtctttctatgcctggcttatttcacttaacataatgacctccagttccattcacattgctacaaatgacaggatttcattcttctttatggttgaataatgtttcattgcgtatatatgccacattttctttatccattaatctggtttgtgggcatttaggttgattccatattttggctattgtgaatagtgctgcagtaaacatgagagtgcagacagctcttcaacatattgattttatttcttttggatacatgcccagtattgaaactgctgaatcgtatggcagtcctgtttttagttttcttaggaacctccacactattttccatagtggctatattagtttacattcctaacaacagcgtataaatgttcctctttctccacatccattattccctttttgataaaagctgttttaactggggtgagacgctatctcattgtggttttgattcgcatttctctgatagttaatgatgcttgagcatttttttttttcatatgcctgttggccactttctgtcttctgagaaatgactactcagatcttttgcccgctttaaaatcagattatttggattttgtttgtttgtttgctattgagttgtttgagctccttatatattatggttatcaatcccttatcagatggataggccaaactaaattttttatcttttcctgctttttcttgaaggcagagaaatggggtttcttttgtgcccttgcttaagtttagatattttaatatctttcatcaaatattattatttaagcatggaatataaagaattagggtaatcactctgtggttctccccatgtatacatgttaaataaatttgtatggttttcctccaattaacctgaaaaaaaatgtacttccctggtggacattatagtttttgggggtggggggtcggtgccacaagacaaggttgcaggtatataataaaatggcaatgtgcagacgtgatagagtaggcacagaaagccaatgagggcttttgttgtcagagaaagtcctatagagaaagcaggatttcagctcatcattaagaatagattgggtttagttggggaaggtgcaaggaaagggtgtttcttctgaaaggtgcagcatagcagatgtgcagggatgaaaataagtgtcatcagctcaaagggctccaagtgactaggatgtcagcatggctgatgcagaagatctgtgctaaagaatactggaaaggaaggtggctgggaatcatgaggtaaggcaatggagagccctgaggaccagcttgaggactctgggctgacatttcctgagcaatggggaggtggtgtgtggtgcttcatctggcatggtctctggatggggagagactggaggtggggacactgattggaggctgcagatgaaggctctgcaagatatgggaatgacagactatgggtggccaagggagagaaggggaaggaataacaggtattaataattccagaatgtttgctctgggtaatcgggaatgactggaggagttcccaccaattaaagttttgtaggaattctgcctttcaaatgtctttcttccttttccaatgaaagtcttttccttcttccatgtatggaacctgatagtaaaaaaagcatttatctttctattttctagcattgctttcctacaaaaagcactaaaatcctgccaattatgaaatggcccatcttcagagagtaatcataaatagtcaaaacaacttgaagatcttatcagcttcccgctttccctctctcaccaaaattaaaccccatgactagcaatgaaacagcaagtatttcaactagatgtatttgttatagatgtcttttctcctctgtttttattgccaggcagcaagttagcagttaagtccatgggtctgttttcttaacctattcaccctgaactggaatttttgagtttatactaaaaacaataatatcctacccttattatttctctttgttgtttaaatattgccttcattaatgttatctcatttgattctttatcaaaaagccatatgattcaaatctggcacatatcattattcccctttaatatatgagcaagctgagaatgagaaataatatgttcatttacattgccaacaagtgtcatccctggaaataatgtctaggatctgttattcaattacatccaatatactagaagctgttacagagaaaaaaaaagttcaacatattatcttgaaaatcttcttataaaattgagaaaatcaggcataaatatgtaaacagttaaataacattccatggctaagtgtcccaccagatggcaatagaagccatgagtcagaaggtacagggttaattaccgtgtcataatgtgatacacaaatatgggaagactgtgttcatttctgggctctgtgttttaagagggacactgacaacgtagagcttacccaagtgagactcactgggagtccgaggagacgcagagatcaaagatgaagggacaagtgtccaaacgaagagagaaccccgggagggcattatgcccctccacttattgcaatgcctggtatgtgaaaggcttattctccttggtcccaacagtatgcctgtggctaatgaacagaaattacaggaaggaagcctccaacttcatgtaaggaaggatgctccaaccacaggaacaatgaaaagtatacagaacaagctgcctaatgtggtaatgagctccctatccttaaaggtgatccattatgggttaggtgaacacctatttaggatgttgtagggagaatgcctgtatcacatgccttctgcgtgctgtcaagttcagaaccagcaaaatggtataactgagagatggcatggtgcagggtttaagagcacagtctctagatcctatatgatgctcagtcttggaactcagcaaccatattgtaaagaagccaggccccacggagaagccatttgtaagccccagctaaggtatcagaggacagtcaacatcaaccgtgagatgcatgaatatgcaagctttcagataattccagtgttccacttttcgagccactccatttgataccaagtagagcagggatgacgtgccaccccaaaccctgcccaaattgctgattcataatcaaaataaatgctatcatttaaagccactaagtttgggggtggtgaattcttcagtgctggataactgaaatacaaagtttgtgtgatattcagagttaccattggtcttcacatccattaataggtggtgctgagtcaactagaaggtggtgttatgtgcagaaaggcagttgggtgggttgtgccgatgagagaagagaaggcttggtgaagctattgagagaacatgaataaaagattaatgaggaactttgaagacacagtgaaggacacagtgaaggctagatcttcattgtgtccatggttttgtcattccctccatctcctatatggttttgtgacttttctaattcttcttgaggaggcagctagattgagcaagtgctaggtcctgtcaagaaggtagaagaaatcttaaaagtctgtaggcctgcagaggaggagtgcgtggtggacttcagaagcagaacacccaaggggacagaataaaatcaaagagactcacatattacagaccatgatacattaaacatccacaatgttaatatggaaaggaacccaagagattgttttctagataatgaaatcaagattatgcacttggaaggtaaataagtacttattgtagagtacaaacgctacagttgaacttcaggggagaaatattaggtttgttgagaagaatctgaatggacgacagacctaaaaatgctttctagatagggagaccaggaaaatcaagatagggtggatatgataacgggctgtttttgagtctaagaagggaaaggtacggcagttcttgaaggtgagatcagggaatcacgatggaactttgtatcatcaataccataaaaaacagttctctcatttttggaggaaaaaaaatacactgaacacacagaaggagagaaccagagtgggtggccagatctccagcaagcagcccatgatatattagaatgccaatggtctgctgtcccggtgctctgaatacaggcaagaggaggcaggcaagggaacaaaccaggaggcagttacctggcttcctcgttgcttttcaggctacaataaaacatttctaaattaagatgtaatgctggaaatctgggctccaatcttctgagtggcgattctgatgagggtaactgaagtccccacattgctcatcactcctcctatttcctgcagcacacagcattgattatcctcctaagaaaagccatttcagcatctagagtggaagaggggagtattaatcatagtgaaatttgacaaggcagaagcctccacggacccacttgagccatttaaagcttttatgtgccctctagactggaacaagtgcatgtacatcaggaccataaaagaggggagacgcttatttagaaagcattaatagctcaggactggcctctctcttttgcgtctcttcttagccctggttgtctaagtcaagtcatttcagctgtcagtattgttttattcctcattttaggagatactactaaaatgtcagttcacctggaagttgtgcccagagcatttaattggatttacattaaaatgaaagaaatatgccaatgtggttttaattccaacagttattataatagataaggattctagaaaaaataatgtcaagtctaggtgaaattgttaggctataaaatggactcaggggatttttttgaaaagagaaaggggacatgttcctgctgtaactgtatttaacattcagcgtagggtacaaagctctccaccttttctctgcttgctaagacaagtggatgtatcttagaactggggactagaagggacatgaaagcatcatctacgtaatttctctgctgtcagagaaatcattctatgtgtattaaatatctcttgtttgtaacttattctcctctaaaggacctcacaaatgagccctgagggagctgtttagaattaattcatcacaataaaaaaattgtctttgtgcaacctttcatattttagttttagactcttccattttcagctctatatgaaaagactcaatagtaggttattatcttcctgataaaaagccttcacattgttaatctctttttccaaaaatatatttgtttctgaaatatttcctcatatgctctcttttcctgcccctaaatagtatgtattgctcaatgccaagccctccctcaagttaattgctcttttttagttataaaaccccaactggagtccatctaatcatctaataattcctatacttatgcctccagggccacactagctttttaaactacagcatggtgaaagctggtggtcatttgctgtctcattttcctaccaaacttgtatgaaattgttctgtctttcccaagaaccgtccttcaccaagtatttcttcaatggactgtgattaaatggttttaactgattattcaatgaatatgtggggcccatttttttaaaaaaattagaaaacagatgggttttaaaaaagagaggatatacaatgaaaagttctcttccaatctctgtcccagtcatgtgctcccccaacccattagggtctaaccccatttctgctgcttgtgtattcatggagagagatcatgtacataaaaagaaatgcatgtaatgtatttactacaattggcagcacgttttagcactgctctgcatctttttttattttaataatataccttgggggtctttccactttggtatacatggatctcctttattctatttttttgtttgtttgtttctatttttttcagtagactagcagtctactgactggatgtaccatcatatatttattgcaaacctttctcaatatggatttcattctccaaaatatcctctaccagctgaagattgtggaagactcactatcatacttcactgtgcaactgtttgcagaatgcttactatcagcacagcactgggcttggtgccagagggaatacaagaagcagtgagtttggcagcctctgctccctaggaaactacagccccatcagggagaaaatacatgagaataagggggtcctgattgccaaatctatccccctaaggttttcctttgttttcttgtacatcagacattttttctaatgcatggtacaaatggcctttcccctttattggatgtgtgtatggtggtttgtgtgcctggtcctgcttttatactgggagccacaagaacagaactgtcaaactcaaatacctacagaggccaggagggtgacctaaatgagtgaagggtagagaagtgagccagttgggaaggcataagctctgtttaaagggatccccaactgctactgtgctcgattatttccctgtgaacaaccagatcttttggttgattaaaaggaaccagtgggtaacaggtacatgggacttttctgtattatttcttacaatacatgtgggtctataattatctcaaaataaaaaatttaattttaaagaaaatcagatacctagatcattgtgtgaaatgtcctgatttataaatgtaggcaaaatctgagcattgtgcaggtgcagcaaaactcatctatctgtaagccaaatgcagccttgggtccaccattttgctatctgtgtatagctgaatcattataatttgatttgtataaaacccaaggcaacccaatccatgactttgctttaaaaatacttgctactgtgagtttccattacttttgacagtagccatccccacttacctgttgagttttcctctcattccaggtccttggatgtggctgtgttgcccaagctattctcagtcgaggacgttttggaggggtcatcactatcaatgttggattttcaatggcagttgcaatggccatttatgtggctggcggtgtctctggtaagcagtagaaataatgaatgctgctcttaattcagccactccaatgtgcccgcacagtgctggctgcttcacataccttaatttacttatatctcaaaaatcctgcaagagtgtgtattggatcccatttttatagataaaattaaaatcaccaaccaatggtaagtaacttattatgcaaaggcaacatttaacaagagacagaaatgggggttagagctcataaattattcattcctaagtctggccgctgcatcttattctccaccagtcaacgggattttttacttgctctagtccacatttggaatccaactcccagataccctgtaaatagcccagtgaaggtcactccaacagaggcaagtcctggacctttatctatagcaaacaggaagagtctgggtctcttgcaaaatggatggtgctttcatggacgtgcagatctgatctacaaggtcttagtctaatcctgctctgaaaagtaaactgattagaagccattcacatggttctcaaaagcaaaatgaaatgataatttaatgcagtctgggcacttaggaagcgactgtggaaatgatctggttaccaaagacactcagaatctttcagcagcctggtgtgggtcctcagagcattgagtcaagacttggtgagaagagaagaaagggaactgagaaaccatgcatgctgagcactgtgctaaatgccagactgcctttatctccctctcctttacaacagcactctgaggcgggtattactgtctccattctacaaagaaatccctcctgagagggagtcagtaaataagtggaagccctgcagctggtaaagaagaagagctgagatttgatccaggctcacatgatctgcatgacagtgaagctcacatcctcattttttgtttgtttgttttttgtttggttttgtttttttttaggagtcttgcttttctccctcaggctggagcacaatggcacgatcttggctcactgcaacctacacctcctgggttcaagcgattctcccgcctcagcctcctgagtagctgggattacaggcacacactgccacacctggctaattattttgtatttttagtagagacggggtttcaccatgttggccaggctggtctcgagctcctgacttcaggtgatccacctgccttggcctcccaaagtgctgggattacaagtgtaagccaccgcaccctgcctgttttttgttttttaagacagagtctcactctgttgcccaggctggagtgtcgtggcaggatcacagctcactgcagccttgacttcccaggctcaaacaattctaccttctgccacctcagccttccaagtagctaggactacaggtacgagccaccacacccagctaatttttgtattttttttgtagagacagggtcttgccatgttgcccaggctggtcttgaactcctggactcaagtgatccacttgcctcagcctcccaaagtgctaggattacaggcatgagccaccacacccagccaacacatccccattcttacacagcatttacaatgctctattatgagaagggcattacaagcaggttagttgggcagaggatcctgcagttaagctggctggcctcctagcaaacactgcctttgtagcactgaagaacgattgcttagactccttgggagctccttgggctgtgcatccggaatggccattgaccagctggcaccctttccatattacagccagactcagatccaagggtcgtccaggttaagacagagtcccactctgttgcccaggctggactgtgatggtgggatcacagaactttgcaaccttgacctcccagattcctatatatgtgatttgttagcatgcacagtgtctccattcaaacttttgtccagcttttgctgtgatcccagcactctactgcttaactcaaggcaaactccaagaagggcacaagaaaaaccaggggactgccctaaatatcccgaggcttaactgctccccctataagctcccactgagttcaccaggtaatgtaggtgtagccatattttcagcctttttttaggtagagacatggttttggagtattttgcctgcataacaccctgaactgaacatcatccaatagttacttagaattaattggccccagaccctaccactgctgttaacacccttcaccactgcaatccatatctgattctgtccattgatatacctggggatttctgcagtcactgtaggaggaaaaaagggaaagaaccagcaggaagaagaaggagtgaagaaaaggtgtgaagtcttctgtggcctctaaactggggttgaatccctatagttacaagagtaagggccaaaactgatgggacttctttcagtagaagctgtttctgggtgagagtcaggacctgaccaaaatgtttacaagcaactatgagaccctaaaatacaaacagagtggtgtctgagactggctggaaaatagtagcactggcttggaaaggcagtccttatctttctatctctatttggaatctagcacagttcaggctccctaaggactggtggctttgggaacattttgggaacatgcagcacaacacactggcccatcatgacatctgtgtggacaacagcatcagtatcagctttggcaggtgcaggaataggggaagattgtgtgtgggaggtgactttctcatcgtggctgcagctaacatactggtttgtgggcacatactggcttgtaaggtctgcttgaagattccaaaagtaaccaatttatactgagcataaatgtgaatgggatcctattttgtggtcaccgactagtgaggtcagcagatatttgtcttttaaatataaaacatcaccttattgtaaagaagttacagcatcctgctcccctgggaatgttccagtcaaatccagtcagtcagaggtcataataccctaattatgtagcatcctgcacaaagcatttaggctgtctaatggcaattgctattgatgtagtattttatgactaaaataaacttattgaaaaagcaaatagacttgctttcattacaaaatatagggttatcttgatcacaggattgtgtatgggtggcgaggaggagaggatttgcatagaacagaacactgaaggggttccctctgctcacctaagactttcatcttgtgccctgtgggatccagctgtgcccgcgggccctttatgtgaataatctgattgtcccagcaacctcggcaaagccaagagttgagtgcaggactctctgactctagttctcatgttctttccaatacactgctctgccttgtccttgtgataagccagggtgaccactcatctctgcggggagaaatttctagcagccacttggcctctaaccagaggcatcagggtgccacttgtccacctctctttctgaacctttctgcctcctctccagcccttttcctcattgctttgcaaagcacccttccagacataccctctctaacccccaaacaactatttttctctgtagatcactgtagtctgagctatctaaataaggcctcagattatttcttcaactttcttttttttttttaatggagtcttgctctgtcaccaggctggagtgcagtggcataatctcagatcattgcaacctccgcctcccaggttcaagcgattctcctgcctcagcctcccaagtagctgggactacaggcgtatgccactatgcccagcaaatttttgtatttttagtagagacagggtttcaccatgttggccaggattatcttgatttcttgacctcatgatccactcgccttggcctcccaaagtgctgagattacaagcgtgagccaccatgccctgcctatttcttcagcttttatattgaatacctactaagtgccttgcatcatgtgagacccaaggtaaataatagttccagcctcaggatgcttaggaggagccaacaaacagaaacctacttatgggaaattttattttggaagacgaaattgttaaacaaagaaatctcccattggtagactaaactcaagggagttggactggaggtgatcacttacggcccccttggttttttgttgttgttgttgttgtttgtttttgtcgcccaggctggagtgcagtggcgcaatctctgctcactgcaacttctgcctcctgggttcaagcaattctcctgcctcagtctcctgagtagccaggatcacaggtgcgagccaccacgcccaactaatttttttttttttttgagacggagtttcgctcgttgcccaggctggagtacagtggcaccatctcggctcactgcaacctccgcctcctgggttcaagcaattagcctgcctcagcctcccgagtagctgggaatgcaggtgcctgccaccacgcccagctaattttttctatttttagtagagacggcgtttcaccatgttgggcaggctggtctcaaactcctgaccttgtgatccacccaactcagcctcccaaagtgctgggattacaggcatgagccactgcacctggccccgcaccccttttcctttcacaatggctacctccaggcagctggaggcagctactctgcagcaagcctctgggagaagagaaacttcccactgccctgtgtgccccctcctccctcctaacccctgtttctagccttccctggtaaggtgtactgctagtctccagaactgtggctttcctggatttgaaaggaaaacacaacagttgaagtgtttataaggctttaggaaatcatctgtgcttcaatacactggcattttcttgctcattgtagccactgagtggcaggggcaggtgaagaaaggactcttgtggcttagccagcagccagaatcccccacccactgcaagtccattttgccctggagacttggaaaaagcatagctttctccacagggagtggtagaggcttctggaaacaagtgcacctattggttctgtgcaggatcatcatcatcatcatcatcatcatcatcatcaccaccatcatccctcctttgtttacgaccaaggccacagcctgaaagtccacatctcaatgtggtgtctctgattccccctccacaaaaaatgtcatctgtattcctcaaatgaacatctgttttcactaggaagtgagcctatatgctcagacaatttttttaaattctttatttatttttctgcccacaaaagcagagaagcctttataaacacggtgaagatgggctggaagtggtatagcagaaaacacaggctctagaattcagataaacctgaatctgaatcctggttcttccatttactgaccatgtagccttaagaaacttactttccgtccatgcctcagttttctcatccgttaactggagtagtactcatctcattgggttcttaagaggatcaaatccacctactaaccctacaatggtgtctggtaagtatttgataaatgctgctgttgttgttactagcaaagccagctggggatgttagagaccaaccacagagcaaactctctatttgattttatggctggatgcaagtcaaggaagccgatttctctagctggagcttgggctgggctcaggaaagggttgctacatttctgtataggagagaaatgcattgtttatgcagaggaactgagaacaagccactcacactgtgaccacctcaaaatcaactatgcccccaccctcagcaacccacagtcattttcctttaaagatacatctcttttgagcttgtttttatatcctctgggcatggccggaggaactgctagatatagatttaaatcttatacatatattggataagcatattgacttttaaaagatatcgaagttaatagctacatcagtctgtttggggtgactgctttatagatagagggattcaagcctattaaaaaaacagccacttgagcacatcaagtttctttttatacctgactctcttttcatcaagataaacattctaaactgttatccaaggatgccatgaaaatgatcattcctccttctctccctgctcagatacacactctctctttcacgtgcatacacacccctctttctcctcataggcagtagttctctgcctagaagactgaattccacctgaggtctctagatggttttctgtatattcttcttcctaacctgccctctcacttctccaatcttcccttgcaggtggtcacatcaacccagctgtgtctttagcaatgtgtctctttggacggatgaaatggttcaaattgccattttatgtgggagcccagttcttgggagcctttgtgggggctgcaaccgtctttggcatttactatggtgagtaaagtccctgagtcctaaggttaggaagagctgggcataatttgaaagtcagaattacttggtaggcacaggcgagaaaaagcaaggacgggaagcattctacaagtgacagcaagttctaaatttgagaaaatgaggccatattaccattccagcaaattcgtgaatcctgttaatgaaaatggtcatatcttaaatggggagcagaacttaaaggaagcctgtctttgtgccaagccagcactgcaacatacacatccatacaacaagcagtaggccagtggcttccccactgctgtctctgatttggctgggaaaagaggtgttttctggtgacaaaggtgtgaccagtaagtattgagagaaggactctgggaagatgtctttgaaggaagagacacagcagagatgtgggctgggtgggagcctcattgcccttctatcaaacagccagggcttccactctgcaacagtccttgggaaaaacacagtattggtcatagaagaccagactttagctctagctgttcagtaaatacctgtgcaacttaaataagtcatctgatctgactagcctcagtttcctcctctggaaaatgagggaattgaatcagatgactctcgaggcctttcaaactaaaacaccttggtgttcccatgaaatcaactgttaaagcagctccttccttctaacccaaggtaagccttaatacattatgacttacccaaactgaagaccttaggaagtagaatttatgaaccaggccttatagaaatgggtcatggagggctgggtgtgtggtggctcatgcctgtaatcccaacactttgggaggcataagtgggaggatcgtttgagcccagtagttcaagaccagcctgggcaacatagggatacctcgtctcttcagaaaaaaaaattaaaaataagccaggtgtggtaactcacatctgtagtcccagctactcaggaggctgataagaggattgcttcagcccagatcaaggctgcagtgagccaggatcatgccattgcactccagcctgggcagcagagcaagaccctgtctccaaagaagaaaaaaaagaaaaaaaaaaaggaaaagaaataggttatggaaagggacttttccccatagagtgtctctctgagaattagagtctaaaagggctaaaatggaattcaagaacacacaggaaattcagagcccgctgctgtggcaagaatggcctggggtcagtgctagctgtggcagtggctgcaatacccctggctctaagggcatcttcagtatactgcaacaccctcaaattctggagtggtattaggggttactgagagccctctgggaattataaattattcctagagcaagaccaagccccactttggcttacagttgacctcgaaaactccaagaattcccccaaaagagaactcattcccaggaccctatcgaagaataatttgtccaagtttgcagtgggccattcagagaggccaaaggccttcttcctttgaccctccatcctgggattcacctgggcatctctaaaactatcattcctcggaagtagagagagacaaatgacatggctatgccaagctcaatctgagtgactggttgtttagcacaaggcttggcacaggcctccttcaactcttcccagggaacactaatgtgccagagctttctctttcatagatggacttatgtcctttgctggtggaaaactgctgatcgtgggagaaaatgcaacagcacacatttttgcaacatacccagctccgtatctatctctggcgaacgcatttgcagatcaagtaagtgtagattcaacaaagacttaactttggtgaaaagatatgctctcataaggggaatgcattggagaattagagaccaatcagaaaggagagattatatttccaaaggcagaggttgctgggcataacccaagctttcttgataatcgttttacctttacaaggagctgataccaccagaaaagttttctccttcctctttaatacgtacacttgtttaacattagttgagatccagaaaagagtgaatgactaaacatttcaatccctgtagacacaaatggacttcacttcactattgactcttttgaacaagtggtctaagtgtggaggccacaagagaagatatttctatttcaaatcaaagactgtgaagggtggtagttgagcctaggggatcatcaatcctctctgtctcaccatgtgcataggtttatttatttaaactctcaaggtggctagaagtatctctgcttattgacctttaaacaagcttctagcacccaagcatactgtggtgtaaccaatagcacacctatttggtcagcttatggcaggactaacattgtcaactcaaaagactcttcttcccttactgtgtcccaagaaggctgctggttatcttggtcagggcagtggaaggaaggctatatttttcagtatctctaagttcttcctctatgatcaggacttatacagtggagcagatcagaatgggcatgtgtataaaatggatcttccagttcatcagctatgcccacctccattcaatgtatggatgaaaaactatgccctcatgtcctcagcttttttactttccactttcatcaataaatcaaacaggcaacaagtgttcattaaaaactcactatgtgcttggccctttgtctggagcttgaaagactgacgagtaaatgtgagttgtaaagatccaagatgcagagtctgaatgcttgaattaaaatagctgttctgcctccaacttcctgcgtggcttcagcaaagtactgaactttttgggtcgcaagttattcacttgtaaaatggggataatatgagccttcatgaagttacttatgaggataaaaagaataatatgagaaaaacgcattgtaagcagtatctgcccagaaattttgaatctagttgaagagcattgaacaaggaaacaatgcaaaataagatataattagacagagctgggcacggtggttcacatcttactccctgcattttgggaggccaaggtggacaggtcacttgagcccaggagttcactaccagcctgggcaacatggcaaaaccctgcctctaccaaaaatacaaaaattagccatgcgtggtggtgcttgcatgtagtcccagcaacttgggaggctgaggtgggaggatagcttgcacccaggagttggaggctgcagtgagctatgattacaccaccacacctcagcctgggcaaaaaagaagaaaattagataggaaactggagtatagcgaccatattataatagaaatttgaagcagagagcatagaagagcgaggacaattttggttattttggtgtttttctctaagctttttttttttttttttaagactgagcctgttgccccaggctggagtgcagtggcgtgatctcagctcactgcaacctccacctcccaggttcaagcaattctcctgcctcagcctttggagtagctaggattcccagtgtgagccaccactcctggctaatttttgtatttttagtagagacggggtttcaccatgttggccagactggtctcgaactcctgacctcataatccgcctgcctaagcctcccaaagctgtatcttgatcaatgctgacctgcataccatatcctgagcttccttcattgtgtccttgtctcagagtgagttggcaatttctctaaggtctgtggtttctaagcccattattgtgtataaagtcaagtagaaaaagcgaatattagaaaaccctgaggctgtaaccgtcagcaaaccctgaatgctgtccgtattcttaggctttggagagagttccaaaaattgccaattatcagtcagtcaacacccaacagagctcttggccatcatgaccatattttggggtcaccttatttcaagattactaaattgtttagctgtaaatactcttcaagcatattccttggtttagttaataatcaatcatttatttacccaataactatttactgggcacctacttagccaagtacagctctatactcttccttcttcaaggagttcagagtctgaggctcaatctaacaagtaagtagatactcttttgcagagtgacagggacactgatacaagtaaagtcagaatgtcatggaagcccagaggagggagcttttactctgcaagagagagttggagaaggcattccagagaagagtgctcttgggggaacatgtgtcttttaatattaattaaaaattattttaacattattattttggaaagcagatgaaatgataatgaacttaaactttaaaatcagttgcctggttcccatcctgattcttctgcttacaagtttgacattggacaagtgtcttaaccttgttttactcgagttttcttatctataaactgagaataatagcaggatttacctcacagcattactgtgaagttttaatgagataaagcatatgaagtacctggcacataataaatgttcaataaataatagctatcatttaactgttaaagtaaaacagtagtttggaaaactaataacatagaaaccagctataattggccaattcaaatacctattatcatttttgtttattgtcttaaaatttttcttttacgtatgtttttaagtagagtcaaccctacatatccatgaattctgcatccatggattcaaccaaccatggatcaaaaatatttttaaattgcatttgtattggacatgtacagacttttttcttgtcattattccctaaacaataccgtattcacattatatcagatattataataatctagagatgatttaaagtatacaggaggatgtgcataggttatatacaaataatacacaattttatattggggacttgaacatccatggatttttggtacttgcaggaagtcctggaaccaatcccccacagacaccaagggacagctgtataaaacacaaaacaaaacacaaacacaaacaaaaaaatacaaatttttttcacttaatgttataagtatttttaaatattactacatagcttttatgtttatagtttttaatggctatacagtagactaaaaaaagagtaaaagcttccagtttttcagtactataaattttgctaaagtggacattttaaaatgatttttccacgttttagattatttccattgtgtaattctcaaacatccagcagcatcaaattgtataaataaatttatgtttttgaatgcatattgctaaattattgctgaatttccttttaaaagcatagtacttgtttagatctattagaaaagcctgaaataacgggtatctttttcctaacatttgatattgtttggatttattttattactaatgagtttttaccactgctttcctaattctgttttccttttttgaattttctgttcacttcctttgcacactcatctattgatatctcacattgcttttctcaccaatttgaacaagcctttcattcatttttccagcctgcagcttgccgttctacttagctgagtagtagcttagcaggatagtgtggtattaagataacagtctctggagccaaactctctggatccaaacctgactgccacctactaagtgggcagggtaaccaatctgtaaatgagaattatattggcttctcctctagaattgctgtagaaagaagagagactggcacaattattcacccagtacatgccagttattactaggcatattgtaatataagtaaaatagtaatattgtaatattgtaaaatagtaagaagggatgattttgagacatgcaggtgagcagaatgcaggctaaagccctggctcaacttctccctctccaggtggtggccaccatgatactcctcataatcgtctttgccatctttgactccagaaacttgggagcccccagaggcctagagcccattgccatcggcctcctgattattgtcattgcttcctccctgggactgaacagtggctgtgccatgaacccagctcgagacctgagtcccagacttttcactgccttggcaggctgggggtttgaagtcttcaggtaagtaacagtggtggggaagagagagacagagtcatgctgcgcctcaccagtggggcggggctttgacatggagatccagggaagttcagatgacagcgccaacgttaactgcacactctggtcataagcatgagacatttcactggtattttgtggtatgatgtgtgtgtgtgtgtgtgtgtgtgtgtgaaagagagagagagacagagaaagagaggcggctctaggtaaattcaatagagataaaactgtcccacataataataatccctcaattgctatatgattttatgtgaacattttagctgtataacttgtctgcagattcattttgtgacaaagttgggctacaatttgcatcctatgatgccactacagggctcttcctcgtgaaccacgcaacctatgctgcacttattccacagatatttgttgggcattatctgtgctttagtcactgtgctaagcaaggaaatttcaaatcagcaaaagcactcaccttgagcgatataataagtgctctggcaacccctaaaagtgctgcaggaacacagataacgtgtgagtaactgccagggaagcagggaaagacatgactgcaaatgtgagagctgacttggttctggagatatgagtaagaacttccggacaggacagacgtaagattgagcctctgccccagaaatggcaagactccacactttctacttcagaatgtgctgaggaaatacgccaactgttggtacctatcttctaggacaaaatttgccccttcactacttcctggtttccctgcatgcagtcacagaggcgcatcctcatccacccatggcaacttcctatggggaaggagctcactttggttcatcaggaaacagggctctaccatggagaaaagtaggaaaaacatgtgtgagcaaattctactttaatgtttcctgtgagaacaaaggacgtgagtgttcccaaatgatgacctagacatagagtcatcagggtttggcctgcatttcaccagggtgaaaaataagttaaaataactggtaccattgagatcaccaccacagatagcactcagagcatctagagtgtaagtgccatgagtaccttgacagccttttcccctggtatatccacagtgcctagaacagggtgtagctcatagaaaggctcaataaaaaggtattgaataaaagaatgaatgaattctatgtgacggacacttcctaggagttgaaggatgcaaagcaactcagtcaacattccctgtcctcaagaaattgacaatccaataaatttcctagaacagcagttctcaaccagaggtgggcaactgttccccagggcatttttggttgtcacggctggacctgagggaaggggctgctactggcatctaatgggtagacgccaggaatgctgttaagcatcctacaatgcacacggcacacctcccacaacaaagaattattcagtccaaaatgttaataggtttgtggttgagaaattctctcctagaatgatcacaactagaacacgaaaatgtccgcaactaggatggatctcttgtatctttccaatggagcatcaaaaacatatgtcctaaggatctccttattcccagagcccagaattcgcccatgagtttcccttaattccaccatccagagtctcagtcttggagaccaagctcttcttccaccccctttgagacctccttggtgattcattcaaaagtcaaagagacatggtggttccaaaacagatggagatcttgcccaatgtccctttgctctgttaagtatatgttaggagaactcacgtgaaggaaaatgttggaatcacctaatgtcccatgtccttagctagcatcaggatcttttcccttccatggagtcatttattcattcatgcagctacttgatatcttactcaaggctctccactaaagatgataagacctagtttctgtccttcactgttgtgtttatattctacttaagaggttattccagaagctcttacacaaggttgaagcagtcaagtgccattaaagagatttgactaattaactccaggagtttagaggtctacagctccaaatttaagaattatgggagtggaagtgggaacatttatagtatttggatatgcttaaacaagcaacagaggggttatctcaggcagagagaaccgttattcaaagacacaatgttaaaatcacaagtgatctagtctagctggaagggtgcagaaagaaagttggaaagggccgttgtcagatcatagaggacctggactcccagaatgagaattttggcttttcttctgctccctgttttgtaggttatagggagtagcaaagtatttggggcagaagtttggcaggtttagaatgtcaatttagcaaaattaatctggcatcatcctataagactgattgaaaaaggagagagtggggaagtagaaacccattaggaaagagcagggttggctggtaatcctcaaccaaaaatttctccccaaaacctttgagggacaacaggcttccaaagtgttcttagagtgtctcatttcctcagagttctatattcaagggaaatccctcagtagatatcctagcttggacctacacctaaggtcttagaaaaaaaacaaggctaccttgttcccaggtccccatgtactcctgaacggcaagcagtggaatgatggcaggtacagcgagatgccagagggtttgatgagggcaagagtcaacagaccctggccaaagactcttggtttttcctttaaatgttaagatgtttcccatcaaatttctctccacttattcacattgttaatgacttaatcagtgaaagctctcaggagtattattaaatttttcaaatagttggcccaaagtattgaatccctaaagatggtcctttagacagttaacatacacatgttcctggcatctcccgaatcatagtgtgcaaatgatcactatttgtgtgcatataggactaggctaggcaggtaaccacatctgagcgcctccatttgtgacttgtgaacctgatacaacatgtccaatctgataaaataaaaagcatccagccagtgaagaggacgtgtaccctggctgatatcttggaccttcatctgctaaatgacaggaagaaatttgccataaaaggaaatgatggacccaacaggtagcttatactcccttcatgttttgacctctagagaagacttcaagtctttacagccaaacgggagaggcagccaccctccatgtgcctcccctagaaaaatccatctggatttggggcctggggaagggcactgtgagggcggccgagtacagcatcctcgctgccgtgtggtaagagtgacatctggtggctgccatgctcaggctcagtgctcttccatcaaccctgcaagggcaatggaggacttcggagaccacttaggtgccccaggtggaagggaccatgcagatgaaggaaatggagctcacgatggggaatgggggtttgatttgctcaaagaccttctagataatcttgagattagacttagtttcctggattctaggacagggttcttgtgtcctgcttatttcctttatcgttctttctaatcaaagtttgagatatacaaaaggccacatggaacatatgtgtagttattcagcataatgatgaaaagaaaagctatgggtccttctagaatccaactagaacactgaaattcttacatgcctctgggtactcctcttgtctcccatcccctctgcatttatcctgaagttttgtgttttccattcccctgctttttaaaaatacttttatcttaaatgtatatatccttaaacaatatactgtataatttgtttgctttcctgatcctttctaagaaattatttacatggattttctctctccaaagaagatggtgacaactcacaaagatacctgcttttccccagaactatagctttgctgtaatgtactaacattgaggatttctggaagagagagagccatggcatgacagccttatcagagggtagcctcaagctaagatcttaaaaaataagtactgatttaaattatatttagaaggcaattcacacatccaccttagattttttaaaaattcattcattcattgaaaaacatttcctgattgattagtttgattgtcctcatacactaatgatgcccaggaatctctccacattctgcttgtaatccacaggaatcattatttcaggatgagggaaaggcaattctgaataattgacctgaactttcttaggggccctcaatcttaacgatgggctcaggcactatattttataaaaagaaaataaataagacataatatctcacaaatattagtaggacaattaaatagatcatctgaaatcaggtgtagtggaaagtcaaggatggcagttgccatgctgcactgagccccccttatacttagctcttgctgagttaagagggaaccatgagtgtgagaaagactaacaagtgagtgaaaaactagacagtaataccaggaggaaggccaagaaatgtatcactaatttcttgtttgatttcagagctggaaacaacttctggtggattcctgtagtgggccctttggttggtgctgtcattggaggcctcatctatgttcttgtcattgaaatccaccatccagagcctgactcagtctttaagacagaacaatctgaggacaaaccagagaaatatgaactcagtgtcatcatgtagtggcatgctcagctctggatttgcagtcagtttgggattctcttcagaaagatggcatctaagtgtctgtgttcttgtaagcctgaggtggaatccacccagttttgtctgctagccatatgggacatctaattggaaaagcatctgcataaaagtttggaaacaatgaccacttctctaccattgtcccccacccccaccccccagaataacgctgactgtcccctgaaacagccttctctcctgccctgtttatttcatcctcgatgggaattcttgctaggtaagcactaataactcggcatcttgacgatagtcccatttgggtggtttcagctgcactatctgtatgaaatggtgtcaccaaaacccttttcttcagtatcgacaaagattacattctgagtaccaaccaaaccctaaattgaaagacaaaactatggtttcagtcaacatattcatgaattagggagctaatgggttaagcttccagttcccgctatgctactggatttgtataaatactgatattctccaaacctagtggtgtagggagcaagagaatgcagctggaaggcacaaggggaggacattgtggcattcagaaactgcaggagacaagatgaatttgagaagccaaatggaatttttaatggaaaccatttatcagattaatctcttgctctcctgcattttagaggacaccaattaatttcctggtctttagtatataataacctaaaataccattgtaacctcagtcatgaaaaatacatcactctgtctttttagctcaaatgtattttcctaattgcccacttgagaacagacatttgacaagttatatcaacgactgtgcttgtccattattttacacatgccctagaagccaaaactgaaagccactggatcctggtctagctgaatcttcagagtgggaggtctccaaaaagatattaccttattgggcttaacaattcacaaggcactttcacacccattatctaatttaatcctcataatgactatgtgaggcaaatgccacattgcccatttttcagataaagaaacaaaatcttagggaagataagttgagttgtccaagagcacactgaaagttgaatgttatctaatgcattcctctacctttcagaagatcagtagctggctgacaatctttgccaaatcttccttgctagccagaagtggaattggcagcttctagaatatgtacacctctggacaaaatgttcctcaatcttaagatacaaagaccctcattgtctgggtctattcccacacttactgagtacagatgaaggaaagtggtagcaatttaatcataactttcatttgctgaaaaacattatgagaaggcctcccttcctaagccacctctggtcttgctaagtcttgatcttgcttcctgccagcaccaaacattacattcaggggatttcctctggctcagtcttttccccttgaagttctctaatagatgttacttttgacaaaagatcgcctatgagttacaagcaccaggggatgctctacatcaagggatgcaccttcagtcaaactgtcaaaaagcccagaattcccaaaggcattaggtttcccaactgctttgtgctgatatcagaacagcagaaattaaatgtgaaatgtttctgatgacttatgttctacaatctatggacatacgggatttttttttcttgctttgaagctacctggatatttcctatttgaaataaaattgttcggtcattgttg

SEQ ID NO: 44 - DNA, 4267 bp, Homo sapiens
tgttccttatctttgctggaggcaggcagtagctgtgcagtgacagtgtgcctatgcagacagagggagcagtgaatagcaatagggtgtttccaccatggtcttcactcaggccccggctgaaatcatgggccacctccggatacgcagcctcctggcccggcagtgcctggcagagtttctgggtgtgtttgtactcatggtaggtaggatcactgcgggaaggaaagaagggggttgggcaaaagttcctggctccttctgttgtccttatctctttctcttggtgtcccccttcctctcaactccctatcacttttcgtttttatcctctcgtctctcctaatctcctttcctctcctacttcatcatttttattcttgtcatttttattctttctttaataacaagtgtatcatagttcttgaaacataatttttctctcttcagtttcccatttttatgaaagaccattttcatggtaagaccaatgtgctttattatacttggcctgatgattgtataaagtgcagcaagaataattatttttcacataggcttttaaaattggctttgatggaactcttccatagaaagaatcttagataagactttttaaagctgagccctgccatgggtttgtaccctcaaatgcctatgagttgggtaaattcttttcttttttcccacattctttccctttcttcttcctccactttccccatttcttttcccttccctccttctgcttttctccttcccttgcttgtcttctgtgctcattcatctccttggctgctcccctggccttcccaacctttctccctgttggtctcctgggctccctgttcctcatctctcctcttctcagcagctcctcacccaaggagctgtggcccaggctgtcaccagtggagaaaccaaaggcaacttcttcaccatgtttctggctggctctctggccgttacgatagccatctacgtgggtggtaacgtctcaggtgaggagggtggggtctggtcatcagagcacgtgggatgtgcatgccagtttgtctgtctggtgatggtggaaacgcaaatccttctgtgactcgtggacattatccccttgaacagtgtcccagactgaaataagcctttggccccaccagcccctcctctttctgtatctctccatcctgcttcctcacactaatgcttggctcacctagacagaaatcaatggcagggcacgaagggcagctgctatcctcttgtatcccccacctgaagtcctctgggggctcatctccactcaggacctctcctgtttgtaccccagatacacactgcatcggtgtgcttgggtggagccttgagacatagtgtgttgctgtggaaaaagcagacagacctaggttccaactttggattcacagtgtcttcttcttttttttttttttttttctttttttttgagatggagtttcactctgttgcccaggctggagagcagtggtgtgatgtctgttcactgcaacctctgcccctgggttcacgcgattctcctgcctcagcatcccgaatagctgattttacaggcacctgctaccacgcctggctaatatttgtgtttttagtggaaacggggtttcaccatgttggccaggctggtctggaattcctgacctcaagtgatccactgcctcggcctcccaaagtgctgggattacaggcgtgagccactgtgcctggcccacagtgtgactttgctaagtcacttaacatctgtaacatctggagcctcaatttcctaagctgtgtgatgagaattacactatctgccttaagagctataggaaggagcagtagtgttgtctggtgacaatgcctggcagtgacacttaatagatgctctttttctttccttgatacttgaaggggcccacctgaatccagccttctccctggccatgtgcatcgttggacgcctcccctgggtcaagctccccatttacatcttggtgcagttgctgtctgctttctgtgcttcgggagccacctatgttctctaccatggtgacagagggaacagagggagctgtgccttgagagcacctgtgggtgggcaggggtgcctcagaatggttttggatgaatgagcaaagagggaaatcctgggtgttcccttcctccacagatgccctacagaactatacaggtgggaacctgacagtgactggccccaaggagacagcctccatttttgccacctatcctgccccctatctgtccctgaacaatggcttcctggatcaggtaagtgtgagggggagacctggggacactactttggtcctgttcctcggcaccccagcctattgttcagtctctgggtggagtgtgggttgggtctatcttggcactccccaccatccccaactgcctgtgttgcacaaagccagatgacatggaatatttggatgagagagggcagatggagcacctggcagctgacagggaaccctctgcctctgttgactccaaggttctgggcactgggatgctgattgtggggctcttggccatcctggacagacggaacaagggagtccctgcgggtctggagcctgtggtggtggggatgctgatcctggccctcgggttatccatgggtgccaactgcgggattccactcaaccctgcccgggacctgggcccacgtctcttcacctacgtggctggctggggtcctgaagtcttcaggtgggagacagactctcctggtgctggcctccactcaccttcctctgctaagggctctgtccctgggtccacagcactctgcctttaaaatagctctcttggcttcttaggacagtgttctttctccaagtcatattctccccctttctcccttgcttccctcccaacttttcactgaaacagtaagatttagaggttgtgttcttaggcatgccacattacttccatgcacattagtgacttcatcagcaaaattaatatattcagataattccctaaggcaagaggctggaccaagctctcctggggttccctcttctaaatactatgctttggtgacaaggcaatcctcttcctggtgtctaccaccctgggaacaactttagggcactctcttgttactgccccccgtccttggagaggggaaggctaagggagtcactcctgataccttcccactgtccttcttttgcagtgctggtaatggctggtggtgggtgcctgtggtggcccctctggtgggggccaccgttggcacagccacttaccagctgttggtggctctgcaccaccctgagggcccagagccagctcaggatctggtgtctgctcaacacaaagcctcagagttggaaactcctgcctcagctcagatgctggagtgtaagctatgattaggacaaccctcacttcactcatggaccctggagccagccactgaccccgcctgggaacaacagtcattcttcctctttgttaatgtgccagaacctgggaggcttctctgtttatctgtttggcatcccttcctcctaaactaagaaggatcctggacagggagaagtggaggaggataaggtaccaggactcaggcttctcatcccctcctcccgcaaagcggttttctgaccctcagggcctctcggaatgtagttgctcgaggtaaccgctagagggtgcgcacctggatgctggatggggacggctgcgggcatctgcagggtggagggggccaccatccagtgtagggcacaaccctggggactgccctccatagcctgtcccgactgccgactcctagctctcatcgcctcggcgcctcccaccttcaccctctcggggatgcctccccaagagggtagttaggggtggggaagccgcctccacccagggggcgtggtgggggcggagggaaggagggcggcggggcacagagacagagagcaaggctgtgaaactgaggcaccgttcctagacatctcggtgctgtgtcgttcattcaaggagagttgagatacagtgaaatgagccagggcgaggagggagggtgaaggaacggagggcgggcggctccgaggagcgagagtcgggctgagggcaacctggcgccagggaaaattctggttattcaccacttctacagctctcctgccgctccctgcagaggatgctcgttttgcagagaaggcagtgttcctctattcccttcttccgaattaaaaataccccctcagagcga

SEQ ID NO: 45 - DNA, 20404 bp, Homo sapiens
agagccggagcccgcaacccgctcaggcggcgacggagccatgtcgccgctgctggggctccggtccgagctgcaggacacctgcacctcgctgggactgatgctgtcggtggtgctgctcatggggctggcccgcgtagtcgcccggcagcagctgcacaggccggtggcccacgccttcgtcctggagtttctagccaccttccagctctgctgctgcacccacgagctgcaactgctgagcgaacagcaccccgcgcaccccacctggacgctgacgctcgtctacttcttctcgcttgtgcatggcctgactctggtgggcacgtccagcaacccgtgcggcgtgatgatgcagatgatgctggggggcatgtcccccgagacgggtgcggtgaggctattggctcagctggttagtgccctgtgcagcaggtactgcacaagcgccttgtggagcttgggtctgacccagtatcacgtcagcgagaggagcttcgcttgcaagaatcccatccgagtcgacttgctcaaagcggtcatcacagaggccgtctgctcctttctcttccacagcgctctgctgcacttccaggaagtccgaaccaagcttcgtatccacctgctggctgcactcatcacctttttggtctatgcaggtttgtcattctcaccaaatacttggcacttccagagcctctatgacatgaggctgtaagttctttactaaaatacatttgaaaccgtctatcccgctatgatgtaaatgctggtatccccgttattctaaccattttgcagcccgttcttaaccagtagggccgacacgtagagccttcatgcagtgctgctttgaacacccagagaaaggagccacctctgtgctgaaactgagatactgtgagatattgccaagggttcagatatcctacaaagggattgttttaatggtagaaaggaaagataacttattaactgcctctttcttcatgccaaggtggtatagatcgtttcggatactcttactgaaaaatgatcctggctttacaaagttcctttcgatttaaagcacaaggcacatgattggttagtataagagcggagggtagaaattttgtagttccattttagtcctataaatgatttaaagtcatttcttatttccttaataattcattttgagttatgaaatttcttatttccttaataattcattttgagttatacatgaacttttaagtgacctattaagaaaaatgtttaaatgataacttgcttcaaattaaagatgctttttaaaatataactcttgagaaatctgttgatttcttttaaaccatttgtttactttgacagatctcagatgcagtgggcaatctagtcctaagtgggaggaggaaatccaaaacacaagtatttaatgatgaaatgtgtcattttaaatgattacaacagaatatctttattgggactacatctaaattataaaagactgtggaggcagaaataacaacaaaggaaatttgtggtaataatcttctgcccattaaaagtgaaacaatgactagaagcaaacttcaagataaggactcctaaacacgtacttgaccttctagggtaaaaatgaaagccttcagccgggcgcggtggctcacgcctgtaatcccagcactttgggagaccgaggtgggtggatcacctgaggtcaggagtttgagaccagcctcgccaacatggtgaaaccccgtctctactaaaaatacaaaaaaaatttagccaggcatggtggtgggcgcctgtaatctcagctactcgggaatctgaggcgggagaattgctggaacccgggaggcggaggttgcagtgagctgagatcgcgccattttactccagcccaggcgacaacagtgagactctgtctcaaaaaaaagaaagaaagcaagccttcccaatcaatttccccttctttaggtctagtaccactttttaagtagacataaatggaaataagtggtatagaagagtgaagggggccgggcacggtggctcacacctgtcatcccaacactttgggaggccaaggcaggtggatcgcttgagcccaggcgttcgagactagcctgggcaacataagagaaatcccatctctccaaaaaatacaaaaattagccgggcgtgctggtgcgcacttgtagtcccaactacttgggaggctaaggtgggaggatcacccaagtccaggaggttgagattgtggtgagctgtgatagtgccactgcactccagcctgggttatagagaccctgtctgtctcaaaagaaaaaaaaaaagaaaaagagaaaaagagtgacaagtacaggcatatttgagggattggtgggtagttgcttatttattgaagttttagattttagctaaagcagaaacatcccctgtatatgaacagaattgtcactgcaaaaagagttatgctatggtttagctctttatttctacaattaaaagcagtaaaaacttaaaaaagaatttttacatcaatagaacattaactacagttggatttttcaatacctatgttcctcttccctctcttgattaacctgaagttgatattctttctgtctacttttaatacatatttatgattccatagataatactgtatatattagccattccctattttatcagtgacaaaaaatttgagagaagagacacagataatacttggagttatagatccagtcatagatataaggaaagttattagaagtatctataaaatatcaacagatcaaaaacactgccctttttttcttgaaatgagggtactaattgatgggggagagggagagtataaagggaacactagagcagtgtttctctctgtattaaaatcacctggggagctctttaaacgtccttgttctgggcccacagattaaaatccaattgctctggcaaggggcctaggcagcggtgtattgtaaagcttcacaggtgatcccattatgaaaccaaggtaaagaagcactgctttaggagtctagacctgcactgtgcaattcagtagccactagctgcatgtggctgtttacacttaaattaactgaaattagattcagagtagccacatttcaagtggtcagttgtcaaattgtgctgtagctagtgggtacaggattggacagtgctagagtctgtttgcatgggtagaacaaaatagacaattgttgattactattagttacagaaatttataatgcagtatgatcttccctttgtttgttttctgccaatataagggagataagaatattcagttgtcagctgttataggccaagcattttgcaaggaaatttatgagagttatcttaattaattcttacaacaacccatcggccgggcgcggtggctcacgcctgtaatcccagcactttgggaggccgaggcgggcagatcacgaggtcaggtgatcgagactatcctggctaacacggtgaaaccccgtctctactaaaaaatacaaaaaaattagccaggcatggtggcgggtgcctgtagtcccagctactggggaggctgaggcaggagaatggcatgaacccgggaggcagagcttgcagtgagccgagatcgagtcattgcactccagcctgggagacagagcgagagagcgagactccgtctcaaaaaaaaacaaaaacaaaaaaaaacaacccatcgatgaatatattcttcccattttaagtaaagtaagtgaggcccagaaaagtgaaatgacttatctaagcttacataactagactgtgttagaactatggttccaactggcactaaagctcatacggattcattatcagcatttccacagtgtgctcctcaaaagttaacaaaactatacacaattgaatatattcagccataaaaaggaatgaagttatgatgtatactacaacatgaatgaacctcaaaaacaatatgccaagcaaaataagccagacacaaaaagacaaatactgtatggctctacatatacaaaatatctagattaggcaacttcatagagacagaaagtagaatagagttaccagggactgtggggaggagggaggaatggggacttattgtttaatgggtacaaagtttctgtttatgttatgaaaacctttggaaatggtgctgctggtgaaacagcatggtgaatgtaacaccaatgacttatacacttaaaaatggctaaaatggcatactttatggtatatatatatttcaccataatttatacaatttttaagttacaaaatgatgttgttgccaaataaatttggcagacactggattaaacaggttttctaattttaagacttcatagagcctttactatgcttctaagtattgtgaaactccaagaggaggatttggagcacagtttttcaaacttacgagatcctcttgtctaggagtgtgtagcagaagagaacatactttgggaaatgctgcactaagtcccatctctcccttttattttctatggctataagtcataactttggaacagagttgttacaaataggactgtcttgggacagttggctaagtcaggcatcattacaagttattatgatatgtcttttttaagcttctaggatactacaagtacagatttgacaaaggtgcaccttgggagatttctttaatcacttgcattttgagggcactgagaagtcaccaatacagggtacatcaaaataacttatgtattttaggattacagaacataagcatccagcaacatagccacaggctcctaaggatagccagtgaccaagtgtctgtgtatttattgatttagcaccccaaattttgatggcacatggagccccagttatgtcattgccatttatcgtgtattaactggctcccctaatcgtgtaaaaaaaaaaaaagctgtgcgatttattaacatattagagctataaactgttaatcagatttcaagcaaatcttccttactttgggttctggaagaatttagttcaatgaacagtttgagagagagctgcattcattttttatgcataattagatattgaaaaacacctgggccgggtgtggtggctcacgcctgtaatctcagcactttgggaggccgaagtgggtggatcacctgaggtcaggagtttgagaccagcctgaccaacatggagaaaccccacctctactaaaaatacaaaattagctgggcatgcttgcgcatgcctgtaatcccagctactcaggaggcagagacaggagaatcgcttgaacctgggaggcagaggttgcagtgagctgaggtcgtgccattgcactccagcctgggcaacaagagcaaaactccatctcaaaaaaagaaaaaaagaaagaaaaacatctgttattttacacgtcagttgtctttattaccacatactgctcatttctttcatagaattttaaattatattgttttctgacacaatgcctgttttcctctaccagatgacaagctttgagtctgtagaccacatttgttttatttgtcataacatatatcaagatcttagcataggacctggtacaaagtaaatgtatttttagaataagtgaacgaactagcaaatagtctgtttactctaaaaaactgtacaggaagaaataatatggccgggcaggatggctcacgcctgtaatcccagcactttgggaggcagaggcaggcggatcacctgaggtcgggagttcgagaccagcctgaccaacatggagaaaccctgtctctactaaaaatacaaaattagctgggtatggtggtgggcacctgtaatcccagctacttgggaggctgaggcaggagaatcgcttgaacccgggaggtggaggtgggaggatcccctgagccctggaggcggaggttgctgtgagccaagatcatgccactgcacttcagcctgagcaacagagcaagactcagtgtcaaaaaaaaaagagagaagggaacatatgttctttttaaataggagctattatcaatttacaaatgaaaaacaccttcaaaggtccaaaatttattttgttatgccagtataatatagtggttaaggccaggtgtagtggttcatgcctataatcccagcactttgggagacggaggcgggcggatcacctgagttcaggagttcaagaccagcctgttgattcataatctgaatcaacatgaccacccacagcctattcattatcgtcactactctactattctttaaccaattcaacgccaacatggtaaagccccatctctcctaaaaatacaaaaatgagcctggcatggtggcgggcgcctgtaatcccagctgcttgggaggctgaggcagtagaattgcttgaacctgggaggcggaggttgcagcgagctgatatcacaccagtgcactccagcctgggcaacagagcgagactatgtctcaattaaaaaaaaaaaatatatatatatatgtaaatatatatgtgtaaatatatgtgtaaatatatatgtgtaaatatatatgtaaatatatatgtaaatatatatatatatatatatatatatgtatgtaatggttaaggccaggtatggtggctcatgtctgtaatcccaggaccgggacgtcaagacaggaggactgcatgaggccagcagtttgagaccagtctgggcaacatagcaagaccctgtctctacaaaaaaattttaaaattagccaggtatggtggtgcacgcctgtagtcttagctacttaagaggctgagacagtaagactgcttgagccctgaagtttgaggctgtattgagccatgatttcatcactgcattccagcctgggtgaaagagtgagatcttgtttcaatttaaaaaaaaaaagactgtgcccttaggctgaatacttattttgggcctcactttcctcatctctaaaatgaagaaatttcataagactagtatgaggaataataattcaagtttgtagcatagtacctgccatacacgaagtacttgggaaataattattatttattattattatttccctctatatttctaacaggcttccagtataatttgatcatttttgcatataatttgatcagttttattgctcacaactccttttgggataaattcaggaaaatgtaagttgtaacttttataacgaaatttgtccattgaaaataagtaaagattttggtacaaatccctaggtgttccccacaacatctgagaacagtctaatgtcattcttccaggtttgtaagaataaccactaatttgaaaacaggccaggcgcagtggctcatgtctgtaatcccagcactttgggaggccaaggtgggcggatcgcttgaggccaggagttcgagaccagcctggccaaaatggcgaaacccagtctctacttaaaatacaaaaattagctgggcatggtggctcgcgcctgtagtctcagctactcaggaagctgaggcatgagaatcgcttgaacccaggaggtggaggttgcagtgagctgagattgcaccactgcactccagcctggatgacagagtgagaatctctcaagaaaagaaaacagctagctagctagcttagttagtaacattagcatatcagcagaatacaaaccagaaatgaaaagtggagtttggagatgcaaactttgaaattatttgcccagtaatgtccgggagtagagattgaatttggggaaaacaagcaggcttgtcattagagaggtattttctttttttcttttttcttttgagatggcgtctcgctctgtcacccaggctggagtgcagtggcgcgatctcggctcactgcaacctccgacccccaggttcaagcgattctcctgcctcagcctcccgagtagctgggatcataggcgcgctgtaatttttgtacttttagtagagatggggtttcaccatcttggccaggctggtcttgaactcctgaccttgtgatccacccaccttggcctcccaaagtgctgggattacaggcgtgagccaccacgtctggcctagagaggtattttcaactcctgacaatgatattttatcagagctcatcagggtatgataacactccttttgctttgcttacgtgctcattaaaatgggttctctggagagggaagacaaatattccagttactagaaagctagcttccactctaaatgaataaatagaccttccagcttctacatgggtgttatgtgatatttaactagtattcttctgtcacagagaattttattccctttaggaatattgagaaactttagtgcctaaaattgccagatttgagacttagcctctggctgtggtaatttcaccaaatccatgctgtcctctgactcttggcagtggactagctggatctcccggttatacctcttcttcaacatgcatgattgtcatctccccttcaaatgtcaataaatagtaacttgttttttgaagccatcttttgatagttccatgcctgatgattctttaaaaaataattcatttttaattacacaatacgtgatatgttctctatgtaaaaaattcaaagactaaagtgccttttgactgccatcctccatgcctccatgcctgtttcctttccttccctgtctactcttccttccacggtaatccttgtctctgatgtttgtgttacaaccttccaaactggttgtttttgttgttgttttttgggtttttcttgagatggagtcttgctctgtcgcccaggctggagcacagtggtgcaatcttggctcactgcaccctctgtctcgcaggttcaaatgattatcctgcctcagcctcctgagtagctgggattaaaggtgtctgccaccacacctggctaattattttttttttgtttgagatggagtttcactcttgttgcccagactggagtgcaatggcacgatctaggctcactgcaacctctgcctcccaggttcaagcgattctcctgcctcagcctcccgagtagctgggattacaggcatgcagcaccacgcctggttaattttgtatttttagtagagacagggtttctccatgttggtcaggctggtcttgaactcccagcctcaggtgatgtgcccgcctcagcctcccaaagtgctgggattacaggcgtgagccaccatgcctggccacacctggctaattttttttttctttttgcccccggagtagctgggattataggcacctgccaccacagccggctaatttttctttattaattatttttttaattatactttaagatctagggtacatgtgcacaacgtgcaggtttgttacataggtatacatgtgccatgttggtttgctgcacccattaactcatgatttacattaggtatttctcctaatactatccctcccccagccccccaccccatgacaggccccacacctggctactttttgtatttttagtaggggtttcaccatgttgcccaggctggtcttgaactgctgacctcaaatgatccaccagcctcggcctcccaaagtgctgggattataggcgtgaggcaccatgcccagccccaaactgtgtttttatttttatttttttgagacagggcttcactctgtcacccaggctggagcgcagtggtgcaatcaaaacctccgcctccccggctcacaagatcctcccacctcagcctcccgagtagctgggaccacaagtacatgccaccatacccaggcaatttttgtattttttgtagagatgggggttttgctatgttgcccaggctagtcttgaactcctggactcaaccaatccgcccatctcagcctcccaaagtgctgggattacaggaatgagccatcaaacccaggcccaaactcttttcatgtgcatttactatacatatagataaaaccctacaggaatgatatattattactgttatttacttatttatttattttgagactgagacttgctctgttgcccaggctggagtacagtggcgtgatcttggctcactgcaacctctgcctcctgggtacaagcagttctcctgcctgagccttccgagtagctgggactacaggtgcacgccaccatgcccagctattttttttttttttttgtattttagtagaggtggggttttaccatgttgcccaggctggttgcgaactcctgagctcaggcgatacacccacctcgccctcctaaagtgctgggattacaggtgtgagccactgcgcccagccaggaatgacatattattatatatattattgtatatgcgtatacttgtgttaacattaaaaagtatcataccttactcagcaggctttattcctgatagccaagagccggaaacaactatctggatgtccttcaaggggcaagtagttaaacaaatagttggggtagttgtggaatgcagagaagtgtttggagatatatattggaggaagaaagggcaagacacagtcacaggtcatttatgacatggaagatacaggatgaggaaacaatttgctttttttggtggtagttgttcaacgatacatcttgtatatctacctatgttagtaaagagatttctctcattatttcaaaacactgcatgatattccaaagtaatagtaaatttgtttagggctgttttcctatgaatggacatttaggtttttcctagttctttgctacaataaccaatcttgtagggccgggtgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtggtggcgggcgcctgtagtcccagctactcgggaggctgaggcaggagaatggtgtgaacccgggaggtggagcttgcagtgagccgagatcatgccactgcactccagcctgggcgacagagcgagactccgtctcaaaaaaaaaaaaaaaaaatcttgtatatgtcttctcatacacaaataccagtgtttctctaggacagtaattctgaacttagggacagttttccccccaggggacatttttggttgttacgatgtggggagagggaataggccactgccatctgagtagaggccagggatgctgctaagcatgctgcggtgcacaggacagctcacaacaaagaattatccagtaagacattaaagccaatgccatgtttttagtttttgttaacacagaacccacacctgacatggatttctgtatcagttaactattgctgtgtaacacaccattcaaaacttttttttttttttttttttttgagacaggatctcactctgtcacccaggctggagagcggtggcacaatctcgactcactgcagcctcaacctctgcaggctcagtttattctcctgccttagcctcccaagtagctgggaccacaggcatgtggcaccacaaccagctaatttttgtatttcttatagaaacggggttttgctatgttgcctgagctcattacaaactcctgggctcaagtgatccacccgcttcagccttccaaggtgttaggattacaggcatgagccaccatgcctggccccaaaactgagaataacttatttttcatgattctgtgggttatagatgggcagttcctctgagatctctcatatagctgcattcagctgaccagtggtgaggctggaaagcccaaggtagcctcactcatatgtctggcagttgtcagctgtcatctaagatgcctcagttctcttccgtgtggcctttcatcctccagcaacctagaccagcaaacttacatggtcagggcattcttccaagagagcaaaaagtggaagctgatgggtttattgagatgtgaacctagaattcacacagcatcactcctgttacattcagaattcccatagccctactctgctgcattctatcaacacaagttgcaaagccagctcatttttaagagagggaaagtagacaacgtcttgctgggagaagcagctaccactaagtcacactgatcagaactgctgtgtgaggatgagaagattaggaagtgaagtcgtttgagttttaggatggcataagactgatcagtggtgttcatcttagatctagtggaaaatcttcagtataccatggccttgaatactgaagtaaactaagtgagggtaatgattcagtccatttttatcaatacatgcctaaaagtcacaggtcatttatatcatcataggtcatattacattatattgtaaatgtctgtgtgagtctttatctttctgcccatggtatctagtacacaggaactcaataagtttttgtgtaatcaaactgaaatgactagctgccaaccagacaattctgttcacccttagcagtggcaggtttaacattccaggatttgactgttcaagaatgattccaaaggtctatgatatcatttataattgctgagtcatgaatgtgaatcatgctgtgaggctggtacactagaaaaaactcttagctattgagtgagcttagttcattgctaaatatcttcatctcaacacagcttcattgttctcggcctattcaagtgtgaaatctcagtagtgaaatcttatgaaagggccacttaatttccatgaaaacaggctacaatggtgcatgtcaataataaaacttctgaagatctcaggatctatctctgtggctaagtaaaagaggcagagttgtgtgcctaagcttccacttggaaaaactgctgaaaatgtcactgtctgagggatgctcttaggctggactttgaaaagcgaagacacagctctgtggagtactgctggacaggcatccagcctcctcttccattccgcacttgtgtggccagactatagactcctagccccatgccagagaaataccctgggtgcagagctggaacacaaatcctaaaaagtagccaacagttataatttgtgcttatctctcatgactactcacagtcccttcttaagggctctgttgacaagtaaaaatagaaccaatgataaagctttggtctattggtccattttaccttttcaatacaaagttatgcttgtactcaagaggatcttgggtcattgtatctatttaactgtattttcattttacttttattattattcattcatttaaattgacaaatgttcagggcagccaaatctattttataaaacggtaggaataataatagagcacattaactttcagtgtccattttatagtctataggaaaagcactgggttcaaactttaatgtcctctgtctttttttttatagtaagtggcatattatgatttatgattatagtctaaaatatgccaggtgattctgcaaaggaagatcacagactaagaatacctaaaataaaagtcacatttagtaaccatttatttccagttacaaggaaatggaagataaatcatttgaaatcactctgcttaaaatctgttacaggaggaagtctaacaggagctgtatttaatccagctttggcactttcgctacatttcatgtgttttgatgaagcattccctcagttttttatagtatactggctggctccttctttaggtaagcgtatttttatttaatatgtctgaaagattagggtattttaaaatatgatatgtgtatatcattgtaacatgtcaatttccaaagccacagcaggcacaaaacagaaaatgaaaaaggtagctaacatgcctgaaataatactttgtgtagtatcctttaaaactgttaaaggtagtcagtttcagatatgctatcactttaagaaaccttagatctgagcaccaaaaaagcaacgagtgcttacttcccttcaaaaataatcttttttttttttgctaaagaaatttgttttttcacatttgtgagctccagataaagtatcatttagttcatttgactcattttgcagaacagcatctcatgaaacaatgaatacctgtattaagtatagtttaaaaattatccacattacattcctgttatttttattgtttttgttttgttttttttcgagacagggtctctctttgtcacccaggcttgagggcagcggcatgatcatagccatagctcactgtgcttgaactcctgggcacaagcaatcctcctgccttagcctcctgagtagctgggatgacaggcatgtgccaccacacccggctaatttttgtatattttgtagagatgaggtctcgctatgttgctcaggctacattccagttacttctatttagatcagatatataaactctttataaagatgggtcaaataacacacaaaattatacaatatacaaaaaattattatgcatttcttttgcattacgtatataaagttatcagcatttattgattttgtttagtcatctgttttaagcctgattataactcataactgtaagtgaatatgaagatgcttggccctccattaattcactcttctttttatccagttagcatattgttccttgttagtcattttctttaaaacacacacacacatacacagaaggtaacacagaagggaagtgccataacaggtttgaatagtcaatcacactttactcttcctgtttaccttccattcctctatctcacattaatgttattgttatgctcaatatcatattgttatgctcaatattgttatgctcaataatatctataggcctaaacatgaagaatatcagagaaaaactggttttaaatgctagtaatataacttttggtcagttctacagagtgctagtattttccgataacttctttaaagaaaaattttctctgtttttaggccgggcgcagtggctcacgcctgtaatcccagtactttgggaggccgaggcaggcagatcatgaggtcaggggattgagaccatcctggctaacatggtgaaaccccgtctctactaaaaatacaaaaaattagctggtcgtggtggcgggcacctgtagtcccagctacttgggaggctgaggcaagagaatggtgggaacctaggaggcggagcttgcagtgagctgagatcgcaccactgcactccagcttgggcgacagagcgagactccatctcaaaaaatccgtttttaaattttaaagttaaaaaggtcactaatgtaaagaagttaagaagttaacaaacaaggtcactagttgggcaggcctacaggtagtttttatatgaaaaaattcccccagtatgtctggagactaacatatattaacatgatttatttgttagcgtgctccaactactattgtaacagtaggctaaggcaaaggtccccaaaccccaggctgcagaccagtatagtccgtggcctgttagtaaccaggccgcacagcaggaggtgagtggcaatcaagcttacaaagctccgtctcctgtcagatcagcggtggcattagattctcataggaacgtgaaccttactgtgaactcagcatgtgagcaatctaggttacacactccttatgagaatctaactaatgcctgatgatccaaggtggaacagttccatcccaaaaccatgcccccagtccatggaaaaactgtcttccaggaaaccggtccctggtgctaaaaaggttgaggatctctgtgttaaggaattatttcatccattacttgaaagaacattaaaaagtagccaggtaggctgggtgcggtggctcatgcctgtaatcccagcactttgggaggccgaggtgcgtggatcacctcaagtcaggagtttgagaccagcctggccaactctactaaaaatacaaaaattagctgggtgtgatgatgggtgcctgtaatcccagctactcaggaggctgaggcaggagattcacctgaactcaggaggtggaggttgcagtgagccgagatcgcactactgcactctagcctgggcgacagagcgagtctcaaaaaaaaaaaaaaaaaaaaaaaaaaagcacagtggcacatgcctgtagtcccagctacttgggaggctgagatagaactgcttgaggccaggagttcaaggccaccttgggcaacatagcaaaaccacatctaaaaaataaatttttttaaaaagggtctttatggaagaattttaaaaaggaatctgaagtaatgacaatctctgtaaaaggaccttgattttttcttagaacacattttccagccaaagcagcacttaatgacttcctggaagaacatactagaagaaaagcctcacgatagataaaaaaggaatgctaaatcttctccctttacagagcagaaagtgtgataggttggggaaagtgactatattctcttcatgttagtgtggtacaaacaggaagataacacattatatgtagccaaaacattttaaagaaccacttaccccctaataatccgaacattccccctaaccccgccaaaaaaaagccgaaatctcttacaaatagaagtggaatcaaactttttttggtctggctgtgtcacccaggctggagtgcagtggtgcaatcttggctgcaacctctgcctcgcaggcgcaagcgatcctcctgtcccagcctcccaggtagctgggactactggcgtatgcccccacacccagctaagttttgtattttttatagaaatggggtttctataaaaccacacccagctaagttttgtattttttatagaaacggggtttctatgttgctcaggctggtctcgaactcctgggctcaggtgatctgcccgccttggcctgacaaagtgctgagattataggtgtgagccaccatacttggctgagatcaaacattttcgaattcatatgatactttttatatttagcttgttgaaaatatgtattccaagattctcagttggtctaaggacccctaccttttaaaatagacccaacataaaatatttactgtagttttggtttgaagaggcatgtttctatatttctttctgaggtttatgaatgagaataaaggcatccttcagtatatgtagataaaagacatgtatgtgaataaatgatgatttttagaatactgcattccagccagccattgaattttgtcatatattgaagttaccttcccactggaaaaaaaattggctccaaatgaaataaagacataaagcgacacattatataactctgaaaagaatgggaaaaaataaattataaattaaaagaatcattcaaagagttaagagatgaggagtccaaaataaaaataaaaacaaaaaccccaaaacaaacaccactcaactaccaaagctacattaattaggcagtgccagggtgccaggggagtgagacagaggaaagagtaatcccaaggtatccaccttctttccttcttgaccaagcaggaagcaaactattctttggagtcatagtccatattctttcttgtttctcatcacagcagagataattataattcctctaatggcccctgccatcttaccaaattaaaaatttagaaatgtgactataaggatgtatatgtggcctggtgtagtagtacatgcatgtagtctcagctactcggcaggctgaggtaggagaatcacttgagcccaggaagtcgaggctgcagtgagccgagatcgtaccactgcactccagcttgagtgatgggagtgtgaccctgtctcaaaaaaaaaaaaaaaaagtatatagaattctctagtttatacatttcatacaatgaaaagtagaaaattctatttgcttctatgaaactgcaattctatttttggaaaaagttttaaatgtcttattttctagttctgactttataaaaaatgacaaaaaaatctgtgggtcagttaacatagaggttgtttctgatttttttttttttcatttaaagcctttggttgccaacagactaatctctcacaaatatgttaaggtatctaagtggttgccaaaccttaaggttttttgttgttgcaaacaagagaacataatttcaaatgtgcatgtgacaaaacttgttttcaatctgaatttaaactgatctagggctttaagcctgaatacattcagtttaagatgaaaaattaaccactgcagatttacttttcaggatagtgacttgagcccttggctcactctccttagtccctatttcacattgaaaaattaaccaaaagctacatgccagatttctctgttgtaattctcagaaatgaagattaccaatatgtgaattttcaaaacattttgggccgggtgcagcaacttacgcctgtaatcctagcactttgggaggccaaggggggtggatcatgagttcgagaccagactggccaacatggtgaaaccccatctctactaaaaataaaaaaattagccgagcatggtggcgggcgcctgtaatcccatctactcaggaggctgaggcaggagaatcgcttgaacctggcagggagaggttgcagtgagccaagatcacaccacttcactccagcctgggcgaaagagcaaaactctgtctcaaaaacaaacaaataaacaacaattttgtattatatttcagatttaaacttccaccatcgactgaaattttgtttagatattttatttgcttgacacatctctaaaaccagttaatggtcagttaaataaggttgagataaaaatgaaaaaactgttaatagttgagattaatgaaaaaagagagactggagaaagtaacctaacattgtttttatttgaacattctaagtgaaaactccgagtgctttcagttgctttattctctaggtatgtccctggggcacggaggaaaaggagagtgttaccattttaaagaggagaaaatgaagacccaagtctagaactaaagagccagaactaaaactacatctttggatttagtttatcctttgaattcattgtttagatcctattggttttaccaaatgcatactgtgtttaaggattcacagttgaagaaaaaataatctgagaataatagatttaaatctgatattagatttaaatctgagaatattagatttaaatatctaacatgtttcacgtagaattgatgtagccaattctttaagcatggctttccaaacatctaggtatataattgtaattttaaaatatacccacctagtgattatgaacctccttaaagattgtttttttattttactgtattttgtctttcaggtatattgttgatgattttgatgttcagctttttccttccatggctgcataacaaccatacaattaataaaaaggaataactgttccaaagactcagactaacatacaggacagtccagctggatgtgataaagattttatcacctcatatggaaaacaccggctgcactggattcatcagtgttaacttcctttgaggaagctgccttatagttttcatcactgggactttaaaaaaaaattactgtgaaaatgaggtattctgtacttctcagttaagacttgttctttgagtgatgtattaaatgctgctagaaaagcctcattacattaaatataaatcaatcttaaatgataattgttaactttgatgaaaaacgagtacaggatgagaagggaagtaaaggtgatagtaagatcaacgaatttgtgtatcaagtgtcacccaaatgaacagaatttttaactataaaagtacaaggcgtcaacttacagaactggggagaggatactttcagccaccacctcacaaaatgataccatcaagcagtgtcatctttcaacaggaaagttgcttttagaagtagaatatatattcactcacatgcaagcgatttcctacattttaagtacattaaaatctaatacctaaaatagtttctctaaatagacaatctcactagctccagccaagtggttattctttaatagaatataataaaacaaaacacccatacaaagtcaggatttaagcttttcacattctcctttgaaccaacagctagctaccttatgaagtagggagggcatgtatttttaatctccattttacagataaggaaactaagcctagatgggtttgctgccctggcttagatcacatagagccaaataccatctctagtcttcaacttcaatccaaaaattcttctcggtcacgattatgactaaagggaaatcaagggttcatagaaacattaaatactttataataacgttaaaacttgtttagaaattaactagtattttttgaggtttaaagaaccttccagttttact

SEQ ID NO: 46 - DNA, 6618 bp, Homo sapiens
gtcccctcaggtgtcctgcaggcacagctcctcggggggcccaggccgatggcaggtcttaacgtgtccctctccttcttctttgccaccttcgccctctgtgaggcggccaggcgggcctccaaggccctgctcccagtgggcgcctatgaagtcttcgcccgggaggcggtgggtgcgggtgcagctcgggccctgctgcctggagatgaggacgctggtcgagctcgggccctgggctggggactttgggcctgacctgctgctcaccctgctcttcctgctcttcctggcgcacggggtcaccttggacggggcctcggccaaccccactgtgtccctgcaggagttcctcatggccgagcagtctctgcctggcacgctgttgaagctggcggcacaggggctgggcatgcaggccgcctgcaccctgatgcgcctctgctgggcctgggagctcagtgacctgcacctgctgcagagcctcatggcccagagctgcagctcggccctgcgcacatccgtgccccacggggcgcttgtggaggccgcctgcgccttttgtttccatctgaccctcctgcacctgcggcacagtcctcccgcctacagcgggcccgctgtggctctgttggtcaccgtcacggcctacacgggtgagcactgcttccccttcccctgggcccctgaggacctctcacctgtgtggcctccatgcacactcaggcccgtttgcacccaggatagggggctgctggcccgggtggccaacaaacccacaagaagaggcggtgatttcctgggatctggcgggtagacgagggcctgcgctggacatggggtgcggtggagcagcctgtcccctgccagggccatcctgcccactgcacctctccccagtcgcagctattcttggtcctcccagaagttctgcctctggggtagtagggacagccgaggcagacgattcaaggcccagcccagtttcctggtctggtgtgggagtccctgcaccctctatcaccccttccagagcgacaggtcctgtctcacctgccccagttggcctccctggcctcctactgacacctgcatcctgcgatctctgcctgcacctggaccgaccgtggcacccagctctcatggtcccatcacccccttgtgagctccctccactcagaccctccgtctgatccagtcccccatgtctgctttcccctccgagcgctaggctccaccaccacgaaagcctttgctcagcaagttcccaggacaggtgtccacagcgctggggctgcagggagagccaggggagccggctcgtgctccaggcaccacaggaagggctgcagggagaatggtgccaggtctccttccgggggtccgaggtcctaatcaggacaggaccagcacttaggtccatggcactgcccacgacccagcactgggcagacaccacggaatctggaccacaaaggccacaggaaggtgccaccagagccctgtacagatggggaaactgaggcacgggtgagcgggagctctgcagggacacggctctgtggcagattgcccgggcctctgtcctgggtggcaccggagagctcccgtcaagggtcctgcctccacgaggcacagggcgcctggtctcagggacggagctgcccatcctcagggtcctggtttacacctgggcggccgcagactccatgtccctgtggtccctgccctccgctaggcccactccctgctcttccgtgcccagaggcaggtccccggtattggtggattctgttatttggggtggtcacccaggcctggggctgcaggacagcggcccggctgccgccgccttcccttgtctcacatcgcacagccatgtcgtcttctctcctctgctgctgctcgtttatttctgagatctctctctgtctctcctctctctctctgtctctcctctctctctctgtctctcctctctctctctgtctctcctctctctctctgtctctcctctctctctgtctccctgtctctctctgtctctcctctctctctctgtctctcctctctctctctgtctctcctctctctctctgtctctcctctctctctctgtctctcctctctctctctgtctctcctctctctctgtctccctgtctctctctgtgtctttttgtctctctctcactatctgtctctcttagtgtctctctccatctctctctgtccgtctttctcgaaggccacatcccacagggtggggtgctggagggcagcatgggcggggcacgggatcccggtgagatgtcgcgagggtgttggacggccctggggggcccccaagctgctgcccctgacccagaccttggctgacccggccttgctgcatcgtgtgtccatcggtgcgggggaccaggcggggcttcaggagccgagagcagctcccaggcctgcacacagtggggctctcggtgtggattcggtggcggggatcaggtcctggcctgcagaggggcgtcaggcagggctggggaccagggctgtctgtgctctgcccacagccgggcccttcacgtctgccttcttcaaccctgccctggccgcctctgtgacctttgcctgctcgggacacaccttactggagtacgtgcaggtgtactggctgggccctctgacaggtaagggcaggggcagggtggggtccctagggctgctggggcccacagagcatcctggcacgtggggtcctgcagggtggaggtgagggtgcctggtcagtgctcctggggaggtctctgcagtgggcatggtttcgggggtaggggctgtgacggcatgtctcactgtggacaccgtcccggtgccgagcaagctgcagccaggtaaggaggccacaggctggggaaaggccaaccttgcaaggggtgctgggcacacgggggatggtgtctgcagtggggctccatgcacctggggttcccgtcccaggtggcatctccacacacccagagccagactccgccgaggggggctcattctggtgtccctaaagcctcaggaggcagagagccccgcttagatgatatagggtgccatcgtgcacttatagggctgtgagcatggggctggagcagccaggccctttgcatcctggtggggcagacccgggactcgctgatgcacacaggggaccccagagttcccccacagtgtgtgcccctcctttcttatatcctggatccaagcagaggagcagcatggggaggtcatggagccccctcttggacctagatctggggattctggcccctggcttcagaggcccaggtggagcccacaccttctcctgcacctgctctcagacactcggctccacctcactgaggtaccagaagggcagctgggttcctatggaattctggaagcttttttcagcttctgctggctctacactggggaggtctcaggggcaatgcctcagcctccccttggggggaaagacaggagctgggggtctctcaggcacctggccctgcgatggtgacactgacgtcatcgtgatttgtcgccatggttctgatgtggtcacagtctctcaggcatgtgcagggtcctaggaagcatatgaagtgcatcaggaaccccctcctgcccccgttgccgaggcagcactgccctctgctaggaacagctccgtgttggcctctctgtccccacacactgggcctgcagggcttctccgagactcttcagttcaggtatcaaccctgggctgtctcctgggatgtggggggcggatgttctttccttgcctccccacgctcctcctgcggatccttcactccgggtgggtcggcctcttcctcctgatcagctccagagccccctctagttccctggcatggaaacacggcccgggtgagctgtggtggccccggaggcctctccgctcctgcacaggccttgcttcctgcgggtgacgaggtcctggactctctcctgcccaggcttctgggtgctttccttagttcagcaccagtgctctgtgtgggcagcgtctcccccgaggatccgcagctccgggttacccgcaggcgtccatctccggtatggtgctgcccttcactgatcctggttgtatttctgtttcctgctttcctcatcgcctcctgtttcggttgattccttctttttgctggtgcccgtctcacagtagcttcctgagaacggggacctggcaggtacacttcagacctcctgtgtctgaaatagtgtcctggttctgacctgcacttgagtgtcggtgaggcctgggcagggttccgggtgggagctcagtttcgtcctgagtttctcaggccccaaccatggcctgtggtggcttcacgggctacaaggcaaaggacgcaaacgaagaggcttcacgtgacagggttgtatgctcagccagctctggaggctggagtctgagctggcagcactgacagggtcagctctcctcggaggctgctggggaggagcctcctgcctcttccgggctccgggggcctctggcacccccggtgtccccgggcttggagacgcagcactcccatgtctgccggttcccctggccgcctcctctgtgtcattgtctgttctcttcatatagggacaccagtcattgaattggaggttcactctactcaagtatgacgtcaccgtgatttcactgattttatgtcccaggccgtattctaacaagggcacatcctgtgttctgggaagggcgtgtcgctggggaaatactcttcacccggctgcaacctctcactgtagaactgcctctgtggagaagcccaaagggcatttgcggcttctaggagccaagtaggaggaggctgggatccgtgtttcaggcgggactccaggcttgggcgggcctgatactcgagtccacatgccccctctagagaggaacctgtctcctgccagggccagggaggggggcactggctgcttctgtattttggggtttggggccctggagcttcccatgcggaattgccgtccctcctcctaggcgagtcccagggccaccccatcccacagggacccgggcgccagcttctgaaagcatggggcatctgcggaagaactgggttgtttcccagctttcgtccctgcggaggggcgatccggcccctccatgtcagcagtgtttggtcgtccacatgcttgtcagccccacgctgtgctcctgcgtctcttcccgtctcatccatctggatgcttgacacctctgacagcatccctttcctgtcatcttagggcagcttcaggaaaccgaaaaacaggcttgtgtccttccattaacccctttatccacaagttcagtatcagcatgagccctggggagctccaaggctgcagccaggagccccgtagccaggtgtggacatggggctatctccctgggtgtgagcggccaccggcacctgctggcagcacccctgagcaggtcatcccgcctccctgggcctgccctcctgggaacagagctaggaccagtgtgggcctggagggccgagtgaggggcaactgtaggcaagctgtaggcacaggggccactgtcaggagccacagcagctgcaggtggtgacaggcagggccccctcaggccagccctagtggagctcacacactgtgggttaaatccttagaggagtgagatcaaacttgaagaggatctactctccccttctgcccggaacacctgcccttcccaatgacctggaaggcaggcggggatttaggagtctctgctcctggagttctgggagaacgttcgcatgaggatttagccaggcccagcctcccacccaggggcatccacatctttcttgtccactcacatcccccacctctacgccaggccacagccctgcaagcagccaccctgggacgccacatcaggtcccggaggaaaatggccacagaagctctgggagtggctcaggacaatgtggggggaggggaattccagtcactcatcccagagggtggtccagaagtacaaggaaccccaccggcttgcccccgtccccatggctggcatacccagagagaggggcatgaccagggcgggaagaccaggaccaggcaggagctgggcctcagggttcttggggctcctggccaggctgcctggctcgcctgtcctggcacaggcctgggagtgggtgtcctgtctgtgtttgggcccaggagccccgcggcccacctgggacctgccttctcttgcagggatggtcctggctgtgctgctgcaccagggccgccttccccaccttttccagaggaacctgttctacggccagaagaacaagtaccgagcaccccgagggaagccggccccggcctcaggggacacccagacccctgcaaaggggtccagtgtccgggagcctgggcgcagtggtgttgaggggccacattccagctgagtggccttgctctgtgtgagccccgtgcgagggccctgcttgtagctggaccctggaaccttctgtagctaagagggaatcctggccccctccccagaagccatttgtcaataaaccatttctaaga

SEQ ID NO: 47 - DNA, 9326 bp, Homo sapiens
tccaggtgcccggcctgggctctgccagaggctgaggtgcccagggtgtcaggacggccaccctgaaggccctacccctttataggaaataaagtcttctttccaaatttatagatgtgggatttgttcagtggacatagctcacccggggcattttaggaatttacccaaaagacataaaagtctgcgtttacagaaagaatagaccaaaatgctaacagcagctttattcatagtagtaagaactaaaatccacccacatgggcatcatcagaaaaacggccaagctgtggctcttcatgacatggcttcaagtcagcaatgaaaaggaacaaaactctcaaccatgttcaagggatatgaaccctcagggacagggccctctctcaaaacaggcaggtccaacccacgtgtagctcgtggtcccattcctctgcaggctccggacagaaagcgggaagctagaatcacataacccacaagagtggaccctcccgctggggaagggcggggaggaccgggaacccacgagagtggaccctcctgctggggaagttcagggaggaccaggaacccacaagagtggaccctccctctggggaagttcagggaggactgggaacccacaagagtggaccctcccgctgcggaagggcagggaggaccagaggactcggggatgatgaagacgttccagctcctgacgctggcgagactcacgctgctgacacctgtcacagtgcacgggctcacacttaacttttgtgtgtttcacgttgttttcatattctggtgtaagtttgttttatttctctagaaagaagcaagggctgtgaatggtgattaaacgttccttctcaggtttgcctgtgcgtggggtagaggtggaccacgcagaccctccctgctgtgagctccgtggagggtcaatgcagatgcagggagactgtggggcccggtatccgtgacgctggacaagggggttcccgttctggaaggaggagtcaaggtctccagtggtgtcggattagaatatgaggaatccgtctgagctcattctccacaggatcccacaagggaaaacctggcttttccctgtgcagaggccaggcttctatggcttccagaaggttcctctgctactctgagagggcagggcacttgcacagggaacaggcaaccgaaggcagccacagccctactggaaccggggtttctctgccttctggggagaggtcagtggacgggttggggagggggacaggcaatcttcctgtgggagtcgaagagcctggggcctcccctcctccttggaggcgaccagacaccttccctaactggaggaaattctgaaccccagaactggaaaggtcaaaactgaaacaaaatcgccccttgttttgcgagctgctgcctcctctgtgccctcaggcaggtccattccctcctggtctcagtctccccagctgtgccagggggacgccaggtgggacccaactgggtttccaaaaacaaaaggaaatctaagtctcagagaaaggggagctttgggccaggttgggggaagggcctcgggtgctggagtcagtcctgggggagagggaaagggctgtgagggggacatccagcaggtcccacgtgctgtaccccgtatggccatggctggtgtcccagtgcttgggtgcagacccagtggacaggatccatgctgcagggtgacagacggcccatcgcagggcccacagggctcatgtcccatcccctcggggtccggagctctggatggtcccagcccagctatctcccactgcagacgcccaggctcagcgccttctccacgcagctgtgggtagctggcccctctgcaggaaattcaagtgaatggcgatggcactttcggttttcatctctggaaagcgctttcggggcgggaaggtgggagggacgttgatgtgaggcgtggtcctgagttctcctgagggcaggtgagggcagtacagggcagggaatgtggggctggggcaagggctgctgccaactcttgaccagaggggacacagagcaggtggaggagggtctcacccagaccagaccccaggcaggtcaacaagcttgggggtccctgctgaggtggggacacagtggaggtgcagggagccaagaatgagaggaccttcccataggcagggacgcagccacgtgcagggggcctggcctagctggggacacagccaggagcagggagcagggggcactcactcaagggaagacatctacaggtgcaggcaggagggagcttcacctaggctgggtcaaagaacaggtgcaggggccatgaggggcctcccaggcggacacacagtgcaggttcagtgggggtctcaccccagtggggacacagtgcaggtgcaataagcctggggccctcactgagatggggacacagcatccatgcagggaggcggaaatgggggcccttgttcaggcagggacctggagggggtgcaggatctgggagaccctgactcaagaggggccaggtgcagggagcaggtattggggtccggtacccaagagcgcaggtaggaggagcggagctcggatggaggctctgaagagctgggccagtctccagcgcccagggctggccgagcgtggccagtggatccgggtgtgtcacatgacgggatgatgccaggctgtggggcagggcccctgtggccatcctggctgtgcacctgggtggcagccagtggggcgggagctggggctgcagctgtggtgttcccagaggcccaggtgccctctcccacacgcctgggccctctgggttgtgggaaccggccagctcctgctctgtcccctcaggtgtcctgcaggcacagcatctcggggggcccaggccgatggcaggtcttaacgtgtccctctccttcttctttgccaccttcaccctctgtgaggcagccaggcgggcctccaaggccctgctcccagtgggcgcctatgaagtcttcgcccgggaggcggtgggcgcggtgcagctcggggcctgcttcctggagatgaggacgctggtcgagctcgggccctgggctggggactttgggcctgacctgctgctcaccctgctcttcctgctcttcctggcgcacggggtcaccttggacggggcctcggccaaccccaccgtgtccctgcaggagttcctcatggccgaggagtctctgcctggcacgctgctgaagctggcggcacaggggctgggcatgcaggccgcctgcaccctgacgcgcctctgctgggcctgggagctcagtgacctgcacctgctgcagagcctcatggcccagagctgcagctcggccctgcgcacatccgtgccccacggggcgcttgtggaggccgcctgcgccttttgtttccatctgaccctcctgcacctgcggcacagtcctcccgcctacagcgggcccgctgtggctctgttggtcaccgtcacggcctacacgggtgagcactgcttccccttcccctgggcccctgaggacctctcacctgtgtggcctccatgcacactcaggcccgtttgcacccaggatagggggctgctggcccgggtggccaacaaacccacaagaagaggcggtgatttcctgggatctggcgggtagacgagggcctgcgctggacatggggtgcggtggagcagcctgtcccctgccagggccatcctgcccactgcacctctccccagtcccagctattcttggtcctcccagaagttctgcctctggggtagtagggacagccgaggcagacgattcaaggcccagcccagtttcctggtctggtgtgggagtccctgcaccctctatcaccccttccagagcgacaggtcctgtctcacctgccccagttggcctccctggcctcctactgacacctgcatcctgcgatctctgcctgcacctggaccaaccgtggcacccagctctcatggtcccatcacccccttgtgagctccctccactcagaccctccgtctgatccagtcccccatgtctgctttcccctccgagcgctaggctccaccaccacgaaagcctttgctcagcaagttcccaggacaggtgtccacagcgctggggctgcagggagagccaggggagccggctcgtgctccaggcaccacaggaagggctgcagggagaatggtgccaggtctccttccgggggtccgaggtcctaatcaggacaggaccagcacttaggtccatggcactgcccacgacccagcactgggcagacaccacggaacctggaccacaaaggccacaggaaggtgccaccagagccccgtacagttggggaaactgaggcacgggtgagcgggagctctgcagggacacggctctgtggcagattgcccgggcctctgtcctgggtggcaccggagagctcccgtcaagggtcctgcctccacgaggcacagggcgcctggtctcagggacggagctgcccatcctcagggtcctggtttacacctgggcggccgcagactccatgtccctgtggtccctgccctccgctaggcccactccctgctcttccgtgcccagaggcaggtccccggtattggtggattctgttatttggggtggtcacccaggcctggggctgcaggacagcggcccggctgccgccgccttcccttgtctcacatcgcacagccatgtcgtcttctctcctctgctgctgctcctttatttctgagatctctctctgtctctcctctctctctctgtctctcctctctctctctgtctctcctctctctctctgtctctcctctctctctgtctccctgtctctctctgtgtctttttgtctctctctcactatctgtctctcttagtgtctctctccatctctctctgtccgtctttctcgaaggccacatcccacagggtggggtgctggagggcagcatgggcggggcacgggatcccggtgagatgtcgcgagggtgttggacggccctggggggcccccaagctgctgcccctgacccagaccttggctgacccggccttgctgcatcgtgtgtccatcggtgcgggggaccaggcggggcttcaggagccgagagcagctcccaggcctgcacacagtggggctctcggtgtggattcggtggcggggatcaggtcctggcctgcagaggggcgtcaggcagggctggggaccagggctgtctgtgctctgcccacagccgggcccttcacgtctgccttcttcaaccctgccctggccgcctctgtgacctttgcctgctcgggacacaccttactggagtacgtgcaggtgtactggctgggccctctgacaggtaagggcaggggcagggtggggtccctagggctgctggggcccacagagcatcctggcacgtggggtcctgcagggtggaggtgagggtgcctggtcagtgctcctggggaggtctctgcagtgggcatggtttggggggtaggggctgtgacggcatgtctcactgtggacaccgtcccggtgccgagcaagctgcagccaggtaaggaggccacaggctggggaaaggccaaccttgcaaggggtgctgggcacacgggggatggtgtctgcagtggggctccatgcacctggggttcccgtcccaggtggcatctccacacacccagagccagactccgccgaggggggctcattctggtgtccctaaagcctcaggaggcagagagccccgcttagatgatatagggtgccatcgtgcacttatagggctgtgagcatggggctggagcagccaggccctttgcatcctggtggggcagacccgggactcgctgatgcacacaggggaccccagagttcccccacagtgtgtgcccctcctttcttatatcctggatccaagcagaggagcagcatggggaggtcatggagccccctcttggacctagatctggggattctggcccctggcttcagaggcccaggtggagcccacaccttctcctgcacctgctctcagacactcggctccacctcactgaggtaccagaagggcagctgggttcctatggaattctggaagcttttttcagcttctgctggctctacactggggaggtctcaggggcaatgcctcagcctccccttggggggaaagacaggagctgggggtctctcaggcacctggccctgcgatggtgacactgacgtcatcgtgatttgtcgccatggttctgatgtggtcacagtctctcaggcatgtgcagggtcctaggaagcatacgaagtgcatcaggaaccccctcctgcccccgttgccgaggcagcactgccctctgctaggaacagctccgtgttggcctctctgtccccacacactgggcctgcagggcttctccgagactcttcagttcaggtatcaaccctgggctgtctcctgggatgtggggggcggatgttctttccttgcctccccacgctcctcctgcggatccttcactccgggtgggtcggcctcttcctcctgatcagctccagagccccctctagttccctggcatggaaacacggcccgggtgagctgtggtggccccggaggcctctccgctcctgcacaggccttgcttcctgcgggtgacgaggtcctggactctctcctgcccaggcttctgggtgctttccttagttcagcaccagtgctctgtgtgggcagcgtctcccccgaggatccgcagctccgggttacccgcaggcgtccatctccggtatggtgctgcccttcactgatcctggttgtatttctgtttcctgctttcctcatcgcctcctgtttcggttgattccttctttttgctggtgcccgtctcacagtagcttcctgagaacggggacctggcaggtacacttcagacctcctgtgtctgaaatagtgtcctggttctgacctgcacttgagtgtcggtgaggcctgggcagggttccgggtgggagctcagtttcgtcctgagtttctcaggccccaaccatggcctgtggtggcttcacgggctacaaggcaaaggacgcaaacgaagaggcttcacgtgacagggttgtatgctcagccagctctggaggctggagtctgagctggcagcactgacagggtcagctctcctcggaggctgctggggaggagcctcctgcctcttccgggctccgggggcctctggcacccccggtgtccccgggcttggagacgcagcactcccatgtctgccggttcccctggccgcctcctctgtgtcattgtctgttctcttcatatagggacaccagtcatcgaattggaggttcactctactcaagtatgacgtcaccgtgatttcactgattttatgtcccaggccgtattctaacaagggcacatcctgtgttctgggaagggcgtgtcgctggggaaatactcttcacccggctgcaacctctcactgtagaactgcctctgtggagaagcccaaagggcatttgcggcttctaggagccaagtaggaggaggctgggatccgtgtttcaggcgggactccaggcttgggcgggcctgatactcgagtccacatgccccctctagagaggaacctgtctcctgccagggccagggaggggggcactggctgcttctgtattttggggtttggggccctggagcttcccatgcggaattgccgtccctcctcctaggcgagtcccagggccaccccatcccacagggacccgggcgccagcttctgaaagcatggggcatctgcggaagaactgggttgtttcccagctttcgtccctgcggaggggcgatccggcccctccatgtcagcagtgtttggtcgtccacatgcttgtcagccccacgctgtgctcctgcgtctcttcccgtctcatccatctggatgcttgacacctctgacagcatccctttcctgtcatcttagggcagcttcaggaaaccgaaaaacaggcttgtgtccttccattaacccctttatccacaagttcagtatcagcatgagccctggggagctccaaggctgcagccaggagccccgtagccaggtgtggacatggggctatctccctgggtgtgagcggccaccggcacctgctggcagcacccctgagcaggtcatcccgcctccctgggcctgccctcctgggaacagagctaggaccagtgtgggcctggagggccgagtgaggggcaactgtaggcaagctgtaggcacaggggccactgtcaggagccacagcagctgcaggtggtgacaggcagggccccctcaggccagccctagtggagctcacacactgtgggttaaatccttagaggagtgagatcaaacttgaagaggatctactctccccttctgcccggaacacctgcccttcccaatgacctggaaggcaggcggggatttaggagtctctgctcctggagttctgggagaacgttcgcatgaggatttagccaggcccagcctcccacccaggggcatccacatctttcttgtccactcacatcccccacctctacgccaggccacagccctgcaagcagccaccctgggacgccacatcaggtcccggaggaaaatggccacagaagctctgggagtggctcaggacaatgtggggggaggggaattccagtcactcatcccagagggtggtccagaagtacaaggaaccccaccggcttgcccccgtccccatggctggcatacccagagagaggggcatgaccagggcgggaagaccaggaccaggcaggagctgggcctcagggttcttggggctcctggccaggctgcctggctcgcctgtcctggcacaggcctgggagtgggtgtcctgtctgtgtttgggcccaggagccccgcggcccacctgggacctgccttctcttgcagggatggtcctggctgtgctgctgcaccagggccgccttccccaccttttccagaggaacctgttctacggccagaagaacaagtaccgagcaccccgagggaagccggccccggcctcaggggacacccagacccctgcaaaggggtccagtgtccgggagcctgggcgcagtggtgttgaggggccacattccagctgagtggccttgctctgtgtgagccccgtgcgagggccctgcttgtagctggaccctggaaccttctgtagctaagagggaatcctggccccctccccagaagccatttgtcaataaaccatttctaaga